(19) United States
(12) Patent Application Publication
(10) Pub. No.: US 2024/0292819 A1
(43) Pub. Date: Sep. 5, 2024

(54) ANTI-CRISPR CONSTRUCT AND ITS USE TO COUNTERACT A CRISPR-BASED GENE-DRIVE IN AN ARTHROPOD POPULATION

(71) Applicants: Imperial College Innovations Limited, London (GB); Polo D’Innovazione di Genomica Genetica e Biologia SRL, Terni (IT)

(72) Inventors: Chrysanthi Taxiarchi, London (GB); Roberto Galizi, London (GB); Andrea Crisanti, London (GB); Alekos Simoni, Terni (IT); Rocco D'Amato, Terni (IT)

(73) Assignees: Imperial College Innovations Limited, London (GB); Polo D’Innovazione di Genomica Genetica e Biologia SRL, Terni (IT)

(21) Appl. No.: 18/573,114

(22) PCT Filed: Jun. 23, 2022
(86) PCT No.: PCT/GB2022/051600

§ 371 Date: Dec. 21, 2023

(30) Foreign Application Priority Data
Jun. 24, 2021 (GB) 2109133.5

(51) Int. Cl. A01K 67/033; C12N 15/85

(52) U.S. Cl. CPC A01K 67/0339; C12N 15/8509; A01K 2217/052; A01K 2227/706; A01K 2267/01; C12N 2800/105

(57) ABSTRACT

The present invention relates to an anti-CRISPR construct useful to counteract the spread of a gene-drive in an arthropod population. The invention is also concerned with a system comprising the anti-CRISPR construct and a crispr-based gene-drive construct, a method of producing a genetically modified arthropod, a genetically modified arthropod, and a method for counteracting a CRISPR-based gene-drive in an arthropod population.

The present invention relates to a gene-drive reversal system to counteract the spread of a gene-drive.

A gene drive is a genetic engineering approach that can propagate a particular suite of genes throughout a target population. Gene drives have been proposed to provide a powerful and effective means of genetically modifying specific populations and even entire species. For example, applications of gene drive include either suppressing or eliminating insects that carry pathogens (e.g. mosquitoes that transmit malaria, dengue and zika pathogens), controlling invasive species, or eliminating herbicide or pesticide resistance. For example, WO2019/2423840 discloses methods of suppressing arthropod populations by use of gene drives designed to target a key sequence of the doublesex gene which has been shown to be ultra-conserved and ultra-constrained.

The management of vector and pest populations using nuclease-based gene drives is thus becoming a realistic possibility, particularly after the recent proof-of-principle demonstrations of genetic control technologies based on the broadly applicable CRISPR-Cas nucleases¹. These technologies rely on the release of genetically engineered individuals that can rapidly propagate genetic constructs into wild populations together with the linked genetic modifications (e.g. knockout of sex-determination² or fertility genes³) or introduction of genetic cargos (e.g. pathogen-killing molecules designed to block parasite development within the vector⁴). Several gene drive systems have been proposed and a few potential candidate strains have already been developed in the laboratory for the control of several organisms including invasive rodents⁵, agricultural pests^{6,7} and disease vectors^{2-4,8,9}. Access to effective ways to counteract the spread of gene drive elements remains a key aspect alongside the implementation of these strategies, as a risk mitigation and management approach particularly in the case of unintended releases. This is particularly relevant for self-sustaining strategies showing high potential of spread, especially when these are intended to control nonconfined populations dispersed in large areas across multiple countries.

A first example of gene drive reversal systems is inspired by naturally occurring resistance to gene drives in the form of cleavage-refractory modification of the DNA sequence targeted by the driving endonuclease. Resistant alleles can pre-exist in the population as polymorphisms or be generated de novo through non-homologous end joining (NHEJ) repair of CRISPR-induced cleavage¹⁰⁻¹³. Anti-drive individuals could be genetically engineered to carry similar “drive-refractory alleles” and used to rescue the target population^{8,10}. However, refractory alleles rely on a selective advantage conferred by the higher fitness compared to the drive and therefore will have little effect on gene drives with minimal fitness costs (e.g. population-replacement drives)¹⁴. In addition, there are cases where tight functional constraints at the gene drive target sequence may hinder the development of this type of reversal approach, such as for the dsx-targeting gene drive that was recently developed in the malaria vector Anopheles gambiae². Alternative reversal strategies involve the use of CRISPR components to cleave and replace DNA sequences specific to the gene drive construct with^{15,16} or without¹⁷⁻¹⁹ the use of an additional Cas9 gene. Recently, guide RNA-only systems developed in Drosophila showed the capacity to inactivate or replace gene drives in caged populations¹⁹. Although these strategies may offer the option to replace the drive with one or few “refractory alleles”, or even restore the wild-type population, there are several complications attributable to the “DNA-cleaving” nature of the reversal which remain to be addressed, including formation and selection of resistant alleles and genomic rearrangement at the drive locus targeted by the reversal nuclease.

In view of the above, the aim of the present invention is to provide a widely applicable genetic tool to counteract CRISPR-based gene drives.

Another object of the present invention is to provide an anti-drive tool useful to assist laboratory husbandry of transgenic mosquito lines expressing CRISPR-Cas suppressive gene drives, which usually require continuous backcrossing to wild-type strains for maintenance.

The aim, as well as this and other objects which will become better apparent hereinafter, are achieved by an anti-CRISPR construct comprising a germline specific promoter sequence operably linked to a nucleotide sequence coding for an nuclear localisation signal (NLS)-tagged Acr protein. Alternatively, the anti-CRISPR construct may comprise a germline specific promoter sequence operably linked to a nucleotide sequence coding for an Acr protein.

The aim and objects of the present invention are also achieved by a system comprising:
(i) an anti-CRISPR construct according to the invention; and
(ii) a CRISPR-based gene drive genetic construct comprising a nucleotide sequence encoding a nucleotide sequence that hybridises to the intron-exon boundary of the female-specific exon of the doublesex (dsx) gene in an arthropod, such that the CRISPR-based gene drive genetic construct disrupts the intron-exon boundary of the female specific splice form of the dsx gene in the arthropod.

Moreover, the aim and objects of the invention are achieved by a method of producing a genetically modified arthropod, the method comprising introducing into an arthropod an anti-CRISPR construct comprising a nucleotide sequence encoding an Acr protein.

The aim and object of the invention are achieved also by a genetically modified arthropod comprising an anti-CRISPR construct comprising a nucleotide sequence encoding an Acr protein.

The aim and object of the invention are achieved also by a method for counteracting a CRISPR-based gene-drive in an arthropod population comprising arthropods carrying a CRISPR-based gene-drive construct, said method comprising the release of the genetically modified arthropod according to the invention in the arthropod population.

Finally, the aim and object of the invention are achieved also by the use of the construct according to the invention or of the genetically modified arthropod according to the invention to counteract a CRISPR-based gene-drive in an arthropod population comprising individuals carrying a CRISPR-based gene-drive construct.

Further characteristics and advantages of the invention will become better apparent from the following detailed description of the invention.

In a first aspect of the invention, there is provided an anti-CRISPR construct comprising a germline specific promoter sequence operably linked to a nucleotide sequence coding for an Acr protein.

Preferably, the anti-CRISPR construct comprises a nucleotide sequence coding for a nuclear localisation signal (NLS). Preferably, the NLS is tagged to the Acr protein. The inventors believe that the NLS is important for the activity of the anti-CRISPR construct.

Thus, in a second aspect, the present invention refers to an anti-CRISPR construct comprising a germline specific promoter sequence operably linked to a nucleotide sequence coding for a nuclear localisation signal (NLS)-tagged Acr protein.

Acr proteins are a collective arsenal of natural CRISPRCas antagonists encoded by diverse mobile genetic elements (MGEs), such as plasmids and phages, that inhibit CRISPR-Cas immune function at various stages. Distinct acr genes can often be found next to each other, which has enabled their discovery. The ability of many Acr proteins to directly interfere with CRISPR-Cas functions in heterologous hosts provides genetically encodable, post-translational regulation for CRISPR-Cas-derived technologies⁴⁰.

Characterized Acr proteins inhibit CRISPR-Cas function by interacting directly with a Cas protein to prevent target DNA binding, cleavage, crRNA loading or effector-complex formation

Acr proteins are named for the system that they inhibit in the order in which they were discovered. For example, the widely used AcrIIA4 protein was the fourth type II-A Acr protein discovered.

Several Acr proteins have already proven successful at regulating gene-editing activities in different cell types, most notably two SpyCas9 inhibitors (AcrIIA2 and AcrIIA4)²⁰ and two NmeCas9 inhibitors (AcrIIC1 and AcrIIC3)²¹.

The advent of CRISPR-Cas9-based technologies has accelerated the potential for ecological engineering through the use of ‘gene drives’, which spread engineered traits within a population. Gene drives often feature a transgenic organism with chromosomally encoded Cas9 that is programmed to target the homologous region on the sister chromosome. When the targeted region repairs the cut using the drive sequence as a template, Cas9 and its associated cargo become encoded on both chromosomes. Gene drives have the potential to greatly benefit human health in various ways, including curtailing insect-borne diseases such as malaria or dengue, eliminating invasive species, and increasing agricultural sustainability. However, gene drives have been met with calls for caution, as they could have unforeseen consequences or be co-opted for nefarious purposes, leading to large-scale devastation. For these reasons, multiple robust safety measures are needed before gene drive technologies can be used in the wild.

Acr proteins currently present the most direct and broadly acting (that is, independent of sgRNA sequence) method for inhibiting or modulating drive strength and could be deployed concomitantly with or after a gene drive. It was recently demonstrated that both AcrIIA2 and AcrIIA4 can inhibit gene drives, at varying levels, with AcrIIA4 showing >99.9% suppression in a yeast model system.

Multiple families of Acr proteins have been discovered which impede different types of CRISPR-Cas systems (I-C, I-D, I-E, I-F, II-A, II-C, V-A, VI-B) and are classified in two classes, 1 and 2 and named based on the CRISPR systems they inhibit. The inhibition mechanisms discovered so far both for class 1 and 2 Acrs consist of either DNA binding or DNA cleavage prevention.

Below there is a list of Class 1 and Class 2 anti-CRISPR proteins and the CRISPR-Cas type systems they inhibit.

| | CRISPR-Cas type | | CRISPR-Cas type |
|---|---|---|---|
| Family | initiated [organism] | Family | initiated [organism] |
| A [symbol] | I-C [symbol] | A [symbol] | II-A [symbol] |
| A [symbol] | I-D [symbol] | A [symbol] | II-A [symbol] |
| A [symbol] | I-E [symbol] | A [symbol] | II-A [symbol] |
| A [symbol] | I-E [symbol] | A [symbol] | II-A [symbol] |
| A [symbol] | I-E [symbol] | A [symbol] | II-A [symbol] |
| A [symbol] | I-E [symbol] | A [symbol] | II-A [symbol] |
| A [symbol] | I-E [symbol] | A [symbol] | II-A [symbol] |
| A [symbol] | I-E [symbol] | A [symbol] | II-A [symbol] |
| A [symbol] | I-E [symbol] | A [symbol] | II-A [symbol] |
| A [symbol] | I-F [symbol] | A [symbol] | II-A [symbol] |
| A [symbol] | I-F [symbol] | A [symbol] | II-C [symbol] |
| A [symbol] | I-F [symbol] | A [symbol] | II-C [symbol] |
| A [symbol] | I-F [symbol] | A [symbol] | II-C [symbol] |
| A [symbol] | I-F [symbol] | A [symbol] | II-C [symbol] |
| A [symbol] | I-F [symbol] | A [symbol] | II-C [symbol] |
| A [symbol] | I-F [symbol] | A [symbol] | V-A [symbol] |
| A [symbol] | I-F [symbol] | A [symbol] | V-A [symbol] |
| A [symbol] | I-F [symbol] | A [symbol] | V-A [symbol] |
| A [symbol] | I-F [symbol] | A [symbol] | V-A [symbol] |
| A [symbol] | I-F [symbol] | A [symbol] | V-A [symbol] |
| A [symbol] | I-F [symbol] | [symbol] | Vi-B |
| A [symbol] | I-F [symbol] | | |
| A [symbol] | I-F [symbol] | | |
| A [symbol] | I-E [symbol] | | |

[symbol]

indicates data missing or illegible when filed

In the anti-CRISPR construct according to the invention, the Acr protein is selected from any of the Acr proteins listed in the above table.

In a preferred embodiment of the anti-CRISPR construct according to the invention, the Acr protein is AcrIIA4.

Preferably the the Acr protein is AcrIIA4 derived from the Listeria monocytogenes prophage.

AcrIIA4 is one of the most studied and well-defined Acrs, which inhibits Cas9 activity, broadly used for the development of gene drives. Consequently, this anti-CRISPR protein can be exploited as a natural “off-switch” for the nuclease for genomic editing or even gene drives.

According to the invention, the anti-CRISPR construct comprises a nucleotide sequence coding for a nuclear localisation signal (NLS)-tagged Acr protein. A nuclear localization signal or sequence (NLS) is an amino acid sequence that ‘tags’ a protein for import into the cell nucleus by nuclear transport. Typically, this signal consists of one or more short sequences of positively charged lysines or arginines exposed on the protein surface.

In a preferred embodiment of the anti-CRISPR construct according to the invention, the nucleotide sequence coding for the nuclear localisation signal (NLS)-tagged Acr protein comprises or consists of a sequence substantially as set out in SEQ ID NO:11, or a variant or fragment thereof.

The nucleotide sequence coding for the nuclear localisation signal (NLS)-tagged Acr protein is provided herein as SEQ ID NO:11, as follows:

[SEQ ID NO: 11]
atgccgaagaaaaagaggaaggtgagcggcggtagcaacattaat
gatctcatacgggagatcaaaaacaaggattacaccgttaagctg
agcggtacagactcgaacagtatcacgcagctgataatccgcgtg
aacaacgacggaaacgaatacgtgatctcggagtccgagaacgaa
agcattgtcgagaagtttatctcggccttcaaaaatggctggaat
caagagtacgaagatgaagaggaattctataacgacatgcagacg
atcaccctgaagtccgagttgaattga

According to the invention, the anti-CRISPR construct comprises a germline specific promoter, meaning a promoter that drives germline expression. The inventors have found that male and female arthropods expressing an NLS-tagged Acr protein under a promoter, which is transcriptionally active in the germline, fail to transmit a CRISPR-CAS9 based gene drive in a super-mendelian manner to their offspring.

In a preferred embodiment, the anti-CRISPR construct comprises a germline specific promoter sequence that substantially restricts expression of the nucleotide sequence to germline cells of an arthropod. For example, the promoter sequence comprises or consists of a nucleic acid sequence selected from the group consisting of zpg (SEQ ID NO:7), nos (SEQ ID NO:8), exu (SEQ ID NO:9), and vasa2 (SEQ ID NO:10), or a variant or fragment thereof.

In one embodiment, the promoter sequence referred to as “zero population growth” or “zpg”, is provided herein as SEQ ID No: 7, as follows:

[SEQ ID No: 7]
CAGCGCTGGCGGTGGGGACAGCTCCGGCTGTGGCTGTTCTTGCGA
GTCCTCTTCCTGCGGCACATCCCTCTCGTCGACCAGTTCAGTTTG
CTGAGCGTAAGCCTGCTGCTGTTCGTCCTGCATCATCGGGACCAT
TTGTATGGGCCATCCGCCACCACCACCATCACCACCGCCGTCCAT
TTCTAGGGGCATACCCATCAGCATCTCCGCGGGCGCCATTGGCGG
TGGTGCCAAGGTGCCATTCGTTTGTTGCTGAAAGCAAAAGAAAGC
AAATTAGTGTTGTTTCTGCTGCACACGATAATTTTCGTTTCTTGC
CGCTAGACACAAACAACACTGCATCTGGAGGGAGAAATTTGACGC
CTAGCTGTATAACTTACCTCAAAGTTATTGTCCATCGTGGTATAA
TGGACCTACCGAGCCCGGTTACACTACACAAAGCAAGATTATGCG
ACAAAATCACAGCGAAAACTAGTAATTTTCATCTATCGAAAGCGG
CCGAGCAGAGAGTTGTTTGGTATTGCAACTTGACATTCTGCTGCG
GGATAAACCGCGACGGGCTACCATGGCGCACCTGTCAGATGGCTG
TCAAATTTGGCCCGGTTTGCGATATGGAGTGGGTGAAATTATATC
CCACTCGCTGATCGTGAAAATAGACACCTGAAAACAATAATTGTT
GTGTTAATTTTACATTTTGAAGAACAGCACAAGTTTTGCTGACAA
TATTTAATTACGTTTCGTTATCAACGGCACGGAAAGATTATCTCG
CTGATTATCCCTCTCGCTCTCTCTGTCTATCATGTCCTGGTCGTT
CTCGCGTCACCCCGGATAATCGAGAGACGCCATTTTTAATTTGAA
CTACTACACCGACAAGCATGCCGTGAGCTCTTTCAAGTTCTTCTG
TCCGACCAAAGAAACAGAGAATACCGCCCGGACAGTGCCCGGAGT
GATCGATCCATAGAAAATCGCCCATCATGTGCCACTGAGGCGAAC
CGGCGTAGCTTGTTCCGAATTTCCAAGTGCTTCCCCGTAACATCC
GCATATAACAAACAGCCCAACAACAAATACAGCATCGAG

In another embodiment, the promoter sequence referred to as “nanos” or “nos”, is provided herein as SEQ ID No: 8, as follows:

[SEQ ID No: 8]
GTGAACTTCCATGGAATTACGTGCTTTTTCGGAATGGAGTTGGGC
TGGTGAAAAACACCTATCAGCACCGCACTTTTCCCCCGGCATTTC
AGGTTATACGCAGAGACAGAGACTAAATATTCACCCATTCATCAC
GCACTAACTTCGCAATAGATTGATATTCCAAAACTTTCTTCACCT
TTGCCGAGTTGGATTCTGGATTCTGAGACTGTAAAAAGTCGTACG
AGCTATCATAGGGTGTAAAACGGAAAACAAACAAACGTTTAATGG
ACTGCTCCAACTGTAATCGCTTCACGCAAACAAACACACACGCGC
TGGGAGCGTTCCTGGCGTCACCTTTGCACGATGAAAACTGTAGCA
AAACTCGCACGACCGAAGGCTCTCCGTCCCTGCTGGTGTGTGTTT
TTTTCTTTTCTGCAGCAAAATTAGAAAACATCATCATTTGACGAA
AACGTCAACTGCGCGAGCAGAGTGACCAGAAATACCGATGTATCT
GTATAGTAGAACGTCGGTTATCCGGGGGCGGATTAACCGTGCGCA
CAACCAGTTTTTTGTGCAGCTTTGTAGTGTCTAGTGGTATTTTCG
AAATTCATTTTTGTTCATTAACAGTTGTTAAACCTATAGTTATTG
ATTAAAATAATATTCTACTAACGATTAACCGATGGATTCAAAGTG
AATAAATTATGAAACTAGTGATTTTTTTAAATTTTTATATGAATT
TGACATTTCTTGGACCATTATCATCTTGGTCTCGAGCTGCCCGAA
TAATCGACGTTCTACTGTATTCCTACCGATTTTTTATATGCCTAC
CGACACACAGGTGGGCCCCCTAAAACTACCGATTTTTAATTTATC
CTACCGAAAATCACAGATTGTTTCATAATACAGACCAAAAAGTCA
TGTAACCATTTCCCAAATCACTTAATGTATTAAACTCCATATGGA
AATCGCTAGCAACCAGAACCAGAAGTTCAACAGAGACAACCAATT
TCCGTGTATGTACTTCATGAGATGAGATTGGACGCGCTGGTAAAA
TTTTATATGGGATTTGACAGATAATGTAAGGCGTGCGATTTTTTT
CATACGATGGAATCAATTCAAGAGTCAATTGTGCAGGATTTATAG
AAACAATCTCTTATTTATGTTTTGTTATCGTTACAGTTACAGCCC
TGTCCTAAGCGGCCGCGTGAAGGCCCAAAAAAAAGGGAGTCCCCA
ACGCTCAGTAGCAAATGTGCTTCTCTATCATTCGTTGGGTTAGAA
AAGCCTCATGTGACTTCTATGAACAAAATCTAAACTATCTCCTTT
AAATAGAGAATGGATGTATTTTTTCGTGCCACTGAACTTTCGTTG
GGAAGATTAGATACCTCTCCCTCCCCCCCCCTCCCTTTCAACACT
TCAAAACCTACCGAAAACTACCGATACAATTTGATGTACCTACCG
AAGACCGCCAAAATAATCTGGCCACACTGGCTAGATCTGATGTTT
TGAAACATCGCCAAATTTTACTAAATAATGCACTTGCGCGTTGGT
GAAGCTGCACTTAAACAGATTAGTTGAATTACGCTTTCTGAAATG
TTTTTATTAAACACTTGTTTTTTTTAATACTTCAATTTAAAGCTA
CTTCTTGGAATGATAATTCTACCCAAAACCAAAACCACTTTACAA
AGAGTGTGTGGTTGGTGATCGCGCCGGCTACTGCGACCTGTGGTC
ATCGCTCATCTCACGCACACATACGCACACATCTGTCATTTGAAA
AGCTGCACACAATCGTGTGTTGTGCAAAAAACCGTTCGCGCACAA
ACAGTTCGCACATGTTTGCAAGCCGTGCAGCAAAGGGCTTTTGAT
GGTGATCCGCAGTGTTTGGTCAGCTTTTTAATGTGTTTTCGCTTA
ATCGCTTTTGTTTGTGTAATGTTTTGTCGGAATAATTTTTATGCG
TCGTTACAAATGAAATGTACAATCCTGCGATGCTAGTGTAAAACA
TTGCTAATTCCCGGTAAGAACGTTCATTACGCTCGGATATCATCT
TACGAAGCGTGTGTATGTGCGCTAGTACATTGACCTTTAAAGTGA
TCCTTTTGTTCTAGAAAGCAAG

In another embodiment, the promoter sequence referred to as “exuperantia” or “exu”, is provided herein as SEQ ID No: 9, as follows:

[SEQ ID No: 9]
GGAAGGTGATTGCGATTCCATGTTGATGCCAATATATGATGATTT
TGTTGCATATTAATAGTTGTTGTTATGTTTTATTCAAATTTCAAA
GATAATTTACTTTACATTACAGTTAGTGAGCATATTATCTACTAC
ATAAACACATAGATCAAACTGGTTTACATAAATTCAAAAAGTTTG
GATTAAAATCGCAGCAATTGGTTATGAAAAAATATGTGCATAACG
TAAATATCAAGTAAATTTTTGCATTGCATATTTATAGACTCCTGT
TACAATTTCGGAAAAATGAAAAATGTTAATTAATCAAAGAAGAAA
AAACAAAGAAATTAAATCATTAGGTAGCACAACCACAAGTACATA
TTTTTATGGCATGAATATTCCTCTACACTAACATATTTTATAGCA
ATTCTATTGATCGCCTTAGTATAGCGGAATTACCAGAACGGCACT
ATAGTTGTCTCTGTTTGGCACACGCAATCATTTTTCATCCCAGGG
TTGCCATAGCAGTTTGGCGACGGTCACGTAGCATGCGAAGGATTT
CGTTCGCACAGGATCACTTTTATTCTAACGTTTGAAGAAGGCACA
TCTCAGTGCAAGCGCTCTGGAAGCTGCTTTTACCGAACGAACTAA
CTTTTCAAGTAACCTCAAAAACTTGTCTCTAACGACACCACGTGC
TATCCGCGAGTTTCATTTCCCGTGCAAAGTTCCCCGATTTAGCTA
TCATTCGTGAACATTTCGTAGTGCCTCTACCCTCAGGTAAGACCA
TTCGAGGTTTACCAAGTTTTGTGCAAAGAACGTGCACAGTAATTT
TCGTTCTGGTGAAACCTTCTCTTGTGTAGCTTGTACAAA

In another embodiment, the promoter sequence referred to as “vasa2”, is provided herein as SEQ ID No: 10, as follows:

[SEQ ID No: 10]
ATGTAGAACGCGAGCAAATTCTTTTCCTTCCATGACAGCAGCAGC
TACAGTGGGAAGCCGAACGTCAGACGTGTTTGACATGCCGAACTG
GGCGGGAAAATTACAGCGTGCGCTTTGTTTTCAAGCAAATCACAA
CTCGCTGCAAACAAAACCGTTGAGAAATTGATTGTTTTATAATTT
GTATTGTATTTTATTTGTTATAATAAACTAAAAAGACATACTTTT
TGCATATTTTATACATAAAAACATACATGCAGCATTATAAAACAC
ATATAAACCCTCCCTGTAGAGTCCCGTATCGAAATCTTCCATCCT
AGTTGCACAGTACGACGGACGAGTAGGCCGTGTCCGTGCAAATTC
CAGCTTTTAGCAGTCTTTTGCTCGGAGCACTCGCGGCGAGTCGGA
GGTTTCTGCTGAGGTGCTTAGCGCTAAATTAGCCAATTGCTTTTG
CAAGTGAAATAACCAGCCGAATAGTACTTCAAAACTCAGGTAAGT
GAACTAGTTTTATAGAACAAATGTTTGTTTGTTAGAAGTTAGTGA
AGTGTTTGTGAAAAAAATCTCTCATTTCGGCAAAACTAACGTAAC
TGATTTCAAATTGAATTATTGTTTTGTGATGTTATATTATTTCAT
CCAGTTGATTAGTATTTTCTTAGTTATGTTCAAAATACAGTTAAA
TTAAATTTCATTTCATTTACTCATAAAATAATCTCTTGGCTTATT
TAATTTTTCTCGAATTCGCTTGTATTGTTCAGTAGCACGCGCCAT
TCGCCCTTTGTTTCATTTTGTACCTGCTCCCACTAACACACTGGC
AGTGCGAAACAAAAGCCTTCGCACGCGTTGCTGGTATTAGAGTGT
GTGCGTGTGTGTGTTGAGCGCTCTGTCAAAATCGGCTGTTGCCGC
CGGTACCGAAATTGCCTGTTCGCACGCTGTTCGTAAACATTCCGT
GGTGTGTATCGTGTGTTGTGCATGTTGCGCGCCTCCCCCCTTTTG
ATAGCAGGCTGCCGTGGCTGCCGTGGTGTGTGGCGCAGTTGAGTT
TTTGGATTAATTTTCTAAGGAAATGGCACGAGAAGAGCGGTGGCA
GTGTGTTGGTTTGCTCTGTCCCTTCCTTTCTGTGTGAAGTGTTCT
TACAGCACAGCACGTATCCACCACCGCACACAGAGCAGGCAAGGA
AGTGGAAGTGAACAAGTGTGCTGCGCATGCATGTGTGTGGGGGGC
ATTTTAGCTGAGATCGTCGTTATTTGAGAAGCGGTATAGGGGCCA
GTCGGTGTCGACGTACGGAAGCGGTTTAGTTTTAATCCAAGCGTA
TCCCGTCGTGGAGTGGTTGTGTGGCTCTGTGTGCTCTCATATCAG
TTCCAGAGTGAGGTTAGTAGAATCACAGTCCTTGGCCTTTTTCGT
TACAAGATATCCAGAAGGATGGCGTTATTTCCACAGCTTACCATG
GTGCTCTTGTTTGCTCGAATCAGGGGAGAAAAACAGTTTCGTGTT
TCATGAACCGCAGTTGGCACTGGAGCGGATTCAAAAGTCTTCGAT
ATGCAATAGATAAGAGAGTCGTTGGGGCATAGTTGGGAAGCCTTT
CCGAGATGTGGAGTTTCCGAGAGGAGAAATGGTGCTTTCGTGCAC
GTTCCGGGACAGCGGGCCCCGCGAAGAGCATCTCGTTGTCGTTCA
TCCGGCAATAATTGATGCGAAAAGCGCGCGCGCCACTGGCTTAGC
GCAGTGTACACAGTGATATTCACCTACACACACAGAGGCACACGC
CTTCACACGCGCGCGTGCTTCAAAGGCTACTTCGGTGGCGGTGTG
TGAGGTCGCTTGCAATGGACAATGAAAATTTCGCTGGAAAATACC
ATCGTCTCTTTAGGTTGCAATGGGTGCGGGTAGAGCGGTGGTCGT
CGATATTGGTGGTGTAGTGTGTGTGTGTGTGTGTGTGTGTGTGTG
TGTGTGTGTGTGTGTGTGTGTGTGTGTGTGTGTGTGTGTGTGTGT
GTGTGTGTGTGTGTGTGTGTGTGCAACGGCAATTATTTTTTGTAA
TATTTCGACCATCTTTCTTTCTCTCTCTCCACGTGCTGCTGCTGT
TGCTGCTGCTGCTGCATTGCATGTTCCACTATTCCTCTCGGTTTG
TGCCTGCGGACGCCATTGCTAGTCGAAAGAGAGTCGCCGTTAGTC
GCGCTTCGAGCAACGGACACGTTTTTTGGTTGAAACCAACAGCTT
TTTTCATCTTCGGGAGACACACAGATCTCGAATCGTACATTCCCA
TAAGGAGAATTGTCATCTTCCGGTGAATAAAGAAAGGAAAC

In a preferred embodiment of the construct according to the invention, the promoter sequence is vasa2.

In a preferred embodiment, the construct according to the invention further comprises attB or attP integrase attachment sites which, respectively, flank the nucleotide sequence coding for the Acr protein or the NLS-tagged Acr protein, and the promoter sequence. In another preferred embodiment, the construct according to the invention further comprises piggyBac transposon terminal repeats, which, respectively, flank the nucleotide sequence coding for the Acr protein or the NLS-tagged Acr protein, and the promoter sequence. The piggyBac transposon terminal repeats allow semi-random integration in the genome, mediated by piggyBac transposase.

The anti-CRISPR construct may for example be a plasmid, cosmid or phage and/or be a viral vector. In one embodiment, the anti-CRISPR construct (>C119_pBac[AttP(Vasa:NLS-AcrIIAa4_3xP3:GFP)AttP]pBac) is provided herein as SEQ ID NO:20, as follows:

[SEQ ID NO: 20]
cccccaactgagagaactcaaaggttaccccagttgggggatctc
ggatctgacaatgttcagtgcagagactcggctacgcctcgtgga
ctttgaagttgaccaacaatgtttattcttacctctaatagtcct
ctgtggcaaggtcaagattctgttagaagccaatgaagaacctgg
ttgttcaataacattttgttcgtctaatatttcactaccgcttga
cgttggctgcacttcatgtacctcatctataaacgcttcttctgt
atcgctctggacgtcatcttcacttacgtgatctgatatttcact
gtcagaatcctcaccaacaagctcgtcatcgctttgcagaagagc
agagaggatatgctcatcgtctaaagaactacccattttattata
tattagtcacgatatctataacaagaaaatatatatataataagt
tatcacgtaagtagaacatgaaataacaatataattatcgtatga
gttaaatcttaaaagtcacgtaaaagataatcatgcgtcattttg
actcacgcggtcgttatagttcaaaatcagtgacacttaccgcat
tgacaagcacgcctcacgggagctccaagcggcgactgagatgtc
ctaaatgcacagcgacggattcgcgctatttagaaagagagagca
atatttcaagaatgcatgcgtcaattttacgcagactatctttct
agggttaaaaaagatttgcgaaaatgaagtgaagttcctatactt
tctagagaataggaacttctatagtgagtcgaataagggcgacac
aaaatttattctaaatgcataataaatactgataacatcttatag
tttgtattatattttgtattatcgttgacatgtataattttgata
tcaaaaactgattttccctttattattttcgagatttattttctt
aattctctttaacaaactagaaatattgtatatacaaaaaatcat
aaataatagatgaatagtttaattataggtgttcatcaatcgaaa
aagcaacgtatcttatttaaagtgcgttgcttttttctcatttat
aaggttaaataattctcatatatcaagcaaagtgacaggcgccct
taaatattctgacaaatgctctttccctaaactccccccataaaa
aaacccgccgaagcgggtttttacgttatttgcggattaacgatt
actcgttatcagaaccgcccagggggcccgagcttaagactggcc
gtcgttttacaacacagaaagagtttgtagaaacgcaaaaaggcc
atccgtcaggggccttctgcttagtttgatgcctggcagttccct
actctcgccttccgcttcctcgctcactgactcgctgcgctcggt
cgttcggctgcggcgagcggtatcagctcactcaaaggcggtaat
acggttatccacagaatcaggggataacgcaggaaagaacatgtg
agcaaaaggccagcaaaaggccaggaaccgtaaaaaggccgcgtt
gctggcgtttttccataggctccgcccccctgacgagcatcacaa
aaatcgacgctcaagtcagaggtggcgaaacccgacaggactata
aagataccaggcgtttccccctggaagctccctcgtgcgctctcc
tgttccgaccctgccgcttaccggatacctgtccgcctttctccc
ttcgggaagcgtggcgctttctcatagctcacgctgtaggtatct
cagttcggtgtaggtcgttcgctccaagctgggctgtgtgcacga
accccccgttcagcccgaccgctgcgccttatccggtaactatcg
tcttgagtccaacccggtaagacacgacttatcgccactggcagc
agccactggtaacaggattagcagagcgaggtatgtaggcggtgc
tacagagttcttgaagtggtgggctaactacggctacactagaag
aacagtatttggtatctgcgctctgctgaagccagttaccttcgg
aaaaagagttggtagctcttgatccggcaaacaaaccaccgctgg
tagcggtggtttttttgtttgcaagcagcagattacgcgcagaaa
aaaaggatctcaagaagatcctttgatcttttctacggggtctga
cgctcagtggaacgacgcgcgcgtaactcacgttaagggattttg
gtcatgagcttgcgccgtcccgtcaagtcagcgtattttcgagac
gttacgccccgccctgccactcatcgcagtactgttgtaattcat
taagcattctgccgacatggaagccatcacaaacggcatgatgaa
cctgaatcgccagcggcatcagcaccttgtcgccttgcgtataat
atttgcccatggtgaaaacgggggcgaagaagttgtccatattgg
ccacgtttaaatcaaaactggtgaaactcacccagggattggctg
acacgaaaaacatattctcaataaatcctttagggaaataggcca
ggttttcaccgtaacacgccacatcttgcgaatatatgtgtagaa
actgccggaaatcgtcgtggtattcactccagagcgatgaaaacg
tttcagtttgctcatggaaaacggtgtaacatgggtgaacactat
cccatatcaccagctcaccgtctttcattgccatacggaattctg
gatgagcattcatcaggcgggcaagaatgtgaataaaggccggat
aaaacttgtgcttatttttctttacggtttttaaaaaggccgtaa
tatccagctgaacggtctggttataggtacattgagcaactgact
gaaatgcctcaaaatgttctttacgatgccattgggatatatcaa
cggtggtatatccagtgatttttttctccatattcttcctttttc
aatattattgaagcatttatcagggttattgtctcatgagcggat
acatatttgaatgtatttagaaaaataaacaaataggggtcagtg
ttacaaccaattaaccaattctgatgcgcgtctctcccctttgcc
tggcggcagtagcgcggtggtcccacctgaccccatgccgaactc
agaagtgaaacgccgtagcgccgatggtagtgtggggactcccca
tgcgagagtagggaactgccaggcatcaaataaaacgaaaggctc
agtcgaaagactgggcctttcgcccgggctaattagggggtgtcg
cccttattcgactctatagtgaagttcctattctctagaaagtat
aggaacttctgaagtggggtattcacgacagcaggctgaataata
aaaaaattagaaactattatttaaccctagaaagataatcatatt
gtgacgtacgttaaagataatcatgcgtaaaattgacgcatgtgt
tttatcggtctgtatatcgaggtttatttattaatttgaatagat
attaagttttattatatttacacttacatactaataataaattca
acaaacaatttatttatgtttatttatttattaaaaaaaaacaaa
aactcaaaatttcttctataaagtaacaaaacttttaaacattct
ctcttttacaaaaataaacttattttgtactttaaaaacagtcat
gttgtattataaaataagtaattagcttaacttatacataataga
aacaaattatacttattagtcagtcagaaacaactttggcacata
tcaatattatgctctcgacaaataacttttttgcattttttgcac
gatgcatttgcctttcgccttattttagaggggcagtaagtacag
taagtacgttttttcattactggctcttcagtactgtcatctgat
gtaccaggcacttcatttggcaaaatattagagatattatcgcgc
aaatatctcttcaaagtaggagcttctaaacgcttacgcataaac
gatgacgtcaggctcatgtaaaggtttctcataaattttttgcga
ctttgaaccttttctcccttgctactgacattatggctgtatata
ataaaagaatttatgcaggcaatgtttatcattccgtacaataat
gccataggccacctattcgtcttcctactgcaggccccaactggg
gtaacctttgagttctctcagttgggggttaattaaaagatacat
tgatgagtttggacaaaccacaactagaatgcagtgaaaaaaatg
ctttatttgtgaaatttgtgatgctattgctttatttgtaaccat
tataagctgcaataaacaagttaacaacaacaattgcattcattt
tatgtttcaggttcagggggaggtgtgggaggttttttaaagcaa
gtaaaacctctacaaatgtggtatggctgatttgatctagagtcg
cggccgctttacttgtacagctcgtccatgccgagagtgatcccg
gcggcggtcacgaactccagcaggaccatgtgatcgcgcttctcg
ttggggtctttgctcagggcggactgggtgctcaggtagtggttg
tcgggcagcagcacggggccgtcgccgatgggggtgttctgctgg
tagtggtcggcgagctgcacgctgccgtcctcgatgttgtggcgg
atcttgaagttcaccttgatgccgttcttctgcttgtcggccatg
atatagacgttgtggctgttgtagttgtactccagcttgtgcccc
aggatgttgccgtcctccttgaagtcgatgcccttcagctcgatg
cggttcaccagggtgtcgccctcgaacttcacctcggcgcgggtc
ttgtagttgccgtcgtccttgaagaagatggtgcgctcctggacg
tagccttcgggcatggcggacttgaagaagtcgtgctgcttcatg
tggtcggggtagcggctgaagcactgcacgccgtaggtcagggtg
gtcacgagggtgggccagggcacgggcagcttgccggtggtgcag
atgaacttcagggtcagcttgccgtaggtggcatcgccctcgccc
tcgccggacacgctgaacttgtggccgtttacgtcgccgtccagc
tcgaccaggatgggcaccaccccggtgaacagctcctcgcccttg
ctcaccatggtggcgaccggtggatcccgggcccgcggtaccgtc
gactctagcggtaccccgattgtttagcttgttcagctgcgcttg
tttatttgcttagctttcgcttagcgacgtgttcactttgcttgt
ttgaattgaattgtcgctccgtagacgaagcgcctctatttatac
tccggcggtcgagggttcgaaatcgataagcttggatcctaattg
aattagctctaattgaattagtctctaattgaattagatccccgg
gcgagctcgaattaaccattgtggCCTGCAGGatgtagaacgcga
gcaaattcttttccttccatgacagcagcagctacagtgggaagc
cgaacgtcagacgtgtttgacatgccgaactgggcgggaaaatta
cagcgtgcgctttgttttcaagcaaatcacaactcgctgcaaaca
aaaccgttgagaaattgattgttttataatttgtattgtatttta
tttgttataataaactaaaaagacatactttttgcatattttata
cataaaaacatacatgcagcattataaaacacatataaaccctcc
ctgtagagtcccgtatcgaaatcttccatcctagttgcacagtac
gacggacgagtaggccgtgtccgtgcaaattccagcttttagcag
tcttttgctcggagcactcgcggcgagtcggaggtttctgctgag
gtgcttagcgctaaattagccaattgcttttgcaagtgaaataac
cagccgaatagtacttcaaaactcaggtaagtgaactagttttat
agaacaaatgtttgtttgttagaagttagtgaagtgtttgtgaaa
aaaatctctcatttcggcaaaactaacgtaactgatttcaaattg
aattattgttttgtgatgttatattatttcatccagttgattagt
attttcttagttatgttcaaaatacagttaaattaaatttcattt
catttactcataaaataatctcttggcttatttaatttttctcga
attcgcttgtattgttcagtagcacgcgccattcgccctttgttt
cattttgtacctgctcccactaacacactggcagtgcgaaacaaa
agccttcgcacgcgttgctggtattagagtgtgtgcgtgtgtgtg
ttgagcgctctgtcaaaatcggctgttgccgccggtaccgaaatt
gcctgttcgcacgctgttcgtaaacattccgtggtgtgtatcgtg
tgttgtgcatgttgcgcgcctccccccttttgatagcaggctgcc
gtggctgccgtggtgtgtggcgcagttgagtttttggattaattt
tctaaggaaatggcacgagaagagcggtggcagtgtgttggtttg
ctctgtcccttcctttctgtgtgaagtgttcttacagcacagcac
gtatccaccaccgcacacagagcaggcaaggaagtggaagtgaac
aagtgtgctgcgcatgcatgtgtgtggggggcattttagctgaga
tcgtcgttatttgagaagcggtataggggccagtcggtgtcgacg
tacggaagcggtttagttttaatccaagcgtatcccgtcgtggag
tggttgtgtggctctgtgtgctctcatatcagttccagagtgagg
ttagtagaatcacagtccttggcctttttcgttacaagatatcca
gaaggatggcgttatttccacagcttaccatggtgctcttgtttg
ctcgaatcaggggagaaaaacagtttcgtgtttcatgaaccgcag
ttggcactggagcggattcaaaagtcttcgatatgcaatagataa
gagagtcgttggggcatagttgggaagcctttccgagatgtggag
tttccgagaggagaaatggtgctttcgtgcacgttccgggacagc
gggccccgcgaagagcatctcgttgtcgttcatccggcaataatt
gatgcgaaaagcgcgcgcgccactggcttagcgcagtgtacacag
tgatattcacctacacacacagaggcacacgccttcacacgcgcg
cgtgcttcaaaggctacttcggtggcggtgtgtgaggtcgcttgc
aatggacaatgaaaatttcgctggaaaataccatcgtctctttag
gttgcaatgggtgcgggtagagcggtggtcgtcgatattggtggt
gtagtgtgtgtgtgtgtgtgtgtgtgtgtgtgtgtgtgtgtgtgt
gtgtgtgtgtgtgtgtgtgtgtgtgtgtgtgtgtgtgtgtgtgtg
tgtgtgtgtgcaacggcaattattttttgtaatatttcgaccatc
tttctttctctctctccacgtgctgctgctgttgctgctgctgct
gcattgcatgttccactattcctctcggtttgtgcctgcggacgc
cattgctagtcgaaagagagtcgccgttagtcgcgcttcgagcaa
cggacacgttttttggttgaaaccaacagcttttttcatcttcgg
gagacacacagatctcgaatcgtacattcccataaggagaattgt
catcttccggtgaataaagaaaggaaacctcgagatgccgaagaa
aaagaggaaggtgagcggcggtagcaacattaatgatctcatacg
ggagatcaaaaacaaggattacaccgttaagctgagcggtacaga
ctcgaacagtatcacgcagctgataatccgcgtgaacaacgacgg
aaacgaatacgtgatctcggagtccgagaacgaaagcattgtcga
gaagtttatctcggccttcaaaaatggctggaatcaagagtacga
agatgaagaggaattctataacgacatgcagacgatcaccctgaa
gtccgagttgaattgattaattaagcggccgccttggggtggggt
tgttatgtgttgcgaacgagagtggatctctctcgacatttcctt
attttttttttctattgttaacacttacaacgaaacttcggaaga
gaagtttcctcattcgaaacgaggagtcaaactcctttccttgct
tttgtgacatgcatgattattctttcatttactgacgtaacgatg
taaaacacacagaagaagcatgacacacagcaaagaattgttcgt
taataaaacgttgatgaaaactttgaaaacataagaacttgcact
tttattctataattcgtgaaagcttgcaccgattgtctttcatta
ttcaatgtaatgtactgaaaggtgatttttcgcacttgtatactc
tagaactgaagtattctaacaatacgtcacctttaggtccattcc
aggataaaatacacaagtgaaggtagttgtacaaagtacttagac
taacccaggtttcaaaaaagataacgacgaattgagcgtaactgc
acaaagcccgttcattgtcaacatatcgttcaggggtcttcaaac
tttattatcagcccgcgggccgcaacaacgttgcttagagcacca
tcttgatgtatcctttatctagtggcttatcgtttatgtctcgtt
tggctaagaaaatactaataacatattcaatttacggagcggtac
catggtacaaacgtcatactgctagacttgacaacatgcccatcg
agggttcttacctcaaatggaccgatcccttgtaactggcggcgt
aaactcagtaaattccaaaagccagtataggccaccatgaccgtc
taactattccaaaaaggagaatatttgaaatttctatatctatag
tattattactatatctatatgtttctttagtacaatattagagct
tgattattttcctgaaaattgtaaaatatgcatgttagatggttg
atacatcgttttaaaaaaaactgctgttaatggccacattttcg

Accordingly, in one embodiment, the anti-CRISPR construct comprises or consists of a nucleic acid sequence substantially as set out in SEQ ID NO: 20, or a fragment or variant thereof.

The inventors identified the insertion locus of the anti-CRISPR construct, and surprisingly discovered that insertion within the first intron of the AGAP004649 gene, at the TTAA site located at 2R:59504269-59504272, resulted in mosquitoes with improved fitness.

Accordingly, in one embodiment, the anti-CRISPR construct is inserted within the Anopheles gambiae gene, referred to as AGAP004649. One embodiment of the nucleotide sequence of the AGAP004649 gene is provided herein as SEQ ID NO:21, as follows:

[SEQ ID NO: 21]
aattagaagttgatggcaatagattaatatttacgagccgtcttgtggagaattaaatgataaaccagttataagcgaaatctggattta
tttggcttgcattttgaaaaaaaaactaaatagtttaagtgtcggaaccgaatgtttttggttggtgtgtttttatgtgcttcttatcat
cgtgcgtagtgatattgagataaaatatggtgaattttgtgcttatttgtgattggtgaacagtgctagtttaatacaagtgatgatgaa
ttaattattgattttattgaaaaaaaaaaataggaaataagtaaaagaattcgataatataaccaccaatATGATAGACATAAGTATGTA
CGGAAATCATTCTTCGCACACAAATGGTTTTCAGAACAGTGATCTTGGGTCCGGAGGGTACCCTTATTTTACAGCAGGTCACCATCATCA
CCACCATTACCACCAGCACCAAAGGCAACCTGTGCAACAACAACAGCTTACTAATTCTGCTACTAACATTGTTAGTGGCCCACCTGTGAA
CAATACCACTAGTAATACCGTAGTGACCTCTAATAGCAGCTCCAACAACAATGCTAATCCGCCCACAGCCAATATCCATCAGACTTCCTA
TGTTCACGCATCTCACCTATACAGTCCGTCCGCAATCGAATACGGAATAACTACCTCTAGCTCTCCCCCAACTGATTTGTACTTTGAAGC
CGATAGCCAAAGTTCTATATACTACGGCCCTAGTCATCCAACACCGGCGGCTCCTTTTCAAGAAAACCGTATCATAAGCGCGGATAACGG
ATTGAGCTATACTAATTTGGACTATGCCATGTACCATCAGGCACAGGAATGCGCAGATCCTAGTTATCTGGCGCATCATCAAGATAACAA
ATTACATCTACATCGCTTTGGAGCCTTTCCTGGAGCTGTAGCGTCCGATGCCACTGTTGATGGGGATCCTCATCATCATAATCTTCTTCA
TCAGCATTCGCATATTTCACATCAGCACAGTTCCACATCCCCTAATGATGCACATGGCGGTGGAACTACCTGGGGACAAGTTTACTCCGA
TAACATTCCTCAGTCACAACATCAGCAACTGCAGCCGTCTCTTAACCAGCATACGGTGGCTCAGTGCAGTCCTAATGTTTCTTCCGCATT
GCTGGACTCGCCCCATGTAATTCTGAGTGCGAGTGTATCCGACGATGGTGACAGTAATGGTAGTGTTGCTAATAGTTTTTCGCGCCTTCA
CCAACATCATCACCATCCCCATCATCACCATCACCATGCCACATTGCAGCAAAATATGCAAGCGCAGCAGCATCCGGCGAATCAGCAATC
GCAGCAACAAACACAGCAGCAACAACAACAGCAGCACGCTCAACAACCCCAGAACCAACAGAATGCACCTACCTACAAGTGGATGCAAGT
AAAACGAAACGTGCCAAAGCCTCCATgtaagtatttttctagctaaagttgtattgttgatgatacaacccgcactatctccagattatc
acaatcccacagaggatagcgtcacactgagcttgctttacggtctcaccataaaacgatacaattcccacagggactttgagtcccaaa
gggtttgctttctcaatggaggctatattttttgtctgtgtgttttcctttctatccagcaatcacatacaacgacaaaaaaactctttt
atatcggtaaaaaatatcaaacgcacctatttcggttagctacctttatcagcatcaaaaattcatacgcgttgcatagtttaaccccgt
gatgaactataaaaatatatctaactggaccagtagagccataaattttgctcctaaaatcttcaatcctgcaccaatggcagtttgata
aaggtgtaataattcaaattttccgtaaacatcttaccttcgccatgagcgttatcgtttgcatgttgataaatattacatatgactgct
ttaaagcaaagaagacaaaaacagtaaatatgaattctagcagacgttagatattaccgttaatcagatatcgttgtcgtattatgaagt
actggcttattactacgtgataacattatgatctccatatgattttttttaatttagagacaattttatataaaagcaattacgctattc
tcgaaatagatttcaaaaatttcgatccttcccataagctttttcagcataccaaacattaaaattatgcctagaccacatactgcttaa
agaacaacgcaaaacaaccccgattttgttaattaaaaactaccatagctggtgaggttaacaaactccccaattttaaccaccaacaat
aaaaacaacgctcacttattcttagtctatgttacgggtcaaagaggttgtacaccgtgcgtgacttgggtcgtttccatttgaatgaaa
ttgccattatcgtccagaagtaagtggaccccaacaaaaacgtgtcgaggacagacgttttttactgcgatgccgtcaaggtcactaacg
aaaaacgacggagcgatgactgaaaacgaaagtggattatgctagcatagaaacaattcttttcgttctctaccctgttcaacttctttg
gtggctgaaatagattcttctataataacgcgcaacagcaaaaaaaaaatacgcctacgatcgatattacaataccaacgaccccatgca
gacgtgttgtggggtctcaatttactttcaattggctaactgtccactgtcaaaaatgtcacaaactgcactgtactgcaaagcttaccc
aattggtagtgagagaaaagacacgaggcagtaatttgaatgtcatagataataaaaacaaatattagattcaaacctccccctttgtag
acactacattatggatacagttctgttatttcaatattaacatgcatgcctctgcatgcgctgttgcactgtacttgctggatatgtaac
aaaatagattagtattgaaattaatatagcacaaccaccggcttacaaattaatttgtgggtagcactactccacactacactactcacc
aatttgacttaatttcattcaatctttccgcgatcacttatcttttctataattccaatgctttcatagtgtcaaattaaaaatatctgt
ggcatagcaaaagatattttttttaacttgtaacttttcacatgttgagattccatttacctaaccgagaaatacatgcaaatacatcag
tgtggcttgtagtcgtagcctgtaactgtagtcgaacattcactacaactcaatttgcatggtccatagtgtatacagaatagattttat
cacaacatataattataatattttcacaacaacaacaacaacaggcaacacgcaaccaatcggtgaaacttctcccttaggggtttcttt
aattattacagcaaaccaaacattttgcagtatcaattgttttacattattaaaataattctaagcgatgccagtttaagcgattgtatt
tgacggataagaaatgccagttagttcataaaaaaaataaaatctgacatatatcatttaaattattttaacacatagtaccacacagta
actttaaagtaccattgtcaaatttgaattactcacacattttaaattgttatatgacatatattgacatatatcctaactagctcttta
tcttttcccaaaataaatatacgtaataaacttacatgtgttctaatccataccgctttgttaattacctacaggcataacttttaggaa
aagaaacaacagtcaacaaacattcaacttctctgcgatgtcaaaaatttcattttcattagcggtaattaatgatatatcagtagctac
aggcgacgctgaccataaaattgtcatatctgatcaactgaccaaacatgttttcatgatttgttgtatcaaacgtctcaatacgtttga
caatagctaatgaatcggtactatacggtcgacaatctactgtaagaagatgccaacgacccccttgacgtttataattcattaacgtac
gaattatgtttcggtgcagaacttctcaaactgatttcttattttttgttttttttttttataaatattactgctcttactagactggtc
agtcgtgtaacatgatctaaatttcttattacacatattcaatgtaaaaccatttccttccagtaatgcaatgatgataaggtgaattat
atgtaattttatattgaatatttttttatttaatcctacaagaataaatttatatctgattgtgtactcccggatactgaccctgtagac
tttttatttacttcatacatttttgttaagctctttcctagccataaatgagctaaactgacaataaatttacagttaccaaggaatttt
gcctttttttaaatgaatgttgacaatgaatttattagttaacctacagcaaaatagccagtcctacgtataaggtgttcgatccatatg
cagcttgatcctatgacggatatattgtgcagtatgacgaaagtaccaagagacctcccttttttaattttttagtgttttttatcaatc
atgaaatattcaagccattagcgtattacttccttaaaagaccaaacaatttgcaaatatttacatacaacaggccatatcaaacaatat
tagttttttttataaaaatcatttttatatttcacatcagtctttacattatgaatatcgatgcagggcgtactggacaaaactaaattg
atttttttttctgtaatgtagcaattaattccgaatttggatgaatcaattgcggagtttattagtaaccttaaatattttgtaatattt
ttttgcagaatgtaataaacctcttcatattcatccttttttcttataagtttataattaagttattagagccattggtaatgcgtaaac
gtaaatgtgttaattgcctttaacaaatttacttagatgatgtagatgacttagatgacttagatagcatgaggtatagttacaatttgc
cagaacaatgtagcagtaaaacaaacgataatttattcttgtggctttttaactaaaatttaatatttaacaatcaaaccttaatgaaca
aaggctagtttctttttcaattatttatatcagtgaacgtaaataaaaaaatgtaataggtaaatttatttaaaaaaaagtttgtataaa
aaaaaatgttcttgaaaattatacggatggaatcaatcaaagacactgcagcataaatgaattactgaccgtcaataaggagtttatcat
taaaatgataaatgatagtattaacatgttttacccactttctttcttactaacctactttactactcacttactaattagattacttat
aaaaagccagattatttgtcaaaaattgtaaaaaattggtaaaactttgcaaatcaaaagagttgcacttaattgggcctttcttatatt
tgatagttacataacttgtgaaataggaaatggagtaatgtgtataacattcttatgtgaatttgaaaaaaaaaaacgaataaaacattg
attatcattttctatgatattctaagtacagcgaacgtgtcattggaacacgttgaaaccgattacaataattgttcaacaaattaaatt
atgtcatttccggtttatgatactatagatcaccatgcatgccatcaaagtatattactttaatacttaaattgtgagctgaaagtgctg
gaacctgtcgtatgatgaattaatgtatttgcgaaactgatcagggatattagagttagtaccgttaactacacctatgctgccgtagtc
tgaaaatcaatattaactttttaaatgaacctcaaataacatttttaattaaaaaaaaaaaaacaaaacaaagtataattacagttgtgc
gagacagtgttaaacgttaaaatatttaaaagcgtataaatttgttcatttcacaagtataacacttttgcacgtaatttatacaattgg
ctataattttaatattgagtataatttatctcaaaatacttgtatattaaagtaaatatattttggaaatatgttgctttaggtatcgaa
gaagtcaaaaatctataatcgtgaaaaaaatgtgcaatgagaacgtatcattcgttcaagagtttaagcggtacgaatatagactgtctt
acagcagccacaagcgtggtaattctatatcatacgtggtgttatattttatcttatagcttgtcggtcatgttacatttttatttacac
cgcactagtaccccaacgttgaaattctgtcaacaacttctctattatgttgatgaaaatcctacgatcgcatgacgtaaccgaaactca
agaaaacttgcacagttcgttcccctaaccgctttgggtaagttcaagcaacacgcgacacgaacgggtttattgtttatcatagatcgt
cattcaatatcacagtctctcatcagcaagtcaagtgcctaagcgaatcactgtgctgtgacttctcttacaaatgtctaagttgaatac
agctacctcagaatgtgtatcgtagcatcgatggttgcaatcgtttttcaaggattacgcttaccgatatgcaatccatataccaccgtt
gcccatcaaaacaatatttttactccctatcccttagcaatagttcctttatcgatcaaagattacgtttaccgtgttgatacagccgta
ctatattctataggtatctattactgtacaaataagaaacgcttttagacttttattctcaatgcaacacattttgtttgcgtatgatgt
gtacaattaaaccaaaactttgcgttaatatcgctcaacaaaaaaaacatgtgcattttcccccattcctattgttgttacatttgagca
atcgaacatgtcaaaggtcatcttgcaatcttaacacaaacatatgataaacctactatgagcatgattaaggtcagaaaaatataacac
ttctacgcgttttcgcttttccatcttccatacaacataaaagaaggcttgaaagtatgtattaaaatgggcaaaagttaaacctccatg
ttgtactttccttgcttaaggaattctccttgtctaacctacctcatcctccgttatttgtgggagtgatccaccatcaacgtccatctt
cgacataatcctaactttcgccagacatccctattatacagtataatctattaattaagcctgtcagttgccgcacttgggtcgccgcgg
gatggttgagtcgtgcgacctatctgactgccggctcgtaaattagcgcttgtttgaaacttcaacaagttcacaactcccttctagctc
ccccttttagcttacttttgtttgtttatggtttggttaaaaggtatcacgcatgtcagtgggttgttcgggaaattgttatatttgcaa
gcgagacaatttccatcgtagcataaacaagaaacccaggttttaaactgatgtttttctgctatcagattgagtgcatttatgttcatg
tagattttacggtttattttgaagcggtagtaaactatttcaacaaaactttaggacattttaaatgacacaattcgcaaatcgaaaacg
aaattgtacaaaaaggctgcgaatgtttatagtgatgatcagtgatcaacattcctggactgatttcaatgcaagatagagataagggca
taagcataaaacttcgattatcatttttgacatgaaaggaaaaagattcgccacatgcaagagaaacaatcaaaagagacctaaagacgt
ctaatacttatgttatatgaagctattatatacattttctgttaatattagtttactccaattaaaattaaaatctgtatgaatcaactc
acaaataactaaaccttgatttgataaggaaaatattccttatatcgaattcctttaaaatataaatcgaagtgggaacgatcgaagcgg
tgtcaaaaattatataaaattgtggtttcttgatgttttagatgttttagagcattgtgccatattgtaggcattgctgaacagggcact
gatgaaataaattaaagtaaccattgtacactggccactgcaactatcgttcatcgtcgatggagctttggaaagtgactgattgtcttc
ttatgcgtaatgatctaaaagttcaagcttataccacatttttaattctgttttggagaaaacattccagtttttatttaaatcttattt
ggtgtgcacaggagaccctcaaaatacgacccatgcgggttacatggcgttagttgttagttgttagttggcgttacaatgatttttata
gctaaaagttgtcatttggaagaaattgttttttttatttaatttttaattattacagtcacatacacttttatgaagaattccaaatat
ccaagcttgaatatttatttagcataaatgacttattcaatataatgttaaacaaattaatgattttctttattaacccagatagctaat
aaatataataagtagttcgggtttccgtttagtcgcaagttttttttttttttgattatagctatagcattacgtgtacttatatatata
tacatgattttccagaagtttttaagctctaaatatgtttcttaactggaataaattgaagtgtttagccatgtaaagtcaagaggacat
ggcggtctattagtgtcagttgaactatttattgtgtattaaatatatgtatctaaatgccatacatcataaagatatggcatataaata
aatatttttatcatacaaacgtaaaataagctgattttttttagtaaagatgcattcattgttgcatgaaatatatctcctatccaaata
ggagcttatataagcaaaggtgtatttttggttatagatacgattaaaatacacaatatcgtaggtctcgattctgaaacttcgattata
aactgttgtcagatatccaaagcgtcactttttggacttacaccttagatttgaacattttttggtgaagagaaaagccgcgtttcgacg
gaacgatttctgcataaaaactcctgtcaaaatcaccgtttcccatggcaacgaaaaatcgagaagagttcagtgaatcttattatacac
actcagacactccatatctctcttggcaaatctttccccttcctctagaaacaagtttcaaataaagatttgccaatattttgcatttac
acaccacgaatctttgcaggattcaattatcgtaaattaacaaaatatttcgttaaacacgggttcttgttgtaagaaatgcacaatata
caacctttagaacatttagaacttggaagcaatcgaaaacaaatgtttaaagacaaaaaaacacgatatttctctcggcctattaaaaat
agcaagcaaatatttttgttatccattccgcgatggatagcttggtttgctcgaaaaagattcgccatagactgctagacttcttctacc
tcagtgaacaagtgtgtttgaaagttatttgctagcagaatatgccgtgtctgaacgcggctattcgcagtgtggatggcacacatcttt
gcttgtaatcttttggcagatgcaaagaaaactgtaaattttgatatttcaacttatttttgggatcgctgcacatgggagaacattctt
ctaaaggttattggtgatatccatgcattaaaaaaacagttcgcgactttttttttactatccagcgagatacctgttttgctatcagct
caaattaagcagaataaatagtttccaaaataaagtcaaaatgctcaaatagaaccaacaaataattcgtttctacgaattattgaattc
gcagaaacgaattatttgttggttctatttcggaaaagaccccaatagcaggatatcttgaaccccgttctaactctatgaacgatagca
atgtgttgtataataatacacattattatttttgacacgaaaaatttcttgaatcaagaaataatttagacaataatcaaaattcaacta
aagtgtatacgtttcacaaattgaagtagcatttttagtttcattttgtttattaacatttgcatgctaactacactaattacaggttat
ctttatatcataataattgtaaattttcaaccttcttatttattttggcaaaaaccgtagtcggtcaaggcgtgcctgtaccactggtgg
gcctggctttctttgacttaaaggttaccatagccggatagccagtccaacgtatgggggcacggtctgtaccacgggttgttacaattg
tgtatccgaccctaaaactataccagatttctcatttcccgacttattgattcttcttgtattatttttgtattctttttattctttctg
cctctacaggctcccgaaattttccgatggcactgtgagcgggcctctacacgtcacggcttagagtaccgtaccgttcctttttgcatg
cagagtttgactgtacggtacaccacacgaccctggtacgtgtggaggcccagtcatgtgctaattcgtgctatatatcgcttatatatc
acaaactgaatcacatatttacaaagggaaagaatgttatattttcgcgttaaaataatacaatattcgaacgtacggtatgagaggacg
ctagggtctgccagtcggacggaaagccaaaattgattcagttgaaaaatattacatcgggctagctcgcgacgtgtgagcccgctaaaa
attgatgaagttatcgaatgtgggagctcattgatacacaaaactcattgatttaaaattagagaactgttattttattaatttcattag
ctatttctaaaatcaatagtgttatcattttttaactctgatcgctatggaaaataagcctaaattaacttctaacaatgcaatctacac
acttcctttgatttatatttgtctatagtatctctgccatcgcattcacaacaattacacagaccacaacctaatgcttgctcaacctat
taaattttgttttagtttatcgttcctaatgtgtttgtatgatcacttttggatgcccgatatggcactatcagcgtatgaaaaaaaaaa
tacacgagcgctgagaatcaacccatcgtgttcgaaacagtagcaccaaggcttatcaataaataaatgtttatcgcaacaactgcagca
ccacacatggctgcagacagttttcaggtttgaggaagggttaagactttgtcacattagatactcgtaatcgcaaacttggtactaggg
actatgaacagtgagggcttacaaccgcgtcacattgcagtaagacataataaagtgaaccaaatatttgatgtgcttcctatagataaa
cacgatgcgcaagctagagtaatgacaaaaaatcttacaaatgttcaggtatacagtccgagtcagggtatttgttcagatagtgaaaac
gactgtaggaagtatcgatgtttcgtgtatccgattgacatcatatcaagataacgttcgtgaagcttcctgtccgcattatttgttata
atcgatggtagggtgctaaaacttagagttgttctgtgtctagataactgtcactgtataaattctatcgcaagcccatttcagtccaca
gcttcattaaagtactttagataaagcgaaaaataatcactttgtatgtatatatttgtacattactaattaaataaaatgttgaacatg
aaaaatagcatccttaagtaaaacatatagtctgattagtgcacttctcttagtcttgctatgaggttggtgcagaatcttctaaatgtc
aactgtagtgcagctgcaattcttttgcatttgtatataattgctgacctattggacaaatgtaaactcatcaacaaatggggctctttg
attcaatggtaatagcaattttgcaacattgaaagtttattgttcagagttttagtacaaggaaggtcgcagaacaagatgtaagtcgaa
ataatcgtgtgtcaaatatccatcaatacattgttcacgcctcaagttgaaaagtcgaaatcaagattatcgggtattgcacttgaagga
acaaaaaataaatctttctaaaaaaatgtcaagctagccaccacctgcagcctttgaggtcatccggtgcgacccgcgaccagtcgatta
aaaactttatttgtaattctgtttccttgattattatcaaagccatcaaccaatgaaattttagaacataagagcaatttcattgaaaat
aatataataataataatataatgatagctgactcgacaaacttagctattccaaaaaaaatattttattattgatatgttacttgggata
gttgtatcgtgcccgtatcccctcagaattcgatgatcgagtgtaaaccgccaggctcctcacaccaagtgtcaaagtgccgtatacaac
acaacaacaatcctgctctttgtatgggagttagaaaatcatttttaaattaaatttagtgtaacatctgaacgatttttaagtcttaaa
acctattctcataccaccataaggtgtgtgcaaagtttcgttgaaaatgatccagccgtttcggagtttgctcgcaacaaacaccgtgac
acgagatttttatatatatagaattcactaaaattttcttacgacaaaatttacaaacgaatgcaattttcatatacacatataaataag
catcagaagtttcttcttctttttcttctttttggcacaacaaccgatgtaggccaagacctgtctgtaccacttgcgaggttgactttc
attgacttattgatttacccccagtcagtctagtcctacgtatggtggcacggtccatttggggattgaacccatgacgggcatgttgtt
aagtcgtacgagtcagcttcagacatttacgagnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnngtggattaaaaatatcaggtggtggattagggcctttggggggtcgccatagttgagggactttacgtcatttta
gaaccgttaaccattggtttccgcacacaaagcttagcaaacagaacagatttctttgttattatgtgtatttttgtgtgtctattgatt
ttatcgaaaaaatgagatatattaacaaaagatttgatgatttttttatgcacgaaatttaaaatcatgtgagtataagttctaaactca
cgtaaattgtccatgcggctcgtagataatgaggaatcaatgagaaaattgaaaaaaataatgatttcttagtttttatcatcaaaaatc
tcgaaaacacaatgaattataaaataaattcatggaataaaacaaaataacacactttaatccatgttttaatgtttaataagaactcgc
aaagtccaaaatatatttttgaagaatatttaatcgaaaaaatggggtagataaattatttgaacaaatttaattaatgtaaacaaagtt
tgaatcctggggaccttacgtcaagtgacgaattgtcaaaatgcactagagtccaccacctgatatttttaaatccaccttgatctagac
tgaaaatttgcaggctagttttattggtagtttttctatggagtgtttatatgatttccacagcctccagctgtcaaactcaatacaaga
aactgactaaaatcgtgaaatcggcccctatgttcgaatattccacaattattttcattattttaatttcatttagttttataattcttt
taatatcacgcagtgtaagtttgcaatgtgaattaatcattccagaacagtctggtaaatcgtataacacaaaatacaaataacacaata
cacaaatagcacacatattctattttttatatcactctaccgatatatttcaatcccaaagccaattattttatcatggtacgattaatt
ttactgatggtgatatttactgtttaataaatgcttgatgcttggatttattatttcatgaaaaggcataaataaactgtctattatgaa
ttgaatataataaagcaataataaaatgttgtttgcgttgatatttcacctattgtaaaggtagcgtcaaaatggcccttgactacgtta
attgtgacgtaatgcggcccgcgaacctaaaagttcggagacnnnnnnnnnnnnnnnnnnnnaaaatcgtttgaattatgtgttttcaaa
aatgtttgaacaatgtcaatgtatttatgggaatgggtaaaacatatgcgattaactgttaataaatcgtttaatatagggttactgtac
ctgtattggacaggttagtgttttttttttatttgcacattttaatttattttttagagatattcgtgaaaattcaaacactgtttttgc
tcctatttttggccgttttttcctattttcggaaggctatgctcctattttcggcaacgcaaatacgagtcggaaaatgtttcaatcaat
acaaatagatagagaaaatgtttaattaggtacgttctcaaaatccttctttcttcgcgaatatttccccttccaccagaaacaagtgtc
aaaaaaagattcgccaatatttttgcgttcacacaccaacaatctttgctatatacaataattataatttaacccaataattcgtaattc
cctacatactcttgaatatgcccatataacggatcactgcatttaactgctctactctgagcaaaaaaaatagcactaccatgttttttt
tagtaattgtagatttaccctaaagataacgatgttttagttgagaatgttggaataacttgtattacacctttcaagcacattgcttta
atgtatttattgaataatttataattatggggccctttcctttttaagttcgctcgctgaaatttcagcctgtcacctgtttgcattgta
tagcagttttcgagcagctatctaagggggtataatatacaagtggggttataaggtgtatgaatttagaagcttctgtttctgaatgaa
gattttaagaatgttttgagtgttcatcaagcatccagaaagcttgtttgagcaaaagttttcacccgttctgtcaaaaagagatgctca
aatttggttatgaaaaacatgatatgagaccaactggattacatacactttgattctatattccatcaccagatctctttaatgcacctt
ggtaaaaatgtaaacaccacattgttttgccatttttcgagcaggtactcgaatctaactctagctttgaggctagaataatctagactg
aaaatttgcaggctagttttattggtagtttttctatggagtgtttatatgatttccacagcctccagctgtcaaactcaatacaagaaa
ctgactaaaatcgtgaaatcggcccctatgttcgaatattccacagttattttcattattttaatttcatttagttttataattctttta
atatcacgcagtgtaagtttgcaatgtgaattaatcattccagaacagtctggtaaatcgtataacacaaaatacaaataacacaataca
caaatagcacacatattctattttttatatcactctaccgatatatttcaatcccaaagccaattattttatcatggtacgattaatttt
actgatggtgatatttactgtttaataaatgcttgatgcttggatttattatttcatgaaaaggcataaataaactgtctattatgaatt
gaatataataaagcaataataaaatgttgtttgcgttaatatttcacctattgtaaaggtagcgtcaaaatggcccttgactacgttaat
tgtgacgtaatgcggcccgcgaacctaaaagttcggagacctctggtgtagagtacttacagggttttccaaggcgcttttaaatgtaaa
cattgttattcatcgcgtgcaacatgttaatccacatcaatctgatatttcgtttccatcatcattatttttaatttcttcagcataatt
ttcaatacgattgggtttgacagtgttttgttatgtgttgaaaatcatgtgatgaaactgcaatttcattttgaaccaaatacaccattt
gacagctgttgaaaaacatcttggatgcagtgaataattatgtgcacattggaaaatgacttggaaaataaattaaattcaatgatttaa
ttcaacaaaaatccggaatcgcctcctaataactccgccaaagtggtagatagatgtatttactataaaaacaaaagtggttatgtgact
gttatccaatgatgaaatcaaatgaattacttacaggattttcaaagtcactttcgaatgtacagtgcaagtatgtacgaatggaagtgt
ttcgaaacacttcatgggtcttctccgtctatcatattgtcacagagggtttgaagccggatttcatcacccgttcaaatgttgaaaatc
atgtggaagaaattaaaaattattttaataaaaatgaaatatcagagagatttggaataatttcttgcatgcggtgaaaaactatgtttt
catttgaaagtgacttgaaaccctgtaataatttttaatttctcaagcacgattttcaacacttcaacgatctattgttggcccaaagtc
tctataaaaataaagaaaaaaaaggtctattgcaatcgttttttcgaggcataaagccgcatctcattaggattttgacaacattttcca
aattgatataacttaaatttgtgacttggaaaaccccgttagtctctgaaatctgctagcggtacaggcaggccttgaccggcaacaatt
gttgttccaaatatataacctataaaatgtcaacatccgttgtatccggaaatcgtcttaactacaatgcgtcctatgtttaggatctcc
tgtatggatgtatgagtgaatttttcatcagacaacgtttctacacaattgagcgagaacaatccaatcattaagtttgagtctgccgcg
cgtgatacttcctaaaaaacggaatcacttgcaaatttggaacgcttgtgtatttttttaaaaattcacaagatcattggcccaaaataa
atcaacttagcgctgctttttggcttaaacaattcatataacagcttaaaaggcatggcaacataacgcttagaacaacgcaatttaaga
ttgtttatttgttaaattaacataataacttttcatcgctacagacaatgagcaggacattttaccatggtgtgcgtgcacccgtaaatt
cactaaaaaggaatactttatccgagtgttttaaaataagaggtgagggcctgaaccagtactctccagcttccataaaaaatatacggt
gcgctctcagcgagaatccacatgacgcccacgggcctgcccacgtccgaaggcagagccgatggcatacgattctatcttcaccgtaaa
cataagagggacagagcttacaggatttcacactttatctcaagaccgcgggaccccttcccgaatctgtcttagccaaagccaagacta
gtgtatgatgcttgaaaacgttgatgatacctgaaaacgttgatgatacctgaattcatcgaatcgttttcatcgaaatcgtgaaaaaca
cgaaaagattaattaggtgtttccagtacactgatgtacacaaaggttatatgtgtaaaaaaatattcgaccgcatgttctcggcgcgga
gcatcgtgttcgagaaatgtgccgcagattcaacattgcatccgatgaattcaggaatcatcaacgttttcaagcatcactccgcagtct
tggcattggctaagacagattcgggaaggggtctcgctgtcttgagataaattgtgaaaccctgtctatgtatctttgataaagggccct
ttggacggtagcaacgcaataagcaatgcaatcagtaacccctgaatatatgtaaattttgaataacgtcttcccgtattaatacacgat
gcgcaatctcggattgcgcatatattcattttatcaaaccatgtgtcatttcgcgtagtcaaccctgagctataaacgtcatttccatac
aatttcagattgcgccaagattgcgcataaatttacaagattatatgtccgagatatataattttttttgacagatcaatatccgtttga
cagataaatatatgcgcaatctgagattgcgcatcgtctattgctacggttcgatttatgtccaattcaaggtgtttggatattaggagg
tgaagtgcttcagagcaaattgacatttcacgacttgacataacaatactgggggcccggtattaaaatagggaagggctggaggggggt
atagaaattttctatgcgatttgacataaccggcctcagcacaatttttcgatcgaaaaaaatcgatcgatccggctgtcattttggtgt
catttgacactcatttcaacgtcaggtctttttgaaaactggtataccatgacactcacgagcaaaatgaactacagtctgttcccgagt
tacgcggtttctgcgttcccaacgaatccgcgactctcgaatatccgcgtaagtcaaatttcacgttttttgaataaagttctattgaat
actccgagtttttgatttaatgtagtacttttatacactttatcatttatttgatatgattcgtgcagaaaattctaatatttctggctt
ttaagcggtttcaatttgttagcgaaaatgcaatttataccgatcttgaagcaaattgtattgatttgacatttaatctgtcaaatttag
aaaaccgcgtatctccgaatccgcgtaagtcgagaaccgtgtaactcgggaacagactgtatgctacaaaaattcgatcgttccgctttg
tatggggaaaaatgggggcgacgctttgagctgcggaaattatcgaatataaaaaaatgtcataaatcaaggttgatgttttgaaaaacg
ttatgtaaaagcaagttggtaaatttgttgattgttttttaatatgttgggtgataaaaaaatatttattaattattttgaaaattgttt
acatggggcgggggcgactttatgtaggcgtataatagaaatagttatactgtcagttttacgtgagcgcctatcgaattttttcgatcg
aaaatttgtgctgatgccgaacaatactcaattatggcgcccggcaaacgcgctaatacttatacgcggattcggagatacgcggttttc
taaatttgacaattctttgagcaaattgtactgatttgacacatcaattgcaaattgccaaataatttccgttttgatcgaatgttaaaa
actatttcaaaaggtttaaaacagttatattcagtcagaatcacatcaaataattcataaagtgactaaaaccaccccctacctgcaaaa
ttacacgaaaatttgtgttattttagctggaaatcacgagattcgacttacgcggaaattcgagatacgcggtattttgcggccgttttc
ggtccccattaaccgtgtatctcggggaccgcctgtactcaattatggcgcccggcaaacgcgctaataattcaccttctaaataaacac
accttggtccaattgacatcaatgtcacgatgcgttctggattgcatacgcacaggctaaacactgttccaaacggttgcattgctaaat
gcgttgctaccgcatgaagtacagtatgttcccgaggtacgcggtttaacgttcccgaggaatccgcgtaactcgaattacacattttnn
nnnnnnnnnnnnnnnnnntgtttccagtacactgatgtacacaaaggttatatgtgtaaaaaaatattcgaccgcatgttctcggcgcgg
agcatcgtgttcgagaaatgtgccgcagattcaacattgcatccgatgaattcaggaatcatcaacgttttcaagcatcactccgcagtc
ttggcattggctaagacagattcgggaaggggtctcgctgtcttgagataaattgtgaaaccctgtctatgtatctttgataaagggccc
tttggacggtagcaacgcaataagcaatgcaatcagtaacccctgaatatatgtaaattttgaataacgtcttcccgtattaaaacacga
tgcgcaatctcggattgcgcatatattcattttatcaaaccatgtgtcatttcgcgtagtcaaccctgagctataaacgtcatttccata
caatttcagattgcgccaagattgcgcataaatttacaagattatatgtccgagatatataattttttttgacagatcaatatccgtttg
acagataaatatatgcgcaatctgagattgcgcatcgtctattgctacggttcgatttatgtccaattcaaggtgtttggatattaggag
gtgaagcaagttggattaaaaatatcaggtggtggattagggcctttggggggtcgccatagttgagggactttacgtcattttagaacc
gttgaccattggtttccgcacaccaagcttagcaaatataacagatttctttgttattatgtgcattttagtgtgtctattgattttatc
gaaaaaacgtaatatattaacaaaagatttgatgatttgtttatgcacgaaatttaaaatcatgctagcatgagttctaatctcaagtaa
attgcggccatgcggctcgtagataatgaggaattaatgtaaaaattgaaaaaaaataatgatttcttaatttttatcatcaaaaatgtc
gaaaacacaaagaattctagaataaattcacagaataacacaaaataacacactttaatgtatgtttcaatgttaaataagaactcacaa
agtccaaaatatatttttaaagaatatttattcgaaaaaatggggtagataaattatatgaacaaatttaattaatgtaaacaaagtttg
aatcctggggaccttacgtcaagtgacgaattgtcaaaatgcactagagtccaccacctgatatttttaaatccaccttggaggtgaagt
gcttcagagcaaattgacatttcacgacttgacataacaatactgggggcccggtattaaaatagggaagggctggaggggggtatagaa
attttctatgcgatttgacataaccggcctcagcacaatttttcgatcgaaaaaaatcgatcgatccggctgtcattttggtgtcatttg
acactcatttcaacgtcaggtctttttgaaaactggtataccatgacactcacgagcaaaatgaactacagtctgttcccgagttacgcg
gtttctgcgttcccaacgaatccgcgactctcgaatatccgcgtaagtcaaatttcacgttttttgaataaagttctattgaatactccg
agtttttgatttaatgtagtacttttatacactttatcatttatttgatatgattcgtgcagaaaattctaatatttctggcttttaagc
ggtttcaatttgttagcgaaaatgcaatttataccgatcttgaagcaaattgtattgatttgacatttaatctgtcaaatttagaaaacc
gcgtatctccgaatccgcgtaagtcgagaaccgtgtaactcgggaacagactgtatgctacaaaaattcgatcgttccgctttgtatggg
gaaaaatgggggcgacgctttgagctgcggaaattatcgaatataaaaaaatgtcannnnnnnnnnnnnnnnnnnncgtgttctacattg
attgcattcccgctgcgcaaagcgacggaagaaatcaaggcgtttggagaattttctcatgccttctttttctctccaacggcaaatttg
tcaagcagctcgtcgagcttcccgttcctggctccgcctcacacccctcgcctgagcgcgctgtcgaaggtttggatgtgttggtgcgtc
aggcggccaatcgctgtcagccgtcgtccgtgctttttccctccatagcgccatctctttctcgcgtgtggtcaatgctcgcgagctgtt
cgatcccttggtaggcttctgctacataggagagccgcagaccaatgatgtttttatcatcagcgtatgccaggatctgggtagacttat
agaagatggttcccatagtctccaccctcgagtcgcggatggccctctctagcgccaggttgaataggaaacaggcaagcccgtccccct
ggcgctgacccttggtggtagcaaaagatcctgagagttttccatccaccctcacctggcatgtgacgttggtcatagtcattctaacta
gccttatcagtttgggccgggacagtgttgttaactgttgacatttttcatgacagtcaccgtaaacagccggtcaaaataatttttggc
tgatgacattcaatgcttccacgacacgactgtcattaaacgaaattcattcgtgtcgctaccgtcagggctgatgcaatgacatgaaat
gtaaacaatgtagtctgaatgtcatttgacattttttgaacaacacaaatcgatacgatcggaggattatgaagtgtatcgtcgctgaat
tctagtgaaatggcaaaaactagtgaaatgaaagcctttgcaccgcacgctctgttgtagtttgacatgaatacggccgtttgcaatacg
acacagttcacgattcatacaggttggcgaatgtcaaatgaatgtatcttgaaaatgtcttcatgtcaaatgacattttttgacgggaat
gtcaaatgacagagagagatgggaatgggaagcttcaaatcgaatgaatgtttacaattttatgtcgatgaacgtcaccgttcgcaacac
tgggccgggattccaaatgagctcatagcgtcgtacagttttaccctggctatgatatcttatgcggctttgaagtcaatgaagagttgg
catgtgtcgtgtctgtattcagccatcttctccaagatctgccccatggtgaagatctgatcagtggtagattttccgtttcggaatcct
ctttgatagtttcctactatctcttcgacgtgcgggacaaggcgatactgaaggatcagggagaatattttatacgcggtaatcaacatc
gtaatacccctgtagttgttgcagtccaacctgtctcccttcttgtatatggggtacatgatgccgagattccagtcacaagtcatcgat
tcgctatcccacacctcagtaacaatttgatgaatctcgttttctagtcgtgcacctcctttcttgaccagtttagcggcaattccgtcg
gttccgggtgccttgttatttttcagccgacggatagcctttcgtgtttcttctatgctaggtggcagtagcatgacactatcggctagt
ggcgcttctagctgctcgttgaactggtcgttgagtaattcatcaaagttcattgagtaattcatctgagcccatcgcaagaggacctct
ggatggttactaaccagatcttcatccttgttgcgacagcaggttaccttaggtacaacgttgtttcggtgacctgctatcgcttggtaa
aactttcgtgtcggtccgtacgcctctctggtttgctcgagttcccgcatgttttgctcttgcaaagcatgcttcttggagcggtgaact
cgtttctcttcgcgtctgagccgtgaatattcctctgcgcatgcccgcgttctatgccattgctgcattgctcggtatgcagtattctta
cgttcggtcacttgtctccattcatcgtcgaaccagccagatttggtgttgccacgacgtggtgggagtatatttcttgcacagtttttt
ttttgtttttagagcgttccacctcttgctcgtagttttatatctgttttctggtagtagagactcttctaaagcggctttgaattcctg
ttggacagtaatgtcccttagagagtccgtgcgattctacaacgtatcactaagccaaccaagtagtgatcagaatcgatattggctcct
cgatatgttctgacatttaacagactcgactgtcgtcggcggcttattaacacgtagtcgatctggtttgaaggattcgccatccgggtg
cgcccacgtcattttgtggatgtccctccgcgcaaatttggtacttcctacaaccagattgttcgctgcggctaactggaccaatctact
atcattatcgttactgtgctcatgcaatactgtgtattggcagtacattggctccctaccgacttttgcgttgaagtcccccaggatgat
taagaggtcatgcctggggcacgcatctatagttctagcgaggccgccgtaaaaaaggtccttctcctcttcttctttttcttcggtagg
ggcgtgaacgttaatgaggcttatattaaagaatgtgtctcacatgcgcagggtacataacctatcgtttatagccttgaaatctatgat
tacggatttcaaccagggaccttcggcgaaacccgtttcgagcacgcggtggcggtcgtggcagctgtagtaaatgtcgtagcattgctt
accacgcctgttgtgcacaccgttctctagccaccgaatctcttgtagagctacgaggttcatgctcagtgtgactagggagtcatcaag
ttgtttcaaggctccggctttgctaagagtgcgtacgttccatgagcccattttgatcgttgtccgggggcattgcgtagggtcggtatc
gttaggtccgttttgtaaatcccttcttccatgctttctgtagtcgtttattccgtgagactgggtgactggccctgcgctcacgtggcc
gttttatttagcgtcgggttgccccccgacgttcaggcacccagtttctcgggatacccaccccgctcctggttcgcccaaggggttgga
tctagcccgtgtacccaagctaggatatatagaggcacatgttaccactctgcacgacgaggacgtgtcggaataggagttagatgggag
agcctgtattatctctcggagggcttcggatcccatcccattgcagagtagtttaatgttttttttaacatatcgttgtaaaattagaat
tttaaaactcctatcaatatttttttcaatcaaaaatgtactcgaactccaacacatcaaccggccttgccacatgttttcagaggatgc
ttatgtctttttattttctttcaaatgttgtattacagtgtaacaatattttgaaattagcactgaaatcatctcttttatatgatacca
accactacacgcaaacgaaatagaattggctgaaagatagcacacacattcatacttttgtcatagtgttactgccgaaaagggatgggt
gttttggagcgcaccagtggctccgaagtcggctctgcacccacggctcccggctctggagcccacggccgcaccaacagctccggagcc
ggctccgtaccaacagccccggagccggcgacgaatcaatggctccggaccaacgaccctggactaacgtttcaatagagacggcattgt
atgatagccgaaaaaacgattctatcttctcgcaagtgtagaagctaacacccgatgtgttgtgtgatgtgatttgaacaatgttcaaca
cttattgatttttttatagattcttttcaacattatttactcagctaatgatgtatcttcccgattgaaaatttattgaaaatgaatttt
ttggtgcaatctgtaaaaaaccggctccggagccgtaagtgcggagtcgttgctggagaggagtcagcataggagccgtgggtgcggtgc
cggctactttgggttggagtggaggaggtttcgaagttgtctggagtcggtcggagccggtttttaatattaaacattatgagcatattt
tatagatcgtcaacatatatttttatacaacaagcaaaacgattgaaaaatgtatataacttttgaaagaaacgtaagtaaaacttagaa
aaagactaatcatccacttaaaacatgcggcctggccagttatggtggtaaagttagaacatatttttgattgaaaaaaatcttgatagg
atttttaacattcttactttatttatatgttataaaactttattatgaacctttaagacagtttacattcaaatcttacaagaaattcaa
aagatatactcttttaaccacgaaaaacatcattgttccatacaaaatgtatggcctacccgggtaggcggttgtacgattgcgtgaagt
tttttggtcgtacacaagacggttaagcagttctggcgtctcatgaaaaatgtcgaatgacattcattctacattttgttcatgtcatcg
caaaagcattgattgttattgcacaaatgattgtcgcttttggaaagtcgtgtcatggtagccaagaatatcatgataaaaaaaatgatt
ttggcagccgcttacttcgaatatcataaaaaatgtcgacaattaacaacactgcccttaactgagaaagctgctccagcagctgttcat
ctgactcttcaaaacggcacggtttggcctggaagagcagggtttgctgtttcctcactcgtctataagcggacgatcttgttcggctgc
agtgctgatggcttgttccaccatacgccaatggtctgcgaggggcatcgctgcgatgttattgtcggccggtggcgtttccccgagcgg
tgtcgcgtatccctccgccaaattagcacacttcaaccgatccaggttgagcattggagtgggctgactccgttggttgttgaccacgga
gagtttctggtgcagctttatcatgaccaggtctgagtcgacgtttgcgcctctataccatttaatgtcaataatatccgagaagtgcct
cctggcaatcagaacgtggtcgatttgatacattgaactaccgctagggtgtctccaggtgtacctttggcggcgtgtatgctgaaagaa
ggtggggatgctcatgtgcttggaggaggcaaagtttatcagcctgagaccgttgtcgtttgccagctggtggacgctgaagcatccaaa
cgctggtttatatgcctcctcctgtccgacctgagcattaaggtctcctatgacgaccttcacgtcatgcttcgggcagcggtcgtactc
cctctccaactgcaagaaggcctccttctcgtcatcggtgctcccaaggtgcgaactgtgcacatttataaagctcaggttgaagaatat
gccatgaatcctcatcctgcacattcgttcgttgatcggccaccacccgatcactctctgttgcatcgcacctagcaccataaatcgagc
tcgcgcgtctcaccaccgctctggtagatcgtgcagttgctacggtaccggcgcaccgtcacgcctttctacacagcaaattttctcatc
ctgcttcagtaaagttttttactgaaacggttcagtattagaatattttactgattttcagcataaatgtcatttttttactgattttca
gtaaagcaatttactgaatgcatttcagtaatacatatattactgaaaatcagtaaaataggtgtcaaattagtcgtttcactggatatc
agtaaaacaaattactgaaaatgcagcaatccattctgtcaaatcgacctgtcagtcagatcatcaaaacaaaacaatgcggtagccact
tgctcgtgatgtgcaaaaagttgattttgtaggcgtttacaggcaccctggtgaaatttcaggtggtatttcggcttacgggagtaattt
aaaacaattggcagccgtgttggtgtgtaaaatgtttgctacaatccactgtgcgtgctgaaattgcgtggtgtctgtgagcgcttactg
aaccatctacacttgcggcggtgaaatacagtacaaaaactttataaaacaacacgaaacatgtatttatatgaactgagcgactggcaa
tatctgcgcatcaataaaaactaaatttaaaagaagtatttcgtcattttttaatcgctaccaactactgaataatcagtaatgtgagaa
tggctttactgggatttcagtaaacgagctgtcattttggtgagcatcggacattactgaatgattcagtaaacattctttttactgaaa
ggattactgaaacttcagttaaaaaaatttcagtaaaatnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnaattagcatgttaagcagtttactgaatattccttggaatatgcccaccaaacacaattttt
ctttcatttatgtatgcttccaccctttatctatggattaaagtcatcacatagaatgaaaataacttaaaagatgaaaaagtattaaat
aaatgttgacgttttacgagaagctattttaacacgcagggcaaaatgggcatatgtaaacatgctgtgaaacgtcaaaatactggggca
atatggtaatatagtggaacgtcgattatccggacagttcgggaccgggcggttgccggttaatcgatttgcacagataatggtccaaga
aatgtcaaattcatataaaaatttaaaaaatccactagttttatgattaattcaccttgaatcaatcgataaatcgttagtagaatatta
ttttaatcaataacgataggtttaaccactgttcatgaacaaaaataaatttcgaaaacaccactagacactacagacctgcacaagaaa
ctggttgcccacttgactatcgcagcaaatgctcgctgtacgtttgaacagctgtcacatttatacgcacggttaagccacccgccggtt
aatccgcccccggatgatcgacgttcattgtataactgcgcttagataatggtctatgcatttgcgcacgtttcgtgaaatgtattttcg
ctttatcttttgttttgctgtccagccctttaaattcttccaaaatacgttttcaaaattaaaacagtttttacacgtttaaaactacaa
aattgaatgttttgaagctgtgcccgttatcattattgaggcgacaaatactacaccatagcctcaccaaacgtcgtatgtcaaaagcat
ctgcccattttgccccacgtgacgcatttttgtattttttgttacacagtagaacgtcgattatccggggacggaataaccggcggacgg
cttaaccgtgcgcataaatctgacagctgttcatacgtacggcgaaagtttgcagcgataatcaattgggtaacgagtttcttgtgcagc
tctgtagtgtctaggggtattttcgaaattcatttttgttaatgaacagttcttaaacctacagttcttgattaaaataatattctacta
acgattaatcgattgattctaggtgaattcatgataaaactagtgaatttcatatgttttataggcgtttgacatttcttggaccattat
ccgtgcaattcgattaaccgccaaccgtccggtaaaatatatcatgtaaaacttcaacgcgtccctgtgacactggtttaagattatttt
cgaagtggaaaaaattaaatgaatatgccaccaaaccctaatttgaaattttcttttttatttgttatgtaagggttatgaaactcacat
ccctacccgcaaatttccgttaaatgccttctaatcgccttgtaatcgccggcgaatttaaggcgatcagcagtgcttttagcagtaatt
aaatgcctttgaaatatgtcaatgaaaaatttcgcttttaatttgaaatttgaaattgcaactgtcaaaagaaaaccttacaagcgtctt
cagcactgcactgttgcacgattgctgataataaaatcatatagttgctgatagcacgatttacgtgttatgttttcttgcgttaagctc
agagctatttcactttattaatgatggtctgcattaatcggatgttaaataccaacccgttgtaaggttttaatatatcaaactcatttc
gataactctaataaaaggagaaatgagatacatatttctcccatcctgataataaaaggtgaacatagtgttattattatgtttttaaaa
agatattaattaatttagcttcacataaattttgtttgtattgatttttttttctatttgggctataatggtggtatggttcctatagtc
gtcgtgttgtgttcctatagtggtggtacacaaagatgcctcaaaaactgtgtaatatacatggaactaagttttgcagctgttttcgta
tgaattttttttataaaacatatatatttgcgagtaaagtctaatgcttatgaactaatttatgtacaaaaacagaatgacacagggaaa
aaaataacatattttataacgttagacaacaattgagtacgcctagatcaatcataccatcgttgttaccaacgatgtggatgatatagt
ttgcaaacaaacgcaaaacacctattctctgttcagtactagctcaggctagtactgggaattgcaaacagttaaaagaacgagcacaac
ctggagctagcgcagtaattttctgttgcaatagctcccaagccgcagaaactcttctgcaacatgaaaatttatgctccagggtttccc
cgaacgaacaaaacaaacacgagttggtcacaattcgccccattcggaaaaggagatcgcatgttggaatttttccgttgaccaacagga
aaagtgtcgaacgttgaagggatgataaggctggacaattgagatttctccagatccttctccagctgtgttgtcgtgtctcacgcacta
tcttagcgtctggtaatctgctcgtaagcatacgccacaacagccgagacgatggcctttgaccgtactctggtggcacgtacgcaannn
nnnnnnnnnnnnnnnnngaaattcctattgacgcgatcaaaagcatgcggaaggtcaaaggagatgagcttaccacactttcgtttcctc
tgcagatcaatcactctctccttaacggaaagagcagcttgaaaaatgttgcttggtttattacaacatttctgcgctgccgaaataatc
tcccacttcccgatgatgccatcgagtctgtgtttgaccatccgcgacagaagcttgtaatctgcgttaaggagtgatatcggtcgaaat
gcagacatggtacagcctcctcccttcttcctaaccaacactatgaccccgtcaacgaaactttcaggaatctttcctaagagggcctca
ttaagaactagaagaagctctctatttatgatgctgaatgcacggtgataaaactctttgggaattccatccgggccaggggactttctc
gaaggtgactttttaattgcatcgaaaagctcaccataagtaaactcgtccatggaattttgttggcttcgcagtcctcgggaataacgc
atgtgcttgtgaatgttgtttctgtggtcattgtggatgtgtcggatgttgtgtatagattcctgaaaaagccctcaacatgaacattta
tatcctttagctcagttaaggacgatccgtcatcaaccgtcagcttgtcgatcaccgttcgtctcctcctttgctcccctaactgaaaga
ccgacatgctctctccgcatacgcgtgtttcattaatgcgggtgaagtcctcggaaatacatctctgaaagagaagcatttgtcctttta
tacgatttatattcactaactcgttagggtccgtgacatatcgatcataggcggacttcaaccccctgtagagcagttcatgatgcaagc
gaaaactttggtaccgttcattcgttttccaactgaaaacgactttattttaggcttcgcgagctcaacccaccactgcattcacgagcc
gtaatttctacgctgtctggtccaatatttccacttgcatccgaactcctccaggtttgcgtcagttaatacatgtggtctcagtttcca
ataaccattgctgtgcgaacagctcggcgggattggaaggcaaacacgaacggtaagcgccttgtgatcggaaaaggacaagacatgcat
actgcttgctcttaactgtgttttaagtgaagaggagacataacaacgatcgatacgtgaacccgaaccacttgtgacataagaaaactc
cactgagttacctctgagaagctcccagctgtcgcacatgcccatgttgctttaagcgttttgcaaagaaagactaaaatttctagcgcc
cgtcacatctttcggctgcaacacgcagttaaagtcacccgcgagaataacatggttacatgcattccgcaaatagaaaggtaacgttcg
cccgaaaaaatcctccctctccgctctccgctgactgcccgaaggagcgtcaacattgcacagggtggcattgttctcgagtcgaacgca
aatcagacgagaatctaagctgcgctcgacatgggaaaattttaaatgttgacgtaaagcgatcgccgtacccctccccgtcacatcaac
attgcaaattacgttatacccaggaagcgatagatctgaaatacatacttcttgcagaaatatgatgtccaggtccatcgtcctaatata
tgttttgagggcttccagctttgtcgcgttagaaatcgcattaatattgatagtagctatattatagctactaccaagattactacttac
cgggttaggatccatcgcaacaaaacttaattaatctgttgtgaagttgtgtgtttttttggcggtcgaccaggcttacgcttggacgcg
ctcataacactaaatgttgacgaacatgaggcgtcactctcattatccgtttccggattttgcttgcgtttaaccgcaagcggttcgtcg
tgggaagggatggaaaaggatgtggaagggttgcgagcggctctttgaacattaaggcacctgaaagcgcccggcattccaccctcatgg
gctcagatgcagccgcatcgttagcatcagtggcagcatccgtagcagcggcagcatcggcaacagcagcggaaacagcggcattgatag
tagcggcagcggcagcatcattggcagcaggagcagtaaaagtaggagcatcgtcattttctacatgcgttgcgacggcaccaacggcag
cacgcatcttctcctcgggttgctcaccactggctttcactggcttcggacccgaaggcggagcgagagttcgcggctcgatcgggatca
caagccgattagccactttcgatgcagtgctcgctggtggcttagtagtgtttgtaagggaagtgattttgaccttccctttacgccgtc
ccgtggtgttagtacctgcggtactagttgatgctgtgttcgcatcagatgtttgtgctggtgcttgtttaacggtacttgcgtacgatt
ttgccgcaccgttgttgaggagctgacccacacggttttggatgcagctaataccgtgatgcaaagcttcattgcagtgacggcaagttt
gccgggcaagttcacccacacagtaatgaaggaagggatgggtttcgccagcttcatgcgcactatgcgcacacccgaaggaatcccggt
aagccgcgtttcggctccccaaacaccgggcacaatagtgagcacttcaccgaagcggttcagctcagcggaaacgttttcgttggggat
tcctggccgaagatctcggattttcacctccatcgcaccatccaccatctcgatcggaatcggatactgcttcccctcgtgcgtgatgca
gtgcttccccgaacgttccttcactttgctttccgcacgttcaagcgttggcatagtgacgtagacggcactttgcgcagtgctgtgttg
caccatcttcacatccacacccgtttctagctcttcaaaaaggaagttcacggtttcttctagggtgggtcgcttaggagcctccgaata
gtcgatacggaaggtgttccttccatatctttcggtcgtacacattatggcttgcaaatctaaacaacaacacgtcctttctcgttgacg
tttgcaagcgaactataattgtagcaagaaaatattaatttaaaaagaaaaaaacacaaaaaagataataaaaatcaggatttgaacaca
cgtattctcggttcggaactaaaagcgttgtcactgctctatttagcagttaaacctgtaagggtgtaaaaccagtatataaataggcta
agatggcggcaagctatctgttctcgttgaatcaaaaagttgaaagatttcgaagagaacccgttacagccgtgaaagtttcggttgaaa
tctttattcgccttctatggcgactaaggccttaaatgccttctgaatgccttcaaagccgtttaatgctggtagggatggtgtttatag
aacactctaatgaatactttttgagcattgccccgctttcccctattaatacggttagagaaatgagatacatcctcctgcgagccatat
ccgatttgcaagggcacgtaagggctcgcgcgccatataagttcacagccccagagcccttgcattaaagagcttgcgagcctaggcatt
tttatccccgataaacatttccgtcaaacattcccgccgtgcaattgacttcaaaatgtaaaattgaataatttcacattttattcgttc
aattataaaaataaaattaaataacatcccaagcgccacagattaagctggaacgactggaaaatcgaatatccatagaaaaaatgaaac
cttcatagcgttactggtttaggcagcggtcagaacctcgccgcatgcgattttgccacaagcttttgtatgggatttgacgttaaagac
aacacttgnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnn
nnnnnnnnnnnnaacgtgagaaaaggtattaaattgatgtggtccactgaaagattgactgtattcattttcacgtattcaaaatgacca
cgtagcatgccaatcttgaccaatatggtccttaaatcattcaataccagtaataccaataagtaaaaggtccatcgactagtttgggtg
gtcatctgtgttatgaattgctcttttactttatttaaacacttaaaactttaacattataataaatcccaatcccaatcccaatcccaa
tcccaatcccaatcccaatcccaatcccaatcccaatcccaatcccaatcccaatcccaatcccaatcccaatcccaatcccaatcccaa
tcccaatcccaatcccaatcccaatcccaatcccaatcccaatcccaatcccaatcccaatcccaatcccaatcccaatcccaatcccaa
tcccaatcccaatcccaatcccaatcccaatcccaatcccaatcccaatcccaatcccaatcccaatcccaatcccaatcccaatcccaa
tcccaatcccaatcccaatcccaatcccaatcccaatcccaatccaatagaaatgggattaagctttaaagtaaaccattttttctactc
aatattcaatataagtcatcgtcaagacatctcgattgcaagaataaataagcctcctaataaaataagtagtgaatatagaaatattat
taaaattaatgctttcttttctttttattctttaatagTGACTAAATCTTTACAGTTATCAACTACACCAGAATATCATATTCCTACGCA
TGTACTCGACCCGTTAAGAGTTCCTAATCACACGACAGTTGgtatgtatatacataaatgtttttttaatgtttttcgactacgaagtcc
tttttcacattgaactttgtagcctgcattactctttgcttcctagggatttataataccaacaaataatgatttcaatgatacaatgat
aatgatactctttcttcatttcagTAGCCCCTAGTGCTGTATCACCTCATCAATCAAGTTTTATGATAAACAATAACAGCACAGGCCGTA
CAAATTTCACCAATAAGCAGTTAACAGAACTGGAGAAAGAATTCCATTTCAACAAGTATTTGACTCGGGCAAGGCGAATAGAAATAGCAA
ATGCTTTACATTTAAACGAAACACAAgtaatgaaatatttcacgcagctgcatatttgtaatcagtgtttatgaaataatttttgttttt
atttcacttagGTAAAAATTTGGTTCCAAAATAGACGCATGAAACAAAAGAAGCGAGTGAAGGAAGGTTTAATACCTCCTGAAATACAGA
ACCAATCTCCCAAACACTCTTCCCTGATAAACAGCAATGATTCCTCCTCGGCAAGCTCGTCAACAGCAACGTTAACAGCTATACCTTCTG
CCTCTGGAGCTTCTGCAACGAACACGTCTACGTTAAACGAAAGCAATGAAAACAGCCGTGAATCAATTACTATGTAGaacaaatgctttt
tcaaaacatgaaaagactctctttttattagttagaaccaattgaaagacattaatttacaacagaatttgcaaattatcagaacacttt
tagggctgatcgtcttattcaaccatacggttaatagatgagtaattttaaaattatcagtcaaattatctttcacattttctttaaatc
actaaactatatgacttcatggatggaagttatatatttattttaagaccaccaggttcatgactgcttgacttgaggtattacgaacaa
ttttgaactacaatatgattgcattgttttgttccatatgccgcatgctagtcttttaaataaggatttatacgtctatacaatatttac
tcgattaatctattagtttccattcgggataaacgatatatacaaagcaagaaacacatacaaaaatgaatagctcaaaagattccaatg
ttcctctttgctagatattaaacaaaccaaatgtttcctatttgataatagacttattaaagctaacccatgtacatgaggcagtgagca
tcgagaattagtaaagtttatttgagaaacacgttttttcttactaaagctatttccaaagctgtgcagttgcatttcaacaaaaccact
agcacatttacgtttcgttgcacaataatgttgaatgcatttaatactgtacaaaataagaattaggatttataaaattgctcatattct
gccggcactggttatatggataatgttaaacatacaaaaccccaattcagcaaaaagacaaaacaaatttaatttaagataattcctatc
gaatacaaagatacattt

Accordingly, in one embodiment, the anti-CRISPR construct is inserted within a nucleic acid sequence comprising or consisting of the nucleotide sequence substantially as set out in SEQ ID NO:21, or a fragment or variant thereof.

Preferably, the anti-CRISPR construct is inserted within the first intron of the AGAP004649 gene. Even more preferably, the anti-CRISPR construct is inserted at the TTAA site located at 2R:59504269-59504272 of the AGAP004649 gene. One embodiment of the 2R:59504269-59504272 site of the AGAP004649 gene is provided herein as SEQ ID NO:22, as follows:

[SEQ ID NO: 22]
GGGATTTGACGTTAAAGACAACACTT

Accordingly, in one embodiment, the anti-CRISPR construct is inserted at the TTAA site of SEQ ID NO:22, or a fragment or variant thereof.

In a third aspect, the present invention refers to a system comprising:
(i) an anti-CRISPR construct according to the invention; and
(ii) a CRISPR-based gene drive genetic construct comprising a nucleotide sequence encoding a nucleotide sequence that hybridises to the intron-exon boundary of the female-specific exon of the doublesex (dsx) gene in an arthropod, such that the CRISPR-based gene drive genetic construct disrupts the intron-exon boundary of the female specific splice form of the dsx gene in the arthropod.

The inventors previously observed that targeting an intron-exon boundary of the female specific splice form of the doublesex (dsx) gene resulted in suppressed reproductive capacity in females which were homozygous for the construct.

For example, the inventors generated a gene drive construct (ii) such that it targets the splice acceptor site at the 5′ boundary of exon 5 of the dsx gene in a mosquito, and were surprised to observe that, in stark contrast to all previous demonstrations of gene drive, no resistance was selected after release into caged populations of the mosquito. Moreover, additional experiments that were designed to reveal rare instances of resistance that were not selected in caged experiments also surprisingly failed to detect putative resistant mutations, thereby indicating that all mutations that were generated did not restore dsx function. The inventors have demonstrated that disruption of a female-specific exon (exon 5) of dsx leads to incomplete sexual dimorphism in female mosquitos, but not males. When female mosquitoes carry this mutation in homozygosity, they display a range of mutant attributes including the inability to produce ovaries and biting mouthparts—an advantageous outcome that is optimally suited for a gene drive aimed at population suppression.

The inventors have therefore demonstrated that the gene drive construct (ii) can be used to spread through, replace and ultimately suppress any arthropod population by using the ultra-conserved, ultra-constrained sites found in different species at the intron/exon boundary of the female specific exon.

The sequence of the doublesex gene in various arthropods, insects, and mosquito species are publicly available and so known to the skilled person. For example, in an embodiment, the doublesex gene is from Anopheles gambiae (referred to as AGAP004050), which is provided herein as SEQ ID No: 1, as follows:

[SEQ ID NO: 1]
GCTAATTTCCAAGTCCCAAATGTTCTGGTGGTATATTCATTTCTTATAA
CAAGAACCCGTTGTTTATGAATAATTTTGTTAAATTACTATAATTTTA
TCCGATGCAAATAGTAAGAACAGATTTTTGGTTTGCAGTGCTTACAGC
ACTTCTCAAAATATTCTCGCGGGCCGCATTCATTATCCACGTGGGCCG
TATGCGGCCCGCGGGCCGCCAGTTTGACATACCTGCATTAAAAGAACC
GTAGCGTTCTTCTCTTGTAAACCGGTTCATTCATTTTTTTCACGTGAA
CCAAATGAACGGTTCTGATTCATTTGGCACACTTCTAGTACAGACAAA
CTTTAATCGACAACAGTTGTTGTGCCAATGAAGAAAAATAATAATAAT
TATAATATTAATAACAATAATAAAAAGTAAGTAGGGATTGTCTGTAAG
AGTATTTTTTCTGTTTATTTATTCGTATTGAAATAATCTAAAAACTAT
TTTCAACTTCTTTATGGTTTAAATTCTTACCTCTTCCTTTTCAATAAA
CAAAGAAAAAACAGTTCAAAATAATATTTTATTTACAAATAATAACCA
ACCATTATAACGAAAGCGTACAGATCTCTTCCTAATGCCATCGGTTTG
ACGCGCATATTGTTACTTGGGACCCTTGCCTCACGCATACATAACAAG
CGAGCGCGTAAGGCTGTGCTCTAGCATATGGAACCGTGCGTCGAACAC
TCTATCGCCCATATTGTGCTGCGTTGGGAAACAACCTATCTTGGCCTT
TGGAAAACCGCTTTCTGGCTGCTCCCGGAAGAACACCACTCAAACATG
CATCGCGAGCAAATAAACACCCAATCGCACACTCTACAACATGCACGT
GTTTGAAAAAGAAACTCGAGCCGTACGACAGTCTCTAGTTACAGCACA
GCCTCAGTAACAATGTTGTGAATGTATTGCAGGGACGTTGTGTTGTGG
CGCAGTCTTTTTTTTAAACAAAACCGAACCCTTAGTGTAAACCGAACG
TGGTTGTGGGGATAGAGCGTTAGAGGGGTGGGCAGGGAAGGGTGGAAA
AATCAAAAACTTGTTGCACACTCCGCCGGACCAGACCGTTGCGATGTG
TGTGCTGACCTACAACAACTTTCCTTTCCCAGCCCTACTGCCCCATCC
TACCGAACCGTCCGCTCCGGTGAGGCAGCGTGCTCATCGATGTGTGCG
AGCTGAAAAGGGCCGTGCGCGTGTGTTTGTGCGAAACGTATGTGTGTG
TGTGTGAGTGTGTTTGCGTAAATGCACATTTATCAGTGCAGTTCCGCG
TACTCGCCGCTTCGCAATCGCAATCTGGTCTTTAATCGAGGAGGCAAC
ATTTGACCATCGCTCGTTGGCAGTTGCCGTTTACTACTGGGGCGGGTG
TAACGAGGCCCACAACAGCAGCACGGATCTTGTGCTTTAACGGTGAGA
CGACGGTAAAGGTAGCGCAAAAAATAATACACAATGTGTGCAAAGTGC
AGTGAAAACAAAAGCGTTATGTAGGTGTTTTAAGCAAAGGTTCTACAA
GTGCGTATACCAAAGTTGACAAAGTGCGCGAAATCGGACTCTGCCAAG
AAGTGCCGGGAACAAAACAAAACAGCTACAACAACACAAGCAATCGAC
ACACACACACAGAGATGTGTCGTCGTGAGTGGTAAAGGGCAGTGAAAG
AATACGAACGTAAAGTGCGCAAAAAAAACATTCAATTTTCAGTGCGAA
TTTGATTATTCAACGATGCAATTGTATTTGAATGTACTGCCGGTTTTG
CACTTCCCAATACACACAAACACACACACACACACACACACACACACA
CACACACACACACACACACACACACACACACACACACACACACACACA
CACACACACACACACACACACACCCCACACTGTCGTTCGTTCTGTTCC
CTTTTTTGTGAAGTCGAGACGAGCCACTCGAGCCGTCAAATGGCGAGG
ACACGCACGTGTGAAGGGGAAGAGCGGTGTAATGGTAATGAGACTGTT
GTAGCGAGGGGCGGGAGGGGAGGGTAGATGAGAGTAGAAAGGGGGAGG
AAGGGCGAGTGCTCCATTGGCGTCGCTGCATCCGCTGCAGCGCGCGGT
GTGTGCATCCAAGACGTTTTCGCTTCGGTCGTTCAATAATAAAAAGTG
TGCATCGAAACCGCACACACCTTTCCTCTCCTCTCCTACGATCAACTT
CTCTCACACACTCCCTCTCTCTCTCTTACACACACACATCCACTCGGG
CGAATCAGCTCCATGGGGCGCAGACGGCTCTTCGATGGTGTGTATGCG
TTGCGCGCCACCTTCACGCACACAACGAACCCGCTCCTTATAATTAAT
GCAACAATGTTGCTCCGTTTTCATTACCTGTTTTGCTTCCCACCGACA
GCACCGCGCTGTGCCTCTCCCTTCGCACGCCCTCTCCCCCCCCCCCCC
TTTTTTGCATCGTTACCCCTTTTTGCGTCGATGCACTTCCATCCTCTC
TCTCTCACACACGCACTGGTATTTCTTTCTCCCCTCCCGTTGCTGCAA
CCCACCTCAATCACCCCCCCCCACACCCTTTCGCACACTTCGCCTACA
GCCCATCCAACTGCTCTAATGCTACCATTTCCCCGTTTTTCGCGTACT
GCTGCTGCTTCGGTTGGAGAGCCGCGTGTTGTCATGGTAGCGTTTGCG
TTTGGCCGTCTTTTTTGCCTTCATCTTTTGCGCCCGCGTGTTTGTATG
CGTGTTTGTCACGCATGTGGTGTGTGTGTGCGTCTATGTGTGACCATA
AAAAAGCATAACGCGACGAAGTGTTTGCTAGCAGGCGGCGGCGGCGGC
TCGCTGGGCAGTGTCGGTTCGTTTTCGCGTTTTCGTTTTGACGGCTTG
TTAGGGCGCTGTTCGGTGTTGTTGTGGTGGCGCCGTCGGTGTACGAAA
ATCAAAACAACAAAACATATGTTTTTCGGAAAGTTCCACCCCAAAGGG
TTGTGCGCGCACGGAGCGCCGCTCGGTGGAGCGCATTGTGTATCTGTG
TGTGAGAGAAACAGAGAGAGAGAGAGAGTGGAAGAGAGGGGGATAGAG
TGTGTGTGTGTGTGGGAGGCAGAGGCTTGCCGCCAAATATTGTTGCAT
TCTGCGTGGCATTGCGTGGGGTTTTGCGGACTGGTGAATATCGGTGTG
AGCGAGCGATCGTGTGTGGGAGGGGGTTGCCGGACGGCCGGTACATTT
ATCAAACGTGAGACACGTGCGTTTTTTTGTTGTCGTTGTTGCGCTTCA
TGTTATCTGTGTGTCGCAGTGATAAGGTTCGAGCAGCTCAGCACCAAT
TGCACTGCAGAGTGGTGTGCAAAAATCATGTTCGTTATACCTACGATG
AAGTTATCAGTCTGGAGAGAAAGATGCAATTATGTTGGATAATGTTGA
TTATTTATCTAACGAGTCGTGTGACGATCAGAGCTGATAAAAAACACT
AGCAGACTATCATTTCAATCAGCTTAATTTATTTCATTTCTCACTGTT
GCTAGGGCTGTTTAGTATCTCTTCTATTTGTACATTTGTCAGTGTAGT
GATTGTAACGAATGATTTAATCAATGATAAATGATTGAAGGAAAGAAT
CGAAAATGAAATTATTTTTTCTTACAAGTATGTTACCCTTTTTCATCG
TCATTTCGCTCGCTTGGATTACAGTCTTACTCTTTGGTATAGTTATAC
AAACTATTATAACTATTGATTATAAATTGAAATTAGCATAATAGTATT
ATTTATCATTTTTCTGCAAATATTCTTTGGATAGATTTTTTTTATCTT
ACTTTGATGAATTATGTTTTGCTCATTCATTATTTGAAAATGTGGCAA
CAGCTTGTAACAGCCGTTAACTTGTTGCATAGCAATTCAATTCTATAC
TTTACAAAAGGGTAAGATTGTGGCATTAAAATCTATGTACGGTACTCG
CAAACCGAAAAATTTAAAATCATTTCGATTGTACAAAGTACGCAATTA
CACTCTTTTTTATTCCTTTACATAACTTCCTATCATTTTCGTCCGTTT
CATTTCATTGCTTGTTAAATATAGGTTAACACTTCGCTCAGGATCCGT
TTATTGTATTGTATTCTATTGTACTAACACCAGTTTTAACACCATTTT
TCCATTCCTTCCTGAGATCCTTCGAATAGTGCGAAATTTGATCCTTGA
GCGGTCCACTTGTCTCACCGTTTATTTCTGCTAATGTTCACCGAGGCA
CATATACACACACACACGCCCCCGGACACACACATTGATAGTTCAACC
CTTGTCTGAATGATTGTAAACGCCTCGTATCACCACCGGGGCGACCCC
ATCCCACATTGACTGCCCTTTGCAAAAAGAAAAGAGAAAAGTACTCAC
TCTATCCGTGCTAAGTGCAACAGTGTGTGTGTACAATACGTGTCCTGG
TGTGAGTGCGAGTAAGCGAGAGTGGGAAAGAGACGGCAAATTGGGGGT
GCAAAATGTGTGAGTGTGTGTGTGTGTGTGCGTTTGTGGGGAGCACGA
TCGTACATGCATACACGTGCTCGGTCGTCTCCATCACGTACAGTGCGC
GCATGCTTGTGTGTGTGTGTGTGTGTGTGTGTGTGTATGTGTGTGATG
GTGTGTGTAAAAGCAGCCGTGAAGATGCAGGGTTCGCTGCCGATGCAA
TGAGGGGGGCACATTGAGTTTGTGCGAAAATGTTTGCCAAAGCTCGAT
CAAAAGGGCAGCAGTTCGTTCACACATACCATCGCAGCGTTAGCAAAC
AGCCGCCACTGCTCACCCTGCCCGCCCTACGACGGAGACGAGCGGCAG
CCGACACGCGGACAGCGTTCCCCGTGCGGGTATGGGGCCGACGCGACG
CGCTGCGAGTGTATGTGTGTACGGGCGCGCGAGCGAGACGGACGGCGA
ACGGTGGCGCGCGAGCGAGACGGACGATTGACTTCGCCTCAACTCTGT
TGCATTGCGTGTCGGCGATGCACTTGGCGAACTGCAGTTTGTTCCGCA
GCATCGTTCCCATCGCATCGCATCGCGCGCTACAACCGAGACGACCGT
AGCTGGCCACGGACGAGCGTCGGGAACACATACAACACTCCTGTGCTG
TCCGCCGTCGACTTCGAAAGGCACCCAAATCGCGCTCGCTCTCTCTGT
GTGTGAAGCACTGCAGAAGCGTGCAGTCGACATTCGAGCATCCGTTCG
GGCAGTGCGTGTGGTACGTGCGGCAGTGCAGTGGGCCGCCGGTAAAAG
TGTATATCGTTGCTATGTCGACGATCGCCTACTAAGGAAATTGCGTCC
AATGTACCAGTGTCAGTAACGCGCGTGTCGGAGAAGCAAACAGCCACG
GCGAACGCAACGGAAAAAAAACGTTTGTAACCGCGTTAGTTGAAGCGA
ACGAGAACTTTAGTGTGTTGGGCAGGATTTCTCTGCTAAAACCCGGAA
ACTTTACGTTCGGATCGGTGAGCTGTGCCGTGTGTGAGAAGAGAGCCT
TGGCGGTGACGGCTTGGCTGAGAAAGGGGCCGCCCAATAATCCTGAAC
GGCCGTGCGTAAATAGAGATAGCCGTGCGCGTGCCGGTGCGGTGGAAT
TTCGTGTGGTTAAATCTGCTTCCAATAAAACTCGTTGACGGCGCTTGA
CAAAATACAGCCGCCCAATCGGTAGCAGCGGCCCAGTCAGTATCGGAC
TGCAAAAAAAAAACTGCCAGTTTTGATAGTGTGAGGAAGAGTGCGGCC
TACGCGCACACGTGTAGTTTACGCCAGCTGATAACGGTTTCGGCGGCA
GGCCCCAAACGCACAACTCGCAGGCGGTACGCAACACAGTTCCAAGTC
AAAAAGCGTGAAAAAACGCCTGCATCCCCAACAAACACATACACGCAT
GCGGCCGATAGAAAAGTAAATATTCACCACCGCCTGGGGAAATTGCGA
TAAGTGAAGGGCGGTGAAGACACGGCACAGATATTCGATTGACCGCAT
ATAGAGGCGCGAAAAGTGTAGAATTAAATGGGTAGAAAATAAACACTC
CGCGTTGCGTTGTGATGTGTGATGTGCGGATTGGAGCGAGTCACAATC
CTCTGGCCCTGCGCCCGTTGCAGTGAAACCCGCGTGGACGGAATGCAA
TTTTTATCTATCTCGTGTGTGTGTGTTGAAGGGGTTTGTTGAAACTGG
AAAATCAATTGTGAAACAAAAAATTATCAGTGATTGTGATGGTGTGTT
TTTGTTGTCGTTAACAGTGTGCTGGGAATGAGATTAAGATTTACGTGT
GCGTGTAGTACTTGCCTGGCGAGCAAGAAGATATGAGATACCCGCTCA
TTCAGTAACAAAATTAGTGTGATCGTGTGTGTTTTATGTGATTGTGCA
GTGATGATTGTCCAATTAACGTAAAGATAGCAGATTTAAGAATTTTAT
CAAAAGGAGTGCTTCAAAAATATATATTTGGTAAGTAAATATGCAAAC
TTTTGTGAAATCCTCCTAAGGACAGTCAGGCCGTGTCGCTTGAAAAAA
GTGTATATTTTCCAGGGAAATCATTAGTCATTTAATGATTGCTAGTTT
TTTTTTTAATGTAAAATTAAATAAATTCTATTAATAAATAAATTAAAT
GTGCAGCATATAAATGAGATAACGAAATTATTTATTTTCTCCTGACAT
GAAATTTTGTAATTTTTTTTTGCTTTTCGTAACCTTAACTATCGAGAA
TTTTTTTTTACAAGACGTTGACTAACTCTAACGTTTGTCTAAGATCGT
AATACACATCGCAATAGAATTTGGTCAAAATATTCCACAGTGATTTAA
ATTTATGAATGCGTTTTGCTGATACAATTCTTTAATTGTTGTTAATTC
TATAAGTATTCCAAGTCGTACTAACGTTTTATTATCCATAATAATTCC
GTTAATTTGGTTTCAATGCTTTTGGAATTTCAAATAAGCTATATCCAG
CATTAATGAACTGAAAAATTCAATAACACAATTTTCATTATTTTCAAT
GGTGTTATGCTTTGGTCATCCTAGCAGAAGTGAAAAAATGCTAATTTT
AAATGTTCCAATGTTTTGAAATATTACAGGAAATCAAATTAATGTATA
TTATGTCTTAAATAAGATGTTAAATGGACAAGATAATAATTAGCCAAA
ATATTGCATTACTTCAAATAAAATATGAGATCTTTGAAAATACCCCCG
TGCAGGCAATTGGCTACAGCAAGAAGCAATTGCGGTTCTTTGTCATTG
AAGTTATATATATTTAAAAGATATATCAACAAAAATATGCTTTTTAAC
ATTTGTTAGATACATATAAACATTCGAGAACAATACAAAATTATGTAA
TTTTGAATTTTAACACCATAACAAATGCAACAAACATAGCCTGTGTGT
TTTGTTTTCTTAACATTTTTTTGTCATAGTATTAAATTATTTGAAATG
ATGTATATGATCCCTTCGATCGAATTCTAATGACACTTGATCGAAACA
AATAAAATATAAAATATATATAGCTAGGCTTGTTTAAAATGTTTTATG
GTGAGCGAAGATCTAGTGTGACCTTAAATTATAAAACAGCTATTTCCA
TATCAAATTTCATTGTTTTTTTTTTTAATTTCAAAGATCGGCCATATT
GCTATTCAAATTTTCTTTTATTCTGAAGAAATGCCAGACTGTAATGTT
CTTACTTACATTAATTATCATGTTCATTATCTTACTGTCATCTGTTAC
CTGTATTAGGTCCGGTTATTTAGGTATATTGAAATGTTAAATGTAATT
TTACGTTGGAACGCCTATATCATCTTAATGAATTAAGTTTAATATGAC
AAAAATTAAGACCATAAAATTTCTAAATGGTTCTTTCGGTACGTTTGA
TTGCAGATCTCCCAAACCCTAGCACCATCGCTTCCTCGACCAACCAAT
ACCGACAGCCCGAGAACGATCGTACCCGAGTGGAAAACACATTGTATT
TTCGCAGCAAAAACAACACAGAAATCTTTAAATATTTTAAGATAAACT
CCATGTCCCGACAAATCTGCTTTTTTGCGATTACATAGTAAAGAAACA
CAGTAGTGAGGAGCTTACTTTTGCTCGTGCTCGTACCACCTTTTAAAA
AAACCCGGAGGGACAATGCCGTCACGCACCACGGCCAACGATTTGCGC
GAGCTCGATGTAGCGCCGGCAAGTGTAACGTTAGATCAAGCTTCCAGA
TGTTGAGAGTCGGAGTCACAATACGTCCACAACTGTCGGTTCGTCCAA
TCTGTACATTGTGTGGTCGGTGTTTGGTGGGAATGACAACGGTGTGTC
CTCTTCGAAGGTGCTAAAAGGAAGCTCGCTGACGAGGCGGTAGGGTGT
GAGAGTTTGGCCAGTTTGTTGTTGCGCTTGTGTGGGGTGCAGCAGGGA
AAGCATTAGCCGAGAGGTAGAGACACACAAGCTATTTGGGACCGTGAA
ATACGCCGCGCGCAACAGTAATAACATAACGTACCGTAAGCCGAAGCG
ATCGAATCGTGTAATCGAAGCGGTCTCGTGTTTTTTTCCTCCTATATC
GAGAGGCCAACCGATACATCCAGGTGCATTCGGCGGCATAGATAACGC
AGCATTAAGAGTCGGAATTGGCTCTCGAACGCAACAGTTTGATTGATA
TATAGGCAAGGCGTAGTCAGAGAGGTGCTGTAAACGAGAAGAAAGTAA
GGCTAGCAGGAGAAGCGCAAGTTGAGGAGGGGTGTCGCAGGGTTGACG
TAGACGTAGAGCTTGTTTGGAAGACATACGCGGAACCACACGGGCGTG
TGGTGCATCTTGAATGGTGTCACAGGACCGCTGGACGGAAGCAATGTC
CGACTCCGGGTACGATTCGCGCACGGACGGCAACGGTGCGGCCAGCTC
GTGCAACAACTCGCTGAACCCGCGGACGCCGCCGAACTGCGCCCGTTG
CCGCAACCACGGGCTGAAGATCGGGCTGAAGGGCCACAAGCGGTACTG
CAAGTATCGCGCCTGCCAGTGCGAGAAGTGCTGCCTGACGGCCGAGCG
GCAGCGCGTGATGGCCCTGCAGACGGCGCTGCGGCGCGCCCAGACCCA
GGACGAGCAGCGGGCACTGAACGAGGGCGAGGTACCTCCCGAGCCGGT
AGCTAACATTCACATACCAAAGCTATCAGAGCTGAAAGACCTGAAGCA
TAATATGATTCATAATTCTCAGCCGAGATCGTTCGATTGCGACTCCTC
CACCGGATCGATGGCGTCCGCACCGGGGACCTCCAGCGTGCCACTGAC
GATACACCGACGGTCGCCGGGCGTACCGCACCACGTTCCCGAGCCGCA
GCATATGGGAGGTAAGTACGATCATGCGTCTTCATTTCTTCGTTTTTT
TACAACTGCTTCAGTCTGTTGAGGATTTAACACACTTTTTCATACATA
TTTACCATTGGGATACAAACTGAGGCTCTCATAGAGCTTCTTCGAATG
GTTCGAATCATGCACCGAAAACACTTGCAAGACTATGATTTGCTCCAA
CATCACGCAAAGTGGATCATCTCCAAAGTGAGCGCATCTTTAATGCTT
AGATTGCGCACCAGAGATCCTCCAGTTCCCACGGATTGGGCCTGTGCT
ACATTTTATTGGTTCGCTTAGGCACTGCCTCAAATTGGAGCATCTCAG
CACGGTACGCACGAGGAACGGCTGCACTCAGACAACGGTCGGAAATCC
GTGCAATCCCGGGAGGGGACCGGTTTTAATGCTGTTTGGTCTACGTTG
CCTCGCTAAACCTACCTTCCGGGATCTCTGCAACATTTTTCGCTCACC
TGCCACTTCGTTAGATTGTAGTTCCCGTCGCGAGGACAGTGCCGGGAG
TTCGGTGGAGCAATGCGCTAGGCTCCAGAGAGGAGGCTACGAATGCCT
TGGAATGGACGCTACACACTCTTTTTGTGCGTACTTCCACCACACGTT
ACCTCGACGATTACCCTGGTGGCCTGGTGTGCCTGGTGTTTGGCGTTT
ACGTCTCACTTCGTATGTGTTTCACCCATCACCCTTCGTTTCGTTGTT
GGGGGCTCTGCTTTTTTTCTGCTTCTTTCGTACTCCCTCTCACACCAC
TGCTGCTTGCTCCAGCACGTCCGATTCTTTTTTCGCATCGTATTACCA
TAATTATATTATTTAATTATCTACTTCTTTTCGAACGGTGGCGTTGGA
GCCCGTCCCTCTCTCTCTTTTTCCCTCTTTTCCCTCTCTTTGTCTGGC
ACTGTGTTCGTTTGTTTTACTTGTTTGCACGCTTGGACAATGCTTGTT
TCTTATGCATCATCCCCCATTGGTACATTCTTTAGCAAGACGCGTATC
CTTTCGCCTGCATGCAGAACCGTTTAAGTGCGCCCAGGTCCGGAGTGA
GACGAAATTGATCAGAATTCAGACACACCTCGTTATGGGGCCGATGAT
GTACCGCCATGCTGTCGGACGCATTGGTTTGGCGACGAAGGTGTTTCG
GTGCCCTGGTACTACAAATAATGGCAAACGGTGCACTGGCGTATGCGT
ATGCTTCTTCGCCCCGGTTCGTTTTAAACGGATCGGTAATAGTAAAAC
AACACGTAAAAGCGATATTTTGTAGTGGACTTTGGTAAACAATAAGGT
TCCGGCTGCAGTTGGATCTTGTTTTTCTAGCTACGGAATGTCCGGTGT
GCAAGGCAGACGTTCTTCAGCAGGTCCTGTGCGTGATAAAACACAAAG
GGACAAACTTTTCATTTGCTCCTATTTGTACAACTGCGTGGAACACAC
CTCATATACACGCACACAGGGTACCCGGGGAAAAATGTCGTGTCGCTT
CCTTGGACGATTGGTATGTATTCGGAAAAAGAAAATACTTTTCGAGCT
CGTGTGCCGGGTGGCGGTGGCTGCCGTTGTTGGAACGGTTATCGCCAA
ATTGCTCTTAACTTTGCCACTTGTGCAATTATTACTTGTTATATCTTT
TCCTGCCGGCTGGCTTCTCTCTATTTCCCCCAACCTACTCTCCCTTTC
CCTTCCTTTCCTCTATCGCCGCCATCATGCCAAAGGAAGCTGCAGTCA
GCACTCCCTACTATCGGTTGAATGTGTGTAGTCAAAGATTAAGCGTTG
CCCGTATATGCTAAATAAAAGTTTGCACGCAATTCCACGCTTTTCCTC
GCCGCCTGCGAACGGTGGGGTTTTGGTGGCGGGGCAATGTTTTCTTCC
TGCACGAGAGGACGATTAGTTGACCTTACTGAGCGCACGGAGGGAACG
CAGGAGTGTGGGTAGGGTAGGTTACTGAATGACCACGTAAGAGACGTT
TTTGCTTTGTTATTGATTATTTTTCAGAGGAAACAGAACAAAATGAGC
AAGTTGAACATTTGATTTACATTCTTGGGCTGTGAGATTGCATTAGAT
TTGTGTTGAGCTGTTTTTTGAAATGTAAAATTATTAGCAATTACTGAA
GGTTTGCTGAAAGGAGAGCTGAAGAAGTATTCTATTGGGAAATATATG
TCTATAAATGTGCAAAATACTTTCCCAGAAGATTCAAAAGGCTCGGAG
AAAGATCTTACATTTTGTGTTGTAAATGTGATCATTGAAAACCTCACA
ACACTAAATATACCTAGTAAATTTAAATTTTTAACGATATTGCCTACA
TAAAACATCTAGAGTCTTAACATCGCTTAGAAATGCCGTTTGGTCCCA
GCTACCAACATGCCAACACGGGTCCGGTCAGCACCAAACCCGCCTATG
GAAGCTCATCTTTGGCTTGTTTTTATTGTTTTCATCCCCTCTAAAACA
CATTCCCGGTGCGGCATGTTAAAACTGTCATTAGAAGCTTTGGCGCGA
ATCGCGCGCGCCCGCTCAGGGGTCTTGCAAACCCGTTCGCTTCAGCTT
CTGGCTGTGTGTGTGTGGCTGGGCGTAGGTACGAATTTGCGGAATGTT
GCAGAATGTGTCGCCAGCAGGACAGTGCGGTGCGGTGTGCATTTGCTA
GAACAGGTTTCGCGAAGGAAGAACGTTTGCTAGCTGGCTGTGTAAGGC
TTTTGAAGGTATTTGATTGATTACGACCGCCAACGTTCATCGTTAATC
ATGCGCCCGCTCAGAATAGCCTACCAGTCATGGGTGGAGGAGTTCGCG
GTGGAGTTCTTTCCAGGCAAAGCAGGGAGCTGCGTGTGACCCGGACCC
GCTTGCACATTGTTCGACAGCCGCAGTCGCTCCATCGAATGTCCCTGG
CTTTGCTGGCCGGCTTTGCGCACCGGCTCGCTCTGGCGCAATGAGTTC
AATTTTCGTTGCGATCGTGAAAAGATCGCCCGAATCATCCGGTAGTCT
GCTCCGGTGCTGCAACTACTTATTAAGCAGCATTATGTATCTTACAGC
TCATTAGGCGGCGTCGAAGGAGCACATCAGCAAACAACCGTACCGTAA
TGTCTTAAATGCGCGTTTATGATGGGGTGACGGACCTGACGGCATGGC
GGCCGTTGCTTTTGTTTTGATTTTGTTTTTGGCACTTATAAGGTGTGG
TGGGGTTGGGCGGATGGGGTCCCCCAAACAGGTAACGACTTTGACCGT
CGCCGTAACTGGTCGCTGGTCACATGTCGAAAGGTGGAGGGCTGCACT
ATCAAATGTCACTGCATCGAAACGACGGGAGGTGTTGTATGTGTACCA
TGTTACTGTTTGTGTGTGTGTGTGTGTGAGTGTATGCTGGCCAATGTT
GCAGAGGTTTTTGCGCGCGTACGATCGCCCTGTAACCGGTTTGAATTT
TTGCACACATTTTTTTGTGTATTTCCAGCATCAGGTCGCGCTGGAAAA
GGTGATTCGATCCCATTTCTCTTCGCTCCAAAATCGAGCGCATGCACC
TCGGTACGCGGTATGTGTGTGTGTGTGTGTGCTTACGTGTTTGATGGG
TCCGGTTACTGCGCACATAAATCCTCGACACAGTCGGACAAGGGCTCT
CGTGTCTCTAGTTTTTGGCGATGGCTTTTCGGCCGCTCGCGCGCAGCT
CCTGACGGCTCCGAGCGGCGATGGTGTTGATTGAGTCATTTACTACCG
AAGCACCGATAGAGATCTCGTTGGTGGTGGTGTGCGCCACAGATCTTG
ACGACAGATTTTTTGGCGTCCGTAGAAGCTCATTTCACGGTGCGATGA
AGACGAATGGCCGGCTAGAGAGCGCCGAGTCGCTCCGAGCGGTATTGT
GGTCAGAGTGAGTAGCTTTGTCAAGGCGTCGTTACCCTTTATTTCTCT
CGCGATCTTCGTTTTTTTTGGTTAATCAAGAAGGGGAAAAGAATGACA
GCAAACTAGCTGTTTGAGAAAAGCGGAGGGTTGGCTTAGCGACAAGGG
TGCTACATAAAAAAAGAAACAGACAAAGAGCGTGTTTAATCCGATTGT
TGTGTTGTTTCCGGTTGAGGGAACCGCCATGCTCTGCCTTCCAAACTT
CCGCACTAAACAACAACTTCCTGCGCATGAGGACTATCACTGCCGCAA
GGCGCACATCTGAAGAAGCCCAAAACTCGTCGTCGAAACACCCCAAAT
CAAAGGTCAAACATGGCGGTTACTGCTTCTTCTTGTAAGGCCGCCGTC
GTCATGCTTTTGTGCCGTACATTGACACCTCAAGTAAAACAGAGCAGC
GGCTAGCAGGGACTTTTGATGAACACTTTCGTCCTCGCCTGATGAGTG
GTAGAGGCACGCAAGCATTTCAGTTTTTCCCCTCCTGTCGAATGGTTT
TTCGCCCCATGCGAAAAATGGTTACAGTGTTCGACCGTGAGTGAGTGA
TATTTTAAAAGATATTTCACATTTACTGCTGCTCCCTTTCCTGCGCTG
CGACGAGCGCACTCGCTCGTACATCCCATTAGCGAGCACGCGGCCCTA
CCAATAGATTGCAAATGCGCCTTTCTGCGGGCGAGTCATGAGTGAGAC
ATCTATGACGGATACCATGTGGACAAAGCGTAAAAAATGCACACAAAC
ACACACACACACACACACACACACACTTGCACTACGGCAAAGATCATC
TTTTACGCGCACCGCACACCGATCGCGGCAGCGCCCAAAGTGCATAGC
GATGGTGGAGGCTTGCGTTTTGGAACAGACCGCGCACACGGGCCGCCG
GTGTGACGTGTGGAATTTCAGCTAATTAGAAAATTATTAATAGTTCCT
TGCGCACATGATCGGTGCGCCATTCTTCTTCCTGGCCAAAGTCACCCG
GGTTCTGCATTTCCGGAGCAGAGTCCTCGACAGGTTTTCACTTTCCCT
GTCACACGTTTGAGTGTGCCTATGTGTGTGTGTGTGACCCCTTCTCGT
CTTGTGCCTTGGGGTCGGCTAGCAATTTCTAAAACTTGCTCAATGGCG
CATCCTTTTCCTCTCTGTGCGGAGAACGTTTTTCCGCGAATCCATCCC
CTCGCCCCAGGTGCTTATGCAATCAGCGCTGCTTTACAAATTAAAACG
TAATTTAGATCCTGTTCATTAAGGCGCGCGCCCGATGCGATCCTTTCC
CCGCGCCACGCGGTGCAATTAAAAGCGTATTTGAATAATTTGATTATT
GTATGAAAATCAAAGAAATTTGTCTTTACCGGCAACAAAGGCTTGGCA
TGTGGAAAACCAGCACACCGACAGAACAGGCCTGTGGGAAAACGGAGA
ACACACACCGGCACACCAAACTGGTTCTTTCCGGGTGCGCGCGCGACA
GCAGATTACATCTGGTGACACGAGATAATTTCCATTCCGCGATGCGTT
TTGCGCTGTTTGGTTGTTGTGCGTGTGTTCGGCCGAAGAGGAGGGGGG
GGGGCTTTGGACAGCAAATGGCTTGTTAATGGGCTTTTACCTTTGAGA
ACTGAACCGCAAAACCCTGCCGAACAGGGGTGAGTCTTGAGACAGTCT
ATCGTCGAAGCTGCTGCGCGTTCACTTCCTCATCACGCAAGCTGGCGC
GCGCACACGGCCTTTATTTTGGCAGCTTCAATCGGAAAGCCAGCACAC
ACACACACACGTTCGACAGCTAACGAGAAGCAGGGTTGGGACCACCGA
TTAGAGATGTGCAATCCGCGCTGTGCACTTTTGCATCGTCCACACACC
CCGCGGACACTTTGCTCGCTTTTCGCCCCGTTGTTCTCGGTTGATTTC
GCCGTTCGGCCGCCGACTTCGATTCCCTCATACGGGTGGAAACCGAAA
ATAATGCGCGAGTTGCGCCGCCACCCGCCTAAATTTAGCACCACGAGC
CGGCCGCGAGAGCGGCAACACTGTTGCGCGGCCAAATGTCTATTTTCG
TCTAATTCCGCACAGCCCGTCGGTACGCTAAGCCGTATTGCGGCCCCG
CCCCCGCTGTACCCGCCGATGCCGATCGCGGAGCAATGTGCGCACTTC
TTGAGCAACTAGGGTGCACTTGCACCCCTGTCGTACTAACCTTTTCCG
TGCGCCGTGCGCTCTCGTGCGCACTGTTCTTCCTCTCTCTCTCACACA
AGCGCATAAAATGTGCAGTTTGCGGGACAGATGTGTGTGTGTGTGTGT
GTGTGTGTGTGTTGCGCTTTCCGGTTCGTTACGTGTGACGTGTGTGCG
CGCGCGCCATTGCTAAAGCGATCGATTATCCTCCGGGAGCGCTGTTCT
GTTCGCTCTTGTTCTTTCAATTTTAACCAACCAAGCAACCCACCCACC
CACCCACCATGCACCCCGCTGCCTGTTCCACATGTGCATCAGTGGTCA
GCTTGCATGCTCGAATGCAGCAAAAAAGTGCAATGCAGAGAGTGCAGC
AAAAACAAAGCACACCATGCGACAATGCAAAGATGTAAAAGTCACACA
CCTCCAACGAACCGCAATAGATGGGATGGCCCCTGCTGGGACGGGCAA
CGGGAGAATAGGGGCAGCGATGATGATTGATACATTCATATTCGTCGC
CGGAGACCACCCGGGCCACCGTGGCAGCCCTTGGGGGGGAATATGAGC
ATCGCGTCACGTCGTACTTAATCAACGCGTGTGCGTTATTTGTCTGCG
GCACTTCCGCGTGCGTATCTGTCGTGTCCGTTCGGTTCGGTCGGTTCT
CGGTTGGCCGTCCCGGTGCTGGACACACGCTTTGCGCGATTGCGGACA
GTCTGCAAACGGCAACGGTATGGTGTGAAGAAGTGGTTCTTTTTTGTG
TGCTTCTTTTCTTTCGGAAATATGAAATTTCTTCCGCTGCCTGCCTGG
ACGCCGGGAACTGGACGAACACAGGCGCGGTCCGCCGTATTTTGCCAT
TTTCGCTCGGATGTGGTCGGATGTGGGGCCAATTGCACACACAAACCG
CGCGAGGTGGAATGTATTTATTTACGTTTTAACGGTGCAGCTGTCTCC
TGCCGGTGCATTTCGTGAGGTTCCTTTTGCCCATCGGGAGTGTTGTGA
GAGGAGTGGCCGAAACAAAACGGACCGAAAAAAACTGCCACAGCAACA
GTTCGAAAAGCACGGACGCACAAAAACGAGATCGCTCGGAAAAGTGCA
ACTGGTGGCGATGGTGCATTATTTCACATTCTTTTGGCCGTACGAATA
AAAACATGAAGCAAGTACCATGCGAAAATTGAACTTAAAAGATCCACC
CGTAACGGTTGCACGGCAGAGCGTGCCCGAGTGGGACGTGCGTTAAGG
TGAAATAAAATAAATTAACTACAAATTTACAATTAAATTGATTCCATC
CATTGCACAGTCGAGGTCTCTGAGCAGGAGTACTAATATTCTACCGGC
AGGTCCGTTTGCAGGCTGCAACACCGTCGTGCAGCTTTCCCCTCGAGC
AGGCAGTTAGTAGGCAAAGTTTATGTGCTAGATAGCGGTGGTTTTGCG
GGGAGAATCAAGTCTAGCACACACAAACAAACACGGGTATGTAAAGGT
TGAAAGGCTGTCTCAGGGGACCGAGTTGCCGATTGGGCGCTGGTTCGT
CCACCGTCCATCGCGCGTCCTGAACGGAAACAATAACACTCATAATAA
TGTTTCAATTAAACACAGGCGGGACGACGACAGGAACCGGTTATGATG
GGACAATTTCACAATTGCACTTGACATTGGGCGCAGAATTGGTTTGCA
CCAGCCATCCAGGGACAGTTGAGCATTGCCCAGTTTGAGCCTTTGGTC
TGGAGCTTTTACATGCTAATTAGATTTCAGTTAGACAACTCTGCGCAA
CATACGAATGCTTTCAATATGTTGCACAAGGGCACAATGCCGCAACAA
GGTAAATGTTTCCTGTTTCTATAAAACAGACTAGACGTACTTTAACCA
AGCTATGGACAGAGTCTATTTTCGGATGTCATAATTTACGTTTGAATG
ATCAATCACATTTAGTGACTGCTAAACCTGCTTGTTATGCTTATCCTG
TGTATCCTAACGCTTAATTGTTCCGTTGTGTCGTTAAACTAGCTTAAA
GCTTCTTGAACCATTGAAGCTACCATTATGAATGCAGTATAAGCATGC
AAGATTTATTTCTTTTCTTCGTTTCGATTATTCTTTCGTAAAAGGCAT
CTTGATTTAATGAATCTTTTGCGATAATCGGCTACACAGCATGGCATC
TGCGGGGCAGAACGGTACTCGATCGAGCAGTCGCCATTATCTAGGAGT
GCGTAATCAAGTTTAGGTTGCCACGTGATTCGATTCATTTCACACCGA
CATGACAGCAGAATAGAATACGGGTGCGCCTTGCCGCACTACCGTTGA
CCGTCGCGCGAGACCTTCTCAATGGCTGCATTCATCTCGCTGCTCGCA
AGTGCGCCGTGAGTGGAGCATAAATCTCGACAAACGTTATTGCATTTC
ATCGACTGTCTTCGATCGGGTTTGGGGGGGGCTGGGTAGACATTTAGG
AAGCAATAACAACTGTCTTATCGTGCAAGGAAACACACCGGCACGCGG
CTAAGCCTGTGGTGCAGTGGTTTAGATTCCTTTTTACTTTTACTTACC
ACCGCACATGCTTTATGTTGGATGTTCAACAGGCAGCGCAGACAGGCT
GAGAGCGGTACAGCATACACACGCCGTCTTGCTTGATAGACAAGGCTT
CGCGGCCTGGCATTGCCGTGGAGTGACGTGTAAGTAGTGCCCCAAAGG
CACCACTCTTCACGGGATAGAATTGAGTGCGTTGATGTGAACGGGGGG
CGAGGAAGCGTAGTGCCGGTTGTCGTCGTAGTTGCAGCTTCTGCCCGA
GCAGCACTGTCAAAATGGGTTTTGCGCTAGGTTGAGAATCGGAGGAGG
GCCTTCGCCGTAGAAGCCGTAGCGATCGTCCTCCGCGAGCACGGGACG
CAATGTTGCCACACATTTTGCCGCGCTTTTTTTTTGCACTCGGCAGAG
TTACGACGGCTCTCCGGTATGGAAGCGAGCAGCACATCTCACGGGCTG
CGTCGAAAATCGAGCATAATTGTATGCTGTCTGATCTATTTCATTTCG
CGTTTTATGTTTTATTCGACTTGCTGTTTTCCGCCGCCCGGCTCAGCT
TCCAGGCAGGGCGGGAGGCTCATTGTAGGTTAGGGCCCCGTTTGACGT
GGGCCAGACAGTCGGCGATGGGGCGAATATGGGGAGAGGTTGGTGACC
GATCCCTACTCCATCGTGTCCTCCTTGAGGACTAGTTTCGCTCTCCGA
CACTCTTGACACTTCTCTTCCTTCGTCTGATCCTCTCCAGGGAAAGGC
TGCTGGGCGAGAAAACCTTGAGACGCGGGAGCAGCCAGAAACCGGCTC
CTCCTGTGCAGCGTGCAACAAACAAAACAGCAAAAGATTCTAGGCTCC
ACACTGTGCACTACTACGAGAGAGAAAGAGTGTGTGTGCGTCCTGGGG
TAGTTCTGTCAATGTTGAAAAAGGTGGCAATGGAAGAAGAGCTAGAAA
AACAGAGGCATTATGGGGTGTTTCAGGCAGGAGGATTGGTGGGTGTTA
GGCCGGGCAGGAAACCGGATGGGAAGTCGAACGGGATACGGATGCTGC
TGTTACGCCACTGAAGCGGAATCGTTTGCGGAATCGGTCAACATTGTT
GAGATGGCCGTGTTCAGCCTGCGGTTGATTTAGTTACTTTTTGATTCT
TTTTTGATTCATTTCGTTTGTGTGTCCAAATGAAGTGTGCTGTTGGGC
CGGCAGATAGGGCTTTCGGCGGGTACGCACTCGAGAGTTCGTGCGCGT
ATTTCTCGAACGTCACGGCATACCCTCATCAAGTGAGGCTGTCCCGCG
ATAGGTCTTGTGTATGTGTGTGTATGTGTATATATTTTTAAATTCTGG
TTTGGGGCATCAGGACCCTGAAAATGTACCACCGAAACCCAACGGAGA
GACGAGCTTGTCTGAGAATGGTTGGGAGCGCAAGCAGTGGTGCTTACG
ATTTATAAAATAAACAACGACGTACGGATACCGTGCGACGGGATTAAG
GTCACGTTCAATGTTACGATTGTCGATCGAGACAGGCATCTTAAGCGG
GCTGAACGGCTTGGTCACACTGGAAGGGATTATTTACCGATATAAGCG
ATTTCACCATTGGCGTTGTCCGTAATGCGAGGGCGCCGATAAGCTGAC
CGAAGCAGGCGCGAAGAGTATTTTTGTAACTTGGTTGAAGAAACAATC
ACAAGCATCTTGATGATAAGGGATAATGAATTAAACATAATTGCATCA
CCTGTGATGAGACAGTTGATAAATGGGACGTCTCGCGAAATTCTGGAA
AGCGAGCAATATCTTCGTACAGCTGCATCTGACATTGACGTGGCTGCC
GGTTGCATTGCGAAACGTCAAAGGTGGCGCTAAAAGTACATGTTTAAA
ATTAGTTTCCATTTTGTTTGTTTGTAATGCGCTCCGGTTTGTGTGCAT
GTGTTCGGGTTTTTAGCTATTAACTGCAATTTCTGCACTGCAAAATGT
AGCCGTTCCGGTATGATCAGCTGCAGACACGTGGTGGACGGATCTTCT
GCTTCGCGCAAAGTGCACTTAAATGGTCGTCGAAGGAGTGGACAGCGC
CCGCGTCTGAGCTCATAATCGGCAGGCCAATTATGTCGACGGGAATGT
GGAAGGATGCTTGCTGCAGCGAACAAGATGCATTAAGCATGGGCAATC
AATCATCCCGTGGCTCTGCAATCGAGGTTTCCGTGACACACACGCGCG
TCCCCGGGTGTCGTCGCTGACGATCGCGTGTTTTACAAGTGCGTCCGT
GCGTTCCGTACGTCCGCTGCGTCGCCGTCGTCCGAGCCACAACATGCC
CACGGCCAATAATCAGTATAATTCGGTTTAACGTTTGGTTAGATTATC
GGGAAAGAAAATAAGCCGAGGTAAAAACGGATCACTTTTCAAACCGAA
CCGAGCGCAGGACTGCAAAGATGGGAAATGTGTGTTCACGTGTTGCGT
GCGTGATCCAGGGTGTATGTTGCGAGAAATTATTGGAATCATTCCAAA
GTTATGTCGGTAACCTCAGCGTTTTTCGTGCGGTGTGTCGGTTTTATG
CAGAAAGCAGAGATCTTAAAGCGAGCTGGCATTTTGATATAGCACATA
TATTCGATGGATGTAGCATTGAGGTATCCTCAATGACCATTCTAAATT
ATCTTATCCTTAAGGCTGTTTTTGGGCCGAGTCCTGCAAGACTAGAAA
AAGTCCGATACCTATTCTAACTGTCCTCCCATGTACACGTTTCTGCAT
CGTTCCTGGAAGTCATGGAAGTCATAGAGAGTCATTCAGTTTCATCAC
AGAAACGAACAGAACATTGCCATCAAATTGGACAGTTTCAAAACTTCA
TTCAAGCAAAGATTAAATTCTAGCGTTAGCTCCATAAGATATTCGACC
TCCAGGTTAAGTTATATTGGTCTCTAGCTAAGGTTGATGTATTGATAT
GGTCTTCAAACCTCTACTACACCCTAAATATCTTTGTCAAAGTCGTTA
ACTCTCACCTGGCATGTAGAGGAACAGGCAACAGACCAATGATTGAAA
AGCCACGCTCATGTCTTCAGACCATAACCTCGGCCAAATTTACCTTCC
AATCCATCGATAAAACCTCATCGTTAATGTCATTAACCTTTTGCAAAG
CTTTTACTCCAGTGCCACCAACAAACATTGCGTCAAAAAACGACCAGT
GTCACGTTCTCCTCCCTGTGTATCGGAGCATCTACGAAAAAAATACCA
AAAGCCTCCCTTAAACTGGGAGGCCCATAATTCCAGCTGAACGCTTAG
ATTGGAACGGAACTGGCGGTGTCTTTCGTAGGGCTCGGAACGTTTTCC
TACCAGCTTCTGTTTGCTCGAACCCGAAGCAGAGCACAAACCGTCTAG
GTTAGCTGACAGAAGAAATTGCAAGATGCACAAAAAATCGCACACACA
TACACACAGACGTTAACAGTGTATTGCGACCGAACGGGCAGCAAAACG
CTGTGGCTATTGTGCCAGACCAGAAGGGAGGAGAACTCAAAAACGGTA
AAGCTAATAAACCTGTTTCTTTCCATTTTTTGCGCATTGATTCATTTC
TTGCGCCGGCGAGAGCTGCCCGGCAGTTCCTGTTGCATACATGCAGGG
AGCGCGGGTTTCTCGATGTGCGCCACCTCTGCCGCCGGCATCGCCACC
ACCGTCACCACAGACCGGCTCGAAGGCTGCGGGATGCAAGCGCGGCAA
CCACTGGAAGGTAACCTCTCGGGGCGATTGTTGTATTTACCAATCGTG
ATGCATGATCAATGTTGTGCGGAGTATTTTATTTCTTGTAAGCAGCAG
TTTGAGGATCGGCCAGAGGTTTGGGTAAACATTTCAGTCGCTCAGTCG
CTCGCGAAACAGAATAAAAAAAACGCACACAGCGTTCAAGAGAAAGGC
GCGCATGGCGGTGGATGTAAAATGCCTCATTTGTGGCGTCTTTTCCCC
TGCGCGCAGCAGAACGTGAATGTGTGCAGAGCATGGTGTAGCGTCGGA
CGAGGAGCATGAATTTTGAGCAAGCGGAGATGGTTTTGAGTAAATCGG
TTTCTATGCAGCCAAGGCAACGGCAGCCGCATAGAACTAGAGCACTGT
GGGCCAAGTCGCAGTCGAGGCACGGAAGCAGGGCAGAATCGCGACTCT
CTATCGCCCTTGTTGGACGACGGATAGGACCGATGCCGGTGCGGGTCA
AGTTCAGTTGGCTTACCGATGCATCATCGGAAGCCATCTTAAGTAAAT
GGAGAGCTGGTTGGCGATGGAGCATGGGGCTCGCTTTACTCTTTTGAG
TGGGCACAGGAGTGTTGTGCTAGAAATAGATTCGGCTCAAATTACGGC
TCGGGCTTGCCTAGAGAAAGGGCAATGAAGGATTGAACACATCAAAGT
TAAGTATTTTTTGTATTTGTGGTTGCTGTCGTTAAATGGTTTATTGAA
GCGTTTCCATTATAAAAGTTGTGAAACAGTTGGAGGATGAACAGAAAA
GCGTGGATGTGGAATTATATTTCAATACAAACACATTGCACATGATCA
CATGGATCAACGGTATATAATTTAGTTGGATATAAAAATGCACATCCA
GCATTGAGGATGGTATTTTGCCATCCTCCACAGCTCATTATGTTCACA
AGGTGATGGTGGCGATGGTTTCACAGTAAAAGTTTCTCAGGCAAAACG
GCTGCGAGGCATTGTGCGAAAGTTTGCAGTACCGTGTTCTATGTTCAC
AATTGGGTTTTAAATGCCCCAAACTGTTCGAACCCTTCTCACATGGAG
TGTGTGTGTGTAGCTGTGTGTGTCAAGGACCGCAAACAGGAAGGGTCA
AGGGACAAGGGAGGGCTTGTGATCGGAAGCGCAACAGAATCATGATGA
GCGCAGACTGGCACCGGGCATAATTTGCCCGTTTTTTTATCGTGTGTT
GCGCATTACGGCCCTATGTTGAAGGAGATCGTTTTCCTCCCCACATAC
ATACACACACACACATCGATCGTAAGGTATGCAAGAGGAATGTTGCCT
TAACACTGCGCGAGTTCGGTTGCAGTCGATAGAATTCGGTGGTTTCGA
GTGCGTGCAGCGCATATTAACGCCAAGGTTGGTCAAGTCGTTTTTCAA
CGCCCCTTGAACTTTGGTGATGCGAGTCAAGGAATAAGAGCAAGAAAA
CAAACACTCCACAGAACTTTAGGATGCATGGACGCTGCTGCAGTGGCG
GTGATGGTGCTGTTGTTTCGTGTGTCACTGTAACACGGCTCATTAACG
GCTGCAGACACAGCGATTGTGTCGTCTGACGAGTTTACTTTAAATTAG
CGATGGCAAAATCAATAGAAACTTTCGTCGCCGCCGCCGCCGCCGTCT
TTTGTATTGATCTCACTGTCCAGCGAAACAAGGTATTAGCACGTCACG
ATCTTATCCCGATTCCTGATCGTGTAAGGTTTACTTACTTTTAATGAG
CCTAAAACAAATAGGAACAATGCTCGTCGGAATGCTCTGCAGCAGCTG
CGTACTGTTTACTGTTAGTGTTCGCTTGTCTTGCGATGTTTTGCTTGA
TCTTAATTATTAATAAGGGCGCGGTACTATTTGTTTGCAAAAAGTCTT
CTATAATGATCGATTGTATTTTTTAAATGAGATGTAAAGTTAAAATAT
TTGCACAATATAAACATCAAATGCAAAACATGCTAAGGAAGAACGTAA
ATATTTCGTGTGGAATAGTTCCTTTTTATTTGAAGTTTTCAATATGAG
TAATTTTTAAAAGGCACTTTGACATATTTGTTTTCACCAATGTTACAG
ACAATCTATCAAATATGCCTATAATTTTATCAGATAACCTGAAATCTT
TTGCAAGATGCTGTTCAGACAATCACTTCAAAGTTTCTAGTGATATTT
GAGATTTAGATTTGCATTTAAAATCGTGCACAGCATAGCCTTTTATGC
ATTTTATGTAAATCGCAATCACCACACCAAACAGAGGCGAAACAGATT
GTAATATTTTCATTTAAATAACATCCCCCGACCACCCATATGTGTGTG
TAATCGAGTGACCTTGATGCATTCAGCGATGCATGGCTTGGCATAGAG
GGGACCACAAAATCGGGACGGGCGGTAGGGCAGTGCTAGCACAAGCGC
AGAAAATTGCCTTATCAAATAACAAACCCTTTCTCCTCATGGTTGCAT
CCGCACTGCCCTACCGCGTCGACCGATGCATCCGATCGTTTTCATGCC
TGAATCAGTTGGAAAAACTTCTCTCTCGTCGGCGTCGCGAATGGAAAA
GCGTTTCACAATTGCTTCCTACTGTGACGCTCGACGGCGTATGTGGAA
AAAGGGTGCGGTGGGAGGCGGGATGTGGAGAGGCTTATCGTCACTCAC
TCTTGGGTGTATGCGTGTGTGTGTTGTTCGCGGGAAAGCCCATATCGT
AATCGATATGCTTGTTAGAGATCCGTTTTGATGCAATGGAAAAACTAA
CGCTCCAGTCTAGAGACCAACAAACACACACACACATCGAAAGAGAAA
GGGAAATGTGTGGGAGGAAGGGAGAGGAGGGGTGAGAGTGGAAATGCA
ATGTAGTGTGAAAGTGTGGCTGACTGGTTAAATGGATGGGAAAACAAG
GAAATGGATGGAAAGGAAGGAAAAAAAAACCGTCCGACGGTTACAGAA
AGACGCAAAAGTGCTCGTACGAATCGTCGTATCGTCGTTGGCGAACAA
ACAGGCGAAGCCAGAGCCTGCCAGCAACGGAGTTCTACGGAGCTGACG
GGACGGCCAGTCCGCCGGTGTGGTGGATTTGTTTGGACAGAAAAAGAT
CGGAACAGGAGAAAAAAACGCACGCCTTCATAATGAAATGATAGACAC
GTGCACGTTTCCAGTTTCAAATCAATTTCACACTCGAAGTGAGAACAA
ACCTCGGAAACAGTCGCACATACACACATACACATTGGGATGGTTGGC
TGGTGGGTGGTTTTGGTTCACTTTGCTCTCCACTACATGTCCAACGCT
GCTGTTGCTGCGTATTTCATCTGCCCTTGTGAAACGAATCACCAGAAG
CGGTTTGGGTTTCGGGAGCTCATGTTGTGTGCGATGCGTCGCCAGTAA
GCATTCTCGCGGAAACGATAACAAATGTGTGTGTGTGTTGGGTGGGAG
TGAGAGAGAACATGAGGTTGGGGGCGACCATGACACTGACCTAGGACA
ATTAGAAACTGATTGACGGAAACGATATGCATCGAAAGCGAGACGCAG
GTTTTCTTCGTTTTATCAGACGCAGGCCGGCCTTAGACACGTTTACTC
TAGGGAGTCATTTTGCTGAGGACAGTGAGCACAGCACTATGTAGGTTA
GATGGGGGGCGTGGTGGGAGCTTGGTGGTCCGTTGGATTTGAAGTTGC
CAGAGGACAACGATGAAAGTAATGGCCAAGGATCAGTGCGAATAAAAC
TCATCCTTGCACTTACATACACACACATACGGTCCTGTGTTGGATTTC
GCAGGACATTGCGAAATGTCTTCGGTGGAGGTTTTACTGGCCACGTTT
GATGACCTTCGGCATTGCTGCCCTGGCTGTCGGTTTCGGTTGCCCGGT
TCCACATTTCCGGTGGCTGGCTGGAGATAATGAACATCAATTTCAAGA
ACGGCAATAATCGTAAAATGCAGGGAAATATTTCTTGATGCATTCCCG
GGCTGGATCTTGAAGAACGCGCCGCACATTGGAGTTGATTTGAGCATG
GGAAAACTCGGAGCGCCGCCCGTGCCAGTACGGCTGTCCTCCGCTCCG
CGTTGTTACAGATCCTGGCAGTTCATACATTTTCATCGAACCAACCAG
AAGCATCAAGCCATTCAGCCACCACCACGTACCACGAGATGGATGCAA
AGGAAGGACAAAAACAAATGTAAAGTCGCCCAGAACAATGTGCACTGC
TCGCGCGAGTCCTGCTTTTCGTCTCCGGTGCGTCTGCTGCCTGCGTCT
TGCCGAGGTCGGGAGGAAGCCAGCACACACACAGAGTCTTATGCCAGT
GATGATGCACCACAATCAATCCCTTCTATGCAGACCGAGGGGATCAAT
CTAGGTTGGTTTCATTTTTTGTTTCTCTCTCCCCCTTCATACTCGTTT
TATGATTAGAGAGCTTTTCCGCTGCTTTTCGTTGTGCGCCGTGCTGTA
TTTTGTCATGCTTTTGTTCGACGTTCCCTTGTCACTGGACCGCTTTTT
TTCTTTCCTCCTTCCTTCCGCTTGTTTCCCGTGGCAGGTTGTTTTTGT
TTTCGAACGACTCGGATTTGCCATGTATAGATGCGCTCAGCTTTTACA
AAAAAAGACAAATAAAACACGAACATACGAGCTAAAAACAATGCTTTT
GATGCACAACAATCACAACTACCAGCGCTCACACACACACAGAGACAC
TCTCTGACGCACATTTGTCGCTTACGCAAAGGGAAGGAAAGAAAATGC
TCGAATGCTGCTGCAGCTGCTGCCTGGGAAAAGAAATTGGATGGTCGT
AAATTTCGGGTTCGGTAGAAGGAAAGCTCTTCCTTGTTTCATTTACAG
TGTAACAGTCGCACACGTTGGCACCACGCTGCCATGGTGGTGGCGTGT
GGATCGAAAATTGAGATGAGGTTTGGAATTTTTCGCTACATAAACTTT
ATCCTGTGCTGGTGTGGACTGTTTGTTTCTGTTGCCCAGTTTTATGAC
GTCCCGGAAACGCGGACAAGCGAACCGTGCGACCGGCTAATTGGTCTC
ATCCGCCTCGTGATTTTTCCGACCAACCGGCTGCAATACAATTTGTCC
AACCATCGTGTTCCGCCGGTGGCTGCTGGGATAAGCAGAAGAACATAA
ATCTGATTGAATGCCATTTCAATGCAACAAATTTTAGGAAAAATGGCT
AAACAACTCCTTGGCAAGCTTCTGGCCAAGAGTAAAGGTAAACAACTT
GCCAGTACTGGTCACTCTTTTGTCCACCCACCTTTCCGGTTGTATGTG
GATTGATGCATTTTAAGCATAATACATTATTAACTCCACAGACAAACA
ACCCCGAAATGGCTTCAGCTCAGCTTAACCAGGCGGCAAACTGATTTC
GATCCGCACGACATCATCTTGCACGGGACGAGAAATTGCCTCCGATAC
CTCCAGCGCGGCGTCAGTCAGCCATCTCTCATATTTGCTCTCTTACAA
ATGATCTCAGCATTGCCTCAGTCGGGCCCTCAGTCGCGCAGCTCGACG
GACAGAAAAGTGGCGATGTGAAATATTAATGTTAAAGAATTCATTTTT
AAATATGCAAATTTTAATTAATATTCACCCTCGTTCCCTTGTGGGGCA
AAAACGCGGGCCTCGGGCAACGAGACTCTGCAGGCTGGTAGCAAGGTT
TCGGTCATCTGTAAATGTGTTCTCGTTAGGCGGTTGCGAAAAACAGGC
CGATTTTGTTTCAGGACAGAACAGGAGGGATAAACATATAAAGAGAGA
GAAGGGTTAATGTAGAAACACAATATGAAGTTATTAGTGTTATTGCTT
TCGACCGATGGCAGTAGATGCCCGGTGGATGCATCAAATCATGACTTC
GACAGGCCCAATGTCCAGCGACAGGGGTGCATTAAAACAGGCTTGATT
CTGGATCCTTTAACTACACATACAGGGTCGGCCAGATCCTGAAAGGCC
TCTACAGACAAGGGCATAAAATATGTATCACGCACGAACGATGTTATT
GAACTCATTTCCTTTTCACAAGGTCAATTTAGTCCAAAGCTGGCATCT
AGAAATCTGATCTCCAGCCCTGATTGATGCAGGCTAGCAGCAAAAGAA
ATTGTTTTCCCGGAATCATTCCTCCGATTAACCATCGTGTGGCATGTA
AATTCCCCACTGTCAATGCTGTTTGAATAATAGCCCCGGTGATATCTC
ATTCCCGCAGGGCGGACAGGCACGATGGCACTATGGTGAAAGCCTTTT
TTTCTTCTCACGTTCTCACGCGATCCTGTTGCATAAAGAAGTGCACTA
ATGAGTGGTGGCTGCGCACATGTTTGCGTTCGGGACGCCGCAGTAAGT
CCTCGTTTTGCAGTTACTTCCAGCTCGTAGGGCCAGTAGCGCTGCTTA
GTCCTTCACGGATTGCGCTCGATGATATAATGCATCACCTGCCCTGTC
CTGCCATGTTGGTTGTTGTTGCTGCGACCGGGACGGATCAACGAGCGG
TAAAATTACTGCACAGTGGCGGCGGTTTCATGCTCGCAAAGGCGAATG
CACAGGATTGTGTGCAATTGTGCGACGATTGCGTGCAGGAAGAGCAGG
AGCTGAAAGTGCGCAGGGGGACAGGCCGCGCTCGACCAAAGTAATAGC
GGGGGTGTATGTTTTCCCTGGTGAATGTGCGGTCCCACAGCGTTACTA
CTTCATTCCACTTGACGGAAGCTAATGAGCAGAATCAGGTTGGCTGGG
TGCATAAGAGCGAAAATCACAAAAGCCGTACACAAAAACACACAAACA
GCGATGGGCTCGGAACGGGTTAAAAAAGAAAGAAAAAAGACAGAACAG
CTCCAGGATCCTTTCACGTGTACACGCAAAACAACTGCAGAAAAGCAA
CAAAAAAAAATGCTCCTATTTTCCGGTGTGCCGAGTTACCGCGTCGGA
GTCATCGTGCAGCTCGATGTCTGTGTGTGTGTGAACGGTCTCGCAGTA
ACGGAACAAAAAATGTCAACGAGAGCTCTCCAGCAGAAAGGAAACCGG
AAAATTCTCCATCGATATAGCAACAGCTCCACTTCGGCGCACAGTCCC
TACCTACCTTCCCCTCACTATTGCCCCAACCCATTGGGCGGCGGTGGT
AAATCGGAACGGGGCATACATCAGCGTCAAGTTCAAGGACAATTGTCA
ACGCTTCCGTCCACAACGATCCGCCACCCACACGTCTTGGGGTGGATG
GGGCGGTCGGGGAAAAAAATAGAAGCAACCGACGCGCACCACCCCCTG
GAAGCTCGCGGAAAAGTGTGCTAGGAGAGAGAGAGGGAGGCAGAGAAA
GAGAGATGGAGAGACGGAAGGGAGTCTCGGAAAAGTGTCTCGGATGTG
GGAAATCGGTTTACACCGTTAACCGATGCCAGCCAGATGGGCCATGTG
GGGCCGATGCCGTTCGATGTGTGCGTGCACAGCGTGTTTGTCATCGTT
GCGTTGTCGACGTCGTCGTCGACGTTCGTGCCGGCTCACCCATACACA
GGCCGCACCGAAGCAAGCAGTTGGGAAAACATGTGGCTACGACGATTC
GTGCCGGGTTTTTCCTCGTGCACTGCAACACAGCCCTCCCCCTTGTTT
CCCTGTCCTGCGTTGAGTCGCATGGCGCACGAAGCTGTTTGTTTGGGT
ACGAGCCGTTGTTATGACGCGGCACGGCAAACGCGTTTTCCACTCCGG
GGGCCGGGGCGCTGTGTGTGTGTATGTATGTGCGCGGGGTTAGGTTAC
GTTTCCGCGCGCGCGATTCGGCCTGACGCTGTTCAGCCAGTGGCCGCA
ACATTGTTGCTAACCGGGCTGATTTTGTGGCCGAAAGGGTAGGTGGGA
TGGGAGGGAAGGGTGCAATGTGCAGACGGGCTAAAGGATTTGGCGAGA
CAAGGAAGGAGTCGAGAGAGAGACGTGTCCTTGGTGTGTGGTGCAGGT
CGCGCTGTGTAGGTTGAGCCGTCTCGTGTACGGTTGACTGTGTAAGTA
AGTGGAAAGTTCTCTCTTTCTCACTTTTTCTCTTTCTTTCTGTTTCTC
TCTCTCTCTCTCTCTCTCTCTCTCTTTCTATCGGTTGAAAATTATCTC
GCGCCACCCGCATACACTTGTCACGGGGGAGTGTGGGGCAGTGAAAAT
GCATACCGGCGAAAGGAGGGGAAAACCTCGGCCAAGAAAGGGAGGCCA
GTTTTTCTCTCAGCTGTTGGTTCTGTCGACTCGGCTGCACACAGCGAA
AGGATGTGTGTTGTATGCCGCCGCACACAAAGCCAAGCGTACCGACAC
GGAACACACGGGCGTTTGTGCATGTGGGTGAGCGCTTTGGACGCATGC
GATGTGGAAAATCGGTGAAAATGCAAGATTGTTGCTGAGTGCAGGCCC
GAAAGTCAGTCGTGGCGCTTCTCGCGTACCCGAAGGACGCAAAAGGCC
CGCCCGGTTTGTTGCTGTTCAGAGCAAGCGGGAAAGGCAAGATATCGT
ATGACACTTAGACGAGATTGAGTTAGGGCATGGCGCTGGGGTGTAACA
GCGGCACCAGACAATAATGCTCGTAGGTATCGCATTAATGCTGCTTGT
TTACTTGGGTTTGAGTGCTTGAAGAGGTGTAGCAGGTTTTTGTTTCAA
CTTTTATCACTCTTATTCGTAAATAAGAATTATTAAAATGTAATGTTA
GGTATTTCTGTTGAACAAAACGGTTTTATAACATACAGAAGCAATTAA
TGCATTGAAATAGTCTTATAGAAAGCAAAACTTCAACGAGGAAACACA
TTTTGGATGTTTCAGAAAAAACATACCATCAACAACTGTAGAGCTTTT
CAGAAAGAGTAAAGTTCCTGCCCAGTTTTGATTGGCCCCGTTATCAAA
AAAGTGAAACAAAAACCTTGAAAGCAGCTTGTTTGTTCGTTTGTCCCT
AATTTATGTTCTTTCCTTGCTTTCGATGATGCGATGGCACGATTTTGG
CTTGCTTTAATGATGCGTTCTGATTAAGGACCGATTAGACGTTTTTTT
TCTTCCTTTTCTCCTCGCTCGCCAGCTTCCTCTAGATTCGCAGAGCAT
CGGTGCGAGACACAACCAACGTTAGCGTTGATAAATAACAAACTCCAA
GGGGGTTGTTGTTGTTATGCGTTCCTTTTTTGCCACAATCTCCAAATG
ATAGCGTAAACCTGCAACTATGGCACATCATAACGTCCCGCTTGAGAG
AGAAAATAGGCAAATTAAAATGCGAATGGGCCATTTTTGCTTTCGTTC
ATTCTGCTACCGATCGGTACGATTTTAGTGTTCACACACACACACACA
CTTCTTGATGATCGCTTCATTCATCGGGGCAACAGAGGGGTGGCCGGA
ATGGTGTTATAACGTATAATTTGTGCTAATGGTTATGGGGTGGCTTTA
TTTATCATTACCCTAACAAATTGATAGATTCCGTTGACTGGCTCACAC
TTTGCTGCGGCCCTGTGAGACCTTTGCTTTGATCAGTCGGCGGCAGTG
TGTTCTGGGTGCGATAGGTTCCAGTTGTTGCCTCCACAAACCGATCAT
TCGTCGATCGTTGATCGCGCATCCCAGGTACATAACTCATCCAATTGC
GAAGCCCCAGCGTGTGGTGATGAAGGAAGTGGCGCAGTCGCCGCTGTT
ACGACCTCTTCTGCTAGCATCGGGCCACGGCACCGGGTGGCACTGGGG
GCTCAACGACGTTTGCCTCATCCGGTGTCCGGCTGTTTGGCTGCCAAA
CCCGCGAGCAAACATAAGCAGACAAACAAAACGCGCACCGCTCGGTCC
CCCTCCCAGCCAGGCCAGGTTCACACACAATAAGCCGGCACCGCGCGT
GCGGCCGAATGCCGCAACTGTTGAATGCATGTCGTAAAATAAAAATTT
ATGATTGTAATTATCATCTCTTCTCTCGCACCCACCGGCTCCGAGCGA
GGATGGGAGGGATGTGGCGAACGCGGCACCGAGCTGGAGCAAATCTTC
GCACACCCGTCTGCATCCCATTTTCTTCGGATCTCACCACATCTCTCG
AGCGCTGGTGCAACCGGAGATTTAAAGACAAAAGGCAAACCATACACA
GACACACAGGAAAAGGAAATCAGTTCGCTTGGGGTAGCTCTTTTTCGC
GGTTTGCAGCACAATGATAATGGGTTATGTATGTGCTTGTGTTAGCCC
TGTTCTTGCTCCCACCTTTCTCTAGCCGTAACGCCACAATGCCAGTAA
GCTTAACTTATCCCCCGGTTGCTGTCTGTGTTGGATTTATTACCGGTG
GCAAGTAAGTTGCAGCCCATTGCTGCGGTGCGCGCGGTGCGTTATGGC
AATGATTTCGCATCTTTTCATCAAGTGGTGTGAGCGGCGGGCCGTCTT
GGACACGCAGAAAAGGTCTTATCTTGTGACTGGCCGTGTGTATGTGTG
TGGTTCTGCGCTTAAAGATATAATTTGTGGCACGCTTTATCGCGACCC
GTACGACATTGTTTCAGCAGCGTTGCAGCAGCACGCGCCCCATCGGAA
AGAACGGCTTGATGGACGGCAGGCGAGGTAAATAAAAGATATAAACGC
CGCCCGCCATGTCCAGTTTAATCAGCTGTGTCCTCTGGAACAGTTTTC
CGGTGGTTTGGATGAGGTTGCATCGTTACTAAGTGCATTGGTGTTACG
CATGCGCGAAGAACAATTCCGTGACCTTGTCGTGCGCAAGCATTCAAA
AGCGAGAAAAGCAGCTTTCTGTTCAGTTAGCTGATGATTTCTTGAAAC
GCTTTCTTCTTTTTGACGGGTTCTTTCTCTTGGAAGATGGTGAACCTT
ATTTTTCATTGGTGTTATTAGATGTCATGTAACCATGAAGTACATTCT
TGCCTAAGATATTACGTCATTCGTAAATATTTATTAGACATTGTAGAA
CTTCTGCTCAGATGATTTATTCACGCAACACGGAAATTTACAAATCTT
TTCCACACTTGTTAAAGTGCTTGAGTAGTTAAGTGAAAGAGAACAAAT
AAAACCCAGCTGTGGAGCACAACAGCCCAAACGAACAGGGCATCCTTT
AGACATCATTATGGGTCGGTTCTGCAGGGCTGTCTGCAATCATAATGA
TCGGTTGGAGGTTGGAGCTCCAAAACGCAATCAGTCCATACGCGCGGT
GCAAGACGTGTGTCCCGGTGCTGGTGAGGTAAAGCCATTCCGGCCGAC
TATCAGTCAACGCAGCAAGCAGACAGGACGAGGGGACACGCTGGATGG
ATGCCTCCAGAGTGTGATGTTCTTTGGTGGGGTCGGCGGGTATGTTGT
GGTAGCATCAAATCGAGCAAATCGAGATGGATAATTTTCGATTATTAC
CGGGTACCGAGGCAAACCGAGGGAAATGATATTGTTTTCTCGAGTTGT
ACGTTTTTATTCGCCGTGTTTTATTTTTCGCCATCCCTCCTGGTACCC
GTTGCTGTCACCGTCCTTTCAAAACTGGAAGGACCCACCAAAGTCGTC
GGTAAGCATTCACATGCAGCCAGGCTCGCTTGCATCTTTCCGCTATAT
CAACCTGGTAATTGCATAGTGTGAGTATGGTGGTGGTGCTGGTGGTGG
TGGCCAAGCCAAAGGGAAAGGGGAGGAAATACGGAGAAAAGCAGGAAC
ACCAACATCCAAATGCGCTTTGCGCTTGCAGGCATTTCGCGCAGCATT
AAGCGAAGCCGACAGACCACGGCCAGCCTGTGCACGGATCGCACGGAT
TGGGCACGGGAAGGGCACGGGGAGAAGAGACATGATTGCTTCACGCCA
CCACGGGCTCTCGGTCCGTGTACCAGACGCCCCGGACGTATCGGAATG
CGGGCTCTGGGCGTGGCTCACCCGGGGAAAAGCTGATAACTTTATGAT
GTGTCGAAGATGAGAAAATCATGACTGTTGTATTTTTATGTGTTTTTA
AATAATACAATTGACGTTATGTTAACGGGCGGTTAGGCTGCCGGTTGG
AGGAAAACGAATAATCGAGTACAGTCCCCCTGTACACGCAGCACAGGG
CAAATGCGAATGTGGCTTTGGAGCGAATATGCGGTTGCGGTTTGCACA
TTGTTGTTTGGTTTGGTGAATTAGTTCGGCTTCAAGGTCTGGCTTTTG
TTTAAGTTAATGTCGTATTTTGAGAGTTTGCATGATAGTTTTTGCATC
CTGTTAAGAACCTTCGCCCGCCGATGTCAATTAATAATGGCAGCTTTA
AAAATGTGCTGCACGTTAGCTCAATCATGCTATTTGTTGTGCGTGTGT
GTGCTTGGCGCGTTGCAGAATGTATTTGCGGTAACTAGAGTACAATGC
TGCATCTGCACTGACCTAGTCGTAGAGCTGCCCTTCTCCAGGCCTTGC
GCACACATGCTATAACACCTACACCACTGAGTACCAACTGAGCGCTTC
TTTATAAATGGGAAGTCATTTCGATTCATTGATTGAATGGATGAGTGA
CGTGAAATAATTGCATTCATTGCAGCTCTCGCAGTAGCAATCTGCGCC
ACCAGGAACCGACCGGGTGGGACCTAGCTCAATGGCTCAATGTCATCA
CAGTTGCGTGAATATCAAATTGCACACGGTTTCCCTTCCAGATATATA
TTCCTATAACAACACGGTGCCCCGCGGTCCTTTTACGGAGGCACGATG
TACGCAAACTGCTCGTTTGGGCAGTTCCAAAAATACGCATTTTTCGAC
GCAATGACGATATAATCCAAAGTTTGTTGGGAGCGCACGGGGTGAAAG
GCGATTTGAGTATTCTACTGCACCGTAGCGTTTCGTTTTGTAGCCAAT
TTTCCAGTCGATACTGGCGCAACAAACGCAACGGCATCAAAGCGCGTG
TCTTGTACCCACTTATTTTCTACGTCAATACGTGCTGCGAATCCGTTG
TCAAAAACACGCGTACTACTACGCCTCCAAAGGATCTGCTTAAGGAAC
GGCTTCCGTGCGAAGTCGGCACTGCTTCTTGGATGGTTTCTTTCGAGG
CAAAGGCTCTGGTTCTGGCATGGGGGTCGAAGGTGGTTGAAGAAAGTT
GCACGGCTATTTGTTTCAAACATGCCCTAGATAGAAGAGAGGCTCTGG
AAGTTCTCGAAGAAGTATGCTTATGCAGATGTTTTACCTTTTTTTCGT
TCCATTGCTACCTGTCTTAAACAGCTACCAATAGTGCACCAATAGTGC
TTTGGTGCATACGAGAACGTTTTTAAACGTGCACTGACGGGGATAACT
GATGGAGATATAACCAGGCTCAAGGATCAAAAACAACTTGATAGTCCA
GAGTTTAGCGTATTGTAGCAGAATCTTGAAGCATATTGCCAATCAACT
CTGTACTTGCGCTCTGAGAAGATGACCTGGTGATGGACAAGAACTCTT
TCTTTTTCTCTTTCGCAACTCACATTCACTCATAATTTGCTTCACAAA
AGAATATGGAATTGATCTGTTTTGATTGAGTGTATTCATATCTTTCCT
AATTTCAATCTACTGACTCTCATCTGTTGCTTTATAACGGAAGCGGAA
GAAAATGATCGATTCTTCTAGCATTAAACGAGCATCGGCATATCGGTC
CAGAGAAACGCCAAAGACAAAAGACGAAAACAGACACAAACAACACTC
AAAACGACCGGGGAAGTACGATCGACAAGGGGCGAAGATACGGGATAC
GGTGTACGACGAGTTCCCAACATCATTATCATCATTACTGAAGTGATC
GCGTCATTTATGATCTGCTAAAGTTATGACCAAGGCGATCGAAAGCAA
AAAAAAACGAAAAATCCGGTGGTTTGGGCGTAGCCGTGCTCCCGAACG
ACCTCGAGAAATGCATAAATTGGACGATGTCCAAACTCACGAGCAGAT
CACTGGGGGCCATCTCACGGTGTGCTCGATACCGGTGTTCCCTGTCCG
AAGCGAAGACACGGGCGAAAGGGAAAGCACAAGCTGCCGGTAGATAAT
GAAGCTGAACAGGCAATGGGGGCCGATGAAGAGCTCGCGTACCGAAGA
GATTGCAACTAAGGAAAACAATTCTGAAGATTGATCGTGTGACGAACA
CAACTTGGGGCGCTCACTCGTACGGAAGAGCAAAAAAAAAACGGTTAG
GCGAAGCGAACGAAACTATGAAGGTACCACTTGAGGCCACTCGGTGGT
GCATCAGTCCCTCCTTCCCCTCGGGGCGAAGGGAACCATTTGGATGGC
GGCTGGAGAGGACCGTTTCAAATCGCCACAAATCGATCAACGACTGTC
GAAGAATCGTCGCGTCGTGTGGACGGAGGTACAGGGGTGGTGTGTGTG
GTGTATGGTACGACCATTGTCTCACCTGAGCGCAGCAGCTCAGCTCAG
TTGGCTGTTGTTCGGGGTGTTGCCAGCCGCTGCAGAGGCAACTGTAGG
CGCACTGTCTGGCGGCGGTACAGGCAGCTTCTTTAAAAATTGATTTCA
ACCGCGAATTGCGGCTCGAGGGGGCCGCTGGCGAGCCGGCGATGCGCA
AAACAAAGGCTCACTGAGAGGGATCCAATAAAATCGACAAATGAACGA
TCTTTCTCTCGGCTCGTGGGTTTTTTGTTGTTGTGGTTGATGTTGTAG
TGCCTTCTTTAGCAATCTTCGTGTGAAGGCTGTTCGCTTAAGTCACGG
CGATGGTCAATGATGCACTGCACACTCAACCGTAATCATCTTCGTCAT
CGTTTCGCCCTCCACAGAACGGAACGGGTCCTTCCCAAGAGGGGGGAT
AGGACCGGTAGTGGCAGTGCATCCACTATTAATGCAGAATCAATCAAC
GGTGGGGGTCGAGATCGAAACACACGGCTATCGCGTCTGGATTGGGTG
CGATCGGGCCGATAGGCCGGCTCTAGGGACCGCTGGCTACATCGTCCT
ATTGAGCTGTCTGGATGCATTGTGTGAATTATATAATTAATTTCCTTT
GCGCCCTCCCACCGGTCGAGCGTCACTGAGAGCAGCGTGTGTGAACGA
TCCTTGGTGCATCGCACGATTATGACTATTGTCCTCGGGCGAGAACAA
GGGTGTGCTGCGCCTGGATCTACCTTGGGCGTGAAGGAGGAGGTTCTT
ATGTGTGTGCTAATCTGTCGGTCGAATATTTGCCACAATAGTCGGCAA
CAGCAGCAGCAGTAGCAGCCGTGACGAATAGGCGCCTGACGGGGTGCT
TTTGGTGTCGCTTTTTGCGAGTCAGTTGTTTTGCCTCATCATTCTCAA
TGTCTCAATGGCTTCGATGCGGCCAACATCAAAAGGGTTTGATGGCAG
CATCTTCACAGCGTCTTCGTTTACTGCATTCGGATTGAAGGTGACCTA
TTTTTTAATTATTTATGGTATTTCATCCAAATGTGATTTTTGAAGCTG
ATTCTTGTTTGTGTTCTTTGTGTATCTGCATGGATGTTTTGTGCGGAT
GGATGTGTTTGATGTGTTGAAATTATTTCACATTTATTGCTGTAACCT
TTCACCGTTCACCGTGACGATTGCATATCTTTTTTTGTGCAAATAATG
TATCCGTAATATCAAAAACATTATTAGAAAAAGAAGTGTTGTAAGGAA
ACATACTAACCAATAGCTTTGAATTAGTCTGAGAAATAAAATAGTCTA
AAAATAAAAATAAAATATTGCACAAACAATTTGTATAGCTATAGGCTT
AGTCTGTCCTTGCTTTAAAGACTACCCCAAGGGTTGATATTCGTAGCA
TAAATTATGTATGAGAGTTATTGATTGACTTAAAATCGCTCACCTGCC
TGTGGCCGTGGCTGTGGTAGTATCGACCGCAGCCAACATGCAATGTCC
CAGGTGTAACGACACAATTGCATACAATATAGAAGAACCAGACACTGG
CTGGCCGGCTCGGGACTGCAAATGAAAGGCAAAATCGAATAACGAAGA
ATCCTTCTAATTTCAACCCCCGTCCTGTTCCTCGTGGCCCCGTGGGGT
CATGGGGTGACAGCTGTGTGTAAACCTCCCGGAGAAAAGTAAGGAAAA
ACGAGTGAGTGAGAAAAAAAAAGAAAAAACAATCCCAGGAAAAAAATA
AAATCCCCGTCAAACGATGGTGTCCGTTGTTGCTGTTGCAGAAGGTTC
GAAAAATAGACACCAGAGCGTTTATTGCCTGCCGGTGGCTTTGCAAAT
GGATAGGATTAAGTGTTGTGCAGGTTAGCCGTATGCAACTGATTCGTA
CTGAATCGATTTACAGTGGAGCAGCAGCAGCAGCAGTACCAAACAGGC
AAGACCATTCCTGCTAGATACACCCTGTTGCTGCAGTTTCGAGGCCAG
GCTTGACGCTAGCTATCTCTCGCTGTAAGCTGTCGGGCTGTTAAACGC
TCGTGTTACCGTTTGCGATGCATTAATTAACGAAGTGAGGGCGAGCAG
ACGGCTGACGGGGCAGGGACCGGCAATAGCGGAGCTGTGAAAATCATT
GACATTGGTAAATTTGCATATATTGTTCGCGATAAAAGAAATGATTAA
GAAATGTGGAGTGGGCCGGGTGGCCGGTTTGGGTGGCTGTTACGATAA
GCGTTTAACGTCGCATTAATTAGTCAGAGGGTATCCGAGCCCAAGTCG
ATCATTTCGTGCTGCCCTGGTCACGGTTATGATGCGGTTTGACGTTCA
ACTGTTTGAAGACGACGCGCGTTGTGACTTTCGCTGATAACGCCGTCT
TAATCGTGCTCAATCACATCGCAAAACTGCCGCGGTGTATGTGCGTTT
CTAAGCGGTGCAACGGTGGGTGGCATTGAATTCCTCCCAGGCCCAGGC
ATTGTGACGCGCACTGCACACTAATCTTATCGCCTTTGATACACGGGT
GTCCTCTATTCTGGTCACTCGCCACTCCGGGGGTAGCCTTTCAGTTTT
TGCCAACCCGCTTCAATTCCTCCGGTCTCAACACCCTCCCTTGCACAT
AGACGTGCTTGTTCATTAGTGTTCCTCTTCACCCTGGTGGTGCCATGA
ACGCACAACTCTTCCGCAAGCGCATCGTCGTCTGTGGATGAGTGTGGG
TTGTGTGGTTTACATTGTACTCATGGTGTTTGAGTTTGCTTTTTTTGT
TCTTCCTTTGCTTGCGTTGTGCAATACTGCTACGAATGTCAGATTTCT
AGTCGTACTCGATTTTGGCCGCAAACACACATACGCGCTGCTCTAACG
CCATGGTCTGGTAGGTCCGAGTGCAATTGTGTTATCAGCTGGCGATTT
TTGCCCTGCATTTTCTTTGCCGCGAGTGACCTCGACTTGGGATTTGCT
ATGTAAACATAACGTGTACGTGTAGCTCGTGCCTGGAATAGATTGCCT
CCCCATACAGCCAGTGACACGCACACACACACACACACACAGACGCGT
GGCACGGCTGTGTTTATGTTGCAAAGATTAGTTTGTGTTGGTGCAGTC
CCCGTTCGCTCAAAGCAATGCAAAGCAGCAGCAGCGACGGCACCCCGG
AACACATTGGCTGGTGACTTTGGTTTTGTGCCCCGTCCCCGTGCATGC
CACCCGGAAATCTAGCCGCCAACGGTGACTAGGTGTATTGATGAATTT
AAATTTTGCACTACAAAAATGCGCTTTGCTTTTTAAATGGTACATGTG
CAGGCGACTGGTTGCTCTCCTTTCCTTCATTGCTGCATTGCCGCTTTT
TCCCAATCACATGCTGGATTTGGTTGTCTTACCCCTCCCTCGCACACA
CACGCTCGCTCGCTGCATCACTAAAGAGCATGCGAAATAACGATAAGT
GACAGTTGAATGTTCAGCTGTTTGCTGCTACCCGGGGTTTCGTAAAGC
CATCTTCCACCGTGCCCGACCCTTGTTGGCGATAAACGCGCGCTCGCG
AAAAATAAAATCAAATACGCCAACTGGAAGAGCAGTTCGGCTGTACAA
CACAACACACACACACTCACAAACCTAGCCGCACTAAACAGAGCGCAG
ACAGCGACGGCGACAAGCGGCCAAAGACGACAACTACCCTATCCCAAC
CCCGCGACTGACAAGTCTCGGGCTCTTGCGTTCCGCTTCTAATTAAGC
GCGGAGGCCCACCTTCAGCGTACAGCGACGACGGTGGCAGTCCTTCGT
ACTCGTTTTTTTCCTTCCTGTGCTGTGCCCTACTATGTGGTAGCACTA
TGTGGCACTGTTGCGAAGGAGCAGTATAGCAACCACCCACGCCAACAC
CCCACCGGGCCGACGGGAGCTAAAAGTCTGACAAGTTCAGGCAGCTCG
CACGGGAGTCGGGAATCGATTGTATCGATAGCAGCCCAAGCGTCCCCA
ATAATCGACGTTAAATTGTTTCCCCCGTTCGCGTTGGATTGTTACCAT
TTGCGTAGTTACACTGCTTAATTTTTAGGCGTAATAGTACCGCATCAC
AGTGTCGTAAACTATCGGTACGTTTTGACATGCAGCGCGTTGAAACGG
CACAGGCAGGAGAGCAGCCAAAACGAACGGGAACGCATAAAATTGGGT
TAGCTGCGGTGGAGGCGTCACGGTAACGAGCTGGAAGCTGGCGTAAAG
CGTAGATGAAGCTGCACAGACAGACAGACCACGTCCACACGAACGGAC
TGGGAAGCGGGAGAATGCACGTTGCAATCTTTGAATCTGATTTGCACG
CAGATCGATGCAAAAATGTTGCATGTCAAGCGTTAATAAAGATTGGTG
TTTACGAGTGTTCGTTTTGGCTGACACCGGCCGGCAGCGGGTGAAACA
TGCGACATCATACCTGGCGGTACTTGGAGCGGAGAGTTGGAGCTGTGC
CAGCAAAGGTGTCAAACGTGCAGCTTATCGAAAGGGTAATGAGGCATT
TACTTGCTCTGTCGCAAGACAATTACTCAAGAATAGAATAAATACAAC
AACCAAAAAAGCCCGCACCAATTTGTAAGGATTCATTCCAGCTCTCCC
CTCGCAGGGTAATGTGTGTAACAATACGAAGTGTGACAGACACTTCGG
GGGAAGTTTTTGACAGCTCCTGGGAATGGCAACCCTTGCGGCTGCACT
GCTGCACACTCGACAGGGGTTTTACACGTGCATGCGCGACTGGTCACT
CCGTAGCACACGGTAAACAATGTTGTAACTGCAACTCGCCCCTTAAGA
ATCCTTTCGCCCCTCAATTTGTAGGCAAGTTTCCGTCTCTTTGCACAC
ACGCTGAAGGAACAGAACGTCGTCCTATGATTATGCTGTCAGGGAGAG
GAAGAAACAGTACGCAGAGCCACGCCGGGGCACAATTCATTCGATCGG
GACCGGGAGGAAAAGCGTCCTCGTGCACATTTGCACCTCAATAGCGAG
CATAATTTAGTCAAATTAAGCGTACTCCGCTGGGAGTGGACGACGTAG
GTCGTCGGTGGTGGCATTGTCCGAGAGGACTGGTGCCACGGTTGCTCA
ATTGTAACAATCGTTGACCTAGGTCGGTGGTGATGTGTGTGGCCATTG
TTTCAACATTCCACTAGCTTCGGGTCCTCCTAAAATCCACTCCCCGGA
CGGATAGGGCGAACGCAAGTCACGGGCAGCGACTGCTCTGTGGCGAGG
TGTTTGTGTGTTGCAAACTTTTGAACCGAAAACTGCTACGACCACCAC
TACTTCGCTGCTGTTTTGAACCAGGAGCTCTGCATCTCCTCGACTAAC
TGACAAAAAAGACCGCATCCGCTCACATTGTTTCTATTTCTGCAGGGA
CAGAGAGGTGGTCTAGTGGTGCCAAAGTTGCCCACGGTGGCCGAATTC
GAGGCCCTACATCCTCCAACTAATAGCAGTGCCAGCGCCTGCTAGATC
CTGCTACTAGCACAAGTGTGTGTGTGTGTGTGGGTGGGAAGTTCAATG
TTGAAATGTTTCACCGATATTTATCCCGACACTGACCCCTTGGATGAG
CCAGCGTTTTGGTGCCATTTCTGGCTGTGTTTTCGCTCAAACCAACCA
GTTCGACAATAACCAGTGATGTTGATATATTCACGTGTGTGTGTGTAT
GTGAACTTTATTTTTCTCGCGTTTTCCCGCTGGAATGTGCATGACATG
TCGCCGCAACTGTCGACACAGATTCGCTCTAGTGGAAGTGCATCGTCG
CGCATTCGCTGCTGCGCGGGCTATCGCGGGTATCTAGACATACGTGTG
TGGCTAGTGTAGGCCAGGGAGTACCATCACCACAGGAAGGAAGTGGTT
CGAGAGGGCGAATGCGCGCCACGGCGTTCCAAAACACAAAAAGCGGTT
TGGATCCAAACTTTACTGCATGTTTTCCACCGGCAGTCCTGCAGACGA
TGGATCCACATGGACACTGGAGGGAACAGCACAGGGTCAGCGTCAGCA
GTAACTGGTCAACGCTGCGTTGCGTTCTAATGTGGGGCTTCCGCTTGT
CTAGAGCCTTCCGCGGAGTGAGTGTGTGTGTGTGTCTGGCTGTCCTGA
AAATTGGATTCAGAGCGGATGTTGACTGTTTCGCGTGTGTGTGTGTGT
GTTTGTCCAGCCGTGGATTGTTGGGAGAATATGTGCTCATCCATCCAT
GCGGCAAGTCGCTCACGGGGTGGAGGTCGCAGCACCGAGAGTTTGTTT
GGCATTAAGTACCTTCAGTTGCAAAGGCAATGCAAAGAAGAATCATTT
ATCAAACCTAACCATCTTCGCTCAAGGGTTTGATATTACCCTCGGAGA
ACCACTTTGACTCATGATCCGGCGTTGAGCATTTTTCTAGTTTCACAC
ATTGCAGTAATTGTCATTAGCACTTAAGATTGAAAGCCCGGAATGCTT
TACGGCATTGGCCCGTAGATCGCAGAAAGGCCGCGAGCAAACCAAAGA
AATGGATGTCTTTATCGCAACGAAACGTCGCAAATTTTGCGCCCTTTT
TTACTGCCCCGCAATAGACACTTGCAACAAGACGGCAGCGAAAGAGTA
AAAAAGCCAGAGAAGGCATTCCGCCAATGCTGTAAAAAGCACCAACAA
CAACAACACCAACAAAAAAAAACTCGAACCAAACGCACACTCATCAGT
AACGCGAGACCAGTGCGACCAGGCACCCATCTCCCTTCGAACGCGCGG
CTACTTTCCCAGCCATAAATCATCCACTTCAACCAGATTGAGTCTCCT
GCCGCCGCACCAGGCGTGACCACACGTCTGGTGCGGTGTCTCGTTTGT
TCCGCCGTTTTTGTTGGCGTGTGGGTGGTGGTGGTGGGGGCGGGGGAG
AAGGTAAATTAATTTACACTTGCACACAGCGCAGCTTCAAGTGGGAGA
TGCACTTGTCGTCTCATTGCCTCGTTGCTGCTCCGGCCTGCATTGCCC
GCCGTGCCAATGACGCAGTGGGGTTTTGGTGACGATCGCTACCTTTAC
CGCGCTTGATATAAGGGTTGAAAATCATCATCATCATCATCATCATCA
TCGGATGCTGATCGGACGGGCCACACTCTTGACGGATCGTCTCCATCT
CGTTGCCGGTCCGCTTTCGCCTAGCCCCCTCGTCGCCTTGCCCGTTAG
CAGTTCGTGAAGAAAATGTGCATAAAATTAGAAATCGAACCCTCCGCA
CACACCCCAGGAGGGAGGGGCGGTATGATTGGGTCCCGTGTATGGGTG
TGATGGTGTGGGGCTCGATGTGAGTGGCAATACATTTGCAATATTAGT
GGTTAGATTCCATTTCCTGCACAGGGAGCAGCGCAGCGGAATGTAGAA
AAACAAAACGCCGGCAAGAAGTGCGGATGCAAACTTGCAATTGTTGGT
TCTGCAGCTCGGGTGCGGGTGTGTGTGAGTGTGTCTGTTTGTTTTCTT
TGCACGCTGCCTGGTGGCCCCAGGGAAGGAGAGGGCGTTGTTATGGGA
GAATGTAAAAGCAAAACAAGCCACCCATCCCCGTTCTATTGCATCTCG
TCTCGTGGTCCAAGACCACTCCCTATCCCTCTCGCCTCTTCCCGCCCT
TAATGTCCCTCTGTAAAGAAAGACGATTTGTTCTCACATTCCTGCTTC
CTCCTTCCCCATGTACCACCATCTCTGTCTGGAGAATCGTGCGCACAC
ACACACACAGCCACAGGATTGTGACAGTACCGTCCCCTGCTGGGAGGT
GAGTGAAAAGAAACACATTTCACGCGTGTGTGTACCCTGTGTAATGTC
ACAGTCGATCACACTCGGGCCCCCGGGTGAAGCCGATTGAATCATAAA
TTGCACTTACGGAAGCACTTGTTCGCACTGGCCTGTCCGGTGGCCACA
ACCGGGTCCGAGCGGTGTCCATGTGTGCCGCATTTTATTTTGCAGCCA
CTTTTACAACTGTGCTGCTCTGCTCCCGCTCCCGCTGCACCGCCAGTT
CGAGAGATCCGAGCGTACGAGAAGTGATGATGCAATCAACCGGACGGG
AGGCAACCCATCGTTAGCTCGCCGCTGGAGCCGATAGAGCCAACGGGG
CCGGGAGGGAAGGATGGAATGTGTAACGCTGCAGCTAAATGGCGCGTG
CACCAACACCAGCTCGCAGCGGCGAGAAAGGCGTAAATTGTGCGGCGC
GTGTATGATTCTTGGCCGGGGCGCGTTCTCCCTTTCCCCCACTGCCAA
TCGTTCTGCCCTTCTGGATCTGGGCGGGCGGCATGTGACTAGCTAATT
TTCCAACTCAGTGGCTGGCCGGCGGTCCGTAAGATGATCACAATCACT
TTGGAACAGTAATGTGGGCACAAACTTTCGTTGGAAGGTTGAGTTTTT
TTTAAATAAATAAAATTGTTAAATTTCCACCACCAATTTCCCCCGTTT
TCACTGTTCCCTAGTTTGAGTTTGAAGGTCAATCAAGAGGAAAAGAAG
AAGCGAATTCCCTGCGCAATCACCCTTCGCGAGAGTCGGAGGAAGGGA
CGCGCAAAGAATCCTATTGATAGAAGCTACTGCAGCTACTACACTACA
CTTGCGTAATTGTTTAACGTGCAGAATGAATCGGTGCACTATGCGGCC
GGGAAGTGGCCGTGTGGTGGGGCAGCTCTCCCCCGTTCCCGCGGCATT
GGGTTACCAGCGTGAGCGTGAGCGCGCGCGCGCGCGCGAAGAATCGAT
GATGCCGTGGAGGTTGTCGCGCGGCGCAAACATTGTGGTGTGTGGTGT
GGCCTGAGACCGGCTGCTAGGGGAAGATAAAATGTAGCTCGGGTTTGG
GTGGCGGCGCGTGCTGGTTTCGTGATCGCGGCTCACCTTCCCAATCGG
ATGGGCGGCGGTTGATGGTCGGGCGGGGAGTAGTATCTGGTGTTCATT
GCTGCAGTTCGGGGCAGAATCTGAAGGCCCAAGCATGGGCGAGGCAAG
TGACGCAGGCGGGTGCCGATGCACCGGTAAGAAGGGCGCGCGAGGCAA
GCTGATAAGAATGTGCCGGCTGCACAGGCTGCAGTTTTCGGTCTTTGT
CTTTGTCGCACGGCATTCTGGAGCAAAAGAAGAAGAAGAAAATGATGA
AAAAGAAGAAAGATGCGTGTGTTGGATGATTGTAGCCGAGGACCGATG
CGATGGTGCGGTTGGTGGTGTTATTGGTCAGCTAATGGTGAGCCGGTT
TGCCACTGTAAAAGGTAATCGCGACTCGAATCGTCGCGAGACTAAATA
TAGAGCACTTCCTGAGTTCATGCCAAGTGGCGGAAAATGGACGGAACT
GCATCGCTTGCCCCTCCCGTACCCTCCTTCCCCTTTCCACCAGCCACA
CACATGCACACTTATACCAACACAGTGGGGTTGAACAGTGCATTGGAC
AAAATGCACGTGTAAAAAATGCAACAGCCCATGAATGTAGTTGTGTGA
TATGGTGCACTCATTGTGTACGTGTGGTTTTTTTTTACAAATTACAGT
GTGTGTGTTTGTGTGTGTTTGTATAAAAAACACTACTTACACAAACGC
GTTTACTCGTGAAGATCAATTCATTGCAACGCGCCGAATGACTCGCGA
CGATTGTGCCGTTTGGGTGGATGATGAAAAGTAAATAACATTCTTTGG
GTAAATAGTTGCAACCCGAAGCTAGTGCCAACTGTGCTGGCTTGCTCC
TTTGCTGGCGTGTTCGGGCCTCGCGTCTCGTCTCCCGTTACACGGACA
CGTAAATGGTAGATGTAAAAATAAAGTTTCGCGTCGGGGTTGTATTGA
ACGGCCGTCTGGGGTGGGGTTTTGAGGGGGGAACGCGGGTATGGCCAG
GATAAAAGGTGGGTGTGTGTGAGAGCTCCGAGGTGAACAATCGGTCGT
GACCACGGCCGGGTGTTGTGCAGCCAGGCTGTGTGCAAACTGCAGCGA
GATGCAGGAAAGGGGTAACCGTTTTCGGCGAGCCTTCTTGTAGTTTCA
GCACCCTCGGTTACCCACTTCTCCTCTCCTAGCTTCACCACACGTCTG
TTGTTGCGGGCGTTCTGTTCTTCTTTCACTGATGTTTAAACGTTTCTT
GAACGATGCGTTTTGCGTACGATTTTTGAGTTTATAACACGTGGTTTT
GCGACATGTTAACATTTACATTGTAATCAGTTGATTGATGTTAATCTT
TTTTATTTATTTGCTCTCCTTTTCAGCTACTCACTCGTGCGTTTCGCC
AGAACCTGTAAATCTCCTACCTGGTAAGTAAATATAATTAAAAAAAGG
AAATAATATATTTCAAAGCGGTACAACGGTGTTGTAGCAAACATTTAG
TGCTTCACACTGTACGTTTGAATATTTGCTAACACGATATGTTACAGC
CGACATTAAAGCATCTTAAACCAACTGAACCCAACATGTAGTTCTTTG
CAAGCAAATAGGACGTCATTTGAAAAATGTGCATTTATAGCTCATACT
TTATGGAATGATGTATGTTCTTGCCCGATGCAATCTGCTATAGACCAC
ATTGCAGGCTGCATGTTATAAATATCGGCTAACACAATGCGTCACCTT
TTTCTCACCTTACCGCGCTCGGACGCTTAAATCTTGTGGGCGTTTGCT
TTCTTTGACCTTATCCTTGTGCGCTAGGCTAAGCGTATTTCTAAGCCA
GTGGACATGAGGTACTACCGGCTTCCCTTTTTCGATATGTAACACAGT
TAACATCACAAGCACACACACACACACACACAGAAATAATGTCGGTAT
GGCAATTGGACAATATTGTTATTTATCGCCACATTCACCAACCGATCG
AAATTGTCCCAAATCGCTTCGAGTACATAATTCTCCTATCTGTCTGCC
GCTGGTGGCATTTGTACGAAAACGTATAAAATGCCCCGTTCTTAAGGC
GACCGCCACACAATTGTGGGCATTGAGCTGAGGGGCGCGCGAGACTCA
TGTTTGTCGCATGCACATCGCGGCGGCGGCGGTGGGAGCAGCGGCTTT
TCGCGCACCTTTGTCGCCCTGTTAAGCATTTTTCTAGACGACAGATAC
CAGCGCAAATACTGTTGCATTATACACCGGGTGTTTAAGCAGGGACCC
GGTGGTGGACATAAGCAGAACGATAAAATATTTGCAAAACCGATGTTT
CTTTGCGCTGATACTCGGCGGATACGAGCGCTGTGTTTGTACAAAGGT
ACAAACACCGAGAGCGTGTCCGCCATGGGAAACTGCCTCAAACATACG
CCCTTCCGTCCCCCTCGCCTCGCCTTTTACCACCGAAAGGGCAAAAAA
GGGTGTTAATCGTTTCGCTGTGCGATGTGATGATTGGAGATCACGAAG
ATCAAACGGGTGCTGGGGTGAAAAGCACGATGCTACTTTTGCGACATA
ATGCGCTCGCTTCGATGTGTTGCGCGTGGACATGTTCGGCATGCATTC
TTCGCATTAAATGCAATACGCGATTATTTTGAAATGAAAATTGATCGC
AAAGAAAATCTCAAACGCTTGATTTTACTTCCAAAAAGAAAGGAGTGC
GCAATGCGAATACGAGAGTGAAAAAGAGAGCGTTATGACAGTGCGCTT
GATGGCTAATTTGCAAACAATTTACATAGGCCGCATCAGAACAGTTCA
TTACGGATCAAAATAAACAATTTACTTTTTGCTCGTATTTGCTTTTTT
TGTTGCTCCCCGGGCGGTTGTTGCGATGACCCGTCAAAGGGGATCAGC
GGTAACAGCGGCGAATTCGGCGCGCTCTCGTGGCCGTATGGAGATAAG
GCGAGCGTAAAGAGTGCGAAGGGGAGGAAGGGACCTCGAACAAGAACA
CGACTACAATCGCACAGTACGAAAACAGGAAGAAACTCGGAGGCCGAT
GTAAAACTGGCCGCCCAGGGTCTGGACAAAACTCTTTATCCAAGCAAG
CACTGGGAATGGGGGAGGAACAAGGGCGCTCCTTTCCTCGGGGCCTTG
CTGGCTGGTGGGCGGCAGGGACCGGGGGAAATAACACCAATTCATGTC
AATGTCACTGTCACTCAACCCCAACATGCAACTGCATCATGGGGGCAC
GCGCGAGGTTCCCTCGTTCTCCTCCGGGAAGTTGGTTTCCTTTTTTAA
TCGGTGGAGTGTCGAGAAGGGGTGCAGGCACGAGGTTTGGGTAGGTAC
AGTGATGTAGGGGGAGAACGATGCGTGTGCAGTGCAATGATCAAATGA
TACAGGCAAGGAGAGCGAAGAGGTCACGAATGGTGGAAGTACTTGATT
TTCAGGAATCAATATTCCTCGCTGTCTGTCAACCGTTCTGTCCCCAAA
AGCTGGCGGTGGGGGGATCCGGTGGATCACGATGGGTGAGAAAATGAG
TGAATAAAACAAAAAACCCGATTGCAATACTAATAATAAAATAAAATA
AATCTCCTGCCTCGTCCAGCTTTTTTGATTGTGAGCCTGATTTTTCTC
TACATTGTAGCCGATCGTGTGCGGGGGATGTCAGCCTGGGGCAGATGG
CGCAAAAGGGTTGCCGTACGCAGGACAAGCAGAAAATCGTGGCTTGAA
GCCCGCACAATCTATTTCCTTTGGTTGTTTTAAAAATGGGTTGCATCC
AGCTTAGTCTGAGCTGGAAGTTGTCTCACCCGTAGGGGCAACAGGGAA
CACGAACAGGAGACTCGTTTCCGCATCGGCTAGCTTCGGTGGAAATTG
AAGGCATTCACCCCTTTTTTCTTTTTCTAGTCCATAATTGCGGGTGAA
AATAATGCCGCAGTTTTCGTGCCGTCCAGGGGACAGGTTTTCTTCCTA
CAACATGATTAACATTGCAACATTTGTTGTAACAATGCGATTGTGTGT
CCCAGTGCGTAAAACGCACGAGCCTCCGATCATGATGGGCATGGGAAG
GAAAAACCGTTCGACGGTACATTTGTTGCGTTCGATCATTGTCAACTC
CATTAAACGAACCTGAATAAACCGGTGCGTGTGTGTCTGCGGTGATGG
CGATCTTTCTTTATCAAACAAACGTGTTTGAGTGTTCTGGAGGCGTTT
GAGTGAGCAGCGGCCATTTGCATTCACGAAGCCGAGTTGCATCCCAAT
AAAACCAACTGCATGAGATGATTGATGTTGGGAGATGAGCTGCAATAC
ATTCCCAACCGTCCCGTTTGGTGTTTGATTGATTTTTCTTGCACCGAG
CTGCTGCAAACCGGGCCCCTGGATGCGCACTGATTTGTTTGCTTGCTG
GTTGCAACAAAGCCACACCACCGTTAAACCTGGTGATGGTGATGCACC
TGTGGCGGATCGTTGCGATGGAGCGACTGATGGTGTGAGCTTTGTAAA
TGGAATTTCACGCGTAGCGCGTCTAGACAAACCCCAATTGCGGCTGCA
GCCCCGTCATGCGGGCACGACCGACCGGACGGCCGAGACCGGTAAGAC
AGTGTTAAGTGGAAATGAGCTGCGGAATGGCTGGCATGGTCGTCGTGG
CAAATAACGTTGGCCATGTTAGGGACACAAGAAGATGCCGGTATTTGG
CAGAAGGTGCAAACGCACACAAACCTACGTGAATGCGATGTCTTCTGA
AATTAACTGTATCGTTTGATGACACAACGCAAAACGAACCAGTTTGTC
GTTACTTTGAGAGAAGAGGATCATGATGATGATGATGATGGCGGTGGT
GGTGGTTCCTCAAGAAAGATGGAGTGAAGCAAGTGTTAGATCCGGTTA
CCGAAGCGATTTTCAAACGCACAGTAATGATTAGCGAACGGGCCCCTT
ACTGTTTGCCTGTTGGTGGTGCAGTCTTCAATCATGGAACACGCTGGG
CTCATAAGGAAACATGGGGCATAATGGTCATGTGAATAATTTTGCTCT
TTTGATAAATCATTAATTATCTTCAAAATCGTTGAATAATAATTCAAC
AAAAATTGGTGCTTTAACTCTAGATTCATGGTACAACATGAACTGCAC
TCGTTTACAAACAAAATCAGTTTAAAAAAATGTCAGACAAAATTGCAA
GTTGCAAAATTGCCTTAATTATATTTTTTATAATGATGCGAAGCCAAA
TGGTAATCGGCCGATCCCGTCAGATCAGTTGTCAATCACTTACACCGG
TTTCGAGCCCAAGTAAATTATGTAAAGCTGCTTTAGAACGTTGTTCAA
CTGTAAGTAAACAATTAGCGTCCAACTGAAATACTTATGCGTTTCTGA
ACATTGTTCATTTGTAACTAAACAATTGACTCCTCTAAGCTGATACAT
TTGCTCAATAGAGTTTATCAATTTGTTTTTGTTTTCACTTACAACAAT
AATGCGAATTTAGTTGTCAATAATGTGTATAGATTGCTAGAAAATTTC
TCATTTATTATAACTCAAGATCGAAACCAATTAAAACAATTTCAAAAT
AATTTAATTTGAATAGATTCAGAATCAAACAATTCTGATGCCCGACGA
GCTCGGGTAATATAGATGAATGTTTATATTGGCGAAAGCAAATGTTTT
GCTGCGATTTGACAATGTTCAAAAGCACCTTAGCGTTGTTTAGTTGAA
AACTTTCGAAAACTTTAGTTGAAAACGTTGGCTTGAAAACAATATAAT
AACTTGCCCGTCATACCTTACTTTAAACTCTCTTTCTTTGAGTAAATA
AACAAATCGTTGATAGTCAATCCGATTTATGGTTAACGCAAATTGACT
TTCGACTATGGTGTTTGCGTCAAATGAGAAGAAGATAATCACAATTAT
TTCTGTAACTATAGCCAAATGATAATGGTAAAAAGACAACAAAGATAA
TAACAAGTGTCTCAAGTGTCTGGATGTGTATCCTTTATTTGATAAGAC
TGTTTTCTAGACTGTTCTAATAATTCTACAAGAGGCTTTAAACATATA
AATTTGTATATATTGACCCTATGATGATTTTGCTCCGAGTGTCCTTAT
TATTTATTAATTAACTATTTATTTATGATTTATTATAACGGACACAAA
TAGAAAACAGTTATTTTTGCAAGACTGTGCATTTTTGATCCGTAAAAA
CAGTTCCTGGAAAAAAGTATGCAACTCACAGTACAGGTGAAACATAAT
ACAGCGGTTGTAGAGCGTACTGTTTGGACAAGTTAATTAAATTGCACC
CAAGCGTGTATTAATTGTACCCGTGTTCGGCGTGACGGGCACACACAG
GATCAAACCACTACTGAGAAACTGGATCTGCTTCGTTCGCACTCGGCG
GTGGAAAGTCCTTTCCGCACAGCACAGGACAGTGCAGATTTTGAAACA
TTAAGCTCTCGCAACCGGCGTAACCGAATCCATAAAAACGGAGGTTCC
TCGTCCGGGATCTCCTTTCTTCCAAGTTTGTGTTGCTATCTTGGGTCG
TAAATCTTAACAGTAGCAGTAGTTGGACAGTGTATCTAAAAAGGTACG
GATACCAAAAAGGCACGAGTAGAAAGGAGCATGTCTAGATGATGCTGG
TGCTATCATTTGGCTCCAATTCGGACATCCGGATTGACGTCGGCTCGC
GGTGTATGTGCTTTAGTGAGGCGATTGTAGGTAGCAATTCTCCCTCGT
GTTGCTCCTTTCCGGAATAGAATGCAACAAGGCACAATGTTAATCACT
CATCAGAAAAGACGAAACGGGTCCGTTCCGCACCGGCAATTTTCCGGC
TCGGCACAGTCGATTTCTGCAGCCCCCGTGGGGACACATAAACAAGCG
ACCAAACAAACGGAACACACATTCTTCATTCTCGTTGCGCTCCACTCG
TCGTTTTGTACCGTGCTGGAGCTGTCATAAAGCATGTAGTGCAAAGAA
AGTTCTCATCTGAGCGCTTCTTAATGCTCACACTTGCGGTCCCGTCTG
GCCTTCGGCAGCTCCGGCAGCTTTGGGGCAATTGTTGAGCCGTAGGAG
GAAAAGACACGGTACATATAACGCCCGCCTCCCAGTGTGTTGAGGGCA
GCTGCCCGTGCTACTGTGCTGCACTGGGATTCGGCAAAACAATTTCCT
AAATGTGGTCGACCGAAGAACGAACAAGGTTAGTGTGTACCTTCGCTG
CATCGAGAGGTACGCCACTTCTTTGGGAAGCAAGCAACCGCTCAGCTC
CTGGTCCAGACTGCCGAAACTCTCAAGTACGTTTCGGAGATTCCTTCG
GGAGCGTGTGGGTTGTATGTGGCCTCGGTTCAAGAGGTGGGTATAGCA
CATTTTATCTGCCGCACTGCCATTCGTGATGCATACATCAACCGTTGC
TGGAAGTAATCGTACGGAGATGATAGACGAGCGATGAAAAATCGCACA
GAACAAAAGGCCATGACACGAGGACGAATAAAGAGTTGCCAGGGCGCC
ATCCCACCGAGGGGATGCCACAGCTGTCTCGAGGAGCAAGCCGAAATG
ATTTGCATTCAGCTGCATCGTGCAAGATATGGACCGGTGAGCATTGGC
TGATGGAGATGAACGTCCACCAGAGATACCACCGAACGCACTGTCTGG
TGGTGTGCGCAAGGTTCTCTGTGAGTGCGGTTTGCTGCGATCAAAAGA
CTGCCGAGAGCCTGTCGGCTTATTTTTCGGCTCGGCACAACAGGCTTT
GGGGTTGTAAAACAAGCAACAAACAAATGTAAATATCGTGCACAACAT
CAGGCACTGTTTGAGTGTCTGGTTAAATAAAGAAACGGTCCAAAATTT
ACAGTGCGATGGTAGTGAAGTATTGCTTTGAGAATGGTTTGAAAATAA
CGGTTTGTAAGTTATCTATCAAATTTGTCATCATGCACATAACTTACA
AGCCAAGTTATATGTAGTTGATTTTAGAGATCAAATACGTTCCTCCCT
GCCAATGCAATAAAAAAAGCCATCCAAACTTGAGACATTTGCTGTGCA
GTGTTGGGAATCGATCCACCATGTTGTAATTTCAACAATAACAAACCG
AACAATACGCCTATACACCATTTTAACCGACTTTCCCCTTCAGGGCTC
AGTCCCGCTTCCCACTCTTATTGGAGCGTAAGTGCAGCAAACGTCCAA
GCATTCGCTCTGTAGCAAGCGGTGCAATCAACGAGAAATTACAGGCTT
CCAGGCTACCAATACGATCATTTCAGCTGCCACCTCTCTGCCACCTCG
CCGAGTGTAGGTAAAACGCATCGCCTCGAAGCATTTCCCTTACGTCGG
AGAAGGCTATGCTCCATGGATGCCGAGTTGCCGTGGATGCGCTTGTGT
TGCGTTGTTCTTTATGAACGCGTTGAACCTTCCACGTTGAACACAGCT
GAGGCGAGCTTCCAGCGTTGGGGCGAGCCTCTTTTTTTCACCGCCTCC
CCTTTTACCCTTCATCAACGGCAGGGCGAGTGCACTAGTGAGCACTTA
ATTAAAATTAAACTAATTAAGAAAGCTCGTCGTATAATTTTCACACCA
CACCATCATTTTCGGGCTACTGGTAATGAAATTAATATTTCATTCTAT
TTTATTATTAACGTTTACATGGGGGGGGGGGCGGGGGGGGGGGGGGCA
GAACTCGGGGCACAGTTGTTTGGTAACCATCGTACCATTGCAGCTCGA
CCGTTTCGGAGATGTGACCCTTGCAACAGCGTTTCTTTACTTACCATT
AGTGCGAGATTTTCATACGCGCGGGGAGCTCTGCACCACATTAATCTC
AGAACTCGGAACTGCTCCCCTTCGTCCTCGGCCAATGTTACCAATGCT
GTTGATCAAGCGCAGTAGCACGCCGCCCTCCCAGTAGCACACGATCGC
GCGTCTATTAAGTGTTCGCATGTGCAGATCGCTTTAGCAGAACAATTT
ATGGTGCCGGCTGTTTGAGAAGCGGGCTGCCGGCTACTTACTTCCGCT
TCCTCCGATGATTACCAGGCTGGTAGCTGGGGTCCCGGTGGTATAAGA
AAAAGTCGCTCAGTCACGGACGGCAACACATGAATGTTTCATTGAACT
CTTTTGCCGGGTGGGCGGTGGCTAAGGCTGAAAGGGTGCTTCAGCACC
AAAACTGGACCGGTTCAGAGGTTTCGTCGTTTTCCCTTAGAACGTGTG
TGTGTGTTTGTGTGTGTTTATCCAAGAGGTGAGGACGAAAACTGCTGC
ACGATTCTTCGGCACCGAGAGATTCTTACCCGGGTTGGCCTCGTAGTA
GGGTCGCAAGAGCAGGCCAAGGGTTTGGGTCAATTTAAAAAACGGGAT
AAAGTGTGCGAGGATCAAGCTGAAGCTGGTGGTGTGTGTCCACATTGT
TTGATGATTTATCTTCTGTTGCTGTTTGCGATTGGAGCGCGTGCAATC
GAAGCCGTAATGCTAATAAAGCTGGAACAAGCAAGAATCTGGATCAGG
CAGGCAGGCGGGTGTCGGGTGACACACAAGTGCGCCACATTATGAATT
ATTCATCCTCACGTGATGGAAGTTAAACCTCTATCGTGCTGGTGCGAG
TACGGCCTGGGTGGAGAGTTTACAAACTCAAATGTCAAGCGCATGTAA
ACTGTAGAAAGTGTAGATCGCTACAGAAATGTCTCTATTTCATAGTGT
GACCTTCCATTTTGTAGAGCATGTCAAACTTTGGAAGGGAAATTGTGT
ACACGGCCACAATATCTGCCATACAACTCAAATCAGGCTATAGTTTTT
TTTTCCACAAACTGCTGATGTTTAATTATCGTGTTCTACCCATTGCTT
CACGTAACGTTGGAAAATGCTTTACACTTGCAATCCGCCCATTTTCGG
GCGTTTCTACACACTGATTAATCATCGATACCAACGCTGGTAGGTGTT
AAAAGGATAAAGCCGGTAACAATTAATACAGTTTCACGGCAAGAGCGC
AATCAAGGAGGGAAATGATTCTTTCGCTTTCCGTTATAGCCTCGGCAA
GGTGCATCGGGAGAAAATATTGCATGGTAATAAATTCCCCCCTCCCAC
AGTAAACATTGCATCCAACTTCGGGACTACAGTGTAAAGGAGTGCATT
TTTATTCATTTTTTTGATAAATCACTAAATGTGAATCGTACTCATCGT
GGATGCTTTATGCTGATGGCTACCGCTTGCCGAATTAACCTGCGAAGA
CTGTGATAAAACGTTGCTTACGGCTCAATCGAGGAACCGGCTACATAC
CCACTAACTCCACGCGAAGGCTTGACCTCTAGAGTGCTTTCCGTGTTC
AGCACAACCGAATTGTACAAAAGAATATGGTAGGCGGGGGACACAAAA
ACACGTTGGCAATGATTTATCGGTTGGCATTGCCTTCTACATTGAAGA
TACAATTGATCGGTCGGTCGCGCCGGTTCGGTCAACCTTTCTCTTGCC
TCAGTGCATCAAGTGCAGCGTAAATGCAACAATGCCGCGCGTTTCCTC
GTGCCCCCGGCCTTGCGGGTAAAGTACAAATGCAGTTTATTTCCAAAT
TAATTAGATCCGCTGCTAAACAATGTTCTCCTCGAGCAAAAAAGCCTA
ATGAGATCTTCGGCCGCACGAAATTTGTGCCGAGACCGCGGACCCTAC
AATGGCGCTGCAAATTACCGCTTTTTCCGTTCCCTTTTTGTTTGACCC
TTGCGACGTCCTCCCCTCACGCCGATCAACCTGACGGGTTCCTGATGG
GAGGCGCAGAGACAGTGGAGTGACAGTTATCGACACTTGCACGGTGAG
CAAACGCAGGGAGGAGGTCGCTGGTCATTAGTGGGTTTTGGGCTGGAG
ATGGGACGGCGTCACACACTCCACGGAGGAGAGGCAGCATAGTGATGT
TCATTTTGGACTACAATTCAGACAGTCGTTCGCGGTCGGACAGAAAAA
GTGCTAATCGAACGCATTGCATCCAGCGTGGCCGCGAACTTGTGTCCC
GGGGCAGTTTGGGTCGCGCATTGGAAAGTTAGGAGTAATGGAGTGATA
AGGGTGAGTGTGGACAAGGATGATGATGTTGCTTCGGGTATGAGTGCG
CGAGTTGCAAAGTGGCAAAACCAAATATTGTACCGCCAAGGGATGCAT
TTGGTGCGATGCACCAAATCGAGCTGTGGTTGCCTCTACAAGAACCTG
CGCGCTGCCATTAGCGCCTATAAACACAACAAGGTGTGAATGTTCGAA
TTGGGAGGTGAGTTAGCAGTGTGACAAATTGATTTGAAATGACTGTTT
AACATACCAATACGGCATGGGCAATACGTACTGATTACAACAAGTTTA
ATGAGTTAAACAATATACTTAATTTGTTGCATTCAATCCTCAGCTAAC
AATTAAAAGTTTTTTTTGTGTGACGAAACAACAACCCATCTTAACAAA
CAATATTTCACTAGCCAACTAGAAGAATAAAACAAAAAAACAATGCGA
ATGAAAGCTAGATACTACTAACACAGTTCAACTGTTTGGGTATGGTCC
CGTAGTAAAGTCGATATAACGGACGAAATAACAAAATGTTCCATCCAG
GTGTAGGCGCCATAAGACACAATGGTACATCAATCCATTGCTGATGAT
TAAACCCTCTAGTTGCTTAGGCATGTCTTGATCAACTACGCTTGTTAA
TCCAAAGAACAAGAAGAAAAAGTGTTAATCCAAAGAACAAGAAGAACA
AGTGGTTAATTCAAGATGTATCGCTCAAAAAAACCAACTGAGTTGACT
GCAGTACAGGAAAACAAAATCTTACAGCTTGAATATTTTTATTATTAT
TATTATTATTACTATTACACCATTTAGCAGCTGTTGAAAATGTATGAA
AAAATGTGTACAAACACTGTGTCAAACATAATTCCAACGTGTCATCAA
TTCGCGACATAGCTGTCCCGCAAATGGCAGTAAAACCCCTTGAAACGG
TTTTTAAATCCATCAATTAAAAACGAGCCCTTCCCCAACAGAAGAAAC
AGAGAGACAATCAAAAACAATATGCAAAAAAAAGATGACGGAAAGCAA
AAATTTTATCAAAAAAGAAAAAAAAATGCAACAGAAAAACACTCCCAT
GGGGGTAAAAAAAGGAAACAAAACATGCACATTGTACGAAAACGTGTT
ATTCTCTTCCACCTTACCATTGCGTGAACGATATGTTATGCCAAACCG
CTCGAGGCCGATGGGTAGGCGGCCGTGTGTACGTATGAGTGAGTTACC
ACCACCATACCTGTCGGCGGATGTTCAATTTCGATTCTGTGAATGGAT
TTACTTCCGGGTGGAATTGCACCGTTTGAACCGTTTGAACTACCCCAG
AATGCCGGGGCGGTTTTGTTTTTCTTTCCGTTCCGAACGCCGTATGGA
AAGGAAATGGATTGTTGTTAGCACGTAGCGCAAGCCAAAAAAAGCAAA
AAGAGTTGGAAAGAATGAAGGCATGAAACGAAGAGCACAGAACAGCAG
TAGCAGCAAATACGATTCGGCAAAGTAAATTTACATATTCGACGATCG
ACGGCTGGTTTTCCTCTGCCCAGCGATTTGCTATCCATTGCCGCGGTG
TTTGGCGTGGGGAAACAGCATCGGCACAAGGAAATTGGCCACCCATGG
GGGGGGGTACTGCTTCGCTTGTCCATCGTAATCGGTGCCCATTTGCAC
TCACTGGTACATGGCCAACACAGAGAGGGAGAGAGACCGGGGTGGCAT
TATTTGGGGGAGTTGGTGTCGGAGCGTGCACTTGCCAAGGGTGTCATC
ATGTGCCTTGAACGTTGCATTTCCGATTCCCCAGAATGGCTGCGATAC
GGCGAGCAAGAATGGTTAGCGTGAAACAAAACAGTCGTTTGATGATTT
TGATTCCGTTTCGATCGGAAGAGTTGGTGTGCGATATTGAATGTGTGG
GACGGGGGTGGCGAACGTTTTTGTTCCCTGTACAGATGGACTGTCACA
AATTTATGCAAAATGTATTAAAGGATGACGTTTCGAGTGATGGAGCCA
GTTCGTGTTGTTTTTTCGCGCAAGCTCTACCATTTTCGGTGGTCGAAT
TTTTGCGCCACGTTTACTAAATCGCCAAACAACGCGATCCAAAAATGT
GTCAGCTCTCTTTGTTTTGATTTTGGCTGGCGTTGGAGGTAAAACCAA
CAAGAAAAAAGAAAACTTAAATCAAATAAATAAAACCTCTTGGCCGGC
ACTGGCGGGAGAACGGGCCACGGCTAGCTCTGCTAAATTAAACACTTT
GTTATGTTTTGCTGCAACTTATTATATTATAAGCACTGCTCGGCCGAC
AGGAAACGTATTGAAATTTACGATTGCAACAATGTAGAGCTGTTCGTT
TGCAGCACCCCATTTGTGAATGGCACTTGTGCGCTGGAAGTACAAATT
TGAATGTTTACAGTCTAAGCTGTGCGCACAAGAATTGTCACCCGCGAA
GAAACAATCATTTCGACACTTTACCCCCGGTTCCCTTTTCTTCGGCTT
TCTCTCTCTCCCTTGCCGCTGCTGGTTCGTCGCTGGTTCGGTTCCCAC
AGCTGCAAACCATTTAAACACTTACGCAAAACGCGCGTTCCACTTCCA
GGGCACCGGGAACAACGCCCAGAACGAAATATCGTTAATCTCCTTCGG
GCGTGTCCTTGCCTCGCGGGTACTTGTCTCTTGGTTTGCCCAGCGAGA
TCTGTACGGCCGCGTGTACACAGGCTCTTACAATGTTGCGTGTGTGTG
CGGAGAAAATGTGTAATCGATTTAGTGGCGCAACACTATGCGCAACGT
TTTTCTATTAATGCACGTCTGTGCGTTTTGTCCTGCCCGAAGACGCCC
AAGACACTCTTCCCAAGGAATGTGTGTGCACAGGAAGTGTCAACTCGT
CAAACCAAACGCGGTGGAGTGTGTGTGTAAGGTGTCGTAAATGTCATG
CCAGCAAGGATAGGGTATTTGTTGTTCTTAAAATTTACGATTACCCGT
TCTACGCTAGTGCGCAATTCGTTTTGGGCATGTGCTTGTTGGACATGT
TGTGGCGGGCAGTATATGCAAAGCAAACAGAGAGCATAATTGTTATGA
TGACTGCGCTCCTTTCACGGACGGAGCGGTTTCAGCTGGAAGGGCCCA
CAACACTCCCAGCTCAGAAGCAAAACAATTTAATGACGAATCGTGGAA
AAAGAAACCAATTAATGGAAATAAATACTTTGTTGCGAGCAGTAGAGG
GCTGTTTAGAAATTTTGGTAACTAGCGATTGCGTGTGTTTACAATGTA
TTAAAATGTTTATAAGCCGTATAACTATCGAGCAGGAAGCATTGATTC
TTTCAAACAAAGATTCGGATTCAATGTCGCGTCGTTGGATGAACGAAC
AATATTCTTCAAATTCTAGACAGCAACAAAATCGCGCTGCAATACAAC
TATACCGTTGATCGGCGTTAAAAAGTATGCAGACACAAAGTAAGGCAA
CAATAATTACATTAATTCATCAGCGAAGAACATAATCAAGCATAGCTG
GAGTGTTACACTGGTTACATGCCAATCGGTAGAATTCATTAGGAATTG
GTCGGCAACATCGTACCTCCGGCAGAAGAAGCATACTTTGTGCTGACC
AATGCAATTCGTTAGGCGAGCAGTCTCCCTTTGATGTTTTAGCATCGA
TGAAGTGATCAATACACTGACCATGTGTCGGATTTGTGTGTGTATGTA
TGTAGTCTGGCATGCTCTCTCTCCTGTCTAGCGAAAATTTCAAATATC
AGTCAAATGTGTTCCAGCAGCACATTATCGGGACCCGTCTAGCTAGTC
TCCACACTCACACTTTCCATATTTTTCACACCTTGGTCTGAATTTGTA
GTCGTCCCCGTGCGGGCATGGAAAATTACTGTGCAACTCCGGACGGTA
GGTGTTGATGTATGCATCCAATAAACACTTCACGTGTTTTGCCAGGTT
TCGCGTACTGCAAACACGGGCTTTGGCGTGCCGTACGCGTACGGCTGA
CAAGCGCGTGCGACAAATGTTAACTCGCCACCTCAATCAACACCGTAG
CGTAGGACGGCGAACGGTAGGCGCACTCCGCCGGGATTGACATGAAAT
TTCGAACGTGGTTCGAACAATCGACCTCACCCTTACCCAATGATTTCG
CGCCGAGCGTTCGAACGGGCTAATTTTCAGAAGGGAAATCGGCAAATG
GATGGATGTGTTTTTCCGGCCGTATTATGACGAATGTGTGCATATCCG
TGTATGTGAGTATGGGAGCATGCCCGCGGTGGTGGTTGGCGGTGGGCA
AATAATAAAATTCAATTTAATTAAAATTGAAATTAAAACTGGAAATAA
TTACAAATAAATCATAATTATATCTGCGGTTAGATTGTGTGCAAGCTA
ATTATAAATCAATACCCGCCCGCGATTGGGACATTCGCTTCATCATTA
ATGGTCACAATAATGCGGGACACCGGAATGCTCGGTAGCATCGGCCTG
GCATACCCCTGTCCCCGGAAGGACAGGCGATACAATTTAACCACCAAA
CCTGACCGTTGTTCGGGCTACGATCGCCATCATCGCTTTGATGTGCAC
TTGAACTGCGGCGGCGTTGGCAAGCATTGGAACGGAACGAAACAAAAA
AAATCAACCAAGTGATAAACACGGCATAACCAGCACAGAACATAACCT
CCAGTACCAACCGGATCAGTACTGAGTTTCGCTCTCTGATCCGTGTCT
TTAATTTTCTTTGCTTTTTTATCATTTTGCTTTTGTTGCCTTTTTGTT
TTTCCCAGCGTGGCTCGATTGGAATGAGCCGTCCGGTTCGGTCGGAAA
ATCATGTAACGGCATAATTACTGTTAATATGTGCGCAAATAAAAGGTG
CGATTGCATAGCGGATCGAGTGTTGTTGCCGCCACCGGGGCCACACTG
TCTACCGTCCGCTGCGATGAAAAGTGCATAATGGTTTCAAAATTGAAT
ATGGCAACGCGTTTGGGGAATGAATGGAAATCTCTTCACACAAGTAGT
TTCCGGTTGATTGAGCCAATCGATTAACACTCGTTTGTGTGTGCTTTT
GATTCGCTCAAGCTGTGAAATAATGCGCCAACTTTGGTAGAATGTTGT
AGTTTTTTCTTCGGCTACTTTATGTGAGCTGATCTGATTGCTGAAACG
CGCTGCTGAGGATGCCGTTTTCTCAAGGGTGACTGTGTTGTGCGGCAG
TGTGACTGTGTGGTAGTAATCCCTACGTCACACACACACACTCCTACT
GTATGCAGCGGCGAAGGTTATGTTTAGCAAAACGCGTCCCAACTGACA
AAGGGCTTCAGGGTTATTCGGTCAAATTCAGATCAACATGCTGCAATA
ATCGCGCTGATAAGTCCCGCACACGGAGCGCCACTTGCATGCATCGTT
GAATCTTCCGGAACAGCAAAACGACACTGGGGCACGTATGTTTGCAGC
AACACGGCTGACCCGTGGCCGTGTGCCAAGCGTGCGCGGCCCAGTACG
TCAGCGACACGGCCACAGCTGGTACGATGGATGCTCAGTACGCTCAGT
TGATATGCGCTGAGTTGTGTCAGTTGGGTGGTTGGGTTGACCAGGCGC
TAGTTTACAGTGTGCTAGGTGGTTGGTCGGGTGTGCCTGTGAAGCCTA
AATGGAACCAAAAAGAAGGTTCGGAGCAAGATAGAAATAACAACAACG
TGCCATAAACAGCTCCGGTGCAAATATGTCTCCTCCAGACGCGATACC
CAATCAGCGCACCCCAGCCCAGCGGGTAGTATCACTTTATCTAGAGCG
GACCGGTGCTACTGGTGCTGCCGATACGTGTCAGAATGTCGTTTCGCG
CGCTCGCGCCCTATGATGCTTCGTGCGCCCAGTCGGCATACACTCCTA
ATTCGTATGGATAACGTTACGACTCGAGCAACACGCACTGCACGATCT
GTCTGACAAACACTCTGCCTTGCTAGAGCAAACCGCTTTATTCTTAGA
AGGAGAGGGAATTTCAATAGATCACGCGTCGTGCTGCAGCACGGTGTC
CGATTGTACAGGTTGGAAATTGTAACGCTCCAGGAAGTAGCGTAGCAA
AAGACCCTCCCGAGTGGATGGCCATGCTAGGTTGATGGACGCCGTAGT
GCGAGCGCTTGCACTGACATTAGCAGGAAGTACCGAGTTCAATTGCTC
TAGTAATGCAATCAGCTAAAAACAGTACAAGAAGGCGGGTGTTAAAGA
CATTTCAAACATGCTGCAGTTGCGGTGTGCGGCCTCGTTCCATTGTAT
GCTTACCATCTGTTCCTCGTCGAGCGTATTGGTGCTGGTGGCGATCGA
TTGCACCAAATTGGCCAGCGCGTTCGGACCGAGCAGACTCACGACGTA
CGTGTAGTTCTCGGTGAGGAATTCGATCAATGCGTCCACCCCTTGGCG
GCTGCTGAGGACGGACTGTAGAATGGATAGCCGTTCCTCGGCGTTGAA
GTTCACCTGCAGCTCGCCACCGATGGCAGCCAGCAGGTACGAGCTCAG
CTGCTCCGTATCGTTGGCACATCCCAGTGCATTGATCAGCAGTTGCCG
TTCACCTCGGTTGTCCGAACCCAGCAGCTTGCCGAACAGATACTGGAA
GGCGACCGTTGGCGCGGTTCGCAAACCGTAACAGTACACCACCGCCGA
AACGTCCGGGTGCACAGGTTCCGCGTCGAACACTTCCCGTTCCAGGGC
GTCGCGGGTCGCCGTCATGCAGCTTTCTATTTCCATTCGGCAGGCCCA
GCTGGAGATTACCTGTCGGAGATACTTCTCCAGCAGTCTCTCGTCCGG
TGCTACCGTTGTGATGTCCAGCGTTACAAACACATCGCCAATCAAGGT
GTCGACAAACAGCTCATAGAGAATGTAATCGGGCTGACCGCGCATTCG
ACCGTGGAAGTAGCTGAGGACCCGATTAGCCGCTTCCCATGGAGGATA
CTCCCGTTCATGGCGCACGTAGCCCAGCAGCTCGAGCGCAATCTCCAG
ATCGAGCCGATTTGAGCGAGCCAAATGGAAGGAATCGTCGATCAGCTG
CGCCCGACTGTGCATTGGAATGGCCGCCGTGTCCTCGAGCAGCGTCCG
AATCAGCATGTACCAGTTCGAGGGATCATAGTTGACGCGATAGAATCC
CGTCTGATTGACGTTGACCAAAATCCACTCGTTGTTCGGTGTGCTGGA
CGGTACACGTACCGCTTTCGAAGTCATCCACTGCCACTCGAGCAGAGC
GTCCTGCGCATCGCCCTGCTCCATCATCGTGTACGGTATTACCCAAAC
CGTGAAATCATTATTAACTATCTTGTTACCGTAGAATCGGTCCTGCGA
GAGGATCATCTCTCCACGGTATGAGCGGCGAACTTCCAGCACGGGATA
GCCGGCTTGATTGACCCAGCTATGAACAAACCGCTCCACATCGGTCCC
CTCGGGCAGCGATACGACACCGTCGAACGCTTCCGTCAGTGCGGCCAC
GAAGTTATCCGTGTTGACCGTGCCGAACTCGTTGCCCTGCACGTACGT
GCGCAACATCTGCCGCCAGGCGGCATCCGGCAGCAGCAGCCGGAACAT
CTGAAGTACCGAGCCACCCTTGGAGTACGCCACGTTGTCGAACAGGCT
GAGGATGGCATTAAACGTTGCGCCGCGGCTGAAAGTCATCGGGCGCGT
GCTTTCCGCGGCGTCTGTGATGAGAACACGCTGCACCACCTGAACGTT
GAACAGGTCCCGATACTGGCGCTCCGGATAAGCCATATCGGCCCCCAG
GAACTCGTACAGCGTCGCGAAGCCCTCGTTAAGCCAGAGATAGCTCCA
CCACTCGTTGGTGATAACGTTGCCGAACCACTGGTGCACGTACTCGTG
CGCGATGATTGTGGTGATGGTCGTTTGCGCTCGATACGTCGTAACGCC
CGGCTCGAACAGGAGGACCTCTTCACTGTACAAGCAGGAAATGGGCGC
AAATGTTACCAGAGAGTAGCGTTGACAAATGAAATGATTCACCACACA
CACACACACACACTCACCGATATTTGCACAGTCCCCAGTTTTCCATGG
CACCGGCAGAAAATTGGGTAAGTGCCACCTGATCCACCTTGGGCATGT
AGGAGCGATAGGGTAGACCGATGTGCTCGTCCAGCGCGTCCATTACGC
GAACGCCTGCTTCTAATGCATACAGCGTTTGGTTGATCGCGTTGGGGC
GAGCATAGACGCGCTGGGCAGCCGCCTCGTTCTCGGTGTACAAGAAGT
CCGACACCAGGAAAGCCAACAGATAGATCGACATGCGCGGAGTAGTTT
CAAAGTACGTAACAACGTTGCCGTCTAGATCACTGAAATTGCAATCGA
AAGTTATTTGTCACAAACACACCTCGCAACGTCAGAGCACTCGACAAT
CGCCATACCCGGCTTCGGCAAAGATCGGCATGTTCGATACGGCCTTAT
AGCTGGGATGATGTTTAATTCCCAACTCCACCGTAGCCTTCAGGGCCG
GCTCGTCCAGACAGGGGAAGGCGGCGCGCGCACTAATCGCCTGGAACT
GCGTCGATGCTACATATTTGCGCGTACCGTTCGCATCGAGATACGAGC
TGAGGTAAAAGCCATCGTCATCGACGCGCAGCTCACCCTCGAAATCGA
GGTGCAAAACGTACGAGGCCGGTGCAAGCGCACGACGGATCGCGAACA
CGGCAAACTCGCGCTCAGCATCCTCGGTATAGCGCAGAGTTTCCAGAA
ACGTGAGGTTCGTGTTGGGATTGGATGCGTATAGCTCGTTGGAGGTAA
TGCGCAGTCCGCGCTGATGCACGTAGATGGTTTTGGCCTGCTGCCGGA
TGTCCAGATGTATGTCCACACTGCCACTGTACGATCGGTTTCCGGTGT
GCACCTGCGTCTCCAGGTACAGCTTGTAGTGCGTCGGCACGATGTAGC
TCGGCAGTCGGTACCGTAGCTCCTGCGCTGCCACTTCCTGCAGGCTGA
CCGGATCGAGCGTGTTCAGTTTCCGCTCGCTATGCTGCACCTTCGGAT
GCGCCGCAATGGCTGCAGAGTGCAGCCCGATTAGAAAAACACCGCACA
GCAAATGTAGCCGCATGTCTACAAACTTGAAGGTTGATTTTGGGACTG
AAATCTCCGGTGCGAAATGTCGACTCCAATATCCGTAATCGCAACAGT
TTCGGATTGTTTTACGACCAGATCGACCACAAACAGTTGCTCGTGTAC
GTACCCCCCGATAACCGAGGTGTGGGGCAAATGCCTTAGGAAAAGCAA
TTTCTCACCTGAGCAATTGAATTATCCATACCTTTGTATAGCAAGCGG
GGCTCGTTTGGATTGAGATAAGAAGTCGATTGAGTGTAATAACTGCCG
AACAAGAGCTAATCGGCCTTAATCGCTTATCGCTCGCTAGTGAGTAAA
TTCGTAGGGGAATAATTGACGTTTACTCAATGACTTGTGTGATTTATA
TTTGATGTTTGATAATTCGCATCTCATCTAAACCAATGCTGTCTAAAA
ACGATTGAATATCTTATTGACGTGGGCCGTTTTTCTACATTTTTGACC
GTTTACTTGCGCAGTCATGATTGAATTTGGCTGATTGTGAATCATTAA
TCATTCCGTAAATATATTGGTGCTATACTACTGTATAAAGGATAGTAG
CTTAGTAGCTCAGAAGCTTAGTACAATATTTGAACGTTAAAGAAACCA
AAACTGAGTTTGTGCATATAACAAATCCCAAGTACTAGCGATAAATAA
CGCTACGCAAGTAATCTATCTGTCCAGTTGTAAACAACATGTAATAAA
ATGGTTCAAAATGGCGCGACGACCGGAAATGGATCGCGTTAAAACGTC
TGCCTAGAGACATCTTCTTTCGTATGGTGTGTGCCATAACACCTCTCT
CGCTCTTTTGTAGTTCGTACCACTTAGACTCCCGATGCCGATGTAATA
CTAGAGTAGGAGGAAATAATTAATATCACAGTTAGGGCACGAATGCTT
GCGTACTTCACGAAACCTTATGTACCGAAGGTGGAGTTGCGATTGCTC
ACGCGTTGTTGCCCCGTTATATGCGAGGTGGGTCGTTTCGGGCCAAGA
TGTAACAACCCCAGCATAAGGTGGGAACGAGAAACCGTGCCCGAGAAA
GGAACGTTCCATCTAAGCCAGCGTGGAGGGCTCTTTGTGGGCATGTGT
ACGGCGATACGGCAACCCAAAAGAGAAAGGGCGAAATTAATGTGTTTG
GCTCGTTGGCCAAACAGCAGTCGGTTTGCACAAAAACCAAAGCGCCTG
CGAAAATTAGTCACACCCTCCCGGGCCAGCTTTTGGGGAGAGTGGGAG
ATAATGTTATGTGTCTAAAATGGTTAGACATTTTTTACACGTGAAGCA
AAGTTTGCATTCGCTCCGAGCGGGAGCAGGTTGTGCCATGTCGGCTTA
GGGTGGGTGGAATGCGCGTGTTTGTGTGTGTTTGATGTGATGAAAAAT
GCAATTGCGAGCAAAGTACGCGCACAAACCCCGCAGGCCAATCCCTCT
TTTTTCCAGCTCCTTTATACATTTAATTCCAGCCAAGCAGAGCCCGCC
GTTAGCCGTGCTGTGTGAGCTTTTTACACGCTTGAGATAGAAATAATG
GCGTAGTGCGCTGGTTTTCGTTACAGTCCGCTGCACAAACCCGGACTA
AGGGAGGGCGGCTGATGGTGGATCGCTGGTGCCGCGTTTACGGTGTGT
TGCATTAACGAGGCCCAGGAATAGGCAGAAATGTATTTATAATTCAGA
TTAGTAACAAAATGGTGGCTCTCAAAGTGCGATTGAAGCGCGAAGAAG
AGTGCAACGAAGAGCGTGTCCGTAATAAATGTGCAAAAAAAAGGAACC
AAACATTTTTGCAATAAATACTGTTTACAGCTGACGGGGTAAAGTTTA
CTTCCAGCGTTGCAATTGCGCTTGAATGCTCGTTCGACCCGGTTGTGT
GCCGAACTCGAAGCTTTCTAGTTTATTTTATGACAAAATAACAAACAA
AATGGTGTCTGTCACACCCTGTAACCTCTCTATTAAACTGATGATGTC
ACGCAGCAGCCATAAAACAGACATCCCACTAAGCTCTCTATGATCGTA
ATTTGTAGTGCAAAAATGTAGCCATATTAATGAGTACCTTGCAATCGG
ACGACAGTGAAGGTCTGCCATAAAAGCGTTACAAAATAGGCACAGCTC
TGGGCAGTCTAGTTTCTGCGCAGCGATCAGGCACACTCATAAGTGCAG
CTTTGAAGCGTAAACTGCACTTACTAACGTCCTGATTCATCGATCGAA
TAGCCCGGCACGCCCCCATCCGTAGGCTTATCCGGGCTGTTTTGCTAC
GAGCGGTTCAGGTCGTTAAAATCGATCGTTAAAATATTATGGGATCTG
TCCTCGGCTCTTCTCACGTGCATTGGAGAAGGTATGGCGCGGTGCAGA
TGAAGGGATGCCGAGGAGGAGGTATGGTTCATATTTGACCACAGTGCG
TATTTGCGAAACCCGAAAGGTGCATCAGCTAAATGGTGGAATGTTTCT
GCTTTTACGAGTCGACAGCTGTGGCTCCTTCGACGGGGCAGTCATTAA
ACTCTCCTCCTAAAATGTCGTTTGCACTCAATAGTGGCAGCACTGCCT
GGCCCGATCGAGCCTTCGCCAAAAGATCGACCGTTAAGGGAGGGGGGA
GGGGTAACCGCGAGCGATGGATAAGGATATCGGTGGCATCGATTTCGT
TTAATGTTTTGCCTGCTGCATCGCAGGCCGTCGTTATGAGCCCTCCGA
TTAGTGCATCGTGATAATAAGGGCAAAACACTCCGTTGGTGGCGCTGC
AACTAACTGTCGGCAAGAATGTGGCATTAATGCCGGCAACGACGGGCC
GTTTTGTTTAATTTCTTTTCGTCGTCACCGGCCGACTGCCCGCTTTGC
CAATAAAACCGTGCGTCGCGTGTGCGAGCGTGTGTTGCCTGGCTTGTA
GCAGTGCACCCCAGCCCAGCCAGAGTGCGCTGATCGCTCCAAACAGTA
GGACTATTAAAAATCAATTTTCCACCGATCCTCACGCAGTCGTTTTTT
ATCTCTACCTCCGCTGGGGGAATGATCCGCGGGCTTGTCTTTACGCAG
GCGATTAAAATGCAAGTGAAAACAAAAAATAAAAACACGAAATAAAAC
ACGATTAAAATGTCAGTGAGTGATCTTTTTTTATTATTTTCGTTCCAC
ACTGCATGCATGCGTACGCTTTTTCAGTTTTGTAAGTTCAGAATTGGT
TCAATGGCCGATACGGTTGGCGCTCGGTTTGAAGTAACGACCCCGCAG
CATAAAATGTGAATCATTTGTGTGCGTGTCTGTCTCTGTGTGTGATGG
CATTCTGGTTTTTCAATGATGCGCTCCTATTTTCACAACCATTACGGA
AGGGCCAGATTCATTAGCCGTTAATCGGAAATTTGCGTGGTGACGTGG
TAATTTGTAGTTTATTTATTTGTGATTGCTTTCGGACGATGCCCTTTT
CCCGGTTTGTTTTTTACTGCGGATGTGGTGCGTGTGCGAAACGGCAGG
AAAGGTCGACTGGTTCCCATCGGAATGGATTCAAATGATAATCTGATT
TATTTAGCAATGGCACTGAGGCTGACACGAGCCCCATTTTGTGTCACA
TTGTAGCTGCAGTGGTAAGTTGCCGTAAAACTTTAATTCAATTTTCAA
CTCACCGGCACCGGAAGCTCGTACAGCCTTGACAAGGAAGAAAAAAAA
GCTTTGATACATTTAGTATTTAAATGGACTGAGCGGAATTTTGTGAAG
TACAACGGGCAATATTTATTATTTATTTTAGTACTTTTATTGAATCGC
TTGCAAAACCAGTCATCATCTTCAGGAAGTAAGAAACGACGTTTTCAA
GATGCTTTGACTCATCTGATGCACGTGATCTCAACACAACTTCCTCAC
ACATAATGCCAAGGAAATAAGTTTCACTCAATCGAAACATGTTTGTGT
GTGTGTGTGTGTGTGCTTGTCGAAAAACGCTGCTGGAAAATATGCGCA
TTTTCAGTTTTTACTACCTCTCCGAAAATTCGGTACGGTTTCGGTGCG
GTGCTCACCAGCCCGCCCAAAAGTTACACGTTGATTCCCCTCGGAGGT
CACGTCACTGTCTAGCACGGTGGCGGCGAGAGACTGGCGGGCTGAAAG
ATTGAACAGCGGTTCGTCCCAAAACTAATCCGTGAATCATCATCCGTG
GCCGAGCGCGAGCACGGCGCTGCCCCCGGGAGCCAAGGGGCAGTAAAA
CATGTTTGGTTTTACGAGCTTGGAAAAGTTTTTCTCATTTTCCTCGCT
CAACCACTTTGCTGTGGAACGGATTGCGCGGCGCTCGTTAGCGTTTTC
GAGATGCGAGCCGTTGCCTCTGTTCTTCGTCTTCGAAACCACTGTTGT
TTCGCCTGTTTGATTTATGTGTGTGTGTGTGTGTGTGTGTGTGTGTGT
GTGTGTAGTTTGTGATGGAAACTAATAAGTTTTGATGCTTCCTTTCCC
TGTTTGTCTGCATGCTCTTTGGTGGCATTTTAAGAAAGCACTGACTGA
CAAAAGCCAAGTTTGTGTACGACTTAGGATGGTCAAACCATAGTTTGG
GAGGGCCTTCATGTGTGTATGTGTGTGTTTTTTCCACACTCCGACCAG
TACGCTAGTGCAATGTAGACATCCTCCCGGTAAGATGCATCTTCCCAG
CGAGCAGCGGTTGCGAACCAACGAACCTTGGCTTGCATGTTTTTGATG
AGTTTTAAATTTTGGCTGATTTGGTAAATTTTTACGACTTTGTTTATG
AAACGATGGAACTGACAAAAGGCACACCAGGCAAACCAGCAGGAATCG
AGCGAAAAGCAAATCGCGTAACGAACCGCACGTCCAACATAACTGCGC
ACCCCATCTCGAACGGTGGACGGTGCGGGGCACGTCTTCGCAGCATTG
CAGTGGATTGATGTCTTCCAGCAGAGTTTTGGCGCCGCCGTCCAGCGC
ATTGTGCTGGCGAAGGTCGGTGCAAATCTGCACCGGAACACGGAAGCA
CGAAAAACGGAATCGAAAGCGCAGACACCGGGAACGATAAAGATGTTT
GAATGCGTCATAAATCTACAAAGACGGTCAGTGAAATGAATTGGAAAC
TCGCATTTGTCGTCGTCAACGTCATCGGGAGTTGTTCATTTTTTTTTT
TGGGAGGATAGCAAACGCACATCAAATGCAGTGGCCCATCACAAGTGT
GATCTACAAGGTGGTGGTGATGACGGCGGTGGTCTTGCTCCGTTTAAA
CGACAATGTAACCAATACGTCTAGCAGTTGACGATGCATATGATTAGT
GAAGTGGAACCGCGCTTTAAAGACACCTTTGCTTGCATGCGTGTGTAT
GTCCGCCAGATCGCACAATTCATCCCAACGACATGTGAAGGCTTTAAA
AACAAATTGAAATCGCTTGAAACACATATTCATAGCGTGCCCGGCCGA
GAATGGGTTTTACTTGCTCGTTAACGAGAAAGAGGGTGTTTCTTCAGC
TGCTCTTCAGCGGGGTTAGTTTTGCATTTGAAGCAAATCGTTACAAAA
TGCAATAAAATCGTCTAATGGTACGGCGTAACGACGTGTAGTTGTACT
TGGACCAATTGGCCACAGCGTGTTCGCCGCGGAACACGGGCAACACGG
GGTGGGGTTTTAGTTTTTATTTTACATTTTTTAAATGCCTCCCTTCGT
TGTGCCAATTGCTGTGCGATCTGTCAGGTTTCGAACACATTTCTTCGC
TCTGTGCAGCGAACGCGTGCAAATGAGCGTAAGCGTGAGTGAATTTCA
ATTCCAAAAGAGGTCCAGCCTGTCATAAAACCTCACTCCACTGGTTCC
CTTTTCCGCGCGGTCGCTCGCCCATCCATCGCTGATGGCATCGAAAAT
CCACTCGTTAAACGCGAAACCACGAACCGATCGGCGCGGGGAAAGGGA
CACCGGTGCCAGCGGCCGGGCGCGCAAGGATCGTAAATTATAATATGA
TTTTTATTACATTTTAGCGTAGCATAAGCCGAGGCCGGCTGAGAGACG
TTCGTAATTTGTTATAATGTTATATGGCTTTCCGTTCCCGAGCCGTGC
ACCGACACACTGGGCGCCGACAAGAAATGGCTCAGGGTGTACTGTGTG
TATGTGTGTGTATGCCTTTGCTGCTATTGTTATTTTTATATTTCCTTC
CAGTCGAAGGAAACGGGTGTCTTTGGAGAATGGGGAAGCTTTGCACAA
TTGTACCCCAGCGGAGACTCACTCTAATAACGTTCATTTTCAACAAAT
AAAAGCATTGCATCAGAACTATCGTCAGAGTGTGTGTGTGTGTGTGTG
TGTGTTTGTGTGCTGCTGCGATAATTTCTGTATCGCTTTCGTCATCAG
TTTTATTTCGTTATTTTATTTTACAATTGCTCGTGAAGTGGCGTGCAA
ACGCAATTGCGAGCCGCTTTGGCGAGCAAGGAACCGCGCCCAAGATCG
GTTTCGGTTCCCTTTTCTTTGTGAATCATGGTTGTGAAGATTTGTTGT
GCAAAAACGCCAAGCTAGTAACGAATTGGTAAAATAACTGCGCCACTG
CATGCACAAACACACACACACACGCACAGGCAGAGGAAAAACGAAGAG
TCCGGATACAAAATTGCGGTTTTGTAGCTTTTATGATCCAATTAGCTG
TAGAACAAGAACCGGGACGATGCGAAAGGGGTGTTGTAAGACGCACAC
AGGCACACTGGTCTGGGCATGCTAGTCGATGGAAATTGAATCAGCGGA
TATGCGTTTTGCGCACATGCCTTTTTTCATCCTTCCCTTTTACCGTTG
AGGCATGGGAAGTGTCATAAACTCGTGTATGCGATTTGTTGTTCCGTC
AAGGTTTCGTTTGACTGAGTTGCTGTAAATCAAAATAAAATAAAGTGG
CCAAAGGGCCGGGACGAGCAGAGGAATGTTTCCAACGCATGTCTTGGT
GGTGTCCAACAATCCTCATTTATGATGCTGCATTGTCAATGGAATGGT
CTCATGTGGTCCGGACACGTCCAATCACATTTATTGCTTCATTATGCC
GAACGAAGTTTTATTTCGGAAGTGTGGAAAGTATGTTTTTTTAACTCA
TTCGAACATGTTCCTTTTCAATATAATTTTGTATAGCTTCGACAAGGA
ATTCGCTAGCAGTTATTCAACAAATAATTACGCATGCAATAATTTGTC
GCATGCAAATTCCGGTTTCAGCAAAAGCTGGTTTTTAAAAGCTCGAGT
AAATGTGTTCAACATCCTGCTATGTAAAATTAACTATGTTTTGTAAGT
GTTCCAATCAGTCACAGAACGCCAAGCTGAGGAAGAGTATAGTGTTAT
AGAACTTTACTAGAAGCCAGTTGGATTTTGTTCATCCCCACACTAATA
AGACAGACACAATTTACATTTGCGTAGTTTGTGCTTTTGCATAATACA
TTTAAATGTAGAAATTTAAATAAATAGAATCATAACATTATGCTTCTG
GGGTAAAGTACAGCTAGCTTCCATCCTTCCCTACATTAAAATCAATTG
AATGCTGCCATATAATTACGTGAAAAGAAGAAGAAATAGTTTATTGCG
GTGTTTTACCGCTATTATTGCATTACCCGCAGCACCGTCAGTAGGAGT
AGTGCTATGCTTTTACCTAATCATAAAACTAGTTATTATATACCTTCT
GCACACCCAAGTGGCATGATTCGTTGTGTTGCCCTTTCTCCCCATGCT
TTGTGCCGATTCCCAACAGCGAGTGTGAGAACACCCGTACAAGAAAAG
CCCTATTCTTCCCACCCAGAGCGGGAATAGTATACGAGAGACCCTTGC
ACACTTTTCCATCGCGATATGGGTGTAATGGTCGGTGTTGGGGTGAAT
TTTCCAGATCCCCTCAATATTGCTCGAGGCTTTCGATTGGCTCGGGCT
GCTGTAATAGTGTGTAATGGGTGTGTGGGCACTCCAGAAGATGGAAAC
CATTTCGTATAAAACAAAAGAAACCACCCCATGCTCGAGACCGGTGCG
ATCGCTCGAATCGCTGAAACTCCACCGTCACGAGCACGACGTTGTCTA
GTTGGGCTGGATCTACACCAACCTGTGCTAGTGCGCGCGACTAGATGT
GCATGTAAAAAAATAAACATATAAATCAACAATGCTCGGCGTGGCAAG
CATCAAAGCAAGTAACGGATAGAAAGAGCAAACTCGAGGGAGCAAACT
TCGACGCCAACAAACCCTCCCGCGCGCGCCCAGCACTAGCTATGCACT
CGAAGCGCATAGCGAAAGATTTACGCGGGGGGATACGGTTGGTGTTGG
TGAGCATGTTTCGATGTTGCGCCCCATGAGCATGTTTTGGGCCGCCAG
AGCGAGACGGGAAGAGCGCGTGCGAAACATAAGACAGAGGCGGAGTCA
ACCCTACCATTGGTTGCGCTCGTCGGTCGTTCTGTTGCTCCCGCTCTG
ATGGGTGGCGCGCGAGCATAGGTCTCCGACTCGCTCTAGCGCGTTGCA
GCCGTTCCACACACCTTTTTGCACGTGCGGCTTTGCCACCACTGGCTG
CGGCACAAATTCCGACCGAGCACGTGGTTCCTCTATCTACATTTCTGC
GCCAACCGGTGGATGTGGACGTCTCCTGGCACATCGGTCGAACTGTGT
GTGTGTGTGCGTAGATACAACATCTCGTTATGTTGTGCCTCCGAAAGC
CGAACACCCTCGACCGTCGTCATCGTCGGTGTCGTCGCGGTTTTATGC
TCCGGCGAAACTGCTGCGAACGTTTCACTCTCACTCTGTCCCAGTGCA
TCCGGCACGGTATCTTTTGCATCCCTTCGGCGGTAAGTTTGGGCGTTG
CAGCACGATGTTACATCGGAGCACTCCGCAAAAAGCAGGCGGAAGAAG
CAGCTAGCCCGAAAATGTGTGTCGGAAAATTTCACCATCAGTTCGGGA
GCGGAGAGGAGGCCGCTTTTCCGAGGGAATCAACAAACGATTTCGCTG
CTTATTTGAAGAAGCAGCAACCATCTACGAACGGTTTCTTCAAACGAT
GAAGCACACAACGACATACCATTCGGCTCTGGGGGAAAACATGTTTTA
GTGCTGCTTTTCGCCACGTATGTCTAAACCGAAAAAGAAGAACTTTCT
CTATCAACGGAAAGACTATTTTTTTCGCCTGTTTCCCAAACCTTACCA
TAGAAAGAAGGACTGCAATGCGCGGATACGACAGGAAAAGAACCATTT
AGCGGCACATACTTGGGAGAGAAGCACGTTCGTAGGAAACAAGGATGT
TTATGTTAGCGCGAATAATTCAGACACGCTCTGAGCGCTTTCGGGTGA
GATTAGCAATGGAGCATTCGGGCAAACGAAAAGAACGTTTGCGTTTCG
AATGGGGCGTTTTTGCTTGTGCAGCGATGACGAGTACCTCGTCTAAAG
GCAGTCAGCTATCCGGAAAACGTTGCTCTCGATTAATGCCCGTTGGTA
GCATCGCACAATAGCATAAAAGCACATAAGACAAGTCACCGGAAGGCT
GCATAACACCGAAAGGTTAGGAGAAAAAAAATAACGGACGATAAACGG
GTACAATCTGAGTTGGTATCTGAGCTGGGAAAAGGGCTGAAGAAAATA
GGAGCAGTAGAAGCTTTATGTAGGATTTGCTCATCGAATGAACAACGT
ACTAAAGATCGTTTTTTACACGGCGGATTTATGTTGGAACAAGTCGTT
AAATAGCGAGCTTTGTTGGGAGTATCAAATAAAAGAAAACCTCATCAC
TTACCAAGAGCACTAAAAGAGATTTAGTCAAGTAGTGTTGTTAGTCTT
TTTATTAGCTTGGGATTTACTATTTATACTTATATATCTTATCTTTAC
TTAAAAAATGGCAAAAAAAGATAAATAGAAAGATGTCAAATCATCAAA
CTTGTTACATTGTTTTATAAACTTGTTTGTTACTACTTGTTTGTTATA
ACATTGCTTTATACACTTGTTTTTACTTTAATGAAAACAAACATACAA
ACAAGTATTTATTTTTCAATAATCCGTTATTTTTAGTTATATGACTAA
AACTAATATTGCAATAAAATGACTGCACTTCTTATTGGTGTTGAAATT
CCCTGATAACGCAAAAAATGTCATTAAAAATTATGTGTTAGCTAACTA
ACTAACGCCATGTTTCAATGTTGAAACAAGCAGATGCCAAAAGTTTTT
TATGATTTTTTATAGTACAGTAGAAACAGACGATATTTTTCCGATTTA
TTAAAGTTAAAGTGCATTCAAACGGCATATTGGTTTACGTTTGAATTG
AATGTATCTTTATGTACAGTTTAATCAGTCGACTGATTGTTTCACTCA
TTGGATTACGTTTGCCTTGAAAGTAACATTTCAACCTGTATGGCATTG
CGCACATCTATTTACTTGTCATGTCGCTCCTATGGCGCTCCATAGTTC
CCACCAGCCCCACCGAAAAGATTGATTAACATCTTGACGGGTCATATA
CTTATTAATGCCGCCCATAAAATTAATCCTGCCCGACTATGAATCGGA
CATTGTACACAGTGCAGCGACTCTCCTCCCATGTACGGTAACAACCAT
GTTACCTCACGAAGGTCATGTCCGCATACGCGCCAAACATGAAGCGTA
CCTAAGCAAGTCGTGCACCAAACTTAAATAAAAATAATTGAATCAATC
GAGCACGGCTTGTGATAAACGATCCGATTGATTCGTTAGCCGGATGCA
GTTGCAGTAGTTGTCTTGCGGTTGTGGAGTTGCAGTAGGGATGGGGGT
TGTGGAGGGGTATGTACGTCAGCGTTGGGTGGCTACGATCGCGCCACG
TGCGTTCGCGAAAACGACCAACCAGCACCGGTCTTATCTGAGATTAAA
CGAACGAGGTGACAGCTAAAAGGAGAAACCGGGCGATTATTTAAATTA
GTTCCCCTACGAATGTTGTACGGCGCGGCGGGCTGCATCGGAGGAGGG
ATCTTATCTCGGGGGTAGCGTTATTTGCGTTATTGTAGGCAAAAAAAG
GATAAGTATGCTGCTGGTAAGAAGGTAAGAAGTATGCGCGCTGCAATA
AGCATCCCCGTGCCCTTTCGGCACCCGGCGTGTGGAGCTCGGTGCATC
GGAAGCTCGGATTTCAGCTGCACCGAAACCAGATGCACACACGCGCAC
CGCTCCGGGGGCGTTGAGGCAATCGAAAGCAATCAACATCAATTAGCA
AGTTTATTTGCAACACCGCCGGTTTCGATGGATTCTTCCGCATCGGCG
ACTGGTACAAATTGCTGCTGCTGCGCCTTTAGCGGGTGGCAGATCGGT
TTTGCCGCTACCGGTACCGCATACTATGAAAGTATGATTTATCGTCTA
CAATCATTTCCCATTACACAGGCGCGGATCGTAAAATCAGCTCCGGAA
ATATGTGTGTGGGTTTATGTGTGTGTGTGTTTCGGTGGGGATGAATCG
AAAATTCATCTTTTGCTAGCGGGACGAAGCTGTTGGTGTGGAGTGCCC
GTGCCAAATACGTTGAAGGTCGCGATGTACGCGATTCTCTAGCCTTGC
TTAGTCATTCAGCGGGAATGGGTTGGTTGTTGCGCTCGCATTGGAAAG
GTGCATTCTGCACCGAAGCATTCCAGTAGCGCACGCCGATCGTTTGCT
CGATTATGGTTTGTTTAGTCTGGATGAATAAAATATTGCTCAATTATT
CAATTTATCGCGGGCCTGGGCCCGGCAGTGGCAAACAGGACTGAAACC
GCCGTTCTGTGCAGGTCTGTTCCGCGATCGATACTATCGTCTGCCAGT
GCATTTGTGTGTTTGTTCTGGCCCGCTTGTTGATATGTTGTGGTTGCC
CGCTTGGCAAATGTGCAACGCATCCGCGAATCGAGATGTTGCAGCATG
GATGGACACGAAACACGAGCCATAACTGTACAAACAAACGATTGGCCC
AAGTTGGTTTATAATTGCGAAGCGTGCGTTAACATGGCGATCAAGAAT
AAGTTCATAATCGATGGATTATGAGCTTGAGCGGAATTGCAAGGACAC
GAAATTGATAAGCACAAACAATGAATGTGTATTGTGAAAGTGAATGGA
ATTTCAGGTGATTCATGTCTGGGAAATGTTTGTACCACAAATTGCATC
ATACCATTGAGAAGCTACAATTACGCAGATTAATTTTACGCACAGAAT
TGCAGAAAGGAACTGTTTTTTTTTGCAAATAAAAAAAAAGATTGAATA
TTCAACAGTTGGTTGGAACTAGCGAAACCAAGGGCCCTTCAACCCGAA
GAATAATGATACGTAATTTTTCACGATCGATGCAAAACATGCACAAAA
TATTGCATTTAATTCTTCACAGCTAGCACCGATCGTTTTGTCATGATC
AGCGATCGGTCGATGTGTGCCGCTGCTTGCAAGTTACTATTCTGGTAT
TCCCATTCTCTCCGGTACTGGAGCAGCCAGCTTCGTGTCATCGACAAA
GCGCTTCAAGTGATGCCCTTTTACTACAACCCACGGCGAACTGAAAAT
GCCAGAAATAGATAGAGGAAGATCGACAATGATCTATTGACTAGTTCA
GGCGCGCGCGTCTCGCTAGGATTTGCTTTTCGGAGGATCCACCTCGGC
ACAATCTCGGAGACGGCGGTGATGGCGGCTCTACCGGTGGATTGACAC
TTTGACAGCTCTGATGCAATACCCATTTCCAGTCGACGGATGACGCGA
AATCGCACAAAATCCACCCTCCAGCCGGGGCGGAAGGAGGACGCTTAT
TTCCACCGTGATCAAATGACAAACGGGCGCGTGCGCTTGTGTTTAGCA
GGCAGGGGAGATGAGCGCAAACTGTGCAAGAAGAAGCATCACTGTGAA
GACGGCAATGCAAAGATAGTGTGCTCAACTTCTCCGCGAAGATTGAAG
CTAAATTAAGCACGAGATTAGCATGACTGAAGTGACTTTTCAAAGTGT
CAGAATGGCTGCACTCGCAAACTAGCTGGATGCAGCGCAATTTTGCCC
CGGTGTGTGCGCGCATGCAAACGAGCAACCGCAGAGGGCAAAGGAGAG
GATGGGAAGGAGGGAGGGAGTGAAAGAGCAGGCTTAAGGTTGCCCTCG
GGCATTGAAGTCGATACAGCGGTTCTATTCCAGTGCCAGTAACGATGA
CGAAGACGATGTTGCTTCTGCTGCTGTTGCTGCTGTTGTTGTTGATGA
TGATGATGATAATAGTGCAAATATAAAATAAATCTTCCGTAAGCTTTG
TGTAGTGGTGCGTGGCTACTATAAGCCCGTCTGGAAGCAAGGAAGCTA
GTCGGGCAGGGTCATGCAAAAGGGAGACACCTTCGGAGCTCCGGAGCT
CCCGCCGGCACTCTCGGGGGGACGTCCGTTATGCGTTGTGATTTATTA
TGGAATATTTATTATAGTGTCTTGTTTTGAAAAAATAACTTCAACGGT
TCGAATTTCCTACACCTCGAGATCGGGGCTGGAGTGGCAACGTGGTAC
GGAACGGTACAGCGGTTTGAGCCGTTCGGTCTTGGGACTCACGGATCG
CAGAATGTTATTGTGCGCGCACTGATGGGAAAGTCATTTTTCACCGAG
TGGTCAGGGCGCGTAGTCCAGTTCGTTTCTGGCTGCTGTTGCTGATGC
TACGATCCTCAGGAATGATTGGAAACGCCTGGAGATGGTGGGAAAAAA
TCAAACACAAAAACGATCCTAATGAACATCGTGTGTTCTCATTCGCTG
CCACGATTGACACCTTCGATAAGACGCACATAATGAGCTAAAGGAGAG
GGGACAGGGTCTTGTCTTTGCCACGAGCGATAAGATTGCAATCACTCG
TGAGCGTGTGCTGCTGGGCTGAAGAAGAAACGCTTTCCACAGCAGTAG
GTGGGAAGTGGGATTGTGGAACGTGGCATTGAAAAGAACCTATTTTCT
AAAGCCCGAGAGCCCGTTCTCGAACTGGAAAACCAGATGCAGAAGTTT
TTTATTGTCCCCCGCCAGGAAAACAAATGTATTTAATGCTTTCTTTGC
CTTTTCCGCCCCGTTTCAGACGACGAGCTAGTGAAGCGAGCCCAATGG
CTGTTGGAGAAACTCGGCTACCCGTGGGAGATGATGCCCCTGATGTAC
GTCATACTAAAGAGCGCCGATGGCGATGTACAAAAAGCACACCAGCGG
ATCGACGAAGGTAAGCTGGCGATGATGGTGTCGTTCGACATCACTTTC
ATCACCGTGTCAGACATCTACTGTGCCTAGCACCGGGTCCAGTGGTCA
CAGGGTGTAGCAAAAACGTGTTCTTTTTTGCGAGAGACTCTACCTCAT
GATGCAGCTGTTAAGGAAAGGTTTCAGATGAAGGCAATTTTTCCTAGG
ATAAGATGATCTTAAGTTACCTGCGTATTAGTGTTTAACATTGTCGTC
TCAACTCCCAAGAATGTTTTAATCGTCTAGGGCTAGTTTATTTATACT
GTTCTCATTGAAATGTCGTTCAATCCAACATGTTAAGTTAGCTAGCTC
AGACACGAGAAGTTAGGAGTATCTGCATCTTGAAGGTAGCGGCATATG
GTGTTATGCCACGTTCACTGACTTCAAAATTCGATACAAAAAAAAAAC
CAAAACATCAAAAACCAAATTGTGAATTCCGTCAGCCAGCAGCAGTGA
CCTTCAAAGCCTTACCTTTCCATTCATTTATGTTTAACACAGGTCAAG
CGGTGGTCAACGAATACTCACGATTGCATAATCTGAACATGTTTGATG
GCGTGGAGTTGCGCAATACCACCCGTCAGAGTGGATGATAAACTTTCC
GCACCACTGTAACTGTCCGTATCTTTGTATGTGGGTGTGTGTATGTGT
GTTTGGTGAAACGAATTCAATAGTTCTGTGCTATTTTAAATCAAGCCG
CGTGCGCAACTGATGCCGATAAGTTCAAACTAGTGTTTAAGGAGTGGA
GCGAGAGAGCCGCACCACGGTACAGAAGGGCAGCAGAATGGGTCGGCA
GCCTAGCTGCACTGGTGCGGTGCGTCCGGCGTCTCGGGGGGAGGGCGA
GGAAATTCTAGTGTTAAATCGGAGCAGCAAAAACAAAACAGTGGTCGT
CCCGTTCAAGAAACGGCCTGTACACACACACAGAAAACACTGCAGCAT
GTTTGTACATAGTAGATCCTAGAGCAGGTGGTCGTTGCTCCTCGAACG
CTCTGGACGCACGGCTTCGCGCGTATTTGCGTAGCGTTCCGCCGATCG
TGGGTATTCGTACTGCCACAAGCCCGCTTTCTCCCATGCAATCTCTGC
AACCAAACCAACAAACAACAACAAAAAACCAATCGACAAAATGAATCA
CACCCCTTTTGTATCATCTGTATATTCTTGTTCTTTGCGTTCTTTTCT
ATGTGGCCCACGCCCCGGCGGGTACGTAATTGCGTCGAAAACCCCGAA
AACCCCGGCACATACAGTGTACATACGGTTTGAGGACAACTTTGACCT
GCAGCCCTTCTGGGGTTGCCACGTGTAGCTATACTTGTGAGATCGGGC
GCCGACGGTGTAAAGCGCGAATGGCCGCCACACAGTGTGTCCACTCCA
ACACTACCCCTCTGGAACTACCCCGTCCAGGGATGCACCGGCTCGGCT
CATGCCCCTGCAAAACAGTCCGGGCTCCACTGTAGTAGCTCCGGCGTT
GCTCTGAGAGAAGGATGCCCTTCGAAGTGTCGAAAGCGTGCATTGGGC
GTTCAAGTGTGTGTGTGTGTGTTAGGTTTAGCGAGAAACAGCAGCAGT
TGCGTGTGCTGAAAAGCGAAGGAGTAATAGAGTGCATAATGAAAATGA
AAATGAAAATGAAGCAAAAGTAGAAGGCGGAGGAGAGCAACCTGTGTT
CCACTAGTAGCGAATAGTTTAGTCTAGTTTCGTCACCAATCAACCTTC
CAACCATCGTTCAACCAATACCTGAGTCAACATCGTCATCGTTATCGT
GCCACAACTTTATTAAAAATGAACCTTGTCCGCGCCACCGTAGGGTGA
TCTAAGGCGACCTTTCTTACGGGCGCGACCCACATGCCATCGTCACCT
TCTCCAATCAAAACCAACAGCCTGTACCGATGGTGTGCAATTGTGCGT
GCGTGTGTGTTATTAGCAAAAAAAGAGAAAGAGTCGACGAGAGAGAGA
TAGATCGAGATCGAGAGTACAAAAGAGCAGTAGAAATGTTCGTTGTTT
GTTTTTCGTAACACAGTTGTTTAGCCAAAATGGGAATTTCCAATAATC
CCGGGGGCGGGGAAATGCGGGAATACTGCGTACACACATACATCAATC
AAAAAGAAAAATCCTTGCGCTACATCACTACCGTTTGCGCGGTGCTGA
TCTAGAGCAGACCACTTTCCACTCCACTCTACAATCAATCAATCTGTG
CAGAAGGTATGGTAAGACGGCCTTTGAGCGAGTCACGGTCGCCACCAT
AACGCCGTCCGACGAGGGCTGAATGCGAACTTTGCTAATCGATTTTCC
GCTTTCTTTTTATCCCACCTCCTTTTCTCTCCCTCTCTCTCTTTTGCA
CTGCCCCTTGTAACCCCCAAAAAGGTAAACGACACATTAAGACCTACG
AAGCGTTGGTGAAGTCATCGCTCGATCCGAACAGCGACCGGCTGACGG
AGGACGACGACGAGGACGAGAACATCTCGGTGACCCGCACCAACTCCA
CTATTCGGTCGAGGTCCAGCTCGCTGTCGCGGTCCCGGTCCTGCTCGC
GCCAGGCCGAAACTCCCCGGGCCGACGATCGGGCCCTGAACCTTGACA
CCAAATTCAAACCATCTGCCAGCAGCAGCAGCACCGGCTGCGATCGGG
ACGACGGTGACTGCAGCGCGTTCGACGACAGTGCCTCGGTGGTGCGGG
GGCACGGGCGGACGGCCCACAGCACCGGTAGCAGGGGCCGCAGCCACT
CGAAACGGTACCACACCCTCCCGGCCGAGCACATCGGGAGCCACATGG
CGGCCGCCCAGAGTCGATCGCCCGCCCCGGACGACGAGCCGGTGGTGT
CGGTGTCCGTGTACGAGAGCCTGGTCGAAGCGGCCAGCAAAAAGACGC
GCACCTTCAGCCCGCCCCGGGGGGAGGCGGAAGATTTGCATGCCGCAC
GGAAAGCATCGCCCCACGACGAGCGGGACGAGCCGACCCCGGCCCAGC
CCTACGAAGCGTACCTGGAGTCGGTGCGGCGGAGTAAAAAGTGCTTCG
CGCTCAAGGACAGCGAGGCGCCGGGCGAGGAGCCGACGGGCTACGAGA
AGGAGAAGGAGCCGCGCATTCCGTACTCGCTGCCGAAGAGCACCTTCG
AGCGGCTCGACCTGCTGAAGAAACCGAACGGGCTGACGTTTCCGATGT
ACAAGTACAGCGGGATCGAGCCGAACAACTTTGCCCTGCCGCTGCTGC
TGCCCGGGCTGGAGGCGGTCAACCGGACGCTCTACTCGACGCCCTTCC
CGGCCCAGCTCCTGCCGTCCAGTCTGTATCCGTCCGTTAGCAGCGAGT
CCACGACAGTGCCCATGTTCCACACGCACTTTCTCGGGTATCAGCCGC
CGCTGCAGCTGCCCCACGTCGAGCACTTCTATCGGAAGGAGCAGCAGC
AGCAGCAGCAGCAGCAGCAGGGATTGGCCGAACCAAAGGAACCGACGT
CGTCGTCTTCGCCGGGCAGCAACCGGCTTACGCCACCGAAGGGTGCAT
TTTTCTACGCGAGTGCGGTGGAAAATTCGCTCACCGCCCAGCAGGCTT
CCATTGCTACCATCCATTAGATCCACACTGCGTCCACTCGCTGTTTGC
TGCAGCGTACCGCGGACAGTGCAGTGTACCGCTGTACAAAAAGGTAAG
TGTGGGTAGTAAGCGGTAGGGTGGGATGGGTAGATTAGACAGTAGGCA
AGTGGGGATGCAAATTTACAGCCCTTTTGGTCACTTTAACAGACACAA
CAGACAAGGGACGCTAGCACGAATCATCGCAACAAAATGGAATGAAGC
AAATGGCCTTTGGACATTCTTTGATCTTCACACTGTTTCCGCGGGCTG
GGGACGTTATTAGAGGAAAAACGCCAATATGTTGTCGTCAACATTGGT
TCCGCTCCCAGCCTGGGGGCTGCTTTACTTCTGCCAGTATCGATCATC
GCCTGGTATCGCTCGGCATTAAATAAATCATTCATGGCCAAATCAACG
TTTAGTTATTGATATGGGCAGGAGGAAGCAAACAAACGAAAAAAAAAC
GGGCACACTCCATCGAACTGGATACTGGAAACTCTGCACCCTACGCTC
ACCCTCATTGCACCCTACCAGAGCCGATATGCTGCAAAATTCTAAATA
AAAATAATCCATGCGGGTCGCGAAGCAAATAATTTATTTCCTATTTAT
ATTTATTTTTAATCACACACAAATATGGGTGCATGCACGTGTGTGTGT
GTGTGTGTGTGTGTGTGTGTGACCGAGTATGGACGGACGATGGACACT
GTGGTGCAAATAGCGGTGAGCGGTCGTGGCCGAAGGTTGGCTAATGCA
ACGCGTTGTGTCGCCCGTTTTTCCGAGCGTGCCTGATTTCCAATGCCT
ATTTTTCACTCCACTGCCGCTTTGGTCGCCATTGCCTTCGGGGGGACC
TTTTTAAGGCAAATGTTGATTTGCACCGACACACACCGAATTGCACAC
TGCACCCAGTCAGTCAGGCAGGTGGTGTTGTTTGAAAATGGCGCTCTG
GAGCAACCAACAAACGAACACAAAACAAAAAAAAAACAAATCAATAGA
AAGAATCGAGCTGTTTCGATTATTCAAAATTTATACACAAAATATGCA
ACGTATTCCCCGGTGGGGTACCCTCATTGTCCGACCTACTCCCCCCCG
GTGCACCTCAAACCCACCGGCAGCAATCAATGTAATAATGGTAAAGGG
TGGCGTGCCAAATACTCCCGGACCATTCCGCGCTCGACGTAGGGACAT
ACAGAGAGCGGGAGCTGCAGTGACACGAGTGAAACAACCTGGAGACCC
CTGCATTCGTCAGGCGGAAATAAACAAATCAAAACAAACCTCCCGTCT
GATCTCGCGACCCTGCCACCCACCGGCAGCCGGCAACCAGTCGTCCAA
TTTCGGCACTTTGGCGGTGTGCAACTTTAGCAGTCTATGCACATGCAT
TGTAAATATGCATATTGCACGAGATAAAGAGAGACGGGCCGAGAGAAA
GGGTCTCTGTGAGCGGGGTAGCCAGAAGTATCGAACGACAAACTATGC
GCGTATTACGAGATGCGATCGGTTTGACACTCGGCATTCGCACTTTGG
TGGCTATTTTTATTCGCCTGCTTAACTCCGTCGCTGTTTGTGCGTGGC
TGCGTGTATGTGGCCGGGCGAGCGTTTGTTTAATCTGGCACGGTGCAG
TATGCAGTTCGGATGCCAGCGCTCGCCGCCCCCTGCACCACTGACCAC
CCGTTCCATGCCCAACGACAGCAACGTCCCGGCAGAGTGATCAGCAGA
AGAAAGGCGTTTCGTGCCAATTCTGTCGTATACATCGTGCACGGACGC
GGATTGTTGACGAAAGGTTTTGTAGCAAACCGGGCGGCGAACAAGTTA
TGAATAAATTTACTCCATTCGTTATCCACTGATGTATCATTAATGGCA
GCCGGTCAGCTATGGGGCGCTATGGGCAGTACAGTCGGTCCCGGGTGT
GCCGATCGGTAAATAAAGTGATTTTTGCATTCCGCTTCCGTGGTAGCT
AATTTTGTGTGGCACACTTTGGAGCGAATTGTTTGATTAGGGCTCGTT
TGTTCGCTTGACTGTAAGCTATCATCCGATGAAAGCGGGCTTAAATGC
TAGATTTACTAGGCCGATCATTTTGACAGGTAGCTCTAGGAGCTTTTC
ATTATGCCTAATTATATTGTAAATATTTAGTTGTGCATTTAATGCAAA
CTTCCAACAAATGAAAAAGTCATTCTGCTCTTTTAAGTATTTTAATCA
GTATTTTCAAAGCTTTAAGCACAAACGCTTAGAACGTTTGATGTTTTT
AGTATTTTATCTACTTATTTGTTTATTGAGTGCCCCTGACATTCGTCG
CTCACAAACAATAAATATTTTTGGACCTGGATCTAGTAAATGTACGAC
ATAGCTCGAATTGAAAATCAACGTCAATATCTCTCTAATTTTATGGTC
TAATTGCATAGAGAAGATAAAAAACTATCTATTATTTACCGATTAGAA
ATTAATTCTAGTATCCTCCTGCTAGTGCTCGAATCGAATTCATTTGCA
TTCCTTCTGCTTGCTAGCCGCAGGTACAGCAATATCGGAAACTCTTTC
TTTAATATAGGTTTAAAGAGCCTCTAATGTGCATCTTTGCGCTGATCG
TAACGTTTCACCGAATCATCAACGAGTGTTGTTTTGCCTTCTGCAATG
AAACCATCCTACACTCTCACGTGTTTGAAAGAGGTCCACGGCACACCG
GGAATGCATTATGCGCTGACGGCGGTGGTGTTTTGTTCGAAGTTCGTG
ATGCAACCGCCGGGGAAGTTGCACACAGGGATTTAACGACTCCTCGTA
AAACGGTATTATATATCGAGGCCGCAGCGAAAGGTAACGCCGCAGCCG
CAGCAAACGGCTACACAAAAGTAAACCCCTCTCTGCCGCACTCGTTGC
GCAGTGCCGGACCGCATGGCGCACATCTTCGACCAGTTCGCGAGGTCG
CTCAATACATTAGGAACTAATATATATTCCAGGCAATAATAATTTTCT
ATTTTACTGCCCTTCGTGGGGAGATGCTTTGCGAGTGGTGCTCTGTGC
CAGGAGAGGCAGAGAAGGCATACCCACCAACCACCTCCAGGGTTTCAA
ACACGTTCCCTGCGCTTATCGTGAATCTTTTGCATCTTTTGATGATCG
ATACTCCTCGGGCCCGGGACAAGACCAACGCCAAGGTGCACCGTGTGG
ACCAACATCGTAGACGACAATCCGTGCGTTGCGTTTTGGCAAGGAGGA
GCTGTACGAGGTGAGATAGAGTGTGTGGGAGAAAGATAGGGATAGCAC
AAAAGAGTGTGTGAGAGAGAGAGAGAGAGCGCACCTAGAATAACAGCT
CGCCTGACTGACTTGACTGACTGGCAGGCCATAGAATGGTGGCGAGAA
AAAGCGTCTTACAAGACGCGCTAAATGCAACTTTACAACGGTCGTAAA
CTAGGTCGTAAATATCTTTGCCAGCATACCTTCTGCAAAAGAGCAGAT
CCCGCAAACACACACTGCGTACGGCGCAACGGCTGCCACTCGTGATGC
ACTTGTAGTAGACCGGGGCCCGATCCGAACCGTCCCGGACGCGTTTTG
CTGACCGAAACAGACACGCACACAGGGTGCATTTTGCTAATTTTTATG
CTAAATTTTTCCACCACCGACATGGGATAGTTTCCAGCTGAGAGTGCA
AGTGCACTTGGGGTGCAAGTTGTCGCATGGAGCGCGATAACGGACGCA
GTCCACTGCTCATCTTAGCCTTATACCTGCTCCTGGAAGATCCGATAT
GTCTCCAATCAGTATCGTCGGCAGTATTTTACGATAATCCGCAGCGAA
CGGGAACCGGCCGCCTTGGTAGCGGTTTGTCAAACGGATCTGCACTCC
GCACTACCGTCATGACGCGATTAGAGGTAGAGCAGCATGCCGTACTAC
GCTACCACTTGCAACGGCAAACGTCGCGGAGCAACATTGTGGCCGCAG
CGCCGAAGCAATAAAAGTTGGAGGACATCTGTGAGCAGATAATTTACA
AGCTACTTTGTATAATGAAAAACGCATTAAAAAACTACGCCTGGCAAA
AGTTCCTAGTTGTTCTTAGGGGGGAGGAAGTTGGAGGGGGGCAATCAT
TTGCGAACCAGACTGCGAAACTGTTACAAGACAAACCCGGAGCATTTC
CGGGCGATCAACTCATGATTATTGTTAGACTCGCGGTGACGAGCTGTG
AAGCGTCCTGCCTTTTCGGACGTTGTGCGAAATGTTTCGCACTGCAGC
ACGGCGGGTGTTCGATGCCGTGGTGTAGTTGCGGTTTTTCTACAGCTC
TCACATACACATAACCGGCATGAAACACGGAATGCGAGCGATGCGAGC
TGGGAGTTGGCGCATCAAACTCCACTAATGTTGCACACTGTGTGGGGT
GGGATCAACTTCTTCGCCGGCGTTTGTTACCGCGGTGGTGCCGATGAA
AAGACGCCATAGATGGATTTTAGCCAAAGACACACCGTTCCATCGTGG
CCGAACAACGGTTGCAACGGTGCGCTGGGCAGAAGGTAATGGAACCGG
TTCCGGTACTGATCGGCCATTACGGGCTAGTGAATTTTACTAGTTTTC
AGAGATAATTTTATGGGTTTCCATTTGTGGGAATTGCTTTTTTTATTG
CCTCAACTGGCTGTGAGGTCTCTCTTCTGGGCCGGTGTGTTGTTTCAG
CAGTTTCGTTCCTTTGTTCGAGCGGTTTTGTGCATTGTGCTTGATGAT
ATGACAAACCCAGAAAACAAAACAAAAAAACGATAACTACATGCGTCT
GGTTTATCTGGCTGTAAATTTAGTTTGCAGTCCTTCAACACACAGACT
TACACAAACCTCATACCCTAATCATTGTGATGGATATCGTTCAGTATC
ACGATGTTATTGAGGTGTGTTCACATATTCCTAATGAATTACATTTTT
TGTTTTATCCATTTTAAATGATGAATAAATATTCTACAAACATGTATA
AACTCATATTAATAAACCTATTGTCCAAATTAATATTAAGTGGCGTGA
AACGATACAGCTTATGCACTACGCAAATATTACGAGAATATGATCTAA
TTTGCAGTGAAAATTTGTTTTCCTTGGTTCCAATATTTCCACAACCTT
ATATATCATGTGAATTATTTTAAAATAAGTTATCATCTTAGAAAAAAA
TCATCATCAGATCAAACATCACTAGATCTCAAAGTTACATCAAGCCGT
TCGCTCTGAATTGTAGTTTTATTTCGAGTGTTTCAAATAATTTACTTT
TTTCTCATCATACTTATACACTTTTTCTCGATTTCTTTCCGCTTCCTC
AAAATAGATCGATTGGAAATTCACGTCAATCATCTGCAAGCCCGAAAG
ATGCTACCTAGTCGTCCCCAGCTGTTGCTACTGGAGCTTTGCAAGAGA
TCCAGCTTTCGTTCCTTATCGATGCACAAAAGGCGCACCCGGAAACAA
AACAAAAATCCAACCCACTCGTCAACGGCCCACATGGCGGGTTGCACT
GGAGAAACTCCCACCCTCGTAAGTGCTATCTAAGCGTTAAATTACCTT
CGCCCTTTGCGGTAGAACAAAATAGAAGCAAATGAAACAAAAAAATCA
TTGCCGGAGGCGCAAGTGAACAGCGGAAAGGGAAAGAAACCCCTGTCG
AACAGAAAACATGATTATTGATATTTTTCGATCGTGCAACGAAGGTCT
ACACTGTGATACAAAATGTTGTGTACAGGATAAATATTAGATTTTTTT
GTTTGGAAAACAAAAACACAGCTAAACGGTAGGAACAAAACAAGGCAA
ACCGAACAAAACGAAACAGTACGCACACGGCTCGTTGTATGTAAATCA
ATCTATGTGAGCGTGTGTGTGTGTGTGATCGTATGTGATTATGTGTGT
GGCGAACGGTTTCCCATTTTCTGTGAGTAACGCCCCGTTACGATCATT
GCTGTTGGAAAAAAAGCTAAAACCAAACCTTCATCGAAACGAATGGCG
CGCGTTCTTTACTTGGCGCCCAATTTCCCACCAAAATTCAAACCTGTT
TTTAATAGTGTAAAACGTAATGAAAATAGTAAACGGGCGTGTGTTGTG
TGTAGCATGGTTCGATCACTTGGAACCAAAATCTCAAAAAAAAGCAAA
CAGAAACTCATTGGCAGAAAGGCAGACACACCGGAATTGCGAAGTTGG
GAAAGCAGATCACTTTCTTGTTATGTCTGCGTTTATTTCTCGTGTGCG
AATGGAAGGCAGGAAATTCAGAGGTTCATCTCCCATGGAAGATGACGG
AAAGAGATTAAGAAATTCGAAGGCAAATCTGTTACAACGGCGAGCGAT
TGTGTTATGGCTAGTAAAGAATTGAATTGTGATACGTGCGCAGTACTG
CATATTTGTTCAATTTGTAGCTTGTAGGTAGATCGCCGTCCTCGTGTT
CCGTGATCCGGGGGCGGGATGATAGACTCCGCCACTTGGAGCGATATC
CCATGTTGCTGTACTCTCGTTTCGGTGCCTTTTTTTCTTGCTCTTTCG
TTTTACAAAAAAAGTAATTATATTGCTTTTGTTTTATGTGCGCACCCG
CACACACAGCTGCACACGATCGTACAAGTTAACGAATGGTTTAGTTTG
CGCTAAGTTTGATTGGTTCTAGTTCGCTAAGTTAGTCTGTAGAGAGAT
TCGTTTATCGTTATGTTCAGCAGCAGTGTCAGGAACGAGATTGGAAGA
TAATTACAGGGGCAGGGCAGATGAGCAAAGGGGGTACGGTTAGGGGCT
GGAAGTCAAAATGCTTTAGCCATCCTGCAGTCGAATTTAAACATTAAA
AAACAGGTCCGCCTTGACGAAACAAATACCCCCGAGGAGTTCCTGCGC
CCGGCCCCTCGAATGTGCACGAAATGGAATAGGTGTTGTACAGGCAGA
AGACAGTTGTAGAAGCAAGGGTGTAATGTTCCAATTGAAAAGCGAAGA
GAAAACCTAATGTAACTACAAGGCAGATATACAGCTGAGAGCTATATT
TTACGCAGCGAAATACAATGTAATCCCATTTTCTCCACTCATCAAACC
TTCATTAGTCCTTCACATTTCACACAAGCAAGTTGTACTATAATGTAG
AAAAAAGTAGAACAAGCAAACCATTTGATGCATCATCGTCATCCAGCT
TGAAAACAAATAGATCAAATTACATAGAACTGGCAATGTCTATTGATA
CGCTGTTCGAGAGACTTTTTTTTAACACAACCGTAACATCAGTGGTGC
CGCGTGAATGTATGTTTATTTCTGAGTATAAAGAAAAAACAACAATGT
GCATATATACTGGTGTGCAGTCAGCTCTTTCTGAGAGAATAAAAACCT
TAACATTTCGCTTTGCACAAACCATGTCTTGTAAAATATTACTCCAAC
AAGAAGGACAGTCAAAGAAAGAAACAAGAAACAAAACGTTAAACTTAA
ATCAAAAGCTAGAAATGCACATGTACCATACATTATTGCCCAGAAATT
ATCTCAACAAAGGGGAGAACAAAACACAGTTACAGCCAACAGAAAACA
GTTACAGCAAAGGTGTACATAGCATAGAGTCACAACACAATATGTACA
TTTTACCCGGTTCAATATCAAAATAAAATGAAAAAAAAAACGTCCCGT
CCGCTGATGACGGAGTAATGAGACGAGGCGTGAAAATGAAAATGCAAC
ATCAACAGTTAAGAATCAAAATAACAAAAAACACCCTTATCCGGCTCC
AGTACACAATCTATTGATGACGAAACGTGTGCTGCGAATAATGTTTTA
ACAAAAGATGAAGTAAGTAGAACGTGTTTGATGGAAGCGATGGGCAGC
AAAGGTAACGAAAACACACATGCTAAACGTCATGTGTAGCATGTGTAT
AATAGCAAGAAGAAATTTCAGAGCAAGACCCAAGGAAAAGTATCTTTG
ATTCGTCAAACGCCGCAAAACGCTGTTTTACTGCTGTAAGTTTGAGGG
AAACAACCTCCGGTAAAAGAGAAATAAAGTGGAACAAAGCAAACAAAC
AAACAAACAAACAAACATAAATAAATTATTAATATTATTACTGAACTC
CGTCGTGCGTGCTGTATTTCGAGTCGCTTTGCTCGCCAATGTATGCGT
CCGAAACGATGTGTTTATTTAGTTATTTTTACCACCAACAACCAGATG
GTGGTGAAGTTCAAGAAAAAAGTAGCTGAACGCAACGCTGCGTCAATT
TCTCTGTCTCCCCACCGCCTTTCTCTCTCTCTCTCTCTCTCTCTCTCT
CTCTCTCTCTCTCTCTCTCGCTCTCTCTCGCTCTCTCTCTCACTCTCT
CTCTCACTCTCTCTCTCTCTCTCTCTCTCTTTGATTTCATCGGATCAG
TCTGAACTTTGCCATCCAAACAACATTTAATTACGGTCGTCGGTATTG
AGGCATAGTTTTATCAATCCTGGCAGCGGGACTCGAATAGAGAGATGC
ACTTTTCCCTTTTCCATCGGAGTAAGGACGTTGTGAGGATGGCAAAAT
TAGGTTGACTAGTTTAGCAAAGCGGAGGAGAAGAGTTTTCAATGGTTT
CACCGTTCTTAGACGCGATTTCTTCTTCCCAGCTGGATGAGCCACAGT
TTGAGCCGGTCGCATTGTACTGTGCAAGGATATGAACCGGAATGGTGG
CGGAGATGAGTCGTGCTGATGCGGTTCCATCCAGTCTCCAGACCCGGT
AATCGGTCCTTGGCCCTCTACCTTTCTGAAACGGTCCTCTGCAAGGTA
GAAAATAGGTGGTTTTCTACCCCGTTTTGTCTTCTCTCACTCTTGCGT
CGTTGTGTGCAAAGTACTACCAGAAGTACAGGCAATCATGATGCTGAG
ATCGTGATGCTGCATATCCGTGGCGCGAGAACGAATCTTCACTTTGCA
CTGTACGGGGGAAATTGCCATAAAATGCGACAAGCGGTACGGTGGAAA
ACAAAACTGTGCATTGTACGCTTCACCGAAAGATGCCAGCGAACGCGG
GCTTGATGCTTTCGTACTTCGGGAAGTTTTCTTTTTTTTATTTCTCTC
TCAATTGGAGTCTGTCCTTCGTGCCGTGGAAACCCCGTAATCATGCAG
CACGGTACCGAGAGCGTGGCTCAGGCACGAACCGTCGCAAACGTGAGC
ATGTGTGTGGGTGCTGTGAAATGGGAAGCATCGATACGATAAGAAACT
CCAGCAATCGATTGTGCCAGGGCGCAAAGCCGGAGCAAACATAAACAT
GCAGCTCATCAAGGATGGGTTAAAGGAGTCGGCAACTAACCGGCTACA
GAACGAAACAGTGAAGCGCGAAGAAGCAATTGCTAACCGTGCGGTCCC
TTGCCTGACCGAACAATAGTGAAGCTCATTTTCCAAGCGACGTTGGTT
GGCTGTGTGGGCTATGGGGTAAATTTTAAAACTTCTTTTGGGGAAGTT
TTTGGAAGGAAAATTTCATTACGTTTCACCCTATTCCTTTGCAAGAGC
GGGTCGTGATAAGATCTCTCGATGGGGACGTGCTGCGAGACAGGTTGA
TAGTGGCGAGAAAACGTTTGACGAGCGATATCATTGAAAACTATCTGC
AAAATGCTTCACCAGCGGTGTGCACTTAGATGCTAGAGTTTAGTTTTC
GTTGCTAGGTGTGCAAGTGTGCAAAAAATATTCTTACAATCGCTTGTT
ACTTAAATTTTATTACAGATAGCGAACAAAGAGGATGTTATGTTTCAG
CTACATAAATTTCATTCAATAAGTACATTTCAATGGTAAAACATCTCC
CTTGTGTTAAAATCTGTACAATTGTTGAGAAATTTCAATGAAGTTTAT
AGGTTACTAATTACCGTTTATTATTCATAAAATAACAACTTAGCCCCT
GGACAATTCACGGATACTAGGATGTCCAAGGGTATGTGTGTAACTTTA
TCATAGAATAATTTGTTATCCTAATTACTTCGTTTTAACAGTGTATCG
CTCAGTTCTACGTCAACTATCCGTGGTTCAGTAGCTGAATTCCCGCGT
TGGAATCGCGTTGGTTCTAGGTTAGTATCTCATATGCAGATTGGTTAA
CATGATAGTCAATAATGTTTAAATCCATGACTGAACATTGAAGAATAT
GATACATTTTATGCTATTGCTATTTTTTTTAATTCATCACATACCACA
CGGTACATTATTGATTTCAGAAAGGCATATTTTGATTATTATATAATT
AAAAATTACAGCTATTTTTCAAGTAAACACCAAGCTCATGCATTAAAC
CACAATAAAATTGATTTTTTAATTACACTCAACACGCTAACATTTTTT
CAAAAAATAACATTACATCCATTACATGCCGTTGATGAATACATAAAT
TACGCCTTGTTTTTGATGCACGATAATTTTTATTTTGCGCACCTTTTG
CCCCCGGTCCTATACAACATTACCATGATTCGTACGTGTTCCCGCTCG
GCAAATCTCGCTAATCAACCGTTCAACAATCCATACATACCCGACGTT
GATCGCACACGATGTAACGCGGACCGGCTGGAGCGATTTTGGCTTGCC
CGACTCGACACAACCGATCGACATCAATTGCAGGGATTACCGGCACGC
CATCATCAACCGACATCGCCTCGGCAAACGCAGCTCCAATCAGCAGGG
GCTAATCACTCGAAGCAGGGATGCCCGGGGAGCAGAGAGACCAGAAAC
GCTACATTATCCACGCGGCTGCTATTAAGTTTCGCCCACAACCAGCGC
GCACACAATAATCGTCATTGATCGGCACCGGCAAAATTAAACATTGGC
AAACACAACGGCAACTACAAAAACTCCGATCAAACGGTCACGGTCTGA
ATTGAGCTCAAGGGGGATGGAGAGCGAGTGAGAAAGAGGTGAGATATC
ATATTCCAATCGATTTTATTCAAATTCTTAAATAACATTTATCTTCCC
GATAGCTGATTCATTGCCGTCGCTCACGCCTGCTTGTCTGCTTCCGCT
CCGTTCGCGTTCTATTTGCTACTGCATTATTTCTGCTGATGCACCCAA
TCATCCTATCTCCCACCCTCTCTATCTGTACTGAGCACCGGGCAGGGC
GAAAAAGGGGGAGCGGCAGCAAAATGCATTCCCCGGAGAGGAACAAGA
AGAAGAAGGCGGTGCAACAAAAAAGCAAACCCGGATCATCCCGGCTCG
GTGGAAAATAGATTACATTATTTGTGTTTCATTTTGTAGTATATACGT
GTGTGTGTGGGTGTGAGTGTTTGTAGTTTGCCTTAAATTGTTTTATAA
TTACTCTTGTGCGACAAAACGCCCCTGACTAGAGTGGGTTGGGAGCGA
ACACCACAATCGTGAACTGGACGGGAGAACATAATCCGATGTCCTCGG
GTGATTTGATGTACGCCAGGGAAAGCGGATCATCAAATGGTGTATACT
GGCAAATATGCAAAAACTTCGGAAAAGGGGAACTGGAACATTGAAACA
AGCTATTATGCACCTTGCACTTTGTCCCACCAACTGTCCAGCAATTCG
AAATAAAATGACAGAAGCGACCGTACATTACACTCCCATTTTTTTGTC
TTATTCTACATTTCAATACTTTTCGCCGGGTGTTTGACGGGAATGGAA
AAGGTGTGAAGCGCGTTCAATCTTCATCATCCTTTGCCCACATCTCGA
CCTGCGGACCTGGCGGGCCATGTCCATCAACGGGCAAGCTGCAGCGCC
CATCACCGCCGCTTTTTGTTACCCGTCGACTCATCTTCCGGTGCGGGC
CAGTGCAGTCTTTTCCTTTTTTACGCTCGCTCTCTCTCTTAAACGCTT
CCAATATTTGTGTTTAATTATTCGAACGGAATCCTCTCTGCGACAGCA
CATCCGTACGGGGTGCCAGTAGTGTGTGCGAGTCCGTGTTTGTGTGTA
GCCGTAATTATGTTGTGATTGTCATTGTCACTCGATGCGCGATAAACA
ATCTACCTACAATTTATGCACCCACTGGGCGGCCTCGCCTCGTGATCC
AGTCCGGTTTGCAAGTCGCCGCAACTCCAATTCAATGTCATCCGTTCT
CACAGCGAACGAACAGAACGGAGGGGACACGAACGCCAACAACAGCAA
CAGCGGCAAAAAATGCACCCAAAGTCCTGGATGCTGGGGATGACAAGA
GCCGCCGATCCGGCCTCCCACCACACACCAAACGCACAATCGCAGTTG
GAATTGCACGGTTTAAATATATACATGTTGTTGCTGTTTTTTTGTTTT
GTTTTTGGCGTGCAACTGTGCTGCTCCTGCTCCTATCGTGCGCTATCG
TGGCTGGATCCCGCGGGGCTACTCGGTGCACGGTCTAACGCATCCGGA
CGAGCGTTTGGTTTGGTTCCAATGTTGCAGTTGCAGTTGGAGTTCGGG
TCGGGGACAAAAAATCACTTACTTCCACTCGAGCGCCACCGCGCCGGA
ACGAACGCGGAAACCCGTTCCACGGTCCATCATACTCTCTTTCCTCCC
TCCCCAACCGTCGCTCAGTTCAACATATGGCCGTGGGGATCGGGATTG
GGAGCTGTCAGGTCCAGGTGCCGCGGGAAGGGATCCTGCAGGGAAGTA
TCAAGCGCCGGAACTGGAAGCACCCGATGACAGATGGTGCTCGAAAGT
GAACTGTAAAACTGGACGCCCATCACCAACAACATCACACCGGCATGC
AGTGCGACAAAAAAAACACACCCACACTGAGAGAGAAACAAAAATCAC
ATCCACGCCCGTCGTCATCAGGGGCGAAAAAACAACAAACCACACAAC
CGGCTGAGCCAACAGAAACTAACACAGCGCGCACTGGGCTGGCCACAA
AATGTAGTACTAACTAAATCCAATCCAAATAATTATATTTCAATTGTT
TATGAACGGCATTATGCGACCGGACCGGAAAGTCGCTGGCTCGACTCG
TCCGTCCAGTCCCAGCAACAATATCAACAATAACACATGCTCCCGGCC
TGGAACGGTGGGTATGCGTCGGCGGCGTATGCTGACCAACATAATCAA
CGTATCCTTTGTGGTGGGATTCCGGGATTCCGGCAGGATCCGC

SEQ ID No: 1 is the whole AGAP004050 gene, plus about 3000 bp upstream of its putative promoter and about 4000 bp downstream of its putative terminator.

Accordingly, in an embodiment the doublesex gene comprises a nucleic acid sequence substantially as set out in SEQ ID NO: 1, or a fragment or variant thereof.

In an embodiment, the intron-exon boundary targeted by the genetic construct is the intro-exon boundary provided herein as SEQ ID No: 2, as follows:

[SEQ ID No: 2]
CCTTTCCATTCATTTATGTTTAACACAGGTCAAGCGGTGGTC
AACGAATACTCACGATTGCATAATCTGAACATGTTTGATGGC
GTGGAGTTGCGCAATACCACCCGTCAGAGTGGATGATAAACT
TTC

The target sequence may include up to 1, 2, 3, 4, 5, 10 or 15 nucleotides 5′ and/or 3′ of SEQ ID No: 2.

In another embodiment, the intron-exon boundary targeted by the gene drive construct is provided herein as SEQ ID No: 3, as follows:

[SEQ ID No: 3]
CCTTTCCATTCATTTATGTTTAACACAGGTCAAGCGGTGGTC
AACGAATACTCA

The target sequence may include up to 1, 2, 3, 4, 5, 10 or 15 nucleotides 5′ and/or 3′ of SEQ ID NO:3.

In another embodiment, the intron-exon boundary targeted by the gene drive construct is provided herein as SEQ ID No: 4, as follows:

[SEQ ID No: 4]
GTTTAACACAGGTCAAGCGGTGG

The target sequence may include up to 1, 2, 3, 4, 5, 10 or 15 nucleotides 5′ and/or 3′ of SEQ ID NO:4.

Preferably, in the system according to the invention, the intron-exon boundary of the female-specific exon of the doublesex (dsx) gene has a sequence comprising or consisting of the nucleotide sequence substantially as set out in any of SEQ ID NO: 2, 3, and 4, or a fragment or variant thereof.

In an embodiment, the nucleotide sequence that hybridises to the intron-exon boundary of the female-specific exon of doublesex (dsx) gene comprises a sequence as provided herein as SEQ ID No: 5, as follows:

[SEQ ID No: 5]
GTTTAACACAGGTCAAGCGG

The part of the nucleotide sequence that is capable of hybridising to the intron-exon boundary (i.e. the guide RNA) is known as a protospacer. In order for the nuclease to function, it also requires a specific protospacer adjacent motif (PAM) that varies depending on the bacterial species of the nuclease encoding gene. The most commonly used Cas9 nuclease recognizes a PAM sequence of NGG that is found directly downstream of the target sequence in the genomic DNA on the non-target strand.

The CRISPR nuclease binding sequence creates a secondary binding structure which complexes with the nuclease, for example a hairpin loop. The PAM on the host genome is recognised by the nuclease.

Preferably, the CRISPR-based gene drive construct is a CRISPR-Cpfi-based or a CRISPR-Cas9-based gene-drive genetic construct.

In a preferred embodiment, the CRISPR-based gene drive construct is a CRISPR-Cas9-based gene-drive genetic construct.

The CRISPR nuclease binding sequence creates a secondary binding structure which complexes with the nuclease, for example a hairpin loop. The PAM on the host genome is recognised by the nuclease.

Accordingly, in an embodiment, the nucleotide sequence encoding a nucleotide sequence that is capable of hybridising to the intron-exon boundary of the doublesex (dsx) gene (i.e. a guide RNA) is provided herein as SEQ ID No: 6, as follows:

[SEQ ID No: 6]
GTTTAACACAGGTCAAGCGGGTTTTAGAGCTAGAAATAGCAA
GTTAAAATAAGGCTAGTCCGTTATCAACTTGAAAAAGTGGCA
CCGAGTCGGTGCT

Preferably, the nucleotide sequence of the CRISPR-based gene drive genetic construct that hybridises to the intron-exon boundary of the female-specific exon of doublesex (dsx) gene comprises a sequence substantially as set out in any of SEQ ID NO: 5 and SEQ ID NO:6, or a fragment or variant thereof.

For example, according to an embodiment of the third aspect of the invention, a system is provided comprising:
(i) an anti-CRISPR construct comprising a vasa2 promoter sequence operably linked to a nucleotide sequence coding for a nuclear localisation signal (NLS)-tagged AcrIIA4 protein; and
(ii) a CRISPR-based gene drive genetic construct comprising a nucleotide sequence encoding a nucleotide sequence that hybridises to the boundary between intron 4 and exon 5 of the doublesex (dsx) gene in Anopheles gambiae, such that the CRISPR-based gene drive genetic construct disrupts the intron 4-exon 5 boundary of the female specific splice form of the dsx gene in the mosquito.

In a fourth aspect, the present invention refers to a method of producing a genetically modified arthropod, the method comprising introducing into an arthropod an anti-CRISPR construct comprising a nucleotide sequence encoding an Acr protein.

Preferably, the anti-CRISPR construct comprising the nucleotide sequence encoding an Acr protein is an anti-CRISPR construct according to any of the embodiments of the invention described above.

The anti-CRISPR construct may be introduced directly into an arthropod host cell, preferably an arthropod host cell present in an arthropod embryo, by suitable means, e.g. direct endocytotic uptake. The construct may be introduced directly into cells of a host arthropod (e.g. a mosquito) by transfection, infection, electroporation, microinjection, cell fusion, protoplast fusion or ballistic bombardment. Alternatively, constructs of the invention may be introduced directly into a host cell using a particle gun.

Preferably, the construct is introduced into a host cell by microinjection of arthropod embryos, preferably an insect embryo and most preferably mosquito embryos.

Preferably, the gene drive genetic construct and the anti-CRISPR construct are introduced into freshly laid eggs, within 2 hours of deposition. More preferably, the anti-drive construct is introduced into an arthropod embryo at the start of melanisation, which the skilled person would understand takes place within 30 minutes after egg laying.

In an embodiment, the arthropod is a mosquito. Preferably, the mosquito is of the subfamily Anophelinae. Preferably, the mosquito is selected from a group consisting of: Anopheles gambiae; Anopheles coluzzi; Anopheles merus; Anopheles arabiensis; Anopheles quadriannulatus; Anopheles stephensi, Anopheles funestus and Anopheles melas.

According to an embodiment of any aspect of the present invention, the arthropod is selected from the group consisting of Aedes aegypti, Ceratitis capitata, Drosophila Suzukii, Aedes albopictus, Bactrocera oleae, Rhynchophorus ferrugineus, Tuta absoluta, Spodoptera Frugiperda, Lucilia cuprina, Ostrinia nubilalis, Diabrotica virgifera, Helicoverpa armigera, Cochliomyia, Solenopsis invicta, Anoplophora glabripennis, Coptotermes formosanus, Lymantria dispar; Plutella xylostella, Pectinophora gossypiella, Philaenus spumarius, Listronotus bonariensis, Adelges tsugae, Anopheles quadrimaculatus, Trogoderma granarium, Pheidole megacephala, Linepithema humile, Bemisia tabaci, Vespula germanica, Anoplolepis gracilipes, Agrilus planipennis, Wasmannia auropunctata, Vespula vulgaris, and Cinara cupressi

In a fifth aspect, the present invention refers to a genetically modified arthropod comprising an anti-CRISPR construct comprising a nucleotide sequence encoding an Acr protein, preferably wherein said anti-CRISPR construct is an anti-CRISPR construct according to any of the embodiments of the invention described above.

In a preferred embodiment, the arthropod is an insect, preferably wherein the insect is a mosquito, more preferably wherein the mosquito is of the subfamily Anophelinae, even more preferably wherein the mosquito is selected from a group consisting of: Anopheles gambiae; Anopheles coluzzi; Anopheles merus; Anopheles arabiensis; Anopheles quadriannulatus; Anopheles stephensi; Anopheles fimestus; and Anopheles melas.

In a preferred embodiment, the genetically modified arthropod is Anopheles gambiae.

In a sixth aspect, the present invention refers to a method for counteracting a CRISPR-based gene-drive in an arthropod population comprising arthropods carrying a CRISPR-based gene-drive construct, said method comprising the release of the genetically modified arthropod according to the invention in the arthropod population.

In a preferred embodiment of the sixth aspect of the invention, the CRISPR-based gene drive genetic construct comprises a nucleotide sequence encoding a nucleotide sequence that hybridises to the intron-exon boundary of the female-specific exon of the doublesex (dsx) in an arthropod, such that the CRISPR-based gene drive genetic construct disrupts the intron-exon boundary of the female specific splice form of the dsx gene in the arthropod.

In a preferred embodiment of the sixth aspect of the invention, the CRISPR-based gene drive genetic construct comprises a nucleotide sequence encoding a nucleotide sequence that hybridises to the intron-exon boundary of the female-specific exon of the doublesex (dsx) in a mosquito, such that the CRISPR-based gene drive genetic construct disrupts the intron-exon boundary of the female specific splice form of the dsx gene in the mosquito.

In a seventh aspect, the present invention refers to the use of the construct according the invention or of the genetically modified arthropod according to the invention to counteract a CRISPR-based gene-drive in an arthropod population comprising individuals carrying a CRISPR-based gene-drive construct.

In a preferred embodiment of the seventh aspect of the invention, the CRISPR-based gene drive genetic construct comprises a nucleotide sequence encoding a nucleotide sequence that hybridises to the intron-exon boundary of the female-specific exon of the doublesex (dsx) in an arthropod, such that the CRISPR-based gene drive genetic construct disrupts the intron-exon boundary of the female specific splice form of the dsx gene in the arthropod.

In a preferred embodiment of the seventh aspect of the invention, the CRISPR-based gene drive genetic construct comprises a nucleotide sequence encoding a nucleotide sequence that hybridises to the intron-exon boundary of the female-specific exon of the doublesex (dsx) in a mosquito, such that the CRISPR-based gene drive genetic construct disrupts the intron-exon boundary of the female specific splice form of the dsx gene in the mosquito.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1 to 26 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, SEQ ID Nos:1 to 26.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:

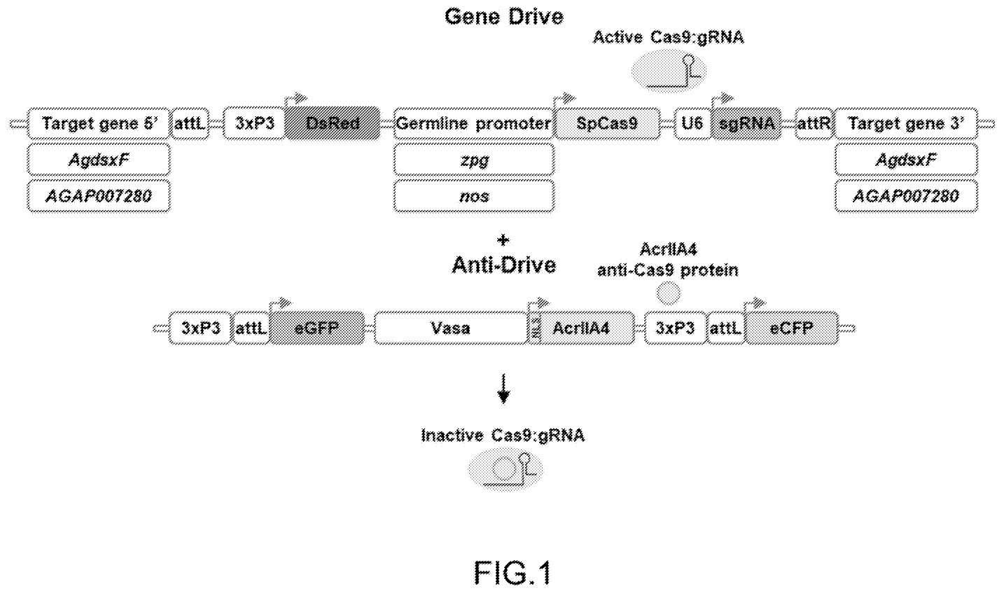

FIG. 1 is a schematic representation of gene drive and anti-drive constructs. Gene drive and anti-drive constructs respectively inserted in the genome of previously generated gene drive lines (zpg:dsxF 2; zpg:7280 and nos:7280 33) and newly generated anti-drive (vasa:A4) line. The gene drive constructs tested in this work are inserted at target sites within the AGAP007280 or AgdsxF (AGAP004050-RB) gene coding sequences and contain: the Streptococcus pyogenes Cas9 nuclease (SpCas9), under the transcriptional control of the male and female germline specific promoters zpg or nos; the gRNA, targeting the respective insertion site, transcribed by the RNA polymerase III responsive promoter (U6) and the DsRed fluorescent protein under the 3xP3 promoter (3xP3:DsRed) for the identification of larvae carrying the drive. The anti-drive construct carries the Listeria monocytogenes anti-CRISPR protein (AcrIIA4) expressed under the vasa2 male and female germline specific promoter with the N-terminus addition of a nuclear localisation signal (NLS) and the eGFP fluorescent protein under the 3xP3 promoter (3xP3:eGFP) used for A+ larvae screening. The construct was inserted in a pre-existing docking line carrying the 3xP3:eCFP marker. The AcrIIA4 protein is expected to interact and inhibit the Cas9-gRNA complex when coexpressed in the mosquito germline cells.

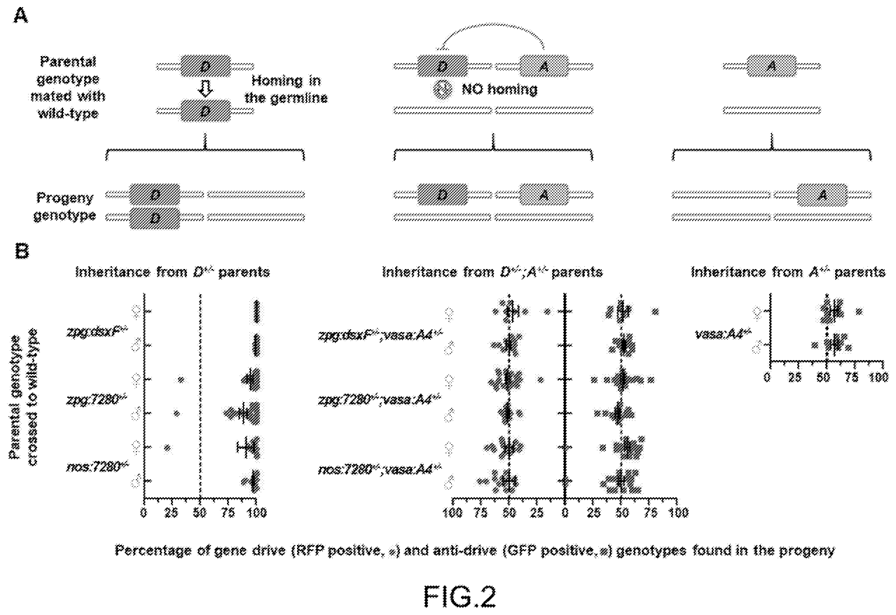

FIG. 2 shows inhibition of gene drive homing by germline expression of anti-CRISPR protein AcrIIA4. (A) Schematic representation of gene drive homing in the germline of individuals carrying one copy of the drive allele (D^{+/-}). Cas9-gRNA directed cleavage of the insertion site on the wild-type homologous chromosome is repaired via homology directed repair (HDR) using the drive-carrying chromosome as template resulting in the D allele being transmitted to most of the progeny. The new drive copy is indicated as dashed purple rectangle (left). Illustration of gene drive homing inhibition in individual carrying one drive and one anti-drive copy (D+/−; A+/−) as consequence of AcrIIA4-directed Cas9-gRNA blockage, resulting in Mendelian inheritance of the D allele (middle). Mendelian inheritance of the anti-drive from A+/− individuals (right). b Scatter plots showing the percentage of larvae carrying the gene drive (RFP positive) and/or the anti-drive (GFP positive) constructs from wild-type mosquitoes crossed to transgenic females or males carrying: only the gene drive construct, confirming high transmission rates (up to 100%) of the D allele from each of the transgenic lines tested (left); both gene drive and anti-drive constructs, showing Mendelian inheritance of both D and A alleles (middle); only the anti-drive construct, showing expected Mendelian rates of the A allele (right). Vertical dashed lines indicate the 50% Mendelian inheritance. Error bars indicate mean percentage values and standard error of the mean of transmission rates from all biological samples assessed for each cross. A minimum of seven biologically independent samples (ovipositing females) were examined over two independent experiments for each cross, with the exception of zpg:dsxF crosses, which were examined only once.

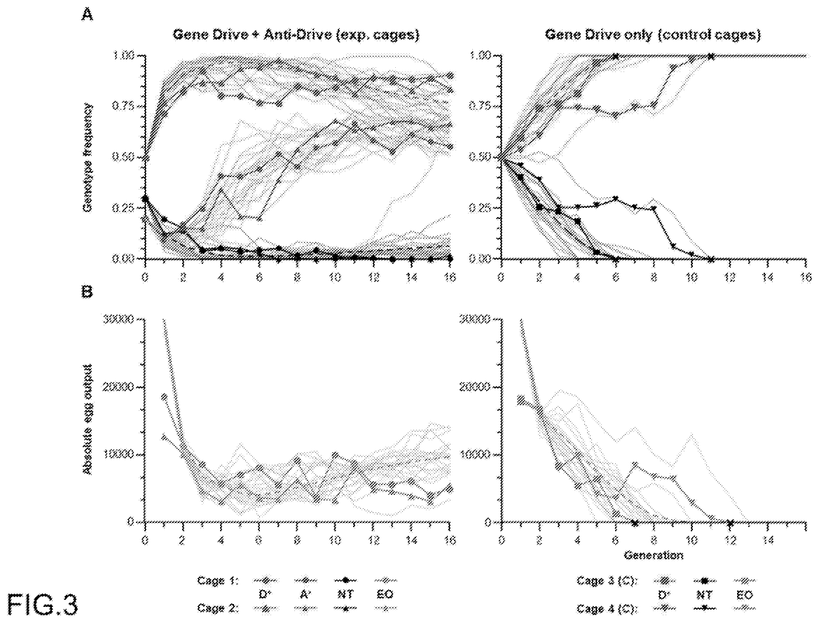

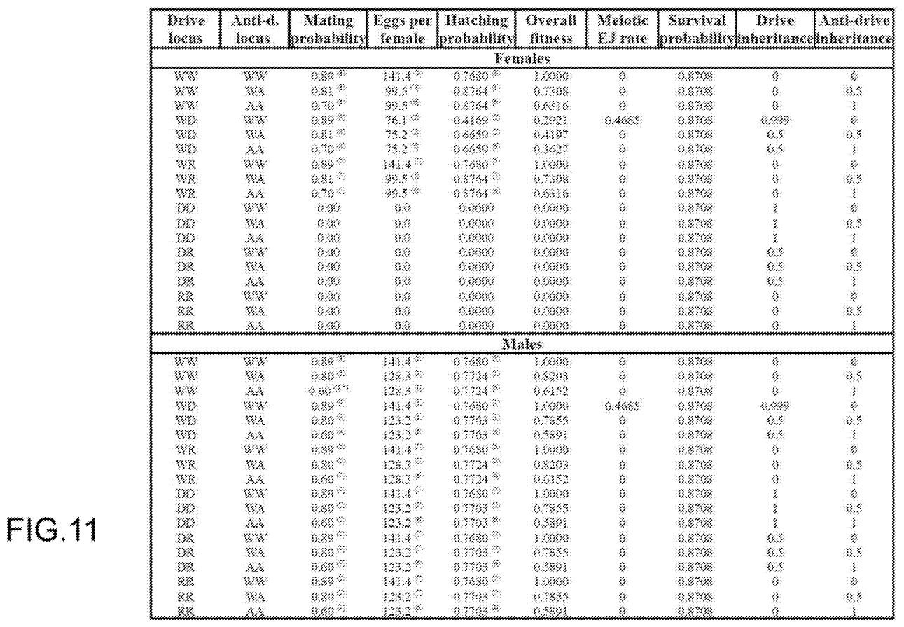

FIG. 3 shows how a single release of AcrIIA4 anti-drive males constrains gene drive spread preventing population suppression in caged mosquitoes. Two cages were initiated with a starting population of 600 A. gambiae mosquitoes of which: 150 males and 150 females heterozygous for the zpg:dsxF driving allele (initial drive allelic frequency of 25%), 120 homozygous-enriched males for the vasa:A4 allele (initial anti-drive allelic frequency of 20%) and 180 wild-type of which 30 males and 150 females to maintain equal sex ratio (left). In parallel, two control cages were established by releasing the same proportion of drive alleles (150 zpg:dsxF^{+/-} males and 150 zpg:dsxF^{+/-} females) and 300 wild-type mosquitoes (150 males and 150 females). (A) The frequency of drive (D+, RFP positive, purple lines), anti-drive (A⁺, GFP positive, green lines) and nontransgenic individuals (NT, black lines) was recorded for each generation by screening larvae for the expression of the respective fluorescent markers. (B) Absolute number of eggs produced each generation (grey lines). Genotype frequencies (D⁺, A⁺ and NT) and egg output (EO) values were overlapped to the respective deterministic (dotted lines) and stochastic (light coloured lines) model simulation based on the parameters provided in FIG. 11.

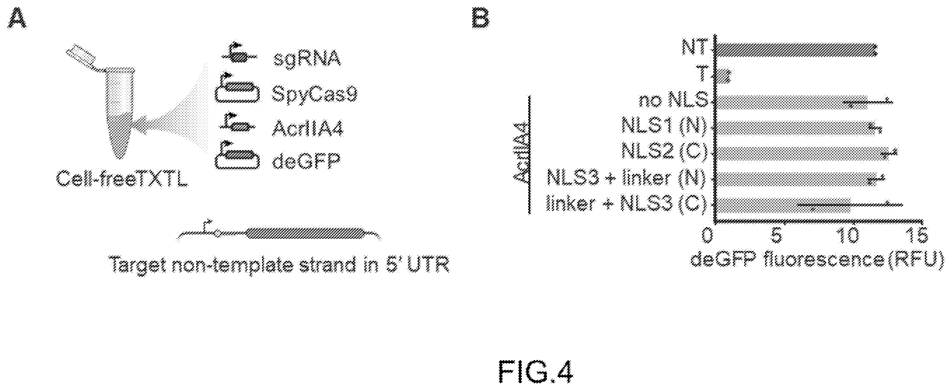

FIG. 4 shows Inhibitory activity of AcrIIA4 unperturbed following the addition of NLS tags. (A) Assessing inhibition of SpyCas9 by AcrIIA4 using an E. coli-based cell-free transcription-translation system (TXTL). As part of the assay, SpyCas9 and an sgRNA targeting the deGFP construct are expressed, leading to cleavage and loss of deGFP expression. The presence of expressed AcrIIA4 inhibits DNA cleavage by SpyCas9, restoring deGFP expression. The components are encoded on linear DNA or on plasmids. (B) Assessing the impact of different NLS tags. Each tag was fused to the N-terminus (N) or C-terminus (C) of AcrIIA4. T: targeting sgRNA expressed without AcrIIA4. NT: non-targeting sgRNA expressed without AcrIIA4. NLS1 sequence: APKKKRKVGIHGVPAA [SEQ ID NO:23]. NLS2 sequence: KRPAATKKAGQAKKKK [SEQ ID NO:24]. NLS3 sequence: MPKKKRKV [SEQ ID NO:25]. Linker: SGGS [SEQ ID NO:26]. NLS sequences at the N-terminus begin with methionine to initiate translation. All NLS tags resulted in full restoration of deGFP expression. Values represent the mean and standard deviation of duplicate measurements.

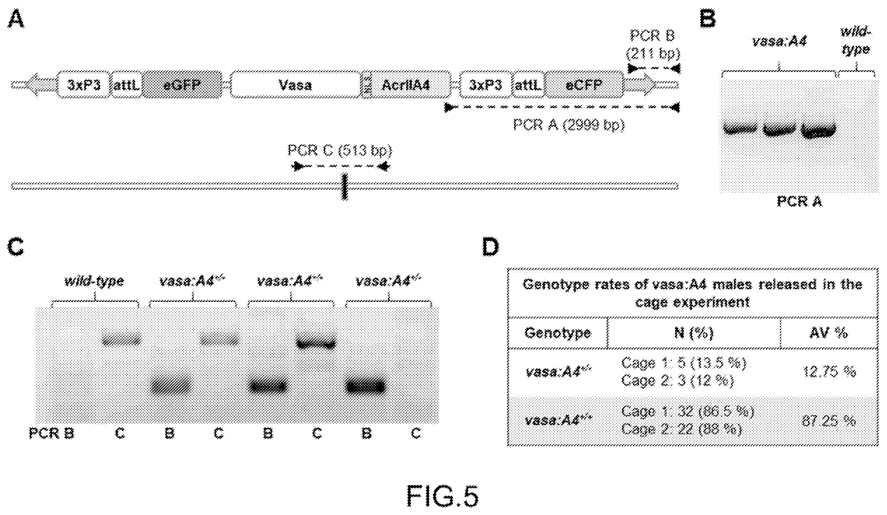

FIG. 5 shows the molecular characterization of the vasa:A4 transgenic line. (A) Schematic representation of the genomic integration of the vasa:A4 construct indicating the expected size of PCR fragments amplified using each set of primers (A, B and C). (B) Molecular confirmation of successful #C31 mediated integration of the vasa:A4 construct. (C) Examples of PCR amplifications from genomic DNA extracted from single mosquitoes carrying one (vasa:A4^{+/-}) or two copies (vasa:A4^{+/+}) of the vasa:A4 construct and wild-type. (D) Proportion of heterozygous (vasa:A4^{+/-}) and homozygous (vasa:A4^{+/+}) anti-drive males released in the cage trial according to the PCR analysis shown in “C”.

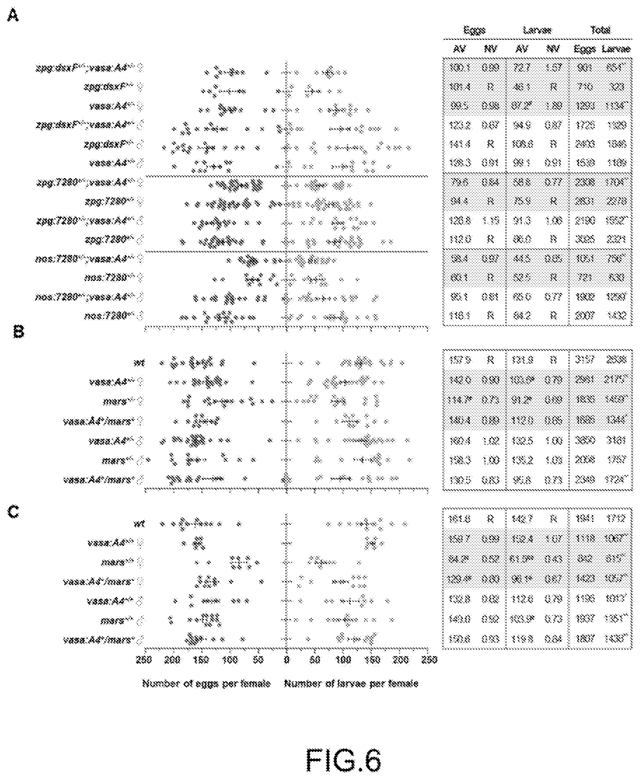

FIG. 6 shows fertility assays of gene drive and anti-drive transgenic lines. Scatter plots of the total number of eggs (dark grey dots) and larvae (light grey dots) counted from individual oviposition assays from wild-type mosquitoes crossed to transgenic females or males carrying: (A) one copy of the gene drive and/or anti-drive constructs; (B) one copy of the anti-drive constructs and/or one copy of a marker construct inserted at the same locus; (C) two copies of the anti-drive constructs or two copies of a marker construct inserted at the same locus (vasa:A4/mars crosses were also repeated for parallel reference). Error bars indicate mean values of number of eggs or larvae for each cross (also reported in the table on the right under average values (AV))±standard error of the mean. Normalised values (NV) were calculated against selected reference crosses (R) performed in parallel. Significance according to Welch's unpaired t-test (for both larval and egg output average values, indicated by “#”) and Fisher's exact test (for the total number of hatched larvae, indicated by “*”) was calculated against the reference cross (“*” or “#” corresponds to P<0.05, (“**” or “##” corresponds to P<0.0001).

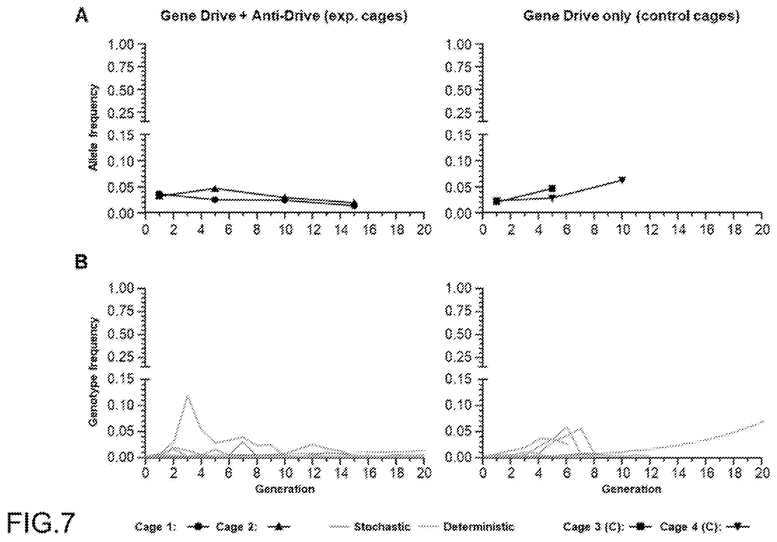

FIG. 7 shows resistance dynamics over generations at the dsx-target sequence. (A) Frequency plots of the total number of mutated alleles (indels and substitutions) among non-drive alleles, detected at the gRNA target sequence from 4 generations of the cage experiment (G1, 5, 10 and 15). (B) Resistant genotype frequency trajectories modelled by deterministic (dotted line) or stochastic simulations (solid lines) over 20 generations.

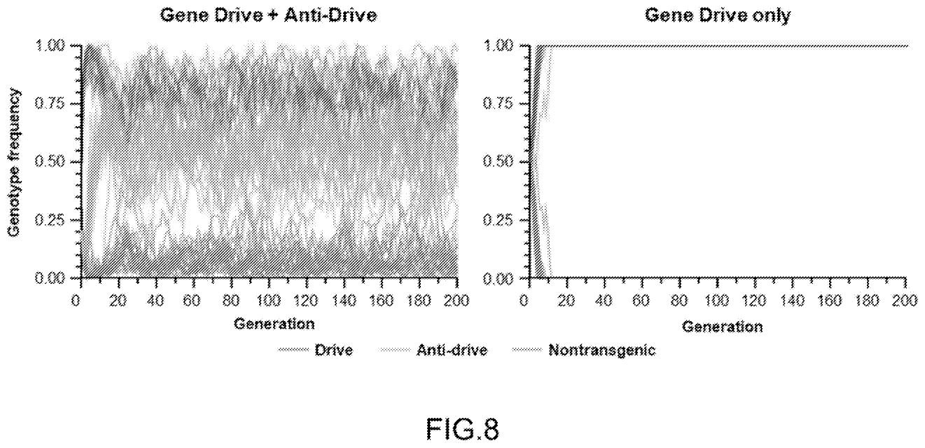

FIG. 8 shows stochastic dynamics of zpg:dsxF drive and AcrIIA4 anti-drive genotypes over extended time. Frequency over 200 generations of drive, anti-drive and nontransgenic individuals according to fitness parameters used for the cage trial models (FIG. 3, and FIG. 11). The same starting frequencies were also applied, including the additional reduction in mating probability assumed for WW; AA males at G0 (0.2225 in G0 and 0.6 from G1 onwards).

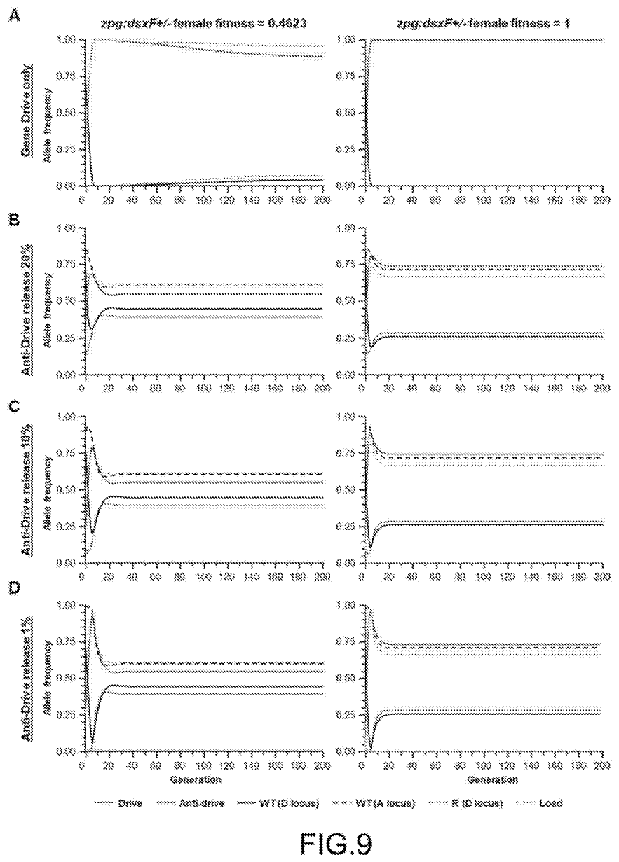

FIG. 9 shows the effect of dive fitness on gene-drive and anti-drive allelic dynamics. Plots showing reproductive load and deterministic dynamics of drive, anti-drive, wild-type and non-functional resistant alleles assuming release of (A) only 25% drive alleles for the control plots, (B) 25% drive and 20% anti-drive alleles, as used for the cage trial and stochastic models, (C) 25% drive and 10% anti-drive alleles or (D) 25% drive and 1% anti-drive alleles, contributed by homozygote males. Two different fitness values (relative to wild-type) of heterozygous gene drive females (WD; WW) were used: (left) equal to zpg:dsxF^{+/-} females analysed in Kyrou et al. (0.4623), or (right) equal to wild-type (1.0).

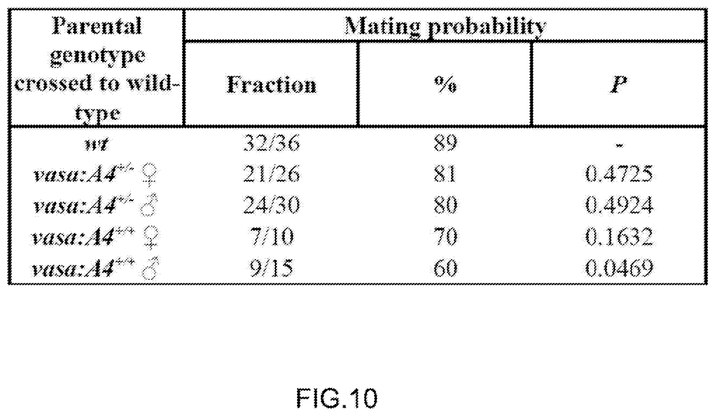

FIG. 10 is a table showing the mating probability of mosquitoes carrying one or two copies of the vasa:A4 construct. Fraction of mated females or males carrying one (vasa:A4+/−) or two copies (vasa:A4+/+) of the vasa:A4 construct scored in fertility assays. Fisher's exact (two-tailed) test was used to calculate significance against the wild-type control.

FIG. 11 is a table showing the parameters used for modeling. “W” indicates the wild-type allele at the drive (left) or anti-drive locus (right). “A” indicates the anti-drive allele. “D” indicates the drive allele. “R” indicates alleles causing non-functional resistance to the drive. ⁽¹⁾ Average values obtained from phenotypic analysis performed in this work. ⁽²⁾ WD fertility values measured in this work were normalised for parental deposition in females measured in Kyrou et al. (maternal/paternal reduction rates: eggs per female=0.50, hatching probability=0.66). ⁽³⁾ Male fertility of WW; WW mosquitoes is considered equal to WD; WW males as in Kyrou et al. (4) Mating probability of WD individuals is considered equal to WW as in Kyrou et al. (5) Mating probability and fertility of WR males and females is considered equal to WW. (6) Fertility of AA mosquitoes is considered equal to WA. (7) Mating probability and fertility of DD, DR and RR males is considered equal to WD males (equal to WW) as in Kyrou et al. (*) An additional reduction in mating probability was assumed for WW; AA males at G0 (0.2225) for the cage trial models (FIG. 3, FIG. 7, FIG. 8 and FIG. 9). Inheritance values were rounded to 0.5 or 1 according to average values obtained from phenotypic analysis performed in this work. A 0.999 (instead of 1) value was used for WD; WW individuals to allow for R generation (0.4685 according to Hammond et al. 2016). Survival probability was also considered equal to Kyrou et al.

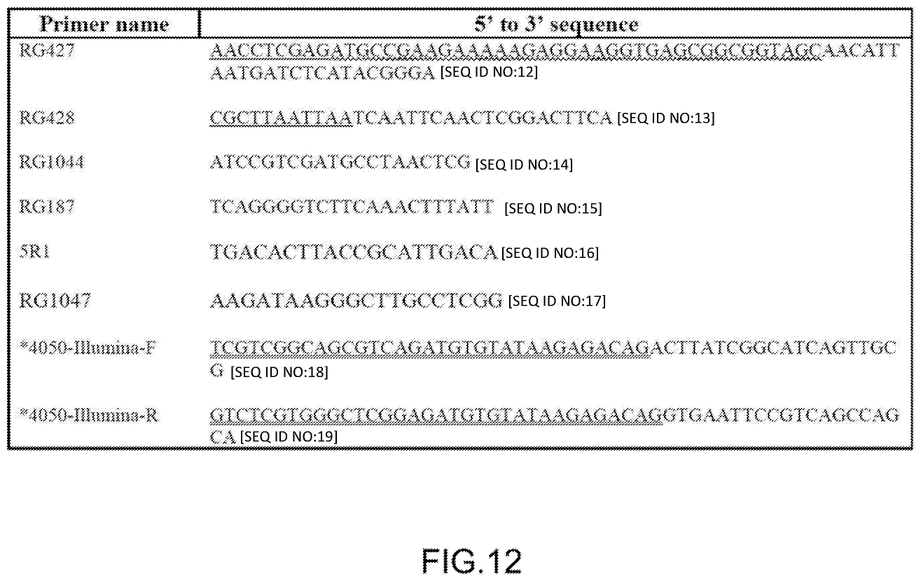

FIG. 12 is a table listing the primers used in this study. Cloning overhangs are underlined with a single line and NLS sequence with wavy line. * Primers used for amplicon sequencing (Illumina adaptors underlined with double line).

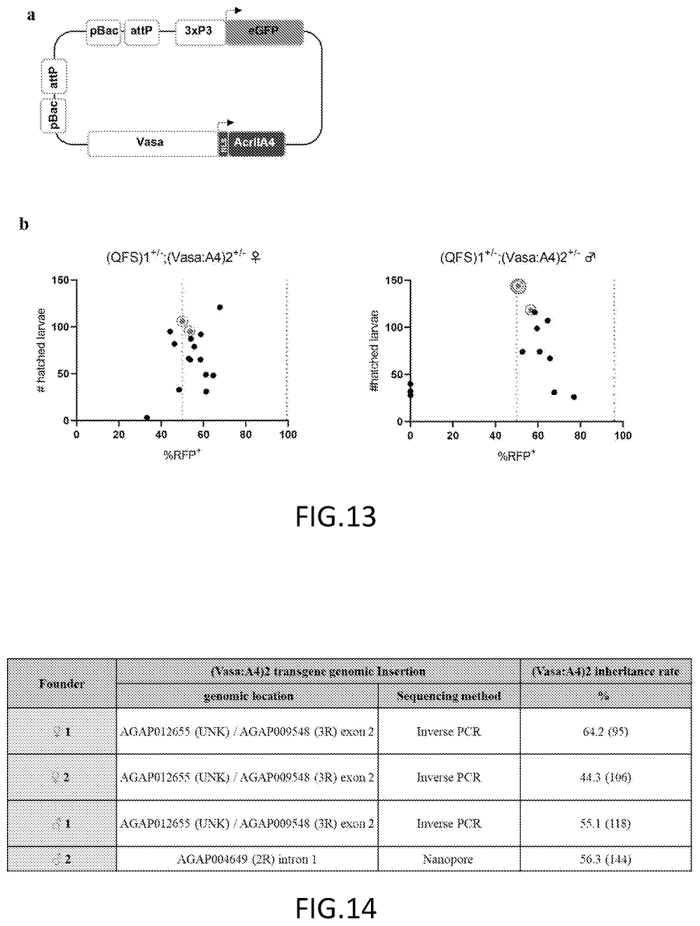

FIG. 13 shows the generation and selection of the Ag(Vasa:A4)2 transgenic line. (A) Schematic representation of the construct used to generate an anti-drive transgenic line; the construct carries the Listeria monocytogenes anti-CRISPR protein (AcrIIA4) expressed under the vasa2 male and female germline-specific promoter with the N-terminus addition of a nuclear localisation signal (NLS) and the eGFP fluorescent protein under the 3xP3 promoter (3xP3:eGFP) used for the screening of anti-drive positive insects. The construct contains piggyBac repeats on either side for semi-random integration in the genome. (B) Fertility and inhibitory activity against dsx targeting gene-drive in female (left) or male (right) transheterozygote parents, presented as number of hatched larvae per parent against % of RFP+ larvae in the progeny of each parent. Blue circled dots represent the progenies selected for further phenotypic analysis. Red dotted lines represent the expected mean GD inheritance rate in the absence of anti-CRISPR protein. Grey dotted lines represent Mendelian inheritance (50%). The double circled progeny was selected for the establishment of the (Vasa:A4)2 transgenic line.

FIG. 14 shows the characterisation of selected transgenic founders carrying (Vasa:A4)2 transgene insertion in transheterozygosity with (QFS)1. The final column shows the inheritance rate of the (Vasa:A4)2 transgene scored in the progeny. The total number of larvae screened is given in parentheses. Male 2 was selected for the establishment of the (Vasa:A4)2 transgenic line.

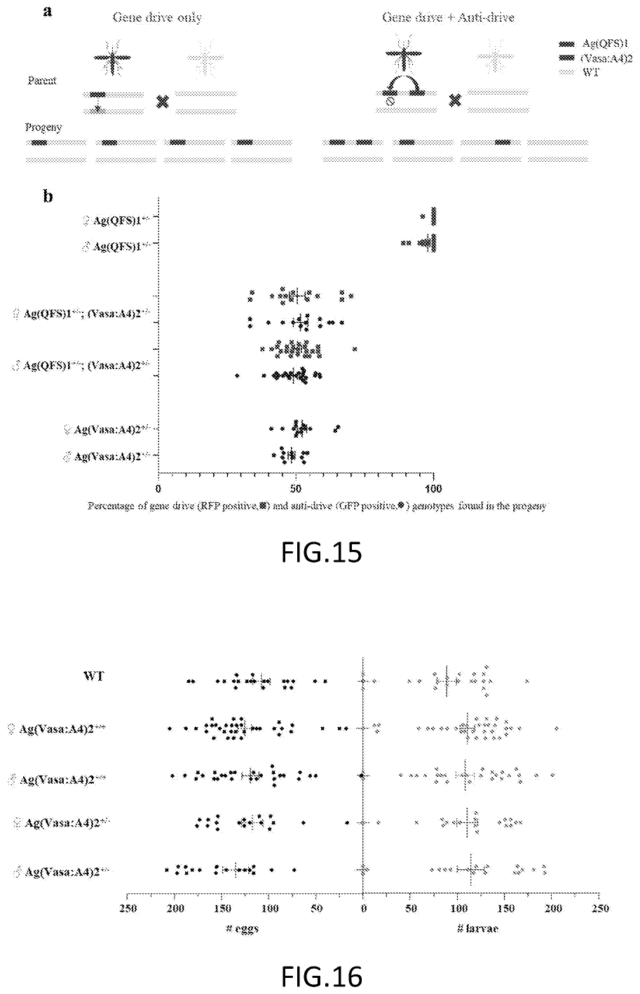

FIG. 15 shows inhibition of Ag(QFS)1 gene drive homing by germline expression of anti-CRISPR protein AcrIIA4 integrated in chromosome 2R via piggyBac transposase mediation. (A) Schematic representation of gene drive homing in the germline of heterozygous Ag(QFS)1 individual: Cas9-gRNA-directed cleavage of the insertion site on the homologous wild-type chromosome is repaired via homology-directed repair (HDR), using the drive-carrying chromosome as template, resulting in the gene drive allele being copied (the new copy is indicated as dimmed red rectangle) and transmitted to most of the progeny (left). Illustration of gene drive homing inhibition in individual transheterozygous individual, carrying both the drive and anti-drive; AcrIIA4-directed Cas9-gRNA blockage results in Mendelian inheritance of the gene drive allele (right). (B) Scatter plots showing the percentage of larvae carrying the gene drive (RFP positive) and/or the anti-drive (GFP positive) constructs from wild-type mosquitoes crossed to transgenic females or males carrying: only the gene drive construct (Ag(QFS)1^{+/-}), confirming high transmission rates (up to 100%) of the gene drive allele; both gene drive and anti-drive constructs (Ag(QFS)1^{+/-}; (Vasa:A4)2^{+/-}), showing Mendelian inheritance of both gene drive and anti-drive alleles; only the anti-drive construct (Ag(Vasa:A4)2^{+/-}), showing expected Mendelian rates of the A allele. Error bars indicate mean percentage values and standard error of the mean of transmission rates from all biological samples assessed for each cross.

FIG. 16 shows fertility assays of the anti-drive transgenic line Ag(Vasa:A4)2. Scatter plots of the total number of eggs (black dots) and larvae (grey dots) counted from individual oviposition assays from wild-type mosquitoes crossed to females or males carrying: a wild-type allele (WT); one copy of the anti-drive construct (Ag(Vasa:A4)2^{+/-}); two copies of the anti-drive construct(Ag(Vasa:A4)2^{+/-}). Error bars indicate mean values of number of eggs or larvae for each cross ±standard error of the mean. Significance according to Welch's unpaired t-test (for both larval and egg output average values) was calculated against the wild-type cross.

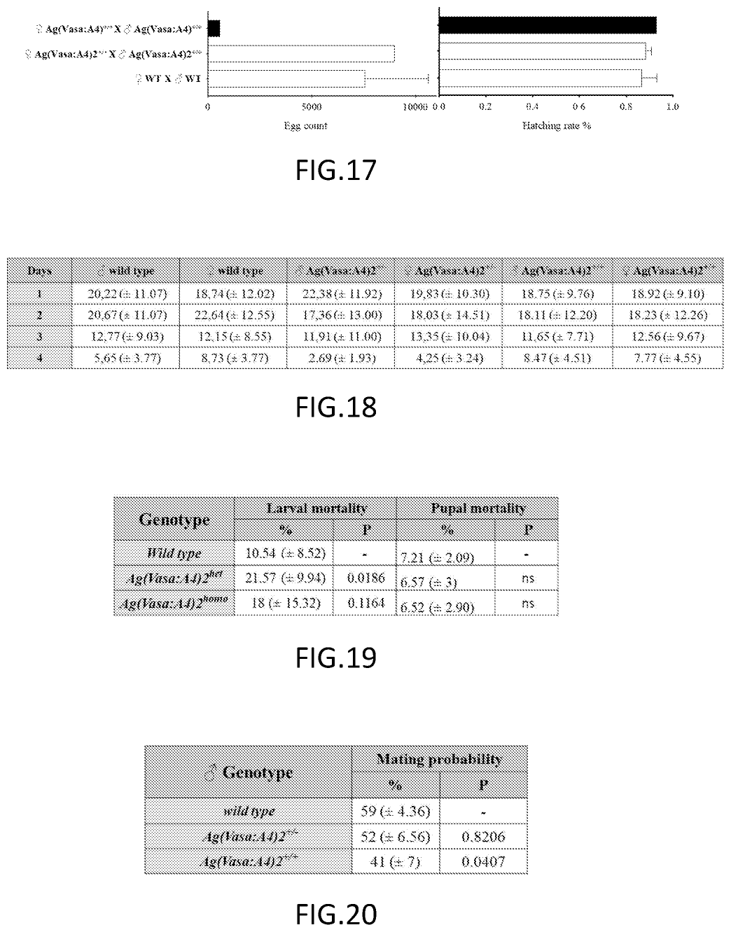

FIG. 17 shows assessment of fertility in bulk for the two anti-drive transgenic lines Ag(Vasa:A4) and Ag(Vasa:A4)2 when homozygote individuals are crossed to each other. The number of eggs and the relative hatching rate was calculated from bulk oviposition assays from the following crosses: Ag(Vasa:A4)2^{+/+} males and females mated with each other, Ag(Vasa:A4)^{+/+} males to Ag(Vasa:A4)^{+/+} females, and wild-type (WT) males to females (controls). No significant reduction in the fertility of the new transgenic line was observed (Ag(Vasa:A4)) that was apparent in the original anti-drive transgenic line (Ag(Vasa:A4)2).

FIG. 18 shows time of pupation of mosquitoes carrying one or two copies of the Ag(Vasa:A4)2 construct. Scoring of the male and the female pupae collected every day for each genotype. Each percentage value represent the average from three biological replicates; Anova test performed did not show statistic differences.

FIG. 19 shows larval and pupal mortality of (Vasa:A4)2 carrying mosquitoes in hetero- or homozygosity. The number of dead larvae and pupae from Ag(Vasa:A4)2^{+/-}, Ag(Vasa:A4)2^{+/+} and wild type strains was recorded, and a two-way Anova test performed. Statistic difference was observed for the larval mortality of the Ag(Vasa:A4)2^{+/-} strain (p=0.0186).

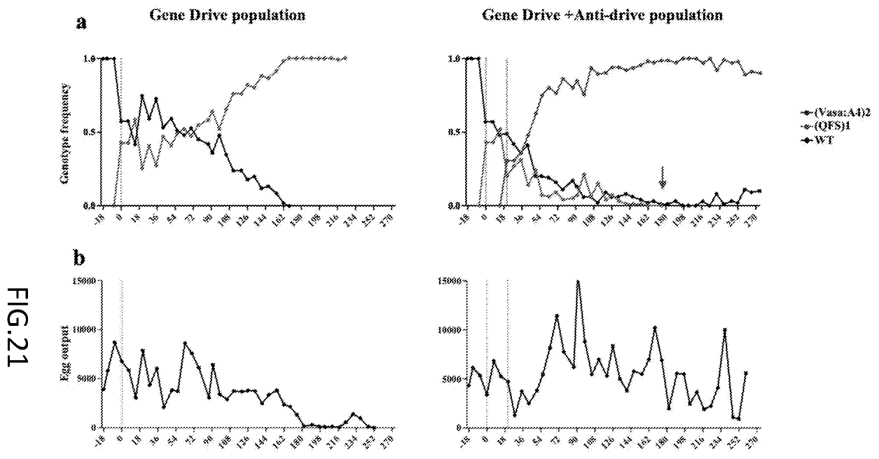

FIG. 20 shows mating competitiveness of (Vasa:A4)2 carrying males in hetero- or homozygosity. Ag(Vasa:A4)2^{+/-}, Ag(Vasa:A4)2^{+/+} and wild type males were crossed to wild type females to measure the mating competitiveness. Three biological replicates were carried out, and statistical difference were observed for Ag(Vasa:A4)2^{+/-} (p=0.0407; Kruskal-Wallis test).

FIG. 21 shows how multiple releases of Ag(Vasa:A4)2 anti-drive males removes Ag(QFS)1 gene drive alleles in caged mosquitoes and prevents population suppression. In two medium-sized cages, a starting population of 400 wild-type A. gambiae mosquitoes were introduced; then, a release of 150 mixed wild-type mosquitoes each were performed over the following two weeks. In the cage named ‘gene drive population’, Ag(QFS)1 heterozygous males were released at 12.5% allelic frequency for three weeks (representing 42.5% of the released individual). For the cage called ‘gene drive+anti-drive population’, following the gene drive release, Ag(Vasa:A4)2 homozygous males were released, at 15% allelic frequency (30.5% of the released individuals), until the end of the experiments.

### EXAMPLE

### Anti-CRISPR Testing in Cell-Free Reactions

E. coli cell-free reaction mixture was sourced from Arbor biosciences (Arbor Biosciences, Cat: 507024). Each 75 μL, 1.25×-concentrated MyTXTL reaction was loaded with the necessary DNA expression templates and ultimately divided into 5-μL individual reaction droplets for incubation, expression, and fluorescence monitoring as described in 34 (FIG. 4 A). To prevent degradation of linear DNA templates, GamS (Arbor Biosciences, Cat: 501024) was added to the 75 μL TXTL reaction master mix at a final concentration of 2 μM 35. The anti-CRISPR protein AcrIIa4 (SPC gb013) and SpyCas9 sgRNAs were expressed from linear template DNA at 1 nM and 4 nM concentrations respectively. SpyCas9 (pCB843) and deGFP (pCB556) were expressed from plasmid DNA templates at 1 nM and 0.5 nM concentrations, respectively. The reactions were mixed by brief vortexing and collected using a benchtop centrifuge. Each reaction was split into two aliquots, each of 5 μL, and loaded into a 96-well V-bottom plate (Corning Costar 3357) and covered with a cap mat. The 96-well plate with TXTL droplets was loaded into a BioTek Synergy H1 plate reader at 29° C. without shaking. Fluorescence of TXTL reaction was measured at Exc. 485 nm, Em. 528 nm every 3 minutes, for 16 hours. Only the fluorescence from the endpoint of the reaction was reported (FIG. 4B).

### Plasmid Construction

The Listeria monocytogenes AcrIIA4 coding sequence, codon-optimised for Anopheles gambiae (ATUM), was amplified using primers containing the XhoI cleavage site followed by a nuclear localization signal (NLS) at the N-terminus side and the PacI site after the C-terminus (RG427: AACCTCGAGATGCCGAAGAAAAAGAGGAAGGTGAGCGGCGGTAGCAACATTAATGA TCTCATACGGGA [SEQ ID NO:12] and RG428: CGCTTAATTAATCAATTCAACTCGGACTTCA [SEQ ID NO:13]) (FIG. 12). The fragment was digested and ligated into a pre-existing vector containing the vasa2 promoter and terminator sequences 24 flanking the XhoI and PacI sites, the eGFP coding sequence under the control of the 3xP3 promoter separated by the #C31 attB recombination sequence.

### Microinjection of Embryos and Selection of Transformed Mosquitoes

All mosquitoes used in this work were reared under standard conditions of 80% relative humidity and 28° C. Adult mosquitoes of a previously generated A. gambiae attP docking line 25 were blood-fed by Hemotek and freshly laid embryos were aligned for microinjections as described previously 36. The injected solution contained 50 ng/μl of the vasa:AcrIIA4 construct and 400 ng/μl of a helper plasmid expressing the pC31 integrase under the vasa2 promoter 37. Hatched larvae were screened for transient expression of the eGFP marker and crossed to wild-type mosquitoes to obtain transgenic individuals expressing both the eGFP and eCFP. Expression of fluorescent markers was analysed on a Nikon inverted microscope (Eclipse TE200).

### Molecular Confirmation of Insertion and Zygosity Assessment

Vasa:A4 and wild-type mosquitoes were used for gDNA extraction using Qiagen blood and tissue kit (Qiagen) followed by PCR amplifications at the insertion locus to confirm the correct integration of the transgene and zygosity of the vasa:A4 released in the cage trial.

The ϕC31 mediated integration of the vasa:A4 construct was confirmed using primers binding the integrated cassette and the neighbouring genomic locus using the RG1044 (ATCCGTCGATGCCTAACTCG [SEQ ID NO:14]) and RG187 (TCAGGGGTCTTCAAACTTTATT [SEQ ID NO:15]) primers (PCR A) (FIGS. 5, A and B). The proportion of heterozygous (vasa:A4+/−) and homozygous (vasa:A4+/+) anti-drive males released in the cage trial was determined using the RG1044 (ATCCGTCGATGCCTAACTCG [SEQ ID NO:14]) and 5R1 (TGACACTTACCGCATTGACA [SEQ ID NO:16]) primers binding the transgene and the flanking genomic region (PCR B) and primers RG1047 (AAGATAAGGGCTTGCCTCGG [SEQ ID NO:17]) and RG1044 (ATCCGTCGATGCCTAACTCG [SEQ ID NO:14]) binding either side of the transgene insertion site (PCR C) (FIGS. 5, A, C and D, and FIG. 12).

### Mosquito Genetic Crosses

Vasa:A4 males carrying one copy of the anti-drive construct (vasa:A4+/−) were crossed to heterozygous females of each gene-drive line (zpg:dsxF+/−, zpg:7280+/− or nos:7280+/−). Larvae carrying one copy of the drive (RFP positive), one copy of the anti-drive (GFP positive) or both (RFP and GFP positive) were selected and crossed to wild-type individuals for phenotypic assays (FIG. 6A).

Vasa:A4 males were crossed to virgin females carrying a 3xP3:DsRed marker in the same locus (mars, 25) to generate individuals carrying either both transgenes (vasa:A4+/mars+) and subsequently homozygous for the disruption of the genetic locus (GFP and RFP positive) or either transgene in heterozygosity (GFP positive vasa:A4+/− and RFP positive mars+/−). For each genotype, transgenic males and females were crossed to wild-type individuals for phenotypic characterisation (FIG. 6B). Transgenic individuals carrying both transgenes (vasa:A4+/mars+) were also crossed to each other to generate individuals homozygous either for the vasa:A4 (vasa:A4+/+) or the mars (mars+/+) construct as well as siblings carrying one copy of each construct (vasa:A4+/mars+). Males and females of each genotype were crossed to wild-type for phenotypic characterisation (FIG. 7C).

### Phenotypic Assays

For each genotype tested, 30 transgenic male or female adults were crossed to an equal number of wild-type mosquitoes for 5 d, blood-fed, and a minimum of 15 females allowed to lay individually. The entire egg and larval progeny were counted for each lay (FIG. 6). Females that failed to give progeny and had no evidence of sperm in their spermathecae were excluded from fertility analysis but considered for mating analysis (FIG. 10). To confirm parental zygosity of the vasa:A4 alleles progenies were also screened for the presence of WT individuals.

Inheritance of gene drive (RFP positive) and anti-drive (GFP positive) transgenes was measured by screening the entire larval progeny obtained from each oviposition. Females that produced less than 10 larvae were excluded from the analysis of transgenic inheritance rates (FIG. 3B).

Statistical differences against selected reference crosses tested in parallel were assessed using Welch's unpaired t-test, for both larval and egg output averages, and Fisher's exact test for the total number of larvae hatched from each cross (FIG. 6).

### Non-Overlapping Generations Cage Trial

To minimise possible parental bias of Cas9-gRNA deposition and consequent generation of alleles resistant to the drive, the gene drive individuals released in the cage trial were obtained from both zpg:dsxF males crossed to wild-type females and zpg:dsxF females crossed to wild-type males in equal numbers, which were subsequently mixed at L1 stage and reared in parallel with offspring of vasa:A4+/− males crossed to vasa:A4+/− females as well as wild-type. RFP positive gene drive and GFP positive anti-drive larvae were screened at L3 stage and the developing male and female pupae were sexed and allowed to emerge in individual cages in parallel with wild-type males and females. Vasa:A4+/+ individuals used for the release were selected based on higher intensity of the eGFP signal from larval progeny of vasa:A4 heterozygous parents. Adult mosquitoes were mixed only when all the pupae had emerged.

Two experimental cages were initiated by releasing 150 zpg:dsxF+/− males and 150 zpg:dsxF+/− females (corresponding to a 25% allelic frequency of gene drive alleles) together with 120 anti-drive males enriched for homozygous (˜20% allelic frequency of anti-drive alleles), 30 wild-type males and 150 wild-type females (contributing 30% to the total of ˜8055% allelic frequency of wild-type alleles for the anti-drive locus and 75% for the drive locus). In parallel, two control cages were initiated by releasing an equal number of gene-drive mosquitoes (150 zpg:dsxF+/− males and 150 zpg:dsxF+/− females) with 150 wild-type males and 150 wild-type females (corresponding to 25% allelic frequency of the gene drive).

Each generation, mosquitoes were left to mate for 5 days before they were blood fed on anesthetized mice. Two days later, egg bowls filled with water and lined with filter paper were added in the cages to allow for overnight oviposition. The following day, eggs laid in the egg bowl were dispersed using gentle water spraying to homogenize the population, and 650 eggs were randomly selected to seed the next generation. The remaining eggs were photographed and counted using JMicroVision V1.27 to obtain the overall egg output from each cage (FIG. 3B). Larvae hatching from the 650 eggs were counted and reared at a density of 200 per tray (in ˜0.5 litre rearing water). L2/L3 larvae were screened for the presence of the RFP and GFP marker to measure gene drive and anti-drive genotype frequencies (FIG. 3A). All the pupae obtained from the 650 eggs were used to seed the following generation.

### Amplicon Sequencing Analysis

Adult mosquitoes were collected at G1, G5, G10 and G15 from each of the four cages after obtaining the respective progenies (FIG. 3). DNA extraction from pooled individuals, PCR amplification and amplicon sequencing were performed for each of the 14 samples 38. The CRISPResso v1.0.8 software 39 was used to analyse the frequency of wild-type and mutated sequences at the zpg:dsxF gene drive target as previously described accounting for all indels and substitutions present at the gRNA sequence and the two invariable nucleotides of the PAM sequence (−GG)38 (FIG. 7A). Exogenous contaminant alleles were removed bioinformatically.

### Modelling

Discrete-generation recursion equations were used for genotype frequencies, with males and females treated separately as in 11,25,38. Here we model two loci: the gene drive locus, where we consider three alleles, W (wildtype), D (drive), and R (non-functional nuclease-resistant), and the anti-drive site with two alleles W (wildtype) and A (anti-drive). F_{ij|kl} (t) and M_{ij|kl} (t) denote the genotype frequency of females (or males) in the total population, where the first set of indices denotes alleles at the target locus ij={WW, WD, WR, DD, DR, RR}, and the second set denotes the anti-drive locus, kl={WW,WA,AA}. For simplicity we assume full recombination and no linkage between the loci. There are eighteen female genotypes and eighteen male genotypes (see list in FIG. 11); six types of eggs in proportions E_{W|W}, E_{W|A}, E_{D|W}, E_{D|A}, E_{R|W}, E_{R|A}, where the first index refers to the target site allele and the second to the anti-drive; and similarly six types of sperm, S_{W|W}, S_{W|A}, S_{D|W}, S_{D|A}, S_{R|W}, S_{R|A}.

Homing of the gene drive is assumed to occur only when the anti-drive is not present. Adults of genotype WD|WW (i.e., with no anti-drive) produce gametes at meiosis in the ratio W|W:D|W:R|W as follows: (1−d_{f})(1−u_{f}):d_{f}:(1−d_{f}) u_{f} in females, (1−dₘ)(1−uₘ):dₘ:(1−dₘ) uₘ in males. Here, d_{f} and dₘ are the rates of transmission of the driver allele in the two sexes and u_{f} and uₘ are the fractions of non-drive gametes at the target site that are repaired by meiotic end-joining and are non-functional and resistant to the drive (R). If the anti-drive is present (WD|WA and WD|AA), drive inheritance is Mendelian. In all other genotypes, inheritance at the target site is also Mendelian. In the deterministic model, fitness effects are manifested as differences in the relative ability of female or male genotypes to participate in mating and reproduction. We let wᵢⱼₖₗ≤1 represent the fitness of genotype ij|kl relative to w_{WW|WW}=1 for the wild-type homozygote (see ‘overall fitness’ in FIG. 11). We assume the dsx target gene is needed for female fertility, thus females with DD, DR and RR at the gene drive locus are sterile.

We firstly consider the gamete contributions from each genotype. The proportions E_{m|n} (t) with allele m={W,D,R} at the gene drive locus and n={W,A} at the anti-drive locus in eggs produced by females participating in reproduction are given in terms of the female genotype frequencies F_{ij|kl}(t):

$${E}_{m \vert n} ( t ) = \frac{{\sum }_{i = 1}^{3} {\sum }_{j = 1}^{3} {\sum }_{k = 1}^{2} {\sum }_{l = k}^{2} {c}_{ij \vert kt}^{m , n} {w}_{ij \vert kt} {F}_{ij \vert k l} ( t )}{{\sum }_{i = 1}^{3} {\sum }_{j = i}^{3} {\sum }_{k = 1}^{2} {\sum }_{l = k}^{2} {w}_{ij \vert kt} {F}_{ij \vert k l} ( t )}$$

where i and j are each summed such that {1,2,3} corresponds to {W, D, R} and k and l such that {1,2} corresponds to {W, A}. The coefficients C_{if″kl}^{m,n} correspond to the proportion of the gametes from female individuals of type (ij|kl) that carry alleles (m|n). For example, assuming no linkage, for a female of genotype WD|WA, the coefficient for alleles of type m|n=W|W, W|A, D|W and D|A is=¼ since inheritance of the drive is Mendelian due to the presence of anti-derive in that genotype, and is zero for alleles of type R|W and R|A since it is assumed that no end-joining resistance is generated with anti-drive present. An analogous expression is used for sperm:

$${S}_{m \vert n} ( t ) = \frac{{\sum }_{i = 1}^{3} {\sum }_{j = i}^{3} {\sum }_{k = 1}^{2} {\sum }_{i = k}^{2} {c}_{ij \vert kt}^{m , n} {w}_{ij \vert kt} {M}_{ij \vert k l} ( t )}{{\sum }_{i = 1}^{3} {\sum }_{j = i}^{3} {\sum }_{k = 1}^{2} {\sum }_{l = k}^{2} {w}_{ij \vert kt} {M}_{ij \vert k l} ( t )}$$

To model cage experiments, she initial frequency of heterozygote drive females and males is F_{WD|WW}=M_{WD|WW}=25, of anti-drive males M_{WW|AA}=0.2, and of wildtype female and males F_{WW|WW}=0.25 and M_{WW,WW}=0.05. For release of gene drive only, M_{WW,WW}=M_{WD,WW}=¼ and F_{WW,WW}=F_{WD,WW}=¼, Assuring random mating, we obtain the following recursion equations for the female genotype frequencies in the next generation (t+1):

$${F}_{ij \vert kl} ( t + 1 ) = \frac{1}{2} ( 1 - \frac{{δ}_{ij}}{2} ) ( 1 - \frac{{δ}_{kl}}{2} ) ( {E}_{i \vert k} ( t ) ( {S}_{j \vert k} ( t ) + {E}_{j \vert k} ( t ) {S}_{i \vert k} ( t ) + {E}_{i \vert l} ( t ) {S}_{j \vert l} ( t ) + {E}_{j \vert i} ( t ) {S}_{i \vert l} ( t ) )$$

Where δᵢⱼ is the Kronecker delta. The factors

$$( 1 - \frac{{δ}_{ij}}{2} ) , ( 1 - \frac{{δ}_{ki}}{2} )$$

account for the factor of ½ for homozygosity at the drive target site (for ij={WW, DD, RR}) and at the anti-drive site (for kl={WW, AA}). Similar equations may be written for the male genotype frequencies M_{ij|kl}(t+1).

In the deterministic model, the load on the population incorporates reductions in female and male fertility and at time t is defined as:

$$L ( t ) = 1 - 2 F ( t ) {\overline{w}}_{f} ( t ) {\overline{w}}_{m} ( t )$$

where w̅_{f}(t)=_{Σk=1}¹³wₖFₖ(t)/Σₖ₌₁¹⁸Fₖ(t) is the average female fitness and w̅ₘ(t)=Σₖ₌₁¹⁸wₖMₖ(t)/Σₖ₌₁¹⁸Mₖ(t) is the average male fitness (here, k is summing over the eighteen genotypes). F(t)=Σₖ₌₁¹⁸Fₖ(t) is the proportion of females in the population (=½ except for the zeroth generation). The load is zero when only wildtypes are present.

In the stochastic version of the model, as in [2, 5], probabilities of mating, egg production, hatching and emergence from pupae are estimated from experiments (FIG. 11) and random numbers for these events are taken from the appropriate multinomial distributions. To model the cage experiments, 150 female and 30 male wild-type adults along with 120 male drive homozygotes (WD|AA), and 150 each female and male drive heterozygotes (WD|WW) are initially present (600 individuals in total). For experiments with gene drive only and no anti-drive, there are 150 each of female and male WD|WW gene-drive heterozygotes and 150 of wild-type adults. Females may fail to mate, or mate once in their life, with a male of a given genotype according to its frequency in the male population times its mating fitness (relative to wildtype), chosen randomly with replacement such that males may mate multiple times. The number of eggs from each mated female is multiplied by the egg production of the male relative to wildtype. To start the next generation, 650 eggs are randomly selected, and their hatching probability depends on the product of larval hatching values from the mother and father. The probability of subsequent survival to adulthood is assumed to be equal across genotypes. Assuming very large population sizes gives results for the genotype frequencies that are indistinguishable from the deterministic model. For the deterministic egg count, we use the large population limit of the stochastic model.

### Plasmid Construction for Ag(Vasa:A4)2 Transgenic Line Generation

The L. monocytogenes AcrIIA4-coding sequence followed by a NLS at the N-terminus side, under the control of the vasa2 promoter²⁴, was amplified from C77 plasmid using primers containing overhangs for Gibson assembly (RG964-RG969). A plasmid backbone containing the piggyBac inverted repeats and two #C31 attP recombination sites, as well as a fragment containing eGFP marker under the control of the 3xP3 promoter were amplified from K101³⁸ using primers also adapted for Gibson assembly (RG970-RG971 and RG968-RG967, respectively; Table 12). The final plasmid was named C119 and was assembled using the standard Gibson assembly protocol⁴¹.

### Microinjection of Embryos and Selection of Transformed Mosquitoes for Ag(Vasa:A4)2 Transgenic Line Generation

All mosquitoes used in this work were reared under standard conditions of 80% relative humidity and 28° C. Adult mosquitoes of the A. gambiae G3 colony were blood-fed by Hemotek and freshly laid embryos were aligned for microinjections, as described previously³⁶. The injected solution contained 50 ng/L of the C119 construct and 400 ng/L of a helper plasmid expressing the piggyBac transposase under the vasa promoter. Hatched larvae were screened for transient expression of the eGFP marker and crossed to wild-type mosquitoes to obtain transgenic individuals expressing eGFP. Expression of fluorescent markers was analysed on a Nikon inverted microscope (Eclipse TE200).

### Ag(Vasa:A4)2 Transgenic Line Selection

All transgenic individuals, offspring of injected embryos, were crossed to heterozygote individuals of the gene drive line targeting the female isoform of doublesex gene in A. Gambiae³⁸ herein referred to as Ag(QFS)1. The transheterozygote offspring were crossed to an equal number of wild-type mosquitoes for 5 days, blood-fed and females were allowed to lay individually. The entire larval progeny was counted and screened for each oviposition, scoring inheritance of gene drive (RFP positive) and anti-drive (GFP positive). Individual families originated from single insertions, indicated by the mendelian inheritance pattern of the anti-drive construct, were selected based on the number of larvae produced by the single mother, the rate of gene drive inhibition. The strains selected were subjected to inverse PCR as previously described⁴², to determine the integration locus of the anti-drive construct.

### (Vasa:A4)2 Transgene Insertion Identification

Targeted nanopore sequencing with Cas9-guided adapter ligation, was used to determine the specific genomic location of the selected transgenic line, as described previously⁴³. Specifically, high molecular weight (HMW) gDNA from −160 male and female transgenic individuals was extracted using an optimised HMW extraction protocol alongside QIAGEN Genomic-tip 20/G cat #10223 and Genomic DNA Buffer Set cat #19060. gRNA probes were designed using CHOPCHOP and synthesised using synthetic CRISPR RNA (crRNA) and trans-activating crRNAs (tracrRNAs) to assemble a duplex. The resulting reads were mapped against a hybrid AgamP4-C119 reference genome, in which the sequence of the C119 transgene is appended to the latest AgamP4 genome file. BLASTn analysis of the reads aligning to the construct sequence was used to identify the insertion locus of the construct, within the first intron of AGAP004649 gene, at the TTAA site located at 2R:59504269-59504272 (GGGATTTGACGTTAAAGACAACACTT [SEQ ID NO:22]) (FIG. 14).

### Mosquito Genetic Crosses for Ag(Vasa:A4)2 Characterisation

Ag(Vasa:A4)2 males carrying one copy of the anti-drive construct ((vasa:A4)2^{+/-}) were crossed to heterozygous females of the gene drive line (Ag(QFS)1^{+/-}). Larvae carrying one copy of the drive (RFP positive), one copy of the anti-drive (GFP positive) or both (RFP and GFP positive) were selected and crossed to wild-type individuals for phenotypic assays (FIG. 15).

Homozygous ((vasa:A4)2^{+/-}) and heterozygous ((vasa:A4)2^{+/-}) individuals of the Ag(Vasa:A4)2 transgenic line were selected using the Complex Object Parametric Analyzer and Sorter (COPAS) according to the eGFP marker expression levels, and were crossed to wild-type individuals for phenotypic characterisation (FIG. 16). The wild-type counterparts were also processed through the COPAS to account for any fitness effect attributed to the sorting process.

### Single Deposition Phenotypic Assays for Ag(Vasa:A4)2

For each genotype tested, 30-50 transgenic male or female adults were crossed to an equal number of wild-type mosquitoes for 5 days, blood-fed and a minimum of 25 females were allowed to lay individually. The entire egg and larval progeny were counted for each lay (FIG. 16). Females that failed to give progeny and had no evidence of sperm in their spermathecae were excluded from fertility analysis. To confirm parental zygosity of the (vasa:A4)2 alleles, progenies were also screened for the presence of wild-type individuals (negative to fluorescence screening).

Inheritance of gene drive (RFP positive) and anti-drive (GFP positive) transgenes was measured by screening the larval progeny obtained from each oviposition. Females that produced less than ten larvae were excluded from the analysis of transgenic inheritance rates (FIG. 15).

Statistical differences against selected reference crosses tested in parallel were assessed using Welch's unpaired t test, for both larval and egg output averages (FIG. 15, 16).

### Measuring Life-History Parameters

Life-history parameters were performed for Ag(Vasa:A4)2 and wild-type G3 in medium cages (BugDorm-4) as described in Hammond, Pollegioni et al., 2021′ assessing egg deposition, hatching rate, larval and pupal mortality, time of pupation, adult mortality and mating success. To determine egg number and hatching rate en masse, three replicate crosses were performed with 150 females and 120 males of the following genotypes: homozygous males to homozygous females of Ag(Vasa:A4) transgenic line; homozygous males to homozygous females of Ag(Vasa:A4)2 transgenic line; and wild-type males to females. Females were blood-fed after four days, and the egg progeny counted using EggCounter v1.0 software⁴⁵. The hatching rate was estimated three days post oviposition, visually checking 200 eggs under a stereomicroscope (Stereo Microscope M60, Leica Microsystems, Germany). Time of pupation, larval and pupal mortality were evaluated by rearing three trays of 200 larvae/tray and counting/sexing the number of surviving pupae, in triplicate.

Mating success of heterozygote Ag(Vasa:A4)2, homozygote Ag(Vasa:A4)2, and wild-type males was assessed in medium-sized cages, by placing 100 virgin 2-day old males of each genotype with 100 2-day old virgin wild-type females, in triplicate. After 4-5 days, females were collected, and mating status was assessed through detection of sperm in the dissected spermatheca.

Sex-specific adult survival of wild-type and Ag(Vasa:A4)2 was performed in medium-sized cages. One hundred pupae were inserted in each cage per genotype and sex. Adult survival assay was performed in triplicate and calculated through daily collection of dead mosquitoes. Daily survival curves and statistical difference between genotypes and genders were calculated using GraphPad Prism 9.

### Ag(Vasa:A4)2 Anti-Drive Release Experiment in Medium-Sized Cage Overlapping Generation Populations

The capacity of the anti-drive Ag(Vasa:A4)2 to stop the invasion of the gene drive Ag(QFS)1 was assessed in age-structured populations in medium-sized cages (30×30×30 cm). The populations were established by the introduction of 400 wild type pupae (200 males and 200 females) as a starting point. Afterwards, 150 randomly selected pupae were introduced each week, to maintain a mean adult population of 425 mosquitoes based on adult mortality, as determined experimentally. Subsequently, three-week releases of 111 heterozygous Ag(QFS)1 male were performed in both cages once a week (26% allelic frequency 66 homozygous Ag(Vasa:A4)2 males (30% of male population) were introduced every restocking, on top of the 150 randomly-selected pupae until the gene drive individuals were completely removed. Then, weekly restocking of random 150 pupae were carried out until the end of the experiment (day 274). Egg output and hatching rate were recorded, and larvae were reared at a density of 200 per tray. Transgenic frequency and sex ratio were recorded by manual screening of 150 pupae every week. The maintenance of the overlapping-generation population was performed by a single feeding and a single restocking per week.

This invention was made with government support under Award No. HR0011-17-2-0042 awarded by the Defense Advanced Research Projects Agency (DARPA). The government has certain rights in the invention.

### REFERENCES

1. A Programmable Dual-RNA-Guided DNA Endonuclease in Adaptive Bacterial Immunity|Science. https://science.sciencemag.org/content/337/6096/816.
2. Kyrou, K. et al. A CRISPR-Cas9 gene drive targeting doublesex causes complete population suppression in caged Anopheles gambiae mosquitoes. Nature Biotechnology 36, 1062-1066 (2018).
3. Hammond, A. et al. A CRISPR-Cas9 gene drive system targeting female reproduction in the malaria mosquito vector Anopheles gambiae. Nature Biotechnology 34, 78-83 (2016).
4. Gantz, V. M. et al. Highly efficient Cas9-mediated gene drive for population modification of the malaria vector mosquito Anopheles stephensi. PNAS 112, E6736-E6743 (2015).
5. Grunwald, H. A. et al. Super-Mendelian inheritance mediated by CRTSPR-Cas9 in the female mouse germline. Nature 566, 105-109 (2019).
6. Simoni, A. et al. Development of synthetic selfish elements based on modular nucleases in Drosophila melanogaster. Nucleic Acids Res 42, 7461-7472 (2014).
7. Gantz, V. M. & Bier, E. Genome editing. The mutagenic chain reaction: a method for converting heterozygous to homozygous mutations. Science 348, 442-444 (2015).
8. Burt, A. Site-specific selfish genes as tools for the control and genetic engineering of natural populations. Proc Biol Sci 270, 921-928 (2003).
9. Windbichler, N. et al. A synthetic homing endonuclease-based gene drive system in the human malaria mosquito. Nature 473, 212-215 (2011).
10. Hammond, A. M. et al. The creation and selection of mutations resistant to a gene drive over multiple generations in the malaria mosquito. PLOS Genetics 13, e1007039 (2017).
11. Beaghton, A. K., Hammond, A., Nolan, T., Crisanti, A. & Burt, A. Gene drive for population genetic control: non-functional resistance and parental effects. Proceedings of the Royal Society B: Biological Sciences 286, 20191586 (2019).
12. Unckless, R. L., Clark, A. G. & Messer, P. W. Evolution of Resistance Against CRISPR/Cas9 Gene Drive. Genetics 205, 827-841 (2017).
13. Champer, J. et al. CRISPR Gene Drive Efficiency and Resistance Rate Is Highly Heritable with No Common Genetic Loci of Large Effect. Genetics 212, 333-341 (2019).
14. Vella, M. R., Gunning, C. E., Lloyd, A. L. & Gould, F. Evaluating strategies for reversing CRISPR-Cas9 gene drives. Scientific Reports 7, 11038 (2017).
15. Esvelt, K. M., Smidler, A. L., Catteruccia, F. & Church, G. M. Concerning RNA-guided gene drives for the alteration of wild populations. eLife 3, e03401 (2014).
16. DiCarlo, J. E., Chavez, A., Dietz, S. L., Esvelt, K. M. & Church, G. M. Safeguarding CRISPR-Cas9 gene drives in yeast. Nat. Biotechnol. 33, 1250-1255 (2015).
17. Gantz, V. M. & Bier, E. The Dawn of Active Genetics. Bioessays 38, 50-63 (2016).
18. Wu, B., Luo, L. & Gao, X. J. Cas9-triggered chain ablation of cas9 as a gene drive brake. Nat Biotechnol 34, 137-138 (2016).
19. Xu, X.-R. S. et al. Active Genetic Neutralizing Elements for Halting or Deleting Gene Drives.

Molecular Cell 80, 246-262.e4 (2020).
20. Pawluk, A. et al. Naturally occurring off-switches for CRISPR-Cas9. Cell 167, 1829-1838.e9 (2016).
21. Rauch, B. J. et al. Inhibition of CRISPR-Cas9 with Bacteriophage Proteins. Cell 168, 150-158.e10 (2017).
22. Dong, D. et al. Structural basis of CRISPR-SpyCas9 inhibition by an anti-CRISPR protein. Nature 546, 436-439 (2017).
23. Yang, H. & Patel, D. J. Inhibition Mechanism of an Anti-CRISPR Suppressor AcrIIA4 Targeting SpyCas9. Mol Cell 67, 117-127.e5 (2017).
24. Papathanos, P. A., Windbichler, N., Menichelli, M., Burt, A. & Crisanti, A. The vasa regulatory region mediates germline expression and maternal transmission of proteins in the malaria mosquito Anopheles gambiae: a versatile tool for genetic control strategies. BMC Mol Biol 10, 65 (2009).
25. Simoni, A. et al. A male-biased sex-distorter gene drive for the human malaria vector Anopheles gambiae. Nature Biotechnology 1-7 (2020) doi:10.1038/s41587-020-0508-1.
26. Hammond, A. et al. Regulating the expression of gene drives is key to increasing their invasive potential and the mitigation of resistance. PLOS Genetics 17, e1009321 (2021).
27. Champer, J. et al. Reducing resistance allele formation in CRISPR gene drive. PNAS 115, 5522-5527 (2018).
28. Girardin, L., Calvez, V. & Débarre, F. Catch Me If You Can: A Spatial Model for a Brake-Driven Gene Drive Reversal. Bull Math Biol 81, 5054-5088 (2019).
29. Heffel, M. G. & Finnigan, G. C. Mathematical modeling of self-contained CRISPR gene drive reversal systems. Scientific Reports 9, 1-10 (2019).
30. Rode, N. O., Courtier-Orgogozo, V. & Débarre, F. Can a population targeted by a CRISPR-based homing gene drive be rescued? http://biorxiv.org/lookup/doi/10.1101/2020.03.17.995829 (2020) doi:10.1101/2020.03.17.995829.
31. Marshall, R. et al. Rapid and Scalable Characterization of CRISPR Technologies Using an E. coli Cell-Free Transcription-Translation System. Molecular Cell 69, 146-157.e3 (2018).
32. Mahendra, C. et al. Broad-spectrum anti-CRISPR proteins facilitate horizontal gene transfer. Nature Microbiology 5, 620-629 (2020).
33. Hammond, A. et al. Regulation of gene drive expression increases invasive potential and mitigates resistance. http://biorxiv.org/lookup/doi/10.1101/360339 (2018) doi:10.1101/360339.
34. Marshall, R. et al. Rapid and Scalable Characterization of CRISPR Technologies Using an E. coli Cell-Free Transcription-Translation System. Molecular Cell 69, 146-157.e3 (2018).
35. Sitaraman, K. et al. A novel cell-free protein synthesis system. Journal of Biotechnology 110, 257-263 (2004).
36. Fuchs, S., Nolan, T. & Crisanti, A. Mosquito transgenic technologies to reduce Plasmodium transmission. Methods Mol. Biol. 923, 601-622 (2013).
37. Bernardini, F. et al. Site-specific genetic engineering of the Anopheles gambiae Y chromosome. Proc Natl Acad Sci USA 111, 7600-7605 (2014).
38. Kyrou, K. et al. A CRISPR-Cas9 gene drive targeting doublesex causes complete population suppression in caged Anopheles gambiae mosquitoes. Nature Biotechnology 36, 1062-1066 (2018).
39. Pinello, L. et al. Analyzing CRISPR genome-editing experiments with CRISPResso. Nature Biotechnology 34, 695-697 (2016).
40. Marino, D et al. Anti-CRISPR protein applications: natural breakes for CRISPR-Cas technologies. Nature Methods 17, 471-479 (2020).
41. Gibson, D. G. et al. Enzymatic assembly of DNA molecules up to several hundred kilobases. Nat Methods 6, 343-345 (2009).
42. Galizi, R. et al. A synthetic sex ratio distortion system for the control of the human malaria mosquito. Nat Commun 5, 3977, (2014).
43. Gilpatrick, T. et al. Targeted nanopore sequencing with Cas9-guided adapter ligation. Nat Biotechnol 38, 433-438 (2020).
44. Hammond, A. et al. Gene-drive suppression of mosquito populations in large cages as a bridge between lab and field. Nat Commun 12, 4589 (2021).
45. Mollahosseini, A. et al. A user-friendly software to easily count Anopheles egg batches. Parasit Vectors 5, 122 (2012).

1. An anti-CRISPR construct comprising a germline specific promoter sequence operably linked to a nucleotide sequence coding for an Acr protein.

2. The construct according to claim 1, wherein the construct comprises a nucleotide sequence coding for a nuclear localisation signal (NLS), preferably wherein the NLS is tagged to the Acr protein.

3. The construct according to claim 1 or claim 2, wherein the Acr protein is AcrIIA4.

4. The construct according to claim 2, wherein the nucleotide sequence coding for the NLS-tagged Acr protein comprises or consists of a sequence substantially as set out in SEQ ID NO:11, or a fragment or variant thereof.

5. The construct according to any one of the preceding claims, wherein the promoter sequence is a promoter sequence that substantially restricts expression of the nucleotide sequence to germline cells of an arthropod.

6. The construct according to claim 5, wherein the promoter sequence comprises or consists of a nucleic acid sequence selected from the group consisting of zpg (SEQ ID NO:7), nos (SEQ ID NO:8), exu (SEQ ID NO:9), and vasa2 (SEQ ID NO:10), or a fragment or variant thereof.

7. The construct according to claim 6, wherein the promoter sequence is vasa2.

8. The construct according to any one of the preceding claims, wherein the construct further comprises attB or attP integrase attachment sites which, respectively, flank the nucleotide sequence coding for the Acr protein or the NLS-tagged Acr protein, and the promoter sequence.

9. The construct according to any one of the preceding claims, wherein the construct further comprises piggyBac transposon terminal repeats, which, respectively, flank the nucleotide sequence coding for the Acr protein or the NLS-tagged Acr protein, and the promoter sequence.

10. The construct according to any one of the preceding claims, wherein the construct comprises or consists of a nucleic acid sequence substantially as set out in SEQ ID NO: 20, or a fragment or variant thereof.

11. The construct according to any one of the preceding claims, wherein the construct is inserted within a nucleic acid sequence comprising or consisting of the nucleotide sequence substantially as set out in SEQ ID NO:21, or a fragment or variant thereof.

12. The construct according to any one of the preceding claims, wherein the construct is inserted at the TTAA site of SEQ ID NO:22, or a fragment or variant thereof.

13. A system comprising:
(i) an anti-CRISPR construct according to any one of claims 1 to 12; and
(ii) a CRISPR-based gene drive genetic construct comprising a nucleotide sequence encoding a nucleotide sequence that hybridises to the intron-exon boundary of the female-specific exon of the doublesex (dsx) gene in an arthropod, such that the CRISPR-based gene drive genetic construct disrupts the intron-exon boundary of the female specific splice form of the dsx gene in the arthropod.

14. The system according to claim 13, wherein the intron-exon boundary of the female-specific doublesex (dsx) gene has a sequence comprising or consisting of the nucleotide sequence substantially as set out in any one of SEQ ID NO:2, 3, and 4, or a fragment or variant thereof.

15. The system according to claim 13 or 14, wherein in (ii) the nucleotide sequence that hybridises to the intron-exon boundary of the female-specific doublesex (dsx) gene comprises a sequence substantially as set out in any one of SEQ ID NO:5 and SEQ ID NO:6, or a fragment or variant thereof.

16. The system according to any one of claims 13 to 15, wherein the CRISPR-based gene drive construct is a CRISPR-Cpfi-based or a CRISPR-Cas9-based gene-drive genetic construct.

17. The system according to claim 16, wherein the CRISPR-based gene drive construct is a CRISPR-Cas9-based gene-drive genetic construct.

18. A method of producing a genetically modified arthropod, the method comprising introducing into an arthropod an anti-CRISPR construct comprising a nucleotide sequence encoding an Acr protein.

19. The method of claim 18, wherein the anti-CRISPR construct is the construct according to any one of claims 1 to 12.

20. A genetically modified arthropod comprising an anti-CRISPR construct comprising a nucleotide sequence encoding an Acr protein.

21. The genetically modified arthropod of claim 20, wherein the anti-CRISPR construct is according to any one of claims 1 to 12.

22. The genetically modified arthropod of claim 21, wherein the arthropod is an insect, preferably wherein the insect is a mosquito, more preferably wherein the mosquito is of the subfamily Anophelinae, even more preferably wherein the mosquito is selected from a group consisting of: Anopheles gambiae; Anopheles coluzzi; Anopheles merus; Anopheles arabiensis; Anopheles quadriannulatus; Anopheles stephensi; Anopheles fimestus; and Anopheles melas.

23. The genetically modified arthropod of claim 22, wherein the arthropod is Anopheles gambiae.

24. A method for counteracting a CRISPR-based gene-drive in an arthropod population comprising arthropods carrying a CRISPR-based gene-drive construct, said method comprising the release of the genetically modified arthropod of any one of claims 20 to 23 in the arthropod population.

25. The method of claim 24, wherein the CRISPR-based gene drive genetic construct is a CRISPR-based gene drive genetic construct as defined in (ii) of claim 13.

26. Use of the construct according to any one of claims 1 to 12 or of the genetically modified arthropod according to any one of claims 20 to 23 to counteract a CRISPR-based gene-drive in an arthropod population comprising individuals carrying a CRISPR-based gene-drive construct.

27. The use of claim 26, wherein the CRISPR-based gene drive genetic construct is a CRISPR-based gene drive genetic construct as defined in (ii) of claim 8.


SEQUENCE LISTING (26 sequences)

SEQ ID NO: 1 - DNA, 94797 bp, Anopheles gambiae
gctaatttccaagtcccaaatgttctggtggtatattcatttcttataacaagaacccgttgtttatgaataattttgttaaattactataattttatccgatgcaaatagtaagaacagatttttggtttgcagtgcttacagcacttctcaaaatattctcgcgggccgcattcattatccacgtgggccgtatgcggcccgcgggccgccagtttgacatacctgcattaaaagaaccgtagcgttcttctcttgtaaaccggttcattcatttttttcacgtgaaccaaatgaacggttctgattcatttggcacacttctagtacagacaaactttaatcgacaacagttgttgtgccaatgaagaaaaataataataattataatattaataacaataataaaaagtaagtagggattgtctgtaagagtattttttctgtttatttattcgtattgaaataatctaaaaactattttcaacttctttatggtttaaattcttacctcttccttttcaataaacaaagaaaaaacagttcaaaataatattttatttacaaataataaccaaccattataacgaaagcgtacagatctcttcctaatgccatcggtttgacgcgcatattgttacttgggacccttgcctcacgcatacataacaagcgagcgcgtaaggctgtgctctagcatatggaaccgtgcgtcgaacactctatcgcccatattgtgctgcgttgggaaacaacctatcttggcctttggaaaaccgctttctggctgctcccggaagaacaccactcaaacatgcatcgcgagcaaataaacacccaatcgcacactctacaacatgcacgtgtttgaaaaagaaactcgagccgtacgacagtctctagttacagcacagcctcagtaacaatgttgtgaatgtattgcagggacgttgtgttgtggcgcagtcttttttttaaacaaaaccgaacccttagtgtaaaccgaacgtggttgtggggatagagcgttagaggggtgggcagggaagggtggaaaaatcaaaaacttgttgcacactccgccggaccagaccgttgcgatgtgtgtgctgacctacaacaactttcctttcccagccctactgccccatcctaccgaaccgtccgctccggtgaggcagcgtgctcatcgatgtgtgcgagctgaaaagggccgtgcgcgtgtgtttgtgcgaaacgtatgtgtgtgtgtgtgagtgtgtttgcgtaaatgcacatttatcagtgcagttccgcgtactcgccgcttcgcaatcgcaatctggtctttaatcgaggaggcaacatttgaccatcgctcgttggcagttgccgtttactactggggcgggtgtaacgaggcccacaacagcagcacggatcttgtgctttaacggtgagacgacggtaaaggtagcgcaaaaaataatacacaatgtgtgcaaagtgcagtgaaaacaaaagcgttatgtaggtgttttaagcaaaggttctacaagtgcgtataccaaagttgacaaagtgcgcgaaatcggactctgccaagaagtgccgggaacaaaacaaaacagctacaacaacacaagcaatcgacacacacacacagagatgtgtcgtcgtgagtggtaaagggcagtgaaagaatacgaacgtaaagtgcgcaaaaaaaacattcaattttcagtgcgaatttgattattcaacgatgcaattgtatttgaatgtactgccggttttgcacttcccaatacacacaaacacacacacacacacacacacacacacacacacacacacacacacacacacacacacacacacacacacacacacacacacacacacacacacacacaccccacactgtcgttcgttctgttcccttttttgtgaagtcgagacgagccactcgagccgtcaaatggcgaggacacgcacgtgtgaaggggaagagcggtgtaatggtaatgagactgttgtagcgaggggcgggaggggagggtagatgagagtagaaagggggaggaagggcgagtgctccattggcgtcgctgcatccgctgcagcgcgcggtgtgtgcatccaagacgttttcgcttcggtcgttcaataataaaaagtgtgcatcgaaaccgcacacacctttcctctcctctcctacgatcaacttctctcacacactccctctctctctcttacacacacacatccactcgggcgaatcagctccatggggcgcagacggctcttcgatggtgtgtatgcgttgcgcgccaccttcacgcacacaacgaacccgctccttataattaatgcaacaatgttgctccgttttcattacctgttttgcttcccaccgacagcaccgcgctgtgcctctcccttcgcacgccctctcccccccccccccttttttgcatcgttacccctttttgcgtcgatgcacttccatcctctctctctcacacacgcactggtatttctttctcccctcccgttgctgcaacccacctcaatcacccccccccacaccctttcgcacacttcgcctacagcccatccaactgctctaatgctaccatttccccgtttttcgcgtactgctgctgcttcggttggagagccgcgtgttgtcatggtagcgtttgcgtttggccgtcttttttgccttcatcttttgcgcccgcgtgtttgtatgcgtgtttgtcacgcatgtggtgtgtgtgtgcgtctatgtgtgaccataaaaaagcataacgcgacgaagtgtttgctagcaggcggcggcggcggctcgctgggcagtgtcggttcgttttcgcgttttcgttttgacggcttgttagggcgctgttcggtgttgttgtggtggcgccgtcggtgtacgaaaatcaaaacaacaaaacatatgtttttcggaaagttccaccccaaagggttgtgcgcgcacggagcgccgctcggtggagcgcattgtgtatctgtgtgtgagagaaacagagagagagagagagtggaagagagggggatagagtgtgtgtgtgtgtgggaggcagaggcttgccgccaaatattgttgcattctgcgtggcattgcgtggggttttgcggactggtgaatatcggtgtgagcgagcgatcgtgtgtgggagggggttgccggacggccggtacatttatcaaacgtgagacacgtgcgtttttttgttgtcgttgttgcgcttcatgttatctgtgtgtcgcagtgataaggttcgagcagctcagcaccaattgcactgcagagtggtgtgcaaaaatcatgttcgttatacctacgatgaagttatcagtctggagagaaagatgcaattatgttggataatgttgattatttatctaacgagtcgtgtgacgatcagagctgataaaaaacactagcagactatcatttcaatcagcttaatttatttcatttctcactgttgctagggctgtttagtatctcttctatttgtacatttgtcagtgtagtgattgtaacgaatgatttaatcaatgataaatgattgaaggaaagaatcgaaaatgaaattattttttcttacaagtatgttaccctttttcatcgtcatttcgctcgcttggattacagtcttactctttggtatagttatacaaactattataactattgattataaattgaaattagcataatagtattatttatcatttttctgcaaatattctttggatagattttttttatcttactttgatgaattatgttttgctcattcattatttgaaaatgtggcaacagcttgtaacagccgttaacttgttgcatagcaattcaattctatactttacaaaagggtaagattgtggcattaaaatctatgtacggtactcgcaaaccgaaaaatttaaaatcatttcgattgtacaaagtacgcaattacactcttttttattcctttacataacttcctatcattttcgtccgtttcatttcattgcttgttaaatataggttaacacttcgctcaggatccgtttattgtattgtattctattgtactaacaccagttttaacaccatttttccattccttcctgagatccttcgaatagtgcgaaatttgatccttgagcggtccacttgtctcaccgtttatttctgctaatgttcaccgaggcacatatacacacacacacgcccccggacacacacattgatagttcaacccttgtctgaatgattgtaaacgcctcgtatcaccaccggggcgaccccatcccacattgactgccctttgcaaaaagaaaagagaaaagtactcactctatccgtgctaagtgcaacagtgtgtgtgtacaatacgtgtcctggtgtgagtgcgagtaagcgagagtgggaaagagacggcaaattgggggtgcaaaatgtgtgagtgtgtgtgtgtgtgtgcgtttgtggggagcacgatcgtacatgcatacacgtgctcggtcgtctccatcacgtacagtgcgcgcatgcttgtgtgtgtgtgtgtgtgtgtgtgtgtgtatgtgtgtgatggtgtgtgtaaaagcagccgtgaagatgcagggttcgctgccgatgcaatgaggggggcacattgagtttgtgcgaaaatgtttgccaaagctcgatcaaaagggcagcagttcgttcacacataccatcgcagcgttagcaaacagccgccactgctcaccctgcccgccctacgacggagacgagcggcagccgacacgcggacagcgttccccgtgcgggtatggggccgacgcgacgcgctgcgagtgtatgtgtgtacgggcgcgcgagcgagacggacggcgaacggtggcgcgcgagcgagacggacgattgacttcgcctcaactctgttgcattgcgtgtcggcgatgcacttggcgaactgcagtttgttccgcagcatcgttcccatcgcatcgcatcgcgcgctacaaccgagacgaccgtagctggccacggacgagcgtcgggaacacatacaacactcctgtgctgtccgccgtcgacttcgaaaggcacccaaatcgcgctcgctctctctgtgtgtgaagcactgcagaagcgtgcagtcgacattcgagcatccgttcgggcagtgcgtgtggtacgtgcggcagtgcagtgggccgccggtaaaagtgtatatcgttgctatgtcgacgatcgcctactaaggaaattgcgtccaatgtaccagtgtcagtaacgcgcgtgtcggagaagcaaacagccacggcgaacgcaacggaaaaaaaacgtttgtaaccgcgttagttgaagcgaacgagaactttagtgtgttgggcaggatttctctgctaaaacccggaaactttacgttcggatcggtgagctgtgccgtgtgtgagaagagagccttggcggtgacggcttggctgagaaaggggccgcccaataatcctgaacggccgtgcgtaaatagagatagccgtgcgcgtgccggtgcggtggaatttcgtgtggttaaatctgcttccaataaaactcgttgacggcgcttgacaaaatacagccgcccaatcggtagcagcggcccagtcagtatcggactgcaaaaaaaaaactgccagttttgatagtgtgaggaagagtgcggcctacgcgcacacgtgtagtttacgccagctgataacggtttcggcggcaggccccaaacgcacaactcgcaggcggtacgcaacacagttccaagtcaaaaagcgtgaaaaaacgcctgcatccccaacaaacacatacacgcatgcggccgatagaaaagtaaatattcaccaccgcctggggaaattgcgataagtgaagggcggtgaagacacggcacagatattcgattgaccgcatatagaggcgcgaaaagtgtagaattaaatgggtagaaaataaacactccgcgttgcgttgtgatgtgtgatgtgcggattggagcgagtcacaatcctctggccctgcgcccgttgcagtgaaacccgcgtggacggaatgcaatttttatctatctcgtgtgtgtgtgttgaaggggtttgttgaaactggaaaatcaattgtgaaacaaaaaattatcagtgattgtgatggtgtgtttttgttgtcgttaacagtgtgctgggaatgagattaagatttacgtgtgcgtgtagtacttgcctggcgagcaagaagatatgagatacccgctcattcagtaacaaaattagtgtgatcgtgtgtgttttatgtgattgtgcagtgatgattgtccaattaacgtaaagatagcagatttaagaattttatcaaaaggagtgcttcaaaaatatatatttggtaagtaaatatgcaaacttttgtgaaatcctcctaaggacagtcaggccgtgtcgcttgaaaaaagtgtatattttccagggaaatcattagtcatttaatgattgctagttttttttttaatgtaaaattaaataaattctattaataaataaattaaatgtgcagcatataaatgagataacgaaattatttattttctcctgacatgaaattttgtaatttttttttgcttttcgtaaccttaactatcgagaatttttttttacaagacgttgactaactctaacgtttgtctaagatcgtaatacacatcgcaatagaatttggtcaaaatattccacagtgatttaaatttatgaatgcgttttgctgatacaattctttaattgttgttaattctataagtattccaagtcgtactaacgttttattatccataataattccgttaatttggtttcaatgcttttggaatttcaaataagctatatccagcattaatgaactgaaaaattcaataacacaattttcattattttcaatggtgttatgctttggtcatcctagcagaagtgaaaaaatgctaattttaaatgttccaatgttttgaaatattacaggaaatcaaattaatgtatattatgtcttaaataagatgttaaatggacaagataataattagccaaaatattgcattacttcaaataaaatatgagatctttgaaaatacccccgtgcaggcaattggctacagcaagaagcaattgcggttctttgtcattgaagttatatatatttaaaagatatatcaacaaaaatatgctttttaacatttgttagatacatataaacattcgagaacaatacaaaattatgtaattttgaattttaacaccataacaaatgcaacaaacatagcctgtgtgttttgttttcttaacatttttttgtcatagtattaaattatttgaaatgatgtatatgatcccttcgatcgaattctaatgacacttgatcgaaacaaataaaatataaaatatatatagctaggcttgtttaaaatgttttatggtgagcgaagatctagtgtgaccttaaattataaaacagctatttccatatcaaatttcattgtttttttttttaatttcaaagatcggccatattgctattcaaattttcttttattctgaagaaatgccagactgtaatgttcttacttacattaattatcatgttcattatcttactgtcatctgttacctgtattaggtccggttatttaggtatattgaaatgttaaatgtaattttacgttggaacgcctatatcatcttaatgaattaagtttaatatgacaaaaattaagaccataaaatttctaaatggttctttcggtacgtttgattgcagatctcccaaaccctagcaccatcgcttcctcgaccaaccaataccgacagcccgagaacgatcgtacccgagtggaaaacacattgtattttcgcagcaaaaacaacacagaaatctttaaatattttaagataaactccatgtcccgacaaatctgcttttttgcgattacatagtaaagaaacacagtagtgaggagcttacttttgctcgtgctcgtaccaccttttaaaaaaacccggagggacaatgccgtcacgcaccacggccaacgatttgcgcgagctcgatgtagcgccggcaagtgtaacgttagatcaagcttccagatgttgagagtcggagtcacaatacgtccacaactgtcggttcgtccaatctgtacattgtgtggtcggtgtttggtgggaatgacaacggtgtgtcctcttcgaaggtgctaaaaggaagctcgctgacgaggcggtagggtgtgagagtttggccagtttgttgttgcgcttgtgtggggtgcagcagggaaagcattagccgagaggtagagacacacaagctatttgggaccgtgaaatacgccgcgcgcaacagtaataacataacgtaccgtaagccgaagcgatcgaatcgtgtaatcgaagcggtctcgtgtttttttcctcctatatcgagaggccaaccgatacatccaggtgcattcggcggcatagataacgcagcattaagagtcggaattggctctcgaacgcaacagtttgattgatatataggcaaggcgtagtcagagaggtgctgtaaacgagaagaaagtaaggctagcaggagaagcgcaagttgaggaggggtgtcgcagggttgacgtagacgtagagcttgtttggaagacatacgcggaaccacacgggcgtgtggtgcatcttgaatggtgtcacaggaccgctggacggaagcaatgtccgactccgggtacgattcgcgcacggacggcaacggtgcggccagctcgtgcaacaactcgctgaacccgcggacgccgccgaactgcgcccgttgccgcaaccacgggctgaagatcgggctgaagggccacaagcggtactgcaagtatcgcgcctgccagtgcgagaagtgctgcctgacggccgagcggcagcgcgtgatggccctgcagacggcgctgcggcgcgcccagacccaggacgagcagcgggcactgaacgagggcgaggtacctcccgagccggtagctaacattcacataccaaagctatcagagctgaaagacctgaagcataatatgattcataattctcagccgagatcgttcgattgcgactcctccaccggatcgatggcgtccgcaccggggacctccagcgtgccactgacgatacaccgacggtcgccgggcgtaccgcaccacgttcccgagccgcagcatatgggaggtaagtacgatcatgcgtcttcatttcttcgtttttttacaactgcttcagtctgttgaggatttaacacactttttcatacatatttaccattgggatacaaactgaggctctcatagagcttcttcgaatggttcgaatcatgcaccgaaaacacttgcaagactatgatttgctccaacatcacgcaaagtggatcatctccaaagtgagcgcatctttaatgcttagattgcgcaccagagatcctccagttcccacggattgggcctgtgctacattttattggttcgcttaggcactgcctcaaattggagcatctcagcacggtacgcacgaggaacggctgcactcagacaacggtcggaaatccgtgcaatcccgggaggggaccggttttaatgctgtttggtctacgttgcctcgctaaacctaccttccgggatctctgcaacatttttcgctcacctgccacttcgttagattgtagttcccgtcgcgaggacagtgccgggagttcggtggagcaatgcgctaggctccagagaggaggctacgaatgccttggaatggacgctacacactctttttgtgcgtacttccaccacacgttacctcgacgattaccctggtggcctggtgtgcctggtgtttggcgtttacgtctcacttcgtatgtgtttcacccatcacccttcgtttcgttgttgggggctctgctttttttctgcttctttcgtactccctctcacaccactgctgcttgctccagcacgtccgattcttttttcgcatcgtattaccataattatattatttaattatctacttcttttcgaacggtggcgttggagcccgtccctctctctctttttccctcttttccctctctttgtctggcactgtgttcgtttgttttacttgtttgcacgcttggacaatgcttgtttcttatgcatcatcccccattggtacattctttagcaagacgcgtatcctttcgcctgcatgcagaaccgtttaagtgcgcccaggtccggagtgagacgaaattgatcagaattcagacacacctcgttatggggccgatgatgtaccgccatgctgtcggacgcattggtttggcgacgaaggtgtttcggtgccctggtactacaaataatggcaaacggtgcactggcgtatgcgtatgcttcttcgccccggttcgttttaaacggatcggtaatagtaaaacaacacgtaaaagcgatattttgtagtggactttggtaaacaataaggttccggctgcagttggatcttgtttttctagctacggaatgtccggtgtgcaaggcagacgttcttcagcaggtcctgtgcgtgataaaacacaaagggacaaacttttcatttgctcctatttgtacaactgcgtggaacacacctcatatacacgcacacagggtacccggggaaaaatgtcgtgtcgcttccttggacgattggtatgtattcggaaaaagaaaatacttttcgagctcgtgtgccgggtggcggtggctgccgttgttggaacggttatcgccaaattgctcttaactttgccacttgtgcaattattacttgttatatcttttcctgccggctggcttctctctatttcccccaacctactctccctttcccttcctttcctctatcgccgccatcatgccaaaggaagctgcagtcagcactccctactatcggttgaatgtgtgtagtcaaagattaagcgttgcccgtatatgctaaataaaagtttgcacgcaattccacgcttttcctcgccgcctgcgaacggtggggttttggtggcggggcaatgttttcttcctgcacgagaggacgattagttgaccttactgagcgcacggagggaacgcaggagtgtgggtagggtaggttactgaatgaccacgtaagagacgtttttgctttgttattgattatttttcagaggaaacagaacaaaatgagcaagttgaacatttgatttacattcttgggctgtgagattgcattagatttgtgttgagctgttttttgaaatgtaaaattattagcaattactgaaggtttgctgaaaggagagctgaagaagtattctattgggaaatatatgtctataaatgtgcaaaatactttcccagaagattcaaaaggctcggagaaagatcttacattttgtgttgtaaatgtgatcattgaaaacctcacaacactaaatatacctagtaaatttaaatttttaacgatattgcctacataaaacatctagagtcttaacatcgcttagaaatgccgtttggtcccagctaccaacatgccaacacgggtccggtcagcaccaaacccgcctatggaagctcatctttggcttgtttttattgttttcatcccctctaaaacacattcccggtgcggcatgttaaaactgtcattagaagctttggcgcgaatcgcgcgcgcccgctcaggggtcttgcaaacccgttcgcttcagcttctggctgtgtgtgtgtggctgggcgtaggtacgaatttgcggaatgttgcagaatgtgtcgccagcaggacagtgcggtgcggtgtgcatttgctagaacaggtttcgcgaaggaagaacgtttgctagctggctgtgtaaggcttttgaaggtatttgattgattacgaccgccaacgttcatcgttaatcatgcgcccgctcagaatagcctaccagtcatgggtggaggagttcgcggtggagttctttccaggcaaagcagggagctgcgtgtgacccggacccgcttgcacattgttcgacagccgcagtcgctccatcgaatgtccctggctttgctggccggctttgcgcaccggctcgctctggcgcaatgagttcaattttcgttgcgatcgtgaaaagatcgcccgaatcatccggtagtctgctccggtgctgcaactacttattaagcagcattatgtatcttacagctcattaggcggcgtcgaaggagcacatcagcaaacaaccgtaccgtaatgtcttaaatgcgcgtttatgatggggtgacggacctgacggcatggcggccgttgcttttgttttgattttgtttttggcacttataaggtgtggtggggttgggcggatggggtcccccaaacaggtaacgactttgaccgtcgccgtaactggtcgctggtcacatgtcgaaaggtggagggctgcactatcaaatgtcactgcatcgaaacgacgggaggtgttgtatgtgtaccatgttactgtttgtgtgtgtgtgtgtgtgagtgtatgctggccaatgttgcagaggtttttgcgcgcgtacgatcgccctgtaaccggtttgaatttttgcacacatttttttgtgtatttccagcatcaggtcgcgctggaaaaggtgattcgatcccatttctcttcgctccaaaatcgagcgcatgcacctcggtacgcggtatgtgtgtgtgtgtgtgtgcttacgtgtttgatgggtccggttactgcgcacataaatcctcgacacagtcggacaagggctctcgtgtctctagtttttggcgatggcttttcggccgctcgcgcgcagctcctgacggctccgagcggcgatggtgttgattgagtcatttactaccgaagcaccgatagagatctcgttggtggtggtgtgcgccacagatcttgacgacagattttttggcgtccgtagaagctcatttcacggtgcgatgaagacgaatggccggctagagagcgccgagtcgctccgagcggtattgtggtcagagtgagtagctttgtcaaggcgtcgttaccctttatttctctcgcgatcttcgttttttttggttaatcaagaaggggaaaagaatgacagcaaactagctgtttgagaaaagcggagggttggcttagcgacaagggtgctacataaaaaaagaaacagacaaagagcgtgtttaatccgattgttgtgttgtttccggttgagggaaccgccatgctctgccttccaaacttccgcactaaacaacaacttcctgcgcatgaggactatcactgccgcaaggcgcacatctgaagaagcccaaaactcgtcgtcgaaacaccccaaatcaaaggtcaaacatggcggttactgcttcttcttgtaaggccgccgtcgtcatgcttttgtgccgtacattgacacctcaagtaaaacagagcagcggctagcagggacttttgatgaacactttcgtcctcgcctgatgagtggtagaggcacgcaagcatttcagtttttcccctcctgtcgaatggtttttcgccccatgcgaaaaatggttacagtgttcgaccgtgagtgagtgatattttaaaagatatttcacatttactgctgctccctttcctgcgctgcgacgagcgcactcgctcgtacatcccattagcgagcacgcggccctaccaatagattgcaaatgcgcctttctgcgggcgagtcatgagtgagacatctatgacggataccatgtggacaaagcgtaaaaaatgcacacaaacacacacacacacacacacacacacacttgcactacggcaaagatcatcttttacgcgcaccgcacaccgatcgcggcagcgcccaaagtgcatagcgatggtggaggcttgcgttttggaacagaccgcgcacacgggccgccggtgtgacgtgtggaatttcagctaattagaaaattattaatagttccttgcgcacatgatcggtgcgccattcttcttcctggccaaagtcacccgggttctgcatttccggagcagagtcctcgacaggttttcactttccctgtcacacgtttgagtgtgcctatgtgtgtgtgtgtgaccccttctcgtcttgtgccttggggtcggctagcaatttctaaaacttgctcaatggcgcatccttttcctctctgtgcggagaacgtttttccgcgaatccatcccctcgccccaggtgcttatgcaatcagcgctgctttacaaattaaaacgtaatttagatcctgttcattaaggcgcgcgcccgatgcgatcctttccccgcgccacgcggtgcaattaaaagcgtatttgaataatttgattattgtatgaaaatcaaagaaatttgtctttaccggcaacaaaggcttggcatgtggaaaaccagcacaccgacagaacaggcctgtgggaaaacggagaacacacaccggcacaccaaactggttctttccgggtgcgcgcgcgacagcagattacatctggtgacacgagataatttccattccgcgatgcgttttgcgctgtttggttgttgtgcgtgtgttcggccgaagaggaggggggggggctttggacagcaaatggcttgttaatgggcttttacctttgagaactgaaccgcaaaaccctgccgaacaggggtgagtcttgagacagtctatcgtcgaagctgctgcgcgttcacttcctcatcacgcaagctggcgcgcgcacacggcctttattttggcagcttcaatcggaaagccagcacacacacacacacgttcgacagctaacgagaagcagggttgggaccaccgattagagatgtgcaatccgcgctgtgcacttttgcatcgtccacacaccccgcggacactttgctcgcttttcgccccgttgttctcggttgatttcgccgttcggccgccgacttcgattccctcatacgggtggaaaccgaaaataatgcgcgagttgcgccgccacccgcctaaatttagcaccacgagccggccgcgagagcggcaacactgttgcgcggccaaatgtctattttcgtctaattccgcacagcccgtcggtacgctaagccgtattgcggccccgcccccgctgtacccgccgatgccgatcgcggagcaatgtgcgcacttcttgagcaactagggtgcacttgcacccctgtcgtactaaccttttccgtgcgccgtgcgctctcgtgcgcactgttcttcctctctctctcacacaagcgcataaaatgtgcagtttgcgggacagatgtgtgtgtgtgtgtgtgtgtgtgtgtgttgcgctttccggttcgttacgtgtgacgtgtgtgcgcgcgcgccattgctaaagcgatcgattatcctccgggagcgctgttctgttcgctcttgttctttcaattttaaccaaccaagcaacccacccacccacccaccatgcaccccgctgcctgttccacatgtgcatcagtggtcagcttgcatgctcgaatgcagcaaaaaagtgcaatgcagagagtgcagcaaaaacaaagcacaccatgcgacaatgcaaagatgtaaaagtcacacacctccaacgaaccgcaatagatgggatggcccctgctgggacgggcaacgggagaataggggcagcgatgatgattgatacattcatattcgtcgccggagaccacccgggccaccgtggcagcccttgggggggaatatgagcatcgcgtcacgtcgtacttaatcaacgcgtgtgcgttatttgtctgcggcacttccgcgtgcgtatctgtcgtgtccgttcggttcggtcggttctcggttggccgtcccggtgctggacacacgctttgcgcgattgcggacagtctgcaaacggcaacggtatggtgtgaagaagtggttcttttttgtgtgcttcttttctttcggaaatatgaaatttcttccgctgcctgcctggacgccgggaactggacgaacacaggcgcggtccgccgtattttgccattttcgctcggatgtggtcggatgtggggccaattgcacacacaaaccgcgcgaggtggaatgtatttatttacgttttaacggtgcagctgtctcctgccggtgcatttcgtgaggttccttttgcccatcgggagtgttgtgagaggagtggccgaaacaaaacggaccgaaaaaaactgccacagcaacagttcgaaaagcacggacgcacaaaaacgagatcgctcggaaaagtgcaactggtggcgatggtgcattatttcacattcttttggccgtacgaataaaaacatgaagcaagtaccatgcgaaaattgaacttaaaagatccacccgtaacggttgcacggcagagcgtgcccgagtgggacgtgcgttaaggtgaaataaaataaattaactacaaatttacaattaaattgattccatccattgcacagtcgaggtctctgagcaggagtactaatattctaccggcaggtccgtttgcaggctgcaacaccgtcgtgcagctttcccctcgagcaggcagttagtaggcaaagtttatgtgctagatagcggtggttttgcggggagaatcaagtctagcacacacaaacaaacacgggtatgtaaaggttgaaaggctgtctcaggggaccgagttgccgattgggcgctggttcgtccaccgtccatcgcgcgtcctgaacggaaacaataacactcataataatgtttcaattaaacacaggcgggacgacgacaggaaccggttatgatgggacaatttcacaattgcacttgacattgggcgcagaattggtttgcaccagccatccagggacagttgagcattgcccagtttgagcctttggtctggagcttttacatgctaattagatttcagttagacaactctgcgcaacatacgaatgctttcaatatgttgcacaagggcacaatgccgcaacaaggtaaatgtttcctgtttctataaaacagactagacgtactttaaccaagctatggacagagtctattttcggatgtcataatttacgtttgaatgatcaatcacatttagtgactgctaaacctgcttgttatgcttatcctgtgtatcctaacgcttaattgttccgttgtgtcgttaaactagcttaaagcttcttgaaccattgaagctaccattatgaatgcagtataagcatgcaagatttatttcttttcttcgtttcgattattctttcgtaaaaggcatcttgatttaatgaatcttttgcgataatcggctacacagcatggcatctgcggggcagaacggtactcgatcgagcagtcgccattatctaggagtgcgtaatcaagtttaggttgccacgtgattcgattcatttcacaccgacatgacagcagaatagaatacgggtgcgccttgccgcactaccgttgaccgtcgcgcgagaccttctcaatggctgcattcatctcgctgctcgcaagtgcgccgtgagtggagcataaatctcgacaaacgttattgcatttcatcgactgtcttcgatcgggtttggggggggctgggtagacatttaggaagcaataacaactgtcttatcgtgcaaggaaacacaccggcacgcggctaagcctgtggtgcagtggtttagattcctttttacttttacttaccaccgcacatgctttatgttggatgttcaacaggcagcgcagacaggctgagagcggtacagcatacacacgccgtcttgcttgatagacaaggcttcgcggcctggcattgccgtggagtgacgtgtaagtagtgccccaaaggcaccactcttcacgggatagaattgagtgcgttgatgtgaacggggggcgaggaagcgtagtgccggttgtcgtcgtagttgcagcttctgcccgagcagcactgtcaaaatgggttttgcgctaggttgagaatcggaggagggccttcgccgtagaagccgtagcgatcgtcctccgcgagcacgggacgcaatgttgccacacattttgccgcgctttttttttgcactcggcagagttacgacggctctccggtatggaagcgagcagcacatctcacgggctgcgtcgaaaatcgagcataattgtatgctgtctgatctatttcatttcgcgttttatgttttattcgacttgctgttttccgccgcccggctcagcttccaggcagggcgggaggctcattgtaggttagggccccgtttgacgtgggccagacagtcggcgatggggcgaatatggggagaggttggtgaccgatccctactccatcgtgtcctccttgaggactagtttcgctctccgacactcttgacacttctcttccttcgtctgatcctctccagggaaaggctgctgggcgagaaaaccttgagacgcgggagcagccagaaaccggctcctcctgtgcagcgtgcaacaaacaaaacagcaaaagattctaggctccacactgtgcactactacgagagagaaagagtgtgtgtgcgtcctggggtagttctgtcaatgttgaaaaaggtggcaatggaagaagagctagaaaaacagaggcattatggggtgtttcaggcaggaggattggtgggtgttaggccgggcaggaaaccggatgggaagtcgaacgggatacggatgctgctgttacgccactgaagcggaatcgtttgcggaatcggtcaacattgttgagatggccgtgttcagcctgcggttgatttagttactttttgattcttttttgattcatttcgtttgtgtgtccaaatgaagtgtgctgttgggccggcagatagggctttcggcgggtacgcactcgagagttcgtgcgcgtatttctcgaacgtcacggcataccctcatcaagtgaggctgtcccgcgataggtcttgtgtatgtgtgtgtatgtgtatatatttttaaattctggtttggggcatcaggaccctgaaaatgtaccaccgaaacccaacggagagacgagcttgtctgagaatggttgggagcgcaagcagtggtgcttacgatttataaaataaacaacgacgtacggataccgtgcgacgggattaaggtcacgttcaatgttacgattgtcgatcgagacaggcatcttaagcgggctgaacggcttggtcacactggaagggattatttaccgatataagcgatttcaccattggcgttgtccgtaatgcgagggcgccgataagctgaccgaagcaggcgcgaagagtatttttgtaacttggttgaagaaacaatcacaagcatcttgatgataagggataatgaattaaacataattgcatcacctgtgatgagacagttgataaatgggacgtctcgcgaaattctggaaagcgagcaatatcttcgtacagctgcatctgacattgacgtggctgccggttgcattgcgaaacgtcaaaggtggcgctaaaagtacatgtttaaaattagtttccattttgtttgtttgtaatgcgctccggtttgtgtgcatgtgttcgggtttttagctattaactgcaatttctgcactgcaaaatgtagccgttccggtatgatcagctgcagacacgtggtggacggatcttctgcttcgcgcaaagtgcacttaaatggtcgtcgaaggagtggacagcgcccgcgtctgagctcataatcggcaggccaattatgtcgacgggaatgtggaaggatgcttgctgcagcgaacaagatgcattaagcatgggcaatcaatcatcccgtggctctgcaatcgaggtttccgtgacacacacgcgcgtccccgggtgtcgtcgctgacgatcgcgtgttttacaagtgcgtccgtgcgttccgtacgtccgctgcgtcgccgtcgtccgagccacaacatgcccacggccaataatcagtataattcggtttaacgtttggttagattatcgggaaagaaaataagccgaggtaaaaacggatcacttttcaaaccgaaccgagcgcaggactgcaaagatgggaaatgtgtgttcacgtgttgcgtgcgtgatccagggtgtatgttgcgagaaattattggaatcattccaaagttatgtcggtaacctcagcgtttttcgtgcggtgtgtcggttttatgcagaaagcagagatcttaaagcgagctggcattttgatatagcacatatattcgatggatgtagcattgaggtatcctcaatgaccattctaaattatcttatccttaaggctgtttttgggccgagtcctgcaagactagaaaaagtccgatacctattctaactgtcctcccatgtacacgtttctgcatcgttcctggaagtcatggaagtcatagagagtcattcagtttcatcacagaaacgaacagaacattgccatcaaattggacagtttcaaaacttcattcaagcaaagattaaattctagcgttagctccataagatattcgacctccaggttaagttatattggtctctagctaaggttgatgtattgatatggtcttcaaacctctactacaccctaaatatctttgtcaaagtcgttaactctcacctggcatgtagaggaacaggcaacagaccaatgattgaaaagccacgctcatgtcttcagaccataacctcggccaaatttaccttccaatccatcgataaaacctcatcgttaatgtcattaaccttttgcaaagcttttactccagtgccaccaacaaacattgcgtcaaaaaacgaccagtgtcacgttctcctccctgtgtatcggagcatctacgaaaaaaataccaaaagcctcccttaaactgggaggcccataattccagctgaacgcttagattggaacggaactggcggtgtctttcgtagggctcggaacgttttcctaccagcttctgtttgctcgaacccgaagcagagcacaaaccgtctaggttagctgacagaagaaattgcaagatgcacaaaaaatcgcacacacatacacacagacgttaacagtgtattgcgaccgaacgggcagcaaaacgctgtggctattgtgccagaccagaagggaggagaactcaaaaacggtaaagctaataaacctgtttctttccattttttgcgcattgattcatttcttgcgccggcgagagctgcccggcagttcctgttgcatacatgcagggagcgcgggtttctcgatgtgcgccacctctgccgccggcatcgccaccaccgtcaccacagaccggctcgaaggctgcgggatgcaagcgcggcaaccactggaaggtaacctctcggggcgattgttgtatttaccaatcgtgatgcatgatcaatgttgtgcggagtattttatttcttgtaagcagcagtttgaggatcggccagaggtttgggtaaacatttcagtcgctcagtcgctcgcgaaacagaataaaaaaaacgcacacagcgttcaagagaaaggcgcgcatggcggtggatgtaaaatgcctcatttgtggcgtcttttcccctgcgcgcagcagaacgtgaatgtgtgcagagcatggtgtagcgtcggacgaggagcatgaattttgagcaagcggagatggttttgagtaaatcggtttctatgcagccaaggcaacggcagccgcatagaactagagcactgtgggccaagtcgcagtcgaggcacggaagcagggcagaatcgcgactctctatcgcccttgttggacgacggataggaccgatgccggtgcgggtcaagttcagttggcttaccgatgcatcatcggaagccatcttaagtaaatggagagctggttggcgatggagcatggggctcgctttactcttttgagtgggcacaggagtgttgtgctagaaatagattcggctcaaattacggctcgggcttgcctagagaaagggcaatgaaggattgaacacatcaaagttaagtattttttgtatttgtggttgctgtcgttaaatggtttattgaagcgtttccattataaaagttgtgaaacagttggaggatgaacagaaaagcgtggatgtggaattatatttcaatacaaacacattgcacatgatcacatggatcaacggtatataatttagttggatataaaaatgcacatccagcattgaggatggtattttgccatcctccacagctcattatgttcacaaggtgatggtggcgatggtttcacagtaaaagtttctcaggcaaaacggctgcgaggcattgtgcgaaagtttgcagtaccgtgttctatgttcacaattgggttttaaatgccccaaactgttcgaacccttctcacatggagtgtgtgtgtgtagctgtgtgtgtcaaggaccgcaaacaggaagggtcaagggacaagggagggcttgtgatcggaagcgcaacagaatcatgatgagcgcagactggcaccgggcataatttgcccgtttttttatcgtgtgttgcgcattacggccctatgttgaaggagatcgttttcctccccacatacatacacacacacacatcgatcgtaaggtatgcaagaggaatgttgccttaacactgcgcgagttcggttgcagtcgatagaattcggtggtttcgagtgcgtgcagcgcatattaacgccaaggttggtcaagtcgtttttcaacgccccttgaactttggtgatgcgagtcaaggaataagagcaagaaaacaaacactccacagaactttaggatgcatggacgctgctgcagtggcggtgatggtgctgttgtttcgtgtgtcactgtaacacggctcattaacggctgcagacacagcgattgtgtcgtctgacgagtttactttaaattagcgatggcaaaatcaatagaaactttcgtcgccgccgccgccgccgtcttttgtattgatctcactgtccagcgaaacaaggtattagcacgtcacgatcttatcccgattcctgatcgtgtaaggtttacttacttttaatgagcctaaaacaaataggaacaatgctcgtcggaatgctctgcagcagctgcgtactgtttactgttagtgttcgcttgtcttgcgatgttttgcttgatcttaattattaataagggcgcggtactatttgtttgcaaaaagtcttctataatgatcgattgtattttttaaatgagatgtaaagttaaaatatttgcacaatataaacatcaaatgcaaaacatgctaaggaagaacgtaaatatttcgtgtggaatagttcctttttatttgaagttttcaatatgagtaatttttaaaaggcactttgacatatttgttttcaccaatgttacagacaatctatcaaatatgcctataattttatcagataacctgaaatcttttgcaagatgctgttcagacaatcacttcaaagtttctagtgatatttgagatttagatttgcatttaaaatcgtgcacagcatagccttttatgcattttatgtaaatcgcaatcaccacaccaaacagaggcgaaacagattgtaatattttcatttaaataacatcccccgaccacccatatgtgtgtgtaatcgagtgaccttgatgcattcagcgatgcatggcttggcatagaggggaccacaaaatcgggacgggcggtagggcagtgctagcacaagcgcagaaaattgccttatcaaataacaaaccctttctcctcatggttgcatccgcactgccctaccgcgtcgaccgatgcatccgatcgttttcatgcctgaatcagttggaaaaacttctctctcgtcggcgtcgcgaatggaaaagcgtttcacaattgcttcctactgtgacgctcgacggcgtatgtggaaaaagggtgcggtgggaggcgggatgtggagaggcttatcgtcactcactcttgggtgtatgcgtgtgtgtgttgttcgcgggaaagcccatatcgtaatcgatatgcttgttagagatccgttttgatgcaatggaaaaactaacgctccagtctagagaccaacaaacacacacacacatcgaaagagaaagggaaatgtgtgggaggaagggagaggaggggtgagagtggaaatgcaatgtagtgtgaaagtgtggctgactggttaaatggatgggaaaacaaggaaatggatggaaaggaaggaaaaaaaaaccgtccgacggttacagaaagacgcaaaagtgctcgtacgaatcgtcgtatcgtcgttggcgaacaaacaggcgaagccagagcctgccagcaacggagttctacggagctgacgggacggccagtccgccggtgtggtggatttgtttggacagaaaaagatcggaacaggagaaaaaaacgcacgccttcataatgaaatgatagacacgtgcacgtttccagtttcaaatcaatttcacactcgaagtgagaacaaacctcggaaacagtcgcacatacacacatacacattgggatggttggctggtgggtggttttggttcactttgctctccactacatgtccaacgctgctgttgctgcgtatttcatctgcccttgtgaaacgaatcaccagaagcggtttgggtttcgggagctcatgttgtgtgcgatgcgtcgccagtaagcattctcgcggaaacgataacaaatgtgtgtgtgtgttgggtgggagtgagagagaacatgaggttgggggcgaccatgacactgacctaggacaattagaaactgattgacggaaacgatatgcatcgaaagcgagacgcaggttttcttcgttttatcagacgcaggccggccttagacacgtttactctagggagtcattttgctgaggacagtgagcacagcactatgtaggttagatggggggcgtggtgggagcttggtggtccgttggatttgaagttgccagaggacaacgatgaaagtaatggccaaggatcagtgcgaataaaactcatccttgcacttacatacacacacatacggtcctgtgttggatttcgcaggacattgcgaaatgtcttcggtggaggttttactggccacgtttgatgaccttcggcattgctgccctggctgtcggtttcggttgcccggttccacatttccggtggctggctggagataatgaacatcaatttcaagaacggcaataatcgtaaaatgcagggaaatatttcttgatgcattcccgggctggatcttgaagaacgcgccgcacattggagttgatttgagcatgggaaaactcggagcgccgcccgtgccagtacggctgtcctccgctccgcgttgttacagatcctggcagttcatacattttcatcgaaccaaccagaagcatcaagccattcagccaccaccacgtaccacgagatggatgcaaaggaaggacaaaaacaaatgtaaagtcgcccagaacaatgtgcactgctcgcgcgagtcctgcttttcgtctccggtgcgtctgctgcctgcgtcttgccgaggtcgggaggaagccagcacacacacagagtcttatgccagtgatgatgcaccacaatcaatcccttctatgcagaccgaggggatcaatctaggttggtttcattttttgtttctctctcccccttcatactcgttttatgattagagagcttttccgctgcttttcgttgtgcgccgtgctgtattttgtcatgcttttgttcgacgttcccttgtcactggaccgctttttttctttcctccttccttccgcttgtttcccgtggcaggttgtttttgttttcgaacgactcggatttgccatgtatagatgcgctcagcttttacaaaaaaagacaaataaaacacgaacatacgagctaaaaacaatgcttttgatgcacaacaatcacaactaccagcgctcacacacacacagagacactctctgacgcacatttgtcgcttacgcaaagggaaggaaagaaaatgctcgaatgctgctgcagctgctgcctgggaaaagaaattggatggtcgtaaatttcgggttcggtagaaggaaagctcttccttgtttcatttacagtgtaacagtcgcacacgttggcaccacgctgccatggtggtggcgtgtggatcgaaaattgagatgaggtttggaatttttcgctacataaactttatcctgtgctggtgtggactgtttgtttctgttgcccagttttatgacgtcccggaaacgcggacaagcgaaccgtgcgaccggctaattggtctcatccgcctcgtgatttttccgaccaaccggctgcaatacaatttgtccaaccatcgtgttccgccggtggctgctgggataagcagaagaacataaatctgattgaatgccatttcaatgcaacaaattttaggaaaaatggctaaacaactccttggcaagcttctggccaagagtaaaggtaaacaacttgccagtactggtcactcttttgtccacccacctttccggttgtatgtggattgatgcattttaagcataatacattattaactccacagacaaacaaccccgaaatggcttcagctcagcttaaccaggcggcaaactgatttcgatccgcacgacatcatcttgcacgggacgagaaattgcctccgatacctccagcgcggcgtcagtcagccatctctcatatttgctctcttacaaatgatctcagcattgcctcagtcgggccctcagtcgcgcagctcgacggacagaaaagtggcgatgtgaaatattaatgttaaagaattcatttttaaatatgcaaattttaattaatattcaccctcgttcccttgtggggcaaaaacgcgggcctcgggcaacgagactctgcaggctggtagcaaggtttcggtcatctgtaaatgtgttctcgttaggcggttgcgaaaaacaggccgattttgtttcaggacagaacaggagggataaacatataaagagagagaagggttaatgtagaaacacaatatgaagttattagtgttattgctttcgaccgatggcagtagatgcccggtggatgcatcaaatcatgacttcgacaggcccaatgtccagcgacaggggtgcattaaaacaggcttgattctggatcctttaactacacatacagggtcggccagatcctgaaaggcctctacagacaagggcataaaatatgtatcacgcacgaacgatgttattgaactcatttccttttcacaaggtcaatttagtccaaagctggcatctagaaatctgatctccagccctgattgatgcaggctagcagcaaaagaaattgttttcccggaatcattcctccgattaaccatcgtgtggcatgtaaattccccactgtcaatgctgtttgaataatagccccggtgatatctcattcccgcagggcggacaggcacgatggcactatggtgaaagcctttttttcttctcacgttctcacgcgatcctgttgcataaagaagtgcactaatgagtggtggctgcgcacatgtttgcgttcgggacgccgcagtaagtcctcgttttgcagttacttccagctcgtagggccagtagcgctgcttagtccttcacggattgcgctcgatgatataatgcatcacctgccctgtcctgccatgttggttgttgttgctgcgaccgggacggatcaacgagcggtaaaattactgcacagtggcggcggtttcatgctcgcaaaggcgaatgcacaggattgtgtgcaattgtgcgacgattgcgtgcaggaagagcaggagctgaaagtgcgcagggggacaggccgcgctcgaccaaagtaatagcgggggtgtatgttttccctggtgaatgtgcggtcccacagcgttactacttcattccacttgacggaagctaatgagcagaatcaggttggctgggtgcataagagcgaaaatcacaaaagccgtacacaaaaacacacaaacagcgatgggctcggaacgggttaaaaaagaaagaaaaaagacagaacagctccaggatcctttcacgtgtacacgcaaaacaactgcagaaaagcaacaaaaaaaaatgctcctattttccggtgtgccgagttaccgcgtcggagtcatcgtgcagctcgatgtctgtgtgtgtgtgaacggtctcgcagtaacggaacaaaaaatgtcaacgagagctctccagcagaaaggaaaccggaaaattctccatcgatatagcaacagctccacttcggcgcacagtccctacctaccttcccctcactattgccccaacccattgggcggcggtggtaaatcggaacggggcatacatcagcgtcaagttcaaggacaattgtcaacgcttccgtccacaacgatccgccacccacacgtcttggggtggatggggcggtcggggaaaaaaatagaagcaaccgacgcgcaccaccccctggaagctcgcggaaaagtgtgctaggagagagagagggaggcagagaaagagagatggagagacggaagggagtctcggaaaagtgtctcggatgtgggaaatcggtttacaccgttaaccgatgccagccagatgggccatgtggggccgatgccgttcgatgtgtgcgtgcacagcgtgtttgtcatcgttgcgttgtcgacgtcgtcgtcgacgttcgtgccggctcacccatacacaggccgcaccgaagcaagcagttgggaaaacatgtggctacgacgattcgtgccgggtttttcctcgtgcactgcaacacagccctcccccttgtttccctgtcctgcgttgagtcgcatggcgcacgaagctgtttgtttgggtacgagccgttgttatgacgcggcacggcaaacgcgttttccactccgggggccggggcgctgtgtgtgtgtatgtatgtgcgcggggttaggttacgtttccgcgcgcgcgattcggcctgacgctgttcagccagtggccgcaacattgttgctaaccgggctgattttgtggccgaaagggtaggtgggatgggagggaagggtgcaatgtgcagacgggctaaaggatttggcgagacaaggaaggagtcgagagagagacgtgtccttggtgtgtggtgcaggtcgcgctgtgtaggttgagccgtctcgtgtacggttgactgtgtaagtaagtggaaagttctctctttctcactttttctctttctttctgtttctctctctctctctctctctctctctctttctatcggttgaaaattatctcgcgccacccgcatacacttgtcacgggggagtgtggggcagtgaaaatgcataccggcgaaaggaggggaaaacctcggccaagaaagggaggccagtttttctctcagctgttggttctgtcgactcggctgcacacagcgaaaggatgtgtgttgtatgccgccgcacacaaagccaagcgtaccgacacggaacacacgggcgtttgtgcatgtgggtgagcgctttggacgcatgcgatgtggaaaatcggtgaaaatgcaagattgttgctgagtgcaggcccgaaagtcagtcgtggcgcttctcgcgtacccgaaggacgcaaaaggcccgcccggtttgttgctgttcagagcaagcgggaaaggcaagatatcgtatgacacttagacgagattgagttagggcatggcgctggggtgtaacagcggcaccagacaataatgctcgtaggtatcgcattaatgctgcttgtttacttgggtttgagtgcttgaagaggtgtagcaggtttttgtttcaacttttatcactcttattcgtaaataagaattattaaaatgtaatgttaggtatttctgttgaacaaaacggttttataacatacagaagcaattaatgcattgaaatagtcttatagaaagcaaaacttcaacgaggaaacacattttggatgtttcagaaaaaacataccatcaacaactgtagagcttttcagaaagagtaaagttcctgcccagttttgattggccccgttatcaaaaaagtgaaacaaaaaccttgaaagcagcttgtttgttcgtttgtccctaatttatgttctttccttgctttcgatgatgcgatggcacgattttggcttgctttaatgatgcgttctgattaaggaccgattagacgttttttttcttccttttctcctcgctcgccagcttcctctagattcgcagagcatcggtgcgagacacaaccaacgttagcgttgataaataacaaactccaagggggttgttgttgttatgcgttccttttttgccacaatctccaaatgatagcgtaaacctgcaactatggcacatcataacgtcccgcttgagagagaaaataggcaaattaaaatgcgaatgggccatttttgctttcgttcattctgctaccgatcggtacgattttagtgttcacacacacacacacacttcttgatgatcgcttcattcatcggggcaacagaggggtggccggaatggtgttataacgtataatttgtgctaatggttatggggtggctttatttatcattaccctaacaaattgatagattccgttgactggctcacactttgctgcggccctgtgagacctttgctttgatcagtcggcggcagtgtgttctgggtgcgataggttccagttgttgcctccacaaaccgatcattcgtcgatcgttgatcgcgcatcccaggtacataactcatccaattgcgaagccccagcgtgtggtgatgaaggaagtggcgcagtcgccgctgttacgacctcttctgctagcatcgggccacggcaccgggtggcactgggggctcaacgacgtttgcctcatccggtgtccggctgtttggctgccaaacccgcgagcaaacataagcagacaaacaaaacgcgcaccgctcggtccccctcccagccaggccaggttcacacacaataagccggcaccgcgcgtgcggccgaatgccgcaactgttgaatgcatgtcgtaaaataaaaatttatgattgtaattatcatctcttctctcgcacccaccggctccgagcgaggatgggagggatgtggcgaacgcggcaccgagctggagcaaatcttcgcacacccgtctgcatcccattttcttcggatctcaccacatctctcgagcgctggtgcaaccggagatttaaagacaaaaggcaaaccatacacagacacacaggaaaaggaaatcagttcgcttggggtagctctttttcgcggtttgcagcacaatgataatgggttatgtatgtgcttgtgttagccctgttcttgctcccacctttctctagccgtaacgccacaatgccagtaagcttaacttatcccccggttgctgtctgtgttggatttattaccggtggcaagtaagttgcagcccattgctgcggtgcgcgcggtgcgttatggcaatgatttcgcatcttttcatcaagtggtgtgagcggcgggccgtcttggacacgcagaaaaggtcttatcttgtgactggccgtgtgtatgtgtgtggttctgcgcttaaagatataatttgtggcacgctttatcgcgacccgtacgacattgtttcagcagcgttgcagcagcacgcgccccatcggaaagaacggcttgatggacggcaggcgaggtaaataaaagatataaacgccgcccgccatgtccagtttaatcagctgtgtcctctggaacagttttccggtggtttggatgaggttgcatcgttactaagtgcattggtgttacgcatgcgcgaagaacaattccgtgaccttgtcgtgcgcaagcattcaaaagcgagaaaagcagctttctgttcagttagctgatgatttcttgaaacgctttcttctttttgacgggttctttctcttggaagatggtgaaccttatttttcattggtgttattagatgtcatgtaaccatgaagtacattcttgcctaagatattacgtcattcgtaaatatttattagacattgtagaacttctgctcagatgatttattcacgcaacacggaaatttacaaatcttttccacacttgttaaagtgcttgagtagttaagtgaaagagaacaaataaaacccagctgtggagcacaacagcccaaacgaacagggcatcctttagacatcattatgggtcggttctgcagggctgtctgcaatcataatgatcggttggaggttggagctccaaaacgcaatcagtccatacgcgcggtgcaagacgtgtgtcccggtgctggtgaggtaaagccattccggccgactatcagtcaacgcagcaagcagacaggacgaggggacacgctggatggatgcctccagagtgtgatgttctttggtggggtcggcgggtatgttgtggtagcatcaaatcgagcaaatcgagatggataattttcgattattaccgggtaccgaggcaaaccgagggaaatgatattgttttctcgagttgtacgtttttattcgccgtgttttatttttcgccatccctcctggtacccgttgctgtcaccgtcctttcaaaactggaaggacccaccaaagtcgtcggtaagcattcacatgcagccaggctcgcttgcatctttccgctatatcaacctggtaattgcatagtgtgagtatggtggtggtgctggtggtggtggccaagccaaagggaaaggggaggaaatacggagaaaagcaggaacaccaacatccaaatgcgctttgcgcttgcaggcatttcgcgcagcattaagcgaagccgacagaccacggccagcctgtgcacggatcgcacggattgggcacgggaagggcacggggagaagagacatgattgcttcacgccaccacgggctctcggtccgtgtaccagacgccccggacgtatcggaatgcgggctctgggcgtggctcacccggggaaaagctgataactttatgatgtgtcgaagatgagaaaatcatgactgttgtatttttatgtgtttttaaataatacaattgacgttatgttaacgggcggttaggctgccggttggaggaaaacgaataatcgagtacagtccccctgtacacgcagcacagggcaaatgcgaatgtggctttggagcgaatatgcggttgcggtttgcacattgttgtttggtttggtgaattagttcggcttcaaggtctggcttttgtttaagttaatgtcgtattttgagagtttgcatgatagtttttgcatcctgttaagaaccttcgcccgccgatgtcaattaataatggcagctttaaaaatgtgctgcacgttagctcaatcatgctatttgttgtgcgtgtgtgtgcttggcgcgttgcagaatgtatttgcggtaactagagtacaatgctgcatctgcactgacctagtcgtagagctgcccttctccaggccttgcgcacacatgctataacacctacaccactgagtaccaactgagcgcttctttataaatgggaagtcatttcgattcattgattgaatggatgagtgacgtgaaataattgcattcattgcagctctcgcagtagcaatctgcgccaccaggaaccgaccgggtgggacctagctcaatggctcaatgtcatcacagttgcgtgaatatcaaattgcacacggtttcccttccagatatatattcctataacaacacggtgccccgcggtccttttacggaggcacgatgtacgcaaactgctcgtttgggcagttccaaaaatacgcatttttcgacgcaatgacgatataatccaaagtttgttgggagcgcacggggtgaaaggcgatttgagtattctactgcaccgtagcgtttcgttttgtagccaattttccagtcgatactggcgcaacaaacgcaacggcatcaaagcgcgtgtcttgtacccacttattttctacgtcaatacgtgctgcgaatccgttgtcaaaaacacgcgtactactacgcctccaaaggatctgcttaaggaacggcttccgtgcgaagtcggcactgcttcttggatggtttctttcgaggcaaaggctctggttctggcatgggggtcgaaggtggttgaagaaagttgcacggctatttgtttcaaacatgccctagatagaagagaggctctggaagttctcgaagaagtatgcttatgcagatgttttacctttttttcgttccattgctacctgtcttaaacagctaccaatagtgcaccaatagtgctttggtgcatacgagaacgtttttaaacgtgcactgacggggataactgatggagatataaccaggctcaaggatcaaaaacaacttgatagtccagagtttagcgtattgtagcagaatcttgaagcatattgccaatcaactctgtacttgcgctctgagaagatgacctggtgatggacaagaactctttctttttctctttcgcaactcacattcactcataatttgcttcacaaaagaatatggaattgatctgttttgattgagtgtattcatatctttcctaatttcaatctactgactctcatctgttgctttataacggaagcggaagaaaatgatcgattcttctagcattaaacgagcatcggcatatcggtccagagaaacgccaaagacaaaagacgaaaacagacacaaacaacactcaaaacgaccggggaagtacgatcgacaaggggcgaagatacgggatacggtgtacgacgagttcccaacatcattatcatcattactgaagtgatcgcgtcatttatgatctgctaaagttatgaccaaggcgatcgaaagcaaaaaaaaacgaaaaatccggtggtttgggcgtagccgtgctcccgaacgacctcgagaaatgcataaattggacgatgtccaaactcacgagcagatcactgggggccatctcacggtgtgctcgataccggtgttccctgtccgaagcgaagacacgggcgaaagggaaagcacaagctgccggtagataatgaagctgaacaggcaatgggggccgatgaagagctcgcgtaccgaagagattgcaactaaggaaaacaattctgaagattgatcgtgtgacgaacacaacttggggcgctcactcgtacggaagagcaaaaaaaaaacggttaggcgaagcgaacgaaactatgaaggtaccacttgaggccactcggtggtgcatcagtccctccttcccctcggggcgaagggaaccatttggatggcggctggagaggaccgtttcaaatcgccacaaatcgatcaacgactgtcgaagaatcgtcgcgtcgtgtggacggaggtacaggggtggtgtgtgtggtgtatggtacgaccattgtctcacctgagcgcagcagctcagctcagttggctgttgttcggggtgttgccagccgctgcagaggcaactgtaggcgcactgtctggcggcggtacaggcagcttctttaaaaattgatttcaaccgcgaattgcggctcgagggggccgctggcgagccggcgatgcgcaaaacaaaggctcactgagagggatccaataaaatcgacaaatgaacgatctttctctcggctcgtgggttttttgttgttgtggttgatgttgtagtgccttctttagcaatcttcgtgtgaaggctgttcgcttaagtcacggcgatggtcaatgatgcactgcacactcaaccgtaatcatcttcgtcatcgtttcgccctccacagaacggaacgggtccttcccaagaggggggataggaccggtagtggcagtgcatccactattaatgcagaatcaatcaacggtgggggtcgagatcgaaacacacggctatcgcgtctggattgggtgcgatcgggccgataggccggctctagggaccgctggctacatcgtcctattgagctgtctggatgcattgtgtgaattatataattaatttcctttgcgccctcccaccggtcgagcgtcactgagagcagcgtgtgtgaacgatccttggtgcatcgcacgattatgactattgtcctcgggcgagaacaagggtgtgctgcgcctggatctaccttgggcgtgaaggaggaggttcttatgtgtgtgctaatctgtcggtcgaatatttgccacaatagtcggcaacagcagcagcagtagcagccgtgacgaataggcgcctgacggggtgcttttggtgtcgctttttgcgagtcagttgttttgcctcatcattctcaatgtctcaatggcttcgatgcggccaacatcaaaagggtttgatggcagcatcttcacagcgtcttcgtttactgcattcggattgaaggtgacctattttttaattatttatggtatttcatccaaatgtgatttttgaagctgattcttgtttgtgttctttgtgtatctgcatggatgttttgtgcggatggatgtgtttgatgtgttgaaattatttcacatttattgctgtaacctttcaccgttcaccgtgacgattgcatatctttttttgtgcaaataatgtatccgtaatatcaaaaacattattagaaaaagaagtgttgtaaggaaacatactaaccaatagctttgaattagtctgagaaataaaatagtctaaaaataaaaataaaatattgcacaaacaatttgtatagctataggcttagtctgtccttgctttaaagactaccccaagggttgatattcgtagcataaattatgtatgagagttattgattgacttaaaatcgctcacctgcctgtggccgtggctgtggtagtatcgaccgcagccaacatgcaatgtcccaggtgtaacgacacaattgcatacaatatagaagaaccagacactggctggccggctcgggactgcaaatgaaaggcaaaatcgaataacgaagaatccttctaatttcaacccccgtcctgttcctcgtggccccgtggggtcatggggtgacagctgtgtgtaaacctcccggagaaaagtaaggaaaaacgagtgagtgagaaaaaaaaagaaaaaacaatcccaggaaaaaaataaaatccccgtcaaacgatggtgtccgttgttgctgttgcagaaggttcgaaaaatagacaccagagcgtttattgcctgccggtggctttgcaaatggataggattaagtgttgtgcaggttagccgtatgcaactgattcgtactgaatcgatttacagtggagcagcagcagcagcagtaccaaacaggcaagaccattcctgctagatacaccctgttgctgcagtttcgaggccaggcttgacgctagctatctctcgctgtaagctgtcgggctgttaaacgctcgtgttaccgtttgcgatgcattaattaacgaagtgagggcgagcagacggctgacggggcagggaccggcaatagcggagctgtgaaaatcattgacattggtaaatttgcatatattgttcgcgataaaagaaatgattaagaaatgtggagtgggccgggtggccggtttgggtggctgttacgataagcgtttaacgtcgcattaattagtcagagggtatccgagcccaagtcgatcatttcgtgctgccctggtcacggttatgatgcggtttgacgttcaactgtttgaagacgacgcgcgttgtgactttcgctgataacgccgtcttaatcgtgctcaatcacatcgcaaaactgccgcggtgtatgtgcgtttctaagcggtgcaacggtgggtggcattgaattcctcccaggcccaggcattgtgacgcgcactgcacactaatcttatcgcctttgatacacgggtgtcctctattctggtcactcgccactccgggggtagcctttcagtttttgccaacccgcttcaattcctccggtctcaacaccctcccttgcacatagacgtgcttgttcattagtgttcctcttcaccctggtggtgccatgaacgcacaactcttccgcaagcgcatcgtcgtctgtggatgagtgtgggttgtgtggtttacattgtactcatggtgtttgagtttgctttttttgttcttcctttgcttgcgttgtgcaatactgctacgaatgtcagatttctagtcgtactcgattttggccgcaaacacacatacgcgctgctctaacgccatggtctggtaggtccgagtgcaattgtgttatcagctggcgatttttgccctgcattttctttgccgcgagtgacctcgacttgggatttgctatgtaaacataacgtgtacgtgtagctcgtgcctggaatagattgcctccccatacagccagtgacacgcacacacacacacacacacagacgcgtggcacggctgtgtttatgttgcaaagattagtttgtgttggtgcagtccccgttcgctcaaagcaatgcaaagcagcagcagcgacggcaccccggaacacattggctggtgactttggttttgtgccccgtccccgtgcatgccacccggaaatctagccgccaacggtgactaggtgtattgatgaatttaaattttgcactacaaaaatgcgctttgctttttaaatggtacatgtgcaggcgactggttgctctcctttccttcattgctgcattgccgctttttcccaatcacatgctggatttggttgtcttacccctccctcgcacacacacgctcgctcgctgcatcactaaagagcatgcgaaataacgataagtgacagttgaatgttcagctgtttgctgctacccggggtttcgtaaagccatcttccaccgtgcccgacccttgttggcgataaacgcgcgctcgcgaaaaataaaatcaaatacgccaactggaagagcagttcggctgtacaacacaacacacacacactcacaaacctagccgcactaaacagagcgcagacagcgacggcgacaagcggccaaagacgacaactaccctatcccaaccccgcgactgacaagtctcgggctcttgcgttccgcttctaattaagcgcggaggcccaccttcagcgtacagcgacgacggtggcagtccttcgtactcgtttttttccttcctgtgctgtgccctactatgtggtagcactatgtggcactgttgcgaaggagcagtatagcaaccacccacgccaacaccccaccgggccgacgggagctaaaagtctgacaagttcaggcagctcgcacgggagtcgggaatcgattgtatcgatagcagcccaagcgtccccaataatcgacgttaaattgtttcccccgttcgcgttggattgttaccatttgcgtagttacactgcttaatttttaggcgtaatagtaccgcatcacagtgtcgtaaactatcggtacgttttgacatgcagcgcgttgaaacggcacaggcaggagagcagccaaaacgaacgggaacgcataaaattgggttagctgcggtggaggcgtcacggtaacgagctggaagctggcgtaaagcgtagatgaagctgcacagacagacagaccacgtccacacgaacggactgggaagcgggagaatgcacgttgcaatctttgaatctgatttgcacgcagatcgatgcaaaaatgttgcatgtcaagcgttaataaagattggtgtttacgagtgttcgttttggctgacaccggccggcagcgggtgaaacatgcgacatcatacctggcggtacttggagcggagagttggagctgtgccagcaaaggtgtcaaacgtgcagcttatcgaaagggtaatgaggcatttacttgctctgtcgcaagacaattactcaagaatagaataaatacaacaaccaaaaaagcccgcaccaatttgtaaggattcattccagctctcccctcgcagggtaatgtgtgtaacaatacgaagtgtgacagacacttcgggggaagtttttgacagctcctgggaatggcaacccttgcggctgcactgctgcacactcgacaggggttttacacgtgcatgcgcgactggtcactccgtagcacacggtaaacaatgttgtaactgcaactcgccccttaagaatcctttcgcccctcaatttgtaggcaagtttccgtctctttgcacacacgctgaaggaacagaacgtcgtcctatgattatgctgtcagggagaggaagaaacagtacgcagagccacgccggggcacaattcattcgatcgggaccgggaggaaaagcgtcctcgtgcacatttgcacctcaatagcgagcataatttagtcaaattaagcgtactccgctgggagtggacgacgtaggtcgtcggtggtggcattgtccgagaggactggtgccacggttgctcaattgtaacaatcgttgacctaggtcggtggtgatgtgtgtggccattgtttcaacattccactagcttcgggtcctcctaaaatccactccccggacggatagggcgaacgcaagtcacgggcagcgactgctctgtggcgaggtgtttgtgtgttgcaaacttttgaaccgaaaactgctacgaccaccactacttcgctgctgttttgaaccaggagctctgcatctcctcgactaactgacaaaaaagaccgcatccgctcacattgtttctatttctgcagggacagagaggtggtctagtggtgccaaagttgcccacggtggccgaattcgaggccctacatcctccaactaatagcagtgccagcgcctgctagatcctgctactagcacaagtgtgtgtgtgtgtgtgggtgggaagttcaatgttgaaatgtttcaccgatatttatcccgacactgaccccttggatgagccagcgttttggtgccatttctggctgtgttttcgctcaaaccaaccagttcgacaataaccagtgatgttgatatattcacgtgtgtgtgtgtatgtgaactttatttttctcgcgttttcccgctggaatgtgcatgacatgtcgccgcaactgtcgacacagattcgctctagtggaagtgcatcgtcgcgcattcgctgctgcgcgggctatcgcgggtatctagacatacgtgtgtggctagtgtaggccagggagtaccatcaccacaggaaggaagtggttcgagagggcgaatgcgcgccacggcgttccaaaacacaaaaagcggtttggatccaaactttactgcatgttttccaccggcagtcctgcagacgatggatccacatggacactggagggaacagcacagggtcagcgtcagcagtaactggtcaacgctgcgttgcgttctaatgtggggcttccgcttgtctagagccttccgcggagtgagtgtgtgtgtgtgtctggctgtcctgaaaattggattcagagcggatgttgactgtttcgcgtgtgtgtgtgtgtgtttgtccagccgtggattgttgggagaatatgtgctcatccatccatgcggcaagtcgctcacggggtggaggtcgcagcaccgagagtttgtttggcattaagtaccttcagttgcaaaggcaatgcaaagaagaatcatttatcaaacctaaccatcttcgctcaagggtttgatattaccctcggagaaccactttgactcatgatccggcgttgagcatttttctagtttcacacattgcagtaattgtcattagcacttaagattgaaagcccggaatgctttacggcattggcccgtagatcgcagaaaggccgcgagcaaaccaaagaaatggatgtctttatcgcaacgaaacgtcgcaaattttgcgcccttttttactgccccgcaatagacacttgcaacaagacggcagcgaaagagtaaaaaagccagagaaggcattccgccaatgctgtaaaaagcaccaacaacaacaacaccaacaaaaaaaaactcgaaccaaacgcacactcatcagtaacgcgagaccagtgcgaccaggcacccatctcccttcgaacgcgcggctactttcccagccataaatcatccacttcaaccagattgagtctcctgccgccgcaccaggcgtgaccacacgtctggtgcggtgtctcgtttgttccgccgtttttgttggcgtgtgggtggtggtggtgggggcgggggagaaggtaaattaatttacacttgcacacagcgcagcttcaagtgggagatgcacttgtcgtctcattgcctcgttgctgctccggcctgcattgcccgccgtgccaatgacgcagtggggttttggtgacgatcgctacctttaccgcgcttgatataagggttgaaaatcatcatcatcatcatcatcatcatcggatgctgatcggacgggccacactcttgacggatcgtctccatctcgttgccggtccgctttcgcctagccccctcgtcgccttgcccgttagcagttcgtgaagaaaatgtgcataaaattagaaatcgaaccctccgcacacaccccaggagggaggggcggtatgattgggtcccgtgtatgggtgtgatggtgtggggctcgatgtgagtggcaatacatttgcaatattagtggttagattccatttcctgcacagggagcagcgcagcggaatgtagaaaaacaaaacgccggcaagaagtgcggatgcaaacttgcaattgttggttctgcagctcgggtgcgggtgtgtgtgagtgtgtctgtttgttttctttgcacgctgcctggtggccccagggaaggagagggcgttgttatgggagaatgtaaaagcaaaacaagccacccatccccgttctattgcatctcgtctcgtggtccaagaccactccctatccctctcgcctcttcccgcccttaatgtccctctgtaaagaaagacgatttgttctcacattcctgcttcctccttccccatgtaccaccatctctgtctggagaatcgtgcgcacacacacacacagccacaggattgtgacagtaccgtcccctgctgggaggtgagtgaaaagaaacacatttcacgcgtgtgtgtaccctgtgtaatgtcacagtcgatcacactcgggcccccgggtgaagccgattgaatcataaattgcacttacggaagcacttgttcgcactggcctgtccggtggccacaaccgggtccgagcggtgtccatgtgtgccgcattttattttgcagccacttttacaactgtgctgctctgctcccgctcccgctgcaccgccagttcgagagatccgagcgtacgagaagtgatgatgcaatcaaccggacgggaggcaacccatcgttagctcgccgctggagccgatagagccaacggggccgggagggaaggatggaatgtgtaacgctgcagctaaatggcgcgtgcaccaacaccagctcgcagcggcgagaaaggcgtaaattgtgcggcgcgtgtatgattcttggccggggcgcgttctccctttcccccactgccaatcgttctgcccttctggatctgggcgggcggcatgtgactagctaattttccaactcagtggctggccggcggtccgtaagatgatcacaatcactttggaacagtaatgtgggcacaaactttcgttggaaggttgagttttttttaaataaataaaattgttaaatttccaccaccaatttcccccgttttcactgttccctagtttgagtttgaaggtcaatcaagaggaaaagaagaagcgaattccctgcgcaatcacccttcgcgagagtcggaggaagggacgcgcaaagaatcctattgatagaagctactgcagctactacactacacttgcgtaattgtttaacgtgcagaatgaatcggtgcactatgcggccgggaagtggccgtgtggtggggcagctctcccccgttcccgcggcattgggttaccagcgtgagcgtgagcgcgcgcgcgcgcgcgaagaatcgatgatgccgtggaggttgtcgcgcggcgcaaacattgtggtgtgtggtgtggcctgagaccggctgctaggggaagataaaatgtagctcgggtttgggtggcggcgcgtgctggtttcgtgatcgcggctcaccttcccaatcggatgggcggcggttgatggtcgggcggggagtagtatctggtgttcattgctgcagttcggggcagaatctgaaggcccaagcatgggcgaggcaagtgacgcaggcgggtgccgatgcaccggtaagaagggcgcgcgaggcaagctgataagaatgtgccggctgcacaggctgcagttttcggtctttgtctttgtcgcacggcattctggagcaaaagaagaagaagaaaatgatgaaaaagaagaaagatgcgtgtgttggatgattgtagccgaggaccgatgcgatggtgcggttggtggtgttattggtcagctaatggtgagccggtttgccactgtaaaaggtaatcgcgactcgaatcgtcgcgagactaaatatagagcacttcctgagttcatgccaagtggcggaaaatggacggaactgcatcgcttgcccctcccgtaccctccttcccctttccaccagccacacacatgcacacttataccaacacagtggggttgaacagtgcattggacaaaatgcacgtgtaaaaaatgcaacagcccatgaatgtagttgtgtgatatggtgcactcattgtgtacgtgtggtttttttttacaaattacagtgtgtgtgtttgtgtgtgtttgtataaaaaacactacttacacaaacgcgtttactcgtgaagatcaattcattgcaacgcgccgaatgactcgcgacgattgtgccgtttgggtggatgatgaaaagtaaataacattctttgggtaaatagttgcaacccgaagctagtgccaactgtgctggcttgctcctttgctggcgtgttcgggcctcgcgtctcgtctcccgttacacggacacgtaaatggtagatgtaaaaataaagtttcgcgtcggggttgtattgaacggccgtctggggtggggttttgaggggggaacgcgggtatggccaggataaaaggtgggtgtgtgtgagagctccgaggtgaacaatcggtcgtgaccacggccgggtgttgtgcagccaggctgtgtgcaaactgcagcgagatgcaggaaaggggtaaccgttttcggcgagccttcttgtagtttcagcaccctcggttacccacttctcctctcctagcttcaccacacgtctgttgttgcgggcgttctgttcttctttcactgatgtttaaacgtttcttgaacgatgcgttttgcgtacgatttttgagtttataacacgtggttttgcgacatgttaacatttacattgtaatcagttgattgatgttaatcttttttatttatttgctctccttttcagctactcactcgtgcgtttcgccagaacctgtaaatctcctacctggtaagtaaatataattaaaaaaaggaaataatatatttcaaagcggtacaacggtgttgtagcaaacatttagtgcttcacactgtacgtttgaatatttgctaacacgatatgttacagccgacattaaagcatcttaaaccaactgaacccaacatgtagttctttgcaagcaaataggacgtcatttgaaaaatgtgcatttatagctcatactttatggaatgatgtatgttcttgcccgatgcaatctgctatagaccacattgcaggctgcatgttataaatatcggctaacacaatgcgtcacctttttctcaccttaccgcgctcggacgcttaaatcttgtgggcgtttgctttctttgaccttatccttgtgcgctaggctaagcgtatttctaagccagtggacatgaggtactaccggcttccctttttcgatatgtaacacagttaacatcacaagcacacacacacacacacacagaaataatgtcggtatggcaattggacaatattgttatttatcgccacattcaccaaccgatcgaaattgtcccaaatcgcttcgagtacataattctcctatctgtctgccgctggtggcatttgtacgaaaacgtataaaatgccccgttcttaaggcgaccgccacacaattgtgggcattgagctgaggggcgcgcgagactcatgtttgtcgcatgcacatcgcggcggcggcggtgggagcagcggcttttcgcgcacctttgtcgccctgttaagcatttttctagacgacagataccagcgcaaatactgttgcattatacaccgggtgtttaagcagggacccggtggtggacataagcagaacgataaaatatttgcaaaaccgatgtttctttgcgctgatactcggcggatacgagcgctgtgtttgtacaaaggtacaaacaccgagagcgtgtccgccatgggaaactgcctcaaacatacgcccttccgtccccctcgcctcgccttttaccaccgaaagggcaaaaaagggtgttaatcgtttcgctgtgcgatgtgatgattggagatcacgaagatcaaacgggtgctggggtgaaaagcacgatgctacttttgcgacataatgcgctcgcttcgatgtgttgcgcgtggacatgttcggcatgcattcttcgcattaaatgcaatacgcgattattttgaaatgaaaattgatcgcaaagaaaatctcaaacgcttgattttacttccaaaaagaaaggagtgcgcaatgcgaatacgagagtgaaaaagagagcgttatgacagtgcgcttgatggctaatttgcaaacaatttacataggccgcatcagaacagttcattacggatcaaaataaacaatttactttttgctcgtatttgctttttttgttgctccccgggcggttgttgcgatgacccgtcaaaggggatcagcggtaacagcggcgaattcggcgcgctctcgtggccgtatggagataaggcgagcgtaaagagtgcgaaggggaggaagggacctcgaacaagaacacgactacaatcgcacagtacgaaaacaggaagaaactcggaggccgatgtaaaactggccgcccagggtctggacaaaactctttatccaagcaagcactgggaatgggggaggaacaagggcgctcctttcctcggggccttgctggctggtgggcggcagggaccgggggaaataacaccaattcatgtcaatgtcactgtcactcaaccccaacatgcaactgcatcatgggggcacgcgcgaggttccctcgttctcctccgggaagttggtttccttttttaatcggtggagtgtcgagaaggggtgcaggcacgaggtttgggtaggtacagtgatgtagggggagaacgatgcgtgtgcagtgcaatgatcaaatgatacaggcaaggagagcgaagaggtcacgaatggtggaagtacttgattttcaggaatcaatattcctcgctgtctgtcaaccgttctgtccccaaaagctggcggtggggggatccggtggatcacgatgggtgagaaaatgagtgaataaaacaaaaaacccgattgcaatactaataataaaataaaataaatctcctgcctcgtccagcttttttgattgtgagcctgatttttctctacattgtagccgatcgtgtgcgggggatgtcagcctggggcagatggcgcaaaagggttgccgtacgcaggacaagcagaaaatcgtggcttgaagcccgcacaatctatttcctttggttgttttaaaaatgggttgcatccagcttagtctgagctggaagttgtctcacccgtaggggcaacagggaacacgaacaggagactcgtttccgcatcggctagcttcggtggaaattgaaggcattcaccccttttttctttttctagtccataattgcgggtgaaaataatgccgcagttttcgtgccgtccaggggacaggttttcttcctacaacatgattaacattgcaacatttgttgtaacaatgcgattgtgtgtcccagtgcgtaaaacgcacgagcctccgatcatgatgggcatgggaaggaaaaaccgttcgacggtacatttgttgcgttcgatcattgtcaactccattaaacgaacctgaataaaccggtgcgtgtgtgtctgcggtgatggcgatctttctttatcaaacaaacgtgtttgagtgttctggaggcgtttgagtgagcagcggccatttgcattcacgaagccgagttgcatcccaataaaaccaactgcatgagatgattgatgttgggagatgagctgcaatacattcccaaccgtcccgtttggtgtttgattgatttttcttgcaccgagctgctgcaaaccgggcccctggatgcgcactgatttgtttgcttgctggttgcaacaaagccacaccaccgttaaacctggtgatggtgatgcacctgtggcggatcgttgcgatggagcgactgatggtgtgagctttgtaaatggaatttcacgcgtagcgcgtctagacaaaccccaattgcggctgcagccccgtcatgcgggcacgaccgaccggacggccgagaccggtaagacagtgttaagtggaaatgagctgcggaatggctggcatggtcgtcgtggcaaataacgttggccatgttagggacacaagaagatgccggtatttggcagaaggtgcaaacgcacacaaacctacgtgaatgcgatgtcttctgaaattaactgtatcgtttgatgacacaacgcaaaacgaaccagtttgtcgttactttgagagaagaggatcatgatgatgatgatgatggcggtggtggtggttcctcaagaaagatggagtgaagcaagtgttagatccggttaccgaagcgattttcaaacgcacagtaatgattagcgaacgggccccttactgtttgcctgttggtggtgcagtcttcaatcatggaacacgctgggctcataaggaaacatggggcataatggtcatgtgaataattttgctcttttgataaatcattaattatcttcaaaatcgttgaataataattcaacaaaaattggtgctttaactctagattcatggtacaacatgaactgcactcgtttacaaacaaaatcagtttaaaaaaatgtcagacaaaattgcaagttgcaaaattgccttaattatattttttataatgatgcgaagccaaatggtaatcggccgatcccgtcagatcagttgtcaatcacttacaccggtttcgagcccaagtaaattatgtaaagctgctttagaacgttgttcaactgtaagtaaacaattagcgtccaactgaaatacttatgcgtttctgaacattgttcatttgtaactaaacaattgactcctctaagctgatacatttgctcaatagagtttatcaatttgtttttgttttcacttacaacaataatgcgaatttagttgtcaataatgtgtatagattgctagaaaatttctcatttattataactcaagatcgaaaccaattaaaacaatttcaaaataatttaatttgaatagattcagaatcaaacaattctgatgcccgacgagctcgggtaatatagatgaatgtttatattggcgaaagcaaatgttttgctgcgatttgacaatgttcaaaagcaccttagcgttgtttagttgaaaactttcgaaaactttagttgaaaacgttggcttgaaaacaatataataacttgcccgtcataccttactttaaactctctttctttgagtaaataaacaaatcgttgatagtcaatccgatttatggttaacgcaaattgactttcgactatggtgtttgcgtcaaatgagaagaagataatcacaattatttctgtaactatagccaaatgataatggtaaaaagacaacaaagataataacaagtgtctcaagtgtctggatgtgtatcctttatttgataagactgttttctagactgttctaataattctacaagaggctttaaacatataaatttgtatatattgaccctatgatgattttgctccgagtgtccttattatttattaattaactatttatttatgatttattataacggacacaaatagaaaacagttatttttgcaagactgtgcatttttgatccgtaaaaacagttcctggaaaaaagtatgcaactcacagtacaggtgaaacataatacagcggttgtagagcgtactgtttggacaagttaattaaattgcacccaagcgtgtattaattgtacccgtgttcggcgtgacgggcacacacaggatcaaaccactactgagaaactggatctgcttcgttcgcactcggcggtggaaagtcctttccgcacagcacaggacagtgcagattttgaaacattaagctctcgcaaccggcgtaaccgaatccataaaaacggaggttcctcgtccgggatctcctttcttccaagtttgtgttgctatcttgggtcgtaaatcttaacagtagcagtagttggacagtgtatctaaaaaggtacggataccaaaaaggcacgagtagaaaggagcatgtctagatgatgctggtgctatcatttggctccaattcggacatccggattgacgtcggctcgcggtgtatgtgctttagtgaggcgattgtaggtagcaattctccctcgtgttgctcctttccggaatagaatgcaacaaggcacaatgttaatcactcatcagaaaagacgaaacgggtccgttccgcaccggcaattttccggctcggcacagtcgatttctgcagcccccgtggggacacataaacaagcgaccaaacaaacggaacacacattcttcattctcgttgcgctccactcgtcgttttgtaccgtgctggagctgtcataaagcatgtagtgcaaagaaagttctcatctgagcgcttcttaatgctcacacttgcggtcccgtctggccttcggcagctccggcagctttggggcaattgttgagccgtaggaggaaaagacacggtacatataacgcccgcctcccagtgtgttgagggcagctgcccgtgctactgtgctgcactgggattcggcaaaacaatttcctaaatgtggtcgaccgaagaacgaacaaggttagtgtgtaccttcgctgcatcgagaggtacgccacttctttgggaagcaagcaaccgctcagctcctggtccagactgccgaaactctcaagtacgtttcggagattccttcgggagcgtgtgggttgtatgtggcctcggttcaagaggtgggtatagcacattttatctgccgcactgccattcgtgatgcatacatcaaccgttgctggaagtaatcgtacggagatgatagacgagcgatgaaaaatcgcacagaacaaaaggccatgacacgaggacgaataaagagttgccagggcgccatcccaccgaggggatgccacagctgtctcgaggagcaagccgaaatgatttgcattcagctgcatcgtgcaagatatggaccggtgagcattggctgatggagatgaacgtccaccagagataccaccgaacgcactgtctggtggtgtgcgcaaggttctctgtgagtgcggtttgctgcgatcaaaagactgccgagagcctgtcggcttatttttcggctcggcacaacaggctttggggttgtaaaacaagcaacaaacaaatgtaaatatcgtgcacaacatcaggcactgtttgagtgtctggttaaataaagaaacggtccaaaatttacagtgcgatggtagtgaagtattgctttgagaatggtttgaaaataacggtttgtaagttatctatcaaatttgtcatcatgcacataacttacaagccaagttatatgtagttgattttagagatcaaatacgttcctccctgccaatgcaataaaaaaagccatccaaacttgagacatttgctgtgcagtgttgggaatcgatccaccatgttgtaatttcaacaataacaaaccgaacaatacgcctatacaccattttaaccgactttccccttcagggctcagtcccgcttcccactcttattggagcgtaagtgcagcaaacgtccaagcattcgctctgtagcaagcggtgcaatcaacgagaaattacaggcttccaggctaccaatacgatcatttcagctgccacctctctgccacctcgccgagtgtaggtaaaacgcatcgcctcgaagcatttcccttacgtcggagaaggctatgctccatggatgccgagttgccgtggatgcgcttgtgttgcgttgttctttatgaacgcgttgaaccttccacgttgaacacagctgaggcgagcttccagcgttggggcgagcctctttttttcaccgcctccccttttacccttcatcaacggcagggcgagtgcactagtgagcacttaattaaaattaaactaattaagaaagctcgtcgtataattttcacaccacaccatcattttcgggctactggtaatgaaattaatatttcattctattttattattaacgtttacatgggggggggggcggggggggggggggcagaactcggggcacagttgtttggtaaccatcgtaccattgcagctcgaccgtttcggagatgtgacccttgcaacagcgtttctttacttaccattagtgcgagattttcatacgcgcggggagctctgcaccacattaatctcagaactcggaactgctccccttcgtcctcggccaatgttaccaatgctgttgatcaagcgcagtagcacgccgccctcccagtagcacacgatcgcgcgtctattaagtgttcgcatgtgcagatcgctttagcagaacaatttatggtgccggctgtttgagaagcgggctgccggctacttacttccgcttcctccgatgattaccaggctggtagctggggtcccggtggtataagaaaaagtcgctcagtcacggacggcaacacatgaatgtttcattgaactcttttgccgggtgggcggtggctaaggctgaaagggtgcttcagcaccaaaactggaccggttcagaggtttcgtcgttttcccttagaacgtgtgtgtgtgtttgtgtgtgtttatccaagaggtgaggacgaaaactgctgcacgattcttcggcaccgagagattcttacccgggttggcctcgtagtagggtcgcaagagcaggccaagggtttgggtcaatttaaaaaacgggataaagtgtgcgaggatcaagctgaagctggtggtgtgtgtccacattgtttgatgatttatcttctgttgctgtttgcgattggagcgcgtgcaatcgaagccgtaatgctaataaagctggaacaagcaagaatctggatcaggcaggcaggcgggtgtcgggtgacacacaagtgcgccacattatgaattattcatcctcacgtgatggaagttaaacctctatcgtgctggtgcgagtacggcctgggtggagagtttacaaactcaaatgtcaagcgcatgtaaactgtagaaagtgtagatcgctacagaaatgtctctatttcatagtgtgaccttccattttgtagagcatgtcaaactttggaagggaaattgtgtacacggccacaatatctgccatacaactcaaatcaggctatagtttttttttccacaaactgctgatgtttaattatcgtgttctacccattgcttcacgtaacgttggaaaatgctttacacttgcaatccgcccattttcgggcgtttctacacactgattaatcatcgataccaacgctggtaggtgttaaaaggataaagccggtaacaattaatacagtttcacggcaagagcgcaatcaaggagggaaatgattctttcgctttccgttatagcctcggcaaggtgcatcgggagaaaatattgcatggtaataaattcccccctcccacagtaaacattgcatccaacttcgggactacagtgtaaaggagtgcatttttattcatttttttgataaatcactaaatgtgaatcgtactcatcgtggatgctttatgctgatggctaccgcttgccgaattaacctgcgaagactgtgataaaacgttgcttacggctcaatcgaggaaccggctacatacccactaactccacgcgaaggcttgacctctagagtgctttccgtgttcagcacaaccgaattgtacaaaagaatatggtaggcgggggacacaaaaacacgttggcaatgatttatcggttggcattgccttctacattgaagatacaattgatcggtcggtcgcgccggttcggtcaacctttctcttgcctcagtgcatcaagtgcagcgtaaatgcaacaatgccgcgcgtttcctcgtgcccccggccttgcgggtaaagtacaaatgcagtttatttccaaattaattagatccgctgctaaacaatgttctcctcgagcaaaaaagcctaatgagatcttcggccgcacgaaatttgtgccgagaccgcggaccctacaatggcgctgcaaattaccgctttttccgttccctttttgtttgacccttgcgacgtcctcccctcacgccgatcaacctgacgggttcctgatgggaggcgcagagacagtggagtgacagttatcgacacttgcacggtgagcaaacgcagggaggaggtcgctggtcattagtgggttttgggctggagatgggacggcgtcacacactccacggaggagaggcagcatagtgatgttcattttggactacaattcagacagtcgttcgcggtcggacagaaaaagtgctaatcgaacgcattgcatccagcgtggccgcgaacttgtgtcccggggcagtttgggtcgcgcattggaaagttaggagtaatggagtgataagggtgagtgtggacaaggatgatgatgttgcttcgggtatgagtgcgcgagttgcaaagtggcaaaaccaaatattgtaccgccaagggatgcatttggtgcgatgcaccaaatcgagctgtggttgcctctacaagaacctgcgcgctgccattagcgcctataaacacaacaaggtgtgaatgttcgaattgggaggtgagttagcagtgtgacaaattgatttgaaatgactgtttaacataccaatacggcatgggcaatacgtactgattacaacaagtttaatgagttaaacaatatacttaatttgttgcattcaatcctcagctaacaattaaaagttttttttgtgtgacgaaacaacaacccatcttaacaaacaatatttcactagccaactagaagaataaaacaaaaaaacaatgcgaatgaaagctagatactactaacacagttcaactgtttgggtatggtcccgtagtaaagtcgatataacggacgaaataacaaaatgttccatccaggtgtaggcgccataagacacaatggtacatcaatccattgctgatgattaaaccctctagttgcttaggcatgtcttgatcaactacgcttgttaatccaaagaacaagaagaaaaagtgttaatccaaagaacaagaagaacaagtggttaattcaagatgtatcgctcaaaaaaaccaactgagttgactgcagtacaggaaaacaaaatcttacagcttgaatatttttattattattattattattactattacaccatttagcagctgttgaaaatgtatgaaaaaatgtgtacaaacactgtgtcaaacataattccaacgtgtcatcaattcgcgacatagctgtcccgcaaatggcagtaaaaccccttgaaacggtttttaaatccatcaattaaaaacgagcccttccccaacagaagaaacagagagacaatcaaaaacaatatgcaaaaaaaagatgacggaaagcaaaaattttatcaaaaaagaaaaaaaaatgcaacagaaaaacactcccatgggggtaaaaaaaggaaacaaaacatgcacattgtacgaaaacgtgttattctcttccaccttaccattgcgtgaacgatatgttatgccaaaccgctcgaggccgatgggtaggcggccgtgtgtacgtatgagtgagttaccaccaccatacctgtcggcggatgttcaatttcgattctgtgaatggatttacttccgggtggaattgcaccgtttgaaccgtttgaactaccccagaatgccggggcggttttgtttttctttccgttccgaacgccgtatggaaaggaaatggattgttgttagcacgtagcgcaagccaaaaaaagcaaaaagagttggaaagaatgaaggcatgaaacgaagagcacagaacagcagtagcagcaaatacgattcggcaaagtaaatttacatattcgacgatcgacggctggttttcctctgcccagcgatttgctatccattgccgcggtgtttggcgtggggaaacagcatcggcacaaggaaattggccacccatggggggagggtactgcttcgcttgtccatcgtaatcggtgcccatttgcactcactggtacatggccaacacagagagggagagagaccggggtggcattatttgggggagttggtgtcggagcgtgcacttgccaagggtgtcatcatgtgccttgaacgttgcatttccgattccccagaatggctgcgatacggcgagcaagaatggttagcgtgaaacaaaacagtcgtttgatgattttgattccgtttcgatcggaagagttggtgtgcgatattgaatgtgtgggacgggggtggcgaacgtttttgttccctgtacagatggactgtcacaaatttatgcaaaatgtattaaaggatgacgtttcgagtgatggagccagttcgtgttgttttttcgcgcaagctctaccattttcggtggtcgaatttttgcgccacgtttactaaatcgccaaacaacgcgatccaaaaatgtgtcagctctctttgttttgattttggctggcgttggaggtaaaaccaacaagaaaaaagaaaacttaaatcaaataaataaaacctcttggccggcactggcgggagaacgggccacggctagctctgctaaattaaacactttgttatgttttgctgcaacttattatattataagcactgctcggccgacaggaaacgtattgaaatttacgattgcaacaatgtagagctgttcgtttgcagcaccccatttgtgaatggcacttgtgcgctggaagtacaaatttgaatgtttacagtctaagctgtgcgcacaagaattgtcacccgcgaagaaacaatcatttcgacactttacccccggttcccttttcttcggctttctctctctcccttgccgctgctggttcgtcgctggttcggttcccacagctgcaaaccatttaaacacttacgcaaaacgcgcgttccacttccagggcaccgggaacaacgcccagaacgaaatatcgttaatctccttcgggcgtgtccttgcctcgcgggtacttgtctcttggtttgcccagcgagatctgtacggccgcgtgtacacaggctcttacaatgttgcgtgtgtgtgcggagaaaatgtgtaatcgatttagtggcgcaacactatgcgcaacgtttttctattaatgcacgtctgtgcgttttgtcctgcccgaagacgcccaagacactcttcccaaggaatgtgtgtgcacaggaagtgtcaactcgtcaaaccaaacgcggtggagtgtgtgtgtaaggtgtcgtaaatgtcatgccagcaaggatagggtatttgttgttcttaaaatttacgattacccgttctacgctagtgcgcaattcgttttgggcatgtgcttgttggacatgttgtggcgggcagtatatgcaaagcaaacagagagcataattgttatgatgactgcgctcctttcacggacggagcggtttcagctggaagggcccacaacactcccagctcagaagcaaaacaatttaatgacgaatcgtggaaaaagaaaccaattaatggaaataaatactttgttgcgagcagtagagggctgtttagaaattttggtaactagcgattgcgtgtgtttacaatgtattaaaatgtttataagccgtataactatcgagcaggaagcattgattctttcaaacaaagattcggattcaatgtcgcgtcgttggatgaacgaacaatattcttcaaattctagacagcaacaaaatcgcgctgcaatacaactataccgttgatcggcgttaaaaagtatgcagacacaaagtaaggcaacaataattacattaattcatcagcgaagaacataatcaagcatagctggagtgttacactggttacatgccaatcggtagaattcattaggaattggtcggcaacatcgtacctccggcagaagaagcatactttgtgctgaccaatgcaattcgttaggcgagcagtctccctttgatgttttagcatcgatgaagtgatcaatacactgaccatgtgtcggatttgtgtgtgtatgtatgtagtctggcatgctctctctcctgtctagcgaaaatttcaaatatcagtcaaatgtgttccagcagcacattatcgggacccgtctagctagtctccacactcacactttccatatttttcacaccttggtctgaatttgtagtcgtccccgtgcgggcatggaaaattactgtgcaactccggacggtaggtgttgatgtatgcatccaataaacacttcacgtgttttgccaggtttcgcgtactgcaaacacgggctttggcgtgccgtacgcgtacggctgacaagcgcgtgcgacaaatgttaactcgccacctcaatcaacaccgtagcgtaggacggcgaacggtaggcgcactccgccgggattgacatgaaatttcgaacgtggttcgaacaatcgacctcacccttacccaatgatttcgcgccgagcgttcgaacgggctaattttcagaagggaaatcggcaaatggatggatgtgtttttccggccgtattatgacgaatgtgtgcatatccgtgtatgtgagtatgggagcatgcccgcggtggtggttggcggtgggcaaataataaaattcaatttaattaaaattgaaattaaaactggaaataattacaaataaatcataattatatctgcggttagattgtgtgcaagctaattataaatcaatacccgcccgcgattgggacattcgcttcatcattaatggtcacaataatgcgggacaccggaatgctcggtagcatcggcctggcatacccctgtccccggaaggacaggcgatacaatttaaccaccaaacctgaccgttgttcgggctacgatcgccatcatcgctttgatgtgcacttgaactgcggcggcgttggcaagcattggaacggaacgaaacaaaaaaaatcaaccaagtgataaacacggcataaccagcacagaacataacctccagtaccaaccggatcagtactgagtttcgctctctgatccgtgtctttaattttctttgcttttttatcattttgcttttgttgcctttttgtttttcccagcgtggctcgattggaatgagccgtccggttcggtcggaaaatcatgtaacggcataattactgttaatatgtgcgcaaataaaaggtgcgattgcatagcggatcgagtgttgttgccgccaccggggccacactgtctaccgtccgctgcgatgaaaagtgcataatggtttcaaaattgaatatggcaacgcgtttggggaatgaatggaaatctcttcacacaagtagtttccggttgattgagccaatcgattaacactcgtttgtgtgtgcttttgattcgctcaagctgtgaaataatgcgccaactttggtagaatgttgtagttttttcttcggctactttatgtgagctgatctgattgctgaaacgcgctgctgaggatgccgttttctcaagggtgactgtgttgtgcggcagtgtgactgtgtggtagtaatccctacgtcacacacacacactcctactgtatgcagcggcgaaggttatgtttagcaaaacgcgtcccaactgacaaagggcttcagggttattcggtcaaattcagatcaacatgctgcaataatcgcgctgataagtcccgcacacggagcgccacttgcatgcatcgttgaatcttccggaacagcaaaacgacactggggcacgtatgtttgcagcaacacggctgacccgtggccgtgtgccaagcgtgcgcggcccagtacgtcagcgacacggccacagctggtacgatggatgctcagtacgctcagttgatatgcgctgagttgtgtcagttgggtggttgggttgaccaggcgctagtttacagtgtgctaggtggttggtcgggtgtgcctgtgaagcctaaatggaaccaaaaagaaggttcggagcaagatagaaataacaacaacgtgccataaacagctccggtgcaaatatgtctcctccagacgcgatacccaatcagcgcaccccagcccagcgggtagtatcactttatctagagcggaccggtgctactggtgctgccgatacgtgtcagaatgtcgtttcgcgcgctcgcgccctatgatgcttcgtgcgcccagtcggcatacactcctaattcgtatggataacgttacgactcgagcaacacgcactgcacgatctgtctgacaaacactctgccttgctagagcaaaccgctttattcttagaaggagagggaatttcaatagatcacgcgtcgtgctgcagcacggtgtccgattgtacaggttggaaattgtaacgctccaggaagtagcgtagcaaaagaccctcccgagtggatggccatgctaggttgatggacgccgtagtgcgagcgcttgcactgacattagcaggaagtaccgagttcaattgctctagtaatgcaatcagctaaaaacagtacaagaaggcgggtgttaaagacatttcaaacatgctgcagttgcggtgtgcggcctcgttccattgtatgcttaccatctgttcctcgtcgagcgtattggtgctggtggcgatcgattgcaccaaattggccagcgcgttcggaccgagcagactcacgacgtacgtgtagttctcggtgaggaattcgatcaatgcgtccaccccttggcggctgctgaggacggactgtagaatggatagccgttcctcggcgttgaagttcacctgcagctcgccaccgatggcagccagcaggtacgagctcagctgctccgtatcgttggcacatcccagtgcattgatcagcagttgccgttcacctcggttgtccgaacccagcagcttgccgaacagatactggaaggcgaccgttggcgcggttcgcaaaccgtaacagtacaccaccgccgaaacgtccgggtgcacaggttccgcgtcgaacacttcccgttccagggcgtcgcgggtcgccgtcatgcagctttctatttccattcggcaggcccagctggagattacctgtcggagatacttctccagcagtctctcgtccggtgctaccgttgtgatgtccagcgttacaaacacatcgccaatcaaggtgtcgacaaacagctcatagagaatgtaatcgggctgaccgcgcattcgaccgtggaagtagctgaggacccgattagccgcttcccatggaggatactcccgttcatggcgcacgtagcccagcagctcgagcgcaatctccagatcgagccgatttgagcgagccaaatggaaggaatcgtcgatcagctgcgcccgactgtgcattggaatggccgccgtgtcctcgagcagcgtccgaatcagcatgtaccagttcgagggatcatagttgacgcgatagaatcccgtctgattgacgttgaccaaaatccactcgttgttcggtgtgctggacggtacacgtaccgctttcgaagtcatccactgccactcgagcagagcgtcctgcgcatcgccctgctccatcatcgtgtacggtattacccaaaccgtgaaatcattattaactatcttgttaccgtagaatcggtcctgcgagaggatcatctctccacggtatgagcggcgaacttccagcacgggatagccggcttgattgacccagctatgaacaaaccgctccacatcggtcccctcgggcagcgatacgacaccgtcgaacgcttccgtcagtgcggccacgaagttatccgtgttgaccgtgccgaactcgttgccctgcacgtacgtgcgcaacatctgccgccaggcggcatccggcagcagcagccggaacatctgaagtaccgagccacccttggagtacgccacgttgtcgaacaggctgaggatggcattaaacgttgcgccgcggctgaaagtcatcgggcgcgtgctttccgcggcgtctgtgatgagaacacgctgcaccacctgaacgttgaacaggtcccgatactggcgctccggataagccatatcggcccccaggaactcgtacagcgtcgcgaagccctcgttaagccagagatagctccaccactcgttggtgataacgttgccgaaccactggtgcacgtactcgtgcgcgatgattgtggtgatggtcgtttgcgctcgatacgtcgtaacgcccggctcgaacaggaggacctcttcactgtacaagcaggaaatgggcgcaaatgttaccagagagtagcgttgacaaatgaaatgattcaccacacacacacacacacactcaccgatatttgcacagtccccagttttccatggcaccggcagaaaattgggtaagtgccacctgatccaccttgggcatgtaggagcgatagggtagaccgatgtgctcgtccagcgcgtccattacgcgaacgcctgcttctaatgcatacagcgtttggttgatcgcgttggggcgagcatagacgcgctgggcagccgcctcgttctcggtgtacaagaagtccgacaccaggaaagccaacagatagatcgacatgcgcggagtagtttcaaagtacgtaacaacgttgccgtctagatcactgaaattgcaatcgaaagttatttgtcacaaacacacctcgcaacgtcagagcactcgacaatcgccatacccggcttcggcaaagatcggcatgttcgatacggccttatagctgggatgatgtttaattcccaactccaccgtagccttcagggccggctcgtccagacaggggaaggcggcgcgcgcactaatcgcctggaactgcgtcgatgctacatatttgcgcgtaccgttcgcatcgagatacgagctgaggtaaaagccatcgtcatcgacgcgcagctcaccctcgaaatcgaggtgcaaaacgtacgaggccggtgcaagcgcacgacggatcgcgaacacggcaaactcgcgctcagcatcctcggtatagcgcagagtttccagaaacgtgaggttcgtgttgggattggatgcgtatagctcgttggaggtaatgcgcagtccgcgctgatgcacgtagatggttttggcctgctgccggatgtccagatgtatgtccacactgccactgtacgatcggtttccggtgtgcacctgcgtctccaggtacagcttgtagtgcgtcggcacgatgtagctcggcagtcggtaccgtagctcctgcgctgccacttcctgcaggctgaccggatcgagcgtgttcagtttccgctcgctatgctgcaccttcggatgcgccgcaatggctgcagagtgcagcccgattagaaaaacaccgcacagcaaatgtagccgcatgtctacaaacttgaaggttgattttgggactgaaatctccggtgcgaaatgtcgactccaatatccgtaatcgcaacagtttcggattgttttacgaccagatcgaccacaaacagttgctcgtgtacgtaccccccgataaccgaggtgtggggcaaatgccttaggaaaagcaatttctcacctgagcaattgaattatccatacctttgtatagcaagcggggctcgtttggattgagataagaagtcgattgagtgtaataactgccgaacaagagctaatcggccttaatcgcttatcgctcgctagtgagtaaattcgtaggggaataattgacgtttactcaatgacttgtgtgatttatatttgatgtttgataattcgcatctcatctaaaccaatgctgtctaaaaacgattgaatatcttattgacgtgggccgtttttctacatttttgaccgtttacttgcgcagtcatgattgaatttggctgattgtgaatcattaatcattccgtaaatatattggtgctatactactgtataaaggatagtagcttagtagctcagaagcttagtacaatatttgaacgttaaagaaaccaaaactgagtttgtgcatataacaaatcccaagtactagcgataaataacgctacgcaagtaatctatctgtccagttgtaaacaacatgtaataaaatggttcaaaatggcgcgacgaccggaaatggatcgcgttaaaacgtctgcctagagacatcttctttcgtatggtgtgtgccataacacctctctcgctcttttgtagttcgtaccacttagactcccgatgccgatgtaatactagagtaggaggaaataattaatatcacagttagggcacgaatgcttgcgtacttcacgaaaccttatgtaccgaaggtggagttgcgattgctcacgcgttgttgccccgttatatgcgaggtgggtcgtttcgggccaagatgtaacaaccccagcataaggtgggaacgagaaaccgtgcccgagaaaggaacgttccatctaagccagcgtggagggctctttgtgggcatgtgtacggcgatacggcaacccaaaagagaaagggcgaaattaatgtgtttggctcgttggccaaacagcagtcggtttgcacaaaaaccaaagcgcctgcgaaaattagtcacaccctcccgggccagcttttggggagagtgggagataatgttatgtgtctaaaatggttagacattttttacacgtgaagcaaagtttgcattcgctccgagcgggagcaggttgtgccatgtcggcttagggtgggtggaatgcgcgtgtttgtgtgtgtttgatgtgatgaaaaatgcaattgcgagcaaagtacgcgcacaaaccccgcaggccaatccctcttttttccagctcctttatacatttaattccagccaagcagagcccgccgttagccgtgctgtgtgagctttttacacgcttgagatagaaataatggcgtagtgcgctggttttcgttacagtccgctgcacaaacccggactaagggagggcggctgatggtggatcgctggtgccgcgtttacggtgtgttgcattaacgaggcccaggaataggcagaaatgtatttataattcagattagtaacaaaatggtggctctcaaagtgcgattgaagcgcgaagaagagtgcaacgaagagcgtgtccgtaataaatgtgcaaaaaaaaggaaccaaacatttttgcaataaatactgtttacagctgacggggtaaagtttacttccagcgttgcaattgcgcttgaatgctcgttcgacccggttgtgtgccgaactcgaagctttctagtttattttatgacaaaataacaaacaaaatggtgtctgtcacaccctgtaacctctctattaaactgatgatgtcacgcagcagccataaaacagacatcccactaagctctctatgatcgtaatttgtagtgcaaaaatgtagccatattaatgagtaccttgcaatcggacgacagtgaaggtctgccataaaagcgttacaaaataggcacagctctgggcagtctagtttctgcgcagcgatcaggcacactcataagtgcagctttgaagcgtaaactgcacttactaacgtcctgattcatcgatcgaatagcccggcacgcccccatccgtaggcttatccgggctgttttgctacgagcggttcaggtcgttaaaatcgatcgttaaaatattatgggatctgtcctcggctcttctcacgtgcattggagaaggtatggcgcggtgcagatgaagggatgccgaggaggaggtatggttcatatttgaccacagtgcgtatttgcgaaacccgaaaggtgcatcagctaaatggtggaatgtttctgcttttacgagtcgacagctgtggctccttcgacggggcagtcattaaactctcctcctaaaatgtcgtttgcactcaatagtggcagcactgcctggcccgatcgagccttcgccaaaagatcgaccgttaagggaggggggaggggtaaccgcgagcgatggataaggatatcggtggcatcgatttcgtttaatgttttgcctgctgcatcgcaggccgtcgttatgagccctccgattagtgcatcgtgataataagggcaaaacactccgttggtggcgctgcaactaactgtcggcaagaatgtggcattaatgccggcaacgacgggccgttttgtttaatttcttttcgtcgtcaccggccgactgcccgctttgccaataaaaccgtgcgtcgcgtgtgcgagcgtgtgttgcctggcttgtagcagtgcaccccagcccagccagagtgcgctgatcgctccaaacagtaggactattaaaaatcaattttccaccgatcctcacgcagtcgttttttatctctacctccgctgggggaatgatccgcgggcttgtctttacgcaggcgattaaaatgcaagtgaaaacaaaaaataaaaacacgaaataaaacacgattaaaatgtcagtgagtgatctttttttattattttcgttccacactgcatgcatgcgtacgctttttcagttttgtaagttcagaattggttcaatggccgatacggttggcgctcggtttgaagtaacgaccccgcagcataaaatgtgaatcatttgtgtgcgtgtctgtctctgtgtgtgatggcattctggtttttcaatgatgcgctcctattttcacaaccattacggaagggccagattcattagccgttaatcggaaatttgcgtggtgacgtggtaatttgtagtttatttatttgtgattgctttcggacgatgcccttttcccggtttgttttttactgcggatgtggtgcgtgtgcgaaacggcaggaaaggtcgactggttcccatcggaatggattcaaatgataatctgatttatttagcaatggcactgaggctgacacgagccccattttgtgtcacattgtagctgcagtggtaagttgccgtaaaactttaattcaattttcaactcaccggcaccggaagctcgtacagccttgacaaggaagaaaaaaaagctttgatacatttagtatttaaatggactgagcggaattttgtgaagtacaacgggcaatatttattatttattttagtacttttattgaatcgcttgcaaaaccagtcatcatcttcaggaagtaagaaacgacgttttcaagatgctttgactcatctgatgcacgtgatctcaacacaacttcctcacacataatgccaaggaaataagtttcactcaatcgaaacatgtttgtgtgtgtgtgtgtgtgtgcttgtcgaaaaacgctgctggaaaatatgcgcattttcagtttttactacctctccgaaaattcggtacggtttcggtgcggtgctcaccagcccgcccaaaagttacacgttgattcccctcggaggtcacgtcactgtctagcacggtggcggcgagagactggcgggctgaaagattgaacagcggttcgtcccaaaactaatccgtgaatcatcatccgtggccgagcgcgagcacggcgctgcccccgggagccaaggggcagtaaaacatgtttggttttacgagcttggaaaagtttttctcattttcctcgctcaaccactttgctgtggaacggattgcgcggcgctcgttagcgttttcgagatgcgagccgttgcctctgttcttcgtcttcgaaaccactgttgtttcgcctgtttgatttatgtgtgtgtgtgtgtgtgtgtgtgtgtgtgtgtgtgtagtttgtgatggaaactaataagttttgatgcttcctttccctgtttgtctgcatgctctttggtggcattttaagaaagcactgactgacaaaagccaagtttgtgtacgacttaggatggtcaaaccatagtttgggagggccttcatgtgtgtatgtgtgtgttttttccacactccgaccagtacgctagtgcaatgtagacatcctcccggtaagatgcatcttcccagcgagcagcggttgcgaaccaacgaaccttggcttgcatgtttttgatgagttttaaattttggctgatttggtaaatttttacgactttgtttatgaaacgatggaactgacaaaaggcacaccaggcaaaccagcaggaatcgagcgaaaagcaaatcgcgtaacgaaccgcacgtccaacataactgcgcaccccatctcgaacggtggacggtgcggggcacgtcttcgcagcattgcagtggattgatgtcttccagcagagttttggcgccgccgtccagcgcattgtgctggcgaaggtcggtgcaaatctgcaccggaacacggaagcacgaaaaacggaatcgaaagcgcagacaccgggaacgataaagatgtttgaatgcgtcataaatctacaaagacggtcagtgaaatgaattggaaactcgcatttgtcgtcgtcaacgtcatcgggagttgttcatttttttttttgggaggatagcaaacgcacatcaaatgcagtggcccatcacaagtgtgatctacaaggtggtggtgatgacggcggtggtcttgctccgtttaaacgacaatgtaaccaatacgtctagcagttgacgatgcatatgattagtgaagtggaaccgcgctttaaagacacctttgcttgcatgcgtgtgtatgtccgccagatcgcacaattcatcccaacgacatgtgaaggctttaaaaacaaattgaaatcgcttgaaacacatattcatagcgtgcccggccgagaatgggttttacttgctcgttaacgagaaagagggtgtttcttcagctgctcttcagcggggttagttttgcatttgaagcaaatcgttacaaaatgcaataaaatcgtctaatggtacggcgtaacgacgtgtagttgtacttggaccaattggccacagcgtgttcgccgcggaacacgggcaacacggggtggggttttagtttttattttacattttttaaatgcctcccttcgttgtgccaattgctgtgcgatctgtcaggtttcgaacacatttcttcgctctgtgcagcgaacgcgtgcaaatgagcgtaagcgtgagtgaatttcaattccaaaagaggtccagcctgtcataaaacctcactccactggttcccttttccgcgcggtcgctcgcccatccatcgctgatggcatcgaaaatccactcgttaaacgcgaaaccacgaaccgatcggcgcggggaaagggacaccggtgccagcggccgggcgcgcaaggatcgtaaattataatatgatttttattacattttagcgtagcataagccgaggccggctgagagacgttcgtaatttgttataatgttatatggctttccgttcccgagccgtgcaccgacacactgggcgccgacaagaaatggctcagggtgtactgtgtgtatgtgtgtgtatgcctttgctgctattgttatttttatatttccttccagtcgaaggaaacgggtgtctttggagaatggggaagctttgcacaattgtaccccagcggagactcactctaataacgttcattttcaacaaataaaagcattgcatcagaactatcgtcagagtgtgtgtgtgtgtgtgtgtgtgtttgtgtgctgctgcgataatttctgtatcgctttcgtcatcagttttatttcgttattttattttacaattgctcgtgaagtggcgtgcaaacgcaattgcgagccgctttggcgagcaaggaaccgcgcccaagatcggtttcggttcccttttctttgtgaatcatggttgtgaagatttgttgtgcaaaaacgccaagctagtaacgaattggtaaaataactgcgccactgcatgcacaaacacacacacacacgcacaggcagaggaaaaacgaagagtccggatacaaaattgcggttttgtagcttttatgatccaattagctgtagaacaagaaccgggacgatgcgaaaggggtgttgtaagacgcacacaggcacactggtctgggcatgctagtcgatggaaattgaatcagcggatatgcgttttgcgcacatgccttttttcatccttcccttttaccgttgaggcatgggaagtgtcataaactcgtgtatgcgatttgttgttccgtcaaggtttcgtttgactgagttgctgtaaatcaaaataaaataaagtggccaaagggccgggacgagcagaggaatgtttccaacgcatgtcttggtggtgtccaacaatcctcatttatgatgctgcattgtcaatggaatggtctcatgtggtccggacacgtccaatcacatttattgcttcattatgccgaacgaagttttatttcggaagtgtggaaagtatgtttttttaactcattcgaacatgttccttttcaatataattttgtatagcttcgacaaggaattcgctagcagttattcaacaaataattacgcatgcaataatttgtcgcatgcaaattccggtttcagcaaaagctggtttttaaaagctcgagtaaatgtgttcaacatcctgctatgtaaaattaactatgttttgtaagtgttccaatcagtcacagaacgccaagctgaggaagagtatagtgttatagaactttactagaagccagttggattttgttcatccccacactaataagacagacacaatttacatttgcgtagtttgtgcttttgcataatacatttaaatgtagaaatttaaataaatagaatcataacattatgcttctggggtaaagtacagctagcttccatccttccctacattaaaatcaattgaatgctgccatataattacgtgaaaagaagaagaaatagtttattgcggtgttttaccgctattattgcattacccgcagcaccgtcagtaggagtagtgctatgcttttacctaatcataaaactagttattatataccttctgcacacccaagtggcatgattcgttgtgttgccctttctccccatgctttgtgccgattcccaacagcgagtgtgagaacacccgtacaagaaaagccctattcttcccacccagagcgggaatagtatacgagagacccttgcacacttttccatcgcgatatgggtgtaatggtcggtgttggggtgaattttccagatcccctcaatattgctcgaggctttcgattggctcgggctgctgtaatagtgtgtaatgggtgtgtgggcactccagaagatggaaaccatttcgtataaaacaaaagaaaccaccccatgctcgagaccggtgcgatcgctcgaatcgctgaaactccaccgtcacgagcacgacgttgtctagttgggctggatctacaccaacctgtgctagtgcgcgcgactagatgtgcatgtaaaaaaataaacatataaatcaacaatgctcggcgtggcaagcatcaaagcaagtaacggatagaaagagcaaactcgagggagcaaacttcgacgccaacaaaccctcccgcgcgcgcccagcactagctatgcactcgaagcgcatagcgaaagatttacgcggggggatacggttggtgttggtgagcatgtttcgatgttgcgccccatgagcatgttttgggccgccagagcgagacgggaagagcgcgtgcgaaacataagacagaggcggagtcaaccctaccattggttgcgctcgtcggtcgttctgttgctcccgctctgatgggtggcgcgcgagcataggtctccgactcgctctagcgcgttgcagccgttccacacacctttttgcacgtgcggctttgccaccactggctgcggcacaaattccgaccgagcacgtggttcctctatctacatttctgcgccaaccggtggatgtggacgtctcctggcacatcggtcgaactgtgtgtgtgtgtgcgtagatacaacatctcgttatgttgtgcctccgaaagccgaacaccctcgaccgtcgtcatcgtcggtgtcgtcgcggttttatgctccggcgaaactgctgcgaacgtttcactctcactctgtcccagtgcatccggcacggtatcttttgcatcccttcggcggtaagtttgggcgttgcagcacgatgttacatcggagcactccgcaaaaagcaggcggaagaagcagctagcccgaaaatgtgtgtcggaaaatttcaccatcagttcgggagcggagaggaggccgcttttccgagggaatcaacaaacgatttcgctgcttatttgaagaagcagcaaccatctacgaacggtttcttcaaacgatgaagcacacaacgacataccattcggctctgggggaaaacatgttttagtgctgcttttcgccacgtatgtctaaaccgaaaaagaagaactttctctatcaacggaaagactatttttttcgcctgtttcccaaaccttaccatagaaagaaggactgcaatgcgcggatacgacaggaaaagaaccatttagcggcacatacttgggagagaagcacgttcgtaggaaacaaggatgtttatgttagcgcgaataattcagacacgctctgagcgctttcgggtgagattagcaatggagcattcgggcaaacgaaaagaacgtttgcgtttcgaatggggcgtttttgcttgtgcagcgatgacgagtacctcgtctaaaggcagtcagctatccggaaaacgttgctctcgattaatgcccgttggtagcatcgcacaatagcataaaagcacataagacaagtcaccggaaggctgcataacaccgaaaggttaggagaaaaaaaataacggacgataaacgggtacaatctgagttggtatctgagctgggaaaagggctgaagaaaataggagcagtagaagctttatgtaggatttgctcatcgaatgaacaacgtactaaagatcgttttttacacggcggatttatgttggaacaagtcgttaaatagcgagctttgttgggagtatcaaataaaagaaaacctcatcacttaccaagagcactaaaagagatttagtcaagtagtgttgttagtctttttattagcttgggatttactatttatacttatatatcttatctttacttaaaaaatggcaaaaaaagataaatagaaagatgtcaaatcatcaaacttgttacattgttttataaacttgtttgttactacttgtttgttataacattgctttatacacttgtttttactttaatgaaaacaaacatacaaacaagtatttatttttcaataatccgttatttttagttatatgactaaaactaatattgcaataaaatgactgcacttcttattggtgttgaaattccctgataacgcaaaaaatgtcattaaaaattatgtgttagctaactaactaacgccatgtttcaatgttgaaacaagcagatgccaaaagttttttatgattttttatagtacagtagaaacagacgatatttttccgatttattaaagttaaagtgcattcaaacggcatattggtttacgtttgaattgaatgtatctttatgtacagtttaatcagtcgactgattgtttcactcattggattacgtttgccttgaaagtaacatttcaacctgtatggcattgcgcacatctatttacttgtcatgtcgctcctatggcgctccatagttcccaccagccccaccgaaaagattgattaacatcttgacgggtcatatacttattaatgccgcccataaaattaatcctgcccgactatgaatcggacattgtacacagtgcagcgactctcctcccatgtacggtaacaaccatgttacctcacgaaggtcatgtccgcatacgcgccaaacatgaagcgtacctaagcaagtcgtgcaccaaacttaaataaaaataattgaatcaatcgagcacggcttgtgataaacgatccgattgattcgttagccggatgcagttgcagtagttgtcttgcggttgtggagttgcagtagggatgggggttgtggaggggtatgtacgtcagcgttgggtggctacgatcgcgccacgtgcgttcgcgaaaacgaccaaccagcaccggtcttatctgagattaaacgaacgaggtgacagctaaaaggagaaaccgggcgattatttaaattagttcccctacgaatgttgtacggcgcggcgggctgcatcggaggagggatcttatctcgggggtagcgttatttgcgttattgtaggcaaaaaaaggataagtatgctgctggtaagaaggtaagaagtatgcgcgctgcaataagcatccccgtgccctttcggcacccggcgtgtggagctcggtgcatcggaagctcggatttcagctgcaccgaaaccagatgcacacacgcgcaccgctccgggggcgttgaggcaatcgaaagcaatcaacatcaattagcaagtttatttgcaacaccgccggtttcgatggattcttccgcatcggcgactggtacaaattgctgctgctgcgcctttagcgggtggcagatcggttttgccgctaccggtaccgcatactatgaaagtatgatttatcgtctacaatcatttcccattacacaggcgcggatcgtaaaatcagctccggaaatatgtgtgtgggtttatgtgtgtgtgtgtttcggtggggatgaatcgaaaattcatcttttgctagcgggacgaagctgttggtgtggagtgcccgtgccaaatacgttgaaggtcgcgatgtacgcgattctctagccttgcttagtcattcagcgggaatgggttggttgttgcgctcgcattggaaaggtgcattctgcaccgaagcattccagtagcgcacgccgatcgtttgctcgattatggtttgtttagtctggatgaataaaatattgctcaattattcaatttatcgcgggcctgggcccggcagtggcaaacaggactgaaaccgccgttctgtgcaggtctgttccgcgatcgatactatcgtctgccagtgcatttgtgtgtttgttctggcccgcttgttgatatgttgtggttgcccgcttggcaaatgtgcaacgcatccgcgaatcgagatgttgcagcatggatggacacgaaacacgagccataactgtacaaacaaacgattggcccaagttggtttataattgcgaagcgtgcgttaacatggcgatcaagaataagttcataatcgatggattatgagcttgagcggaattgcaaggacacgaaattgataagcacaaacaatgaatgtgtattgtgaaagtgaatggaatttcaggtgattcatgtctgggaaatgtttgtaccacaaattgcatcataccattgagaagctacaattacgcagattaattttacgcacagaattgcagaaaggaactgtttttttttgcaaataaaaaaaaagattgaatattcaacagttggttggaactagcgaaaccaagggcccttcaacccgaagaataatgatacgtaatttttcacgatcgatgcaaaacatgcacaaaatattgcatttaattcttcacagctagcaccgatcgttttgtcatgatcagcgatcggtcgatgtgtgccgctgcttgcaagttactattctggtattcccattctctccggtactggagcagccagcttcgtgtcatcgacaaagcgcttcaagtgatgcccttttactacaacccacggcgaactgaaaatgccagaaatagatagaggaagatcgacaatgatctattgactagttcaggcgcgcgcgtctcgctaggatttgcttttcggaggatccacctcggcacaatctcggagacggcggtgatggcggctctaccggtggattgacactttgacagctctgatgcaatacccatttccagtcgacggatgacgcgaaatcgcacaaaatccaccctccagccggggcggaaggaggacgcttatttccaccgtgatcaaatgacaaacgggcgcgtgcgcttgtgtttagcaggcaggggagatgagcgcaaactgtgcaagaagaagcatcactgtgaagacggcaatgcaaagatagtgtgctcaacttctccgcgaagattgaagctaaattaagcacgagattagcatgactgaagtgacttttcaaagtgtcagaatggctgcactcgcaaactagctggatgcagcgcaattttgccccggtgtgtgcgcgcatgcaaacgagcaaccgcagagggcaaaggagaggatgggaaggagggagggagtgaaagagcaggcttaaggttgccctcgggcattgaagtcgatacagcggttctattccagtgccagtaacgatgacgaagacgatgttgcttctgctgctgttgctgctgttgttgttgatgatgatgatgataatagtgcaaatataaaataaatcttccgtaagctttgtgtagtggtgcgtggctactataagcccgtctggaagcaaggaagctagtcgggcagggtcatgcaaaagggagacaccttcggagctccggagctcccgccggcactctcggggggacgtccgttatgcgttgtgatttattatggaatatttattatagtgtcttgttttgaaaaaataacttcaacggttcgaatttcctacacctcgagatcggggctggagtggcaacgtggtacggaacggtacagcggtttgagccgttcggtcttgggactcacggatcgcagaatgttattgtgcgcgcactgatgggaaagtcatttttcaccgagtggtcagggcgcgtagtccagttcgtttctggctgctgttgctgatgctacgatcctcaggaatgattggaaacgcctggagatggtgggaaaaaatcaaacacaaaaacgatcctaatgaacatcgtgtgttctcattcgctgccacgattgacaccttcgataagacgcacataatgagctaaaggagaggggacagggtcttgtctttgccacgagcgataagattgcaatcactcgtgagcgtgtgctgctgggctgaagaagaaacgctttccacagcagtaggtgggaagtgggattgtggaacgtggcattgaaaagaacctattttctaaagcccgagagcccgttctcgaactggaaaaccagatgcagaagttttttattgtcccccgccaggaaaacaaatgtatttaatgctttctttgccttttccgccccgtttcagacgacgagctagtgaagcgagcccaatggctgttggagaaactcggctacccgtgggagatgatgcccctgatgtacgtcatactaaagagcgccgatggcgatgtacaaaaagcacaccagcggatcgacgaaggtaagctggcgatgatggtgtcgttcgacatcactttcatcaccgtgtcagacatctactgtgcctagcaccgggtccagtggtcacagggtgtagcaaaaacgtgttcttttttgcgagagactctacctcatgatgcagctgttaaggaaaggtttcagatgaaggcaatttttcctaggataagatgatcttaagttacctgcgtattagtgtttaacattgtcgtctcaactcccaagaatgttttaatcgtctagggctagtttatttatactgttctcattgaaatgtcgttcaatccaacatgttaagttagctagctcagacacgagaagttaggagtatctgcatcttgaaggtagcggcatatggtgttatgccacgttcactgacttcaaaattcgatacaaaaaaaaaaccaaaacatcaaaaaccaaattgtgaattccgtcagccagcagcagtgaccttcaaagccttacctttccattcatttatgtttaacacaggtcaagcggtggtcaacgaatactcacgattgcataatctgaacatgtttgatggcgtggagttgcgcaataccacccgtcagagtggatgataaactttccgcaccactgtaactgtccgtatctttgtatgtgggtgtgtgtatgtgtgtttggtgaaacgaattcaatagttctgtgctattttaaatcaagccgcgtgcgcaactgatgccgataagttcaaactagtgtttaaggagtggagcgagagagccgcaccacggtacagaagggcagcagaatgggtcggcagcctagctgcactggtgcggtgcgtccggcgtctcggggggagggcgaggaaattctagtgttaaatcggagcagcaaaaacaaaacagtggtcgtcccgttcaagaaacggcctgtacacacacacagaaaacactgcagcatgtttgtacatagtagatcctagagcaggtggtcgttgctcctcgaacgctctggacgcacggcttcgcgcgtatttgcgtagcgttccgccgatcgtgggtattcgtactgccacaagcccgctttctcccatgcaatctctgcaaccaaaccaacaaacaacaacaaaaaaccaatcgacaaaatgaatcacaccccttttgtatcatctgtatattcttgttctttgcgttcttttctatgtggcccacgccccggcgggtacgtaattgcgtcgaaaaccccgaaaaccccggcacatacagtgtacatacggtttgaggacaactttgacctgcagcccttctggggttgccacgtgtagctatacttgtgagatcgggcgccgacggtgtaaagcgcgaatggccgccacacagtgtgtccactccaacactacccctctggaactaccccgtccagggatgcaccggctcggctcatgcccctgcaaaacagtccgggctccactgtagtagctccggcgttgctctgagagaaggatgcccttcgaagtgtcgaaagcgtgcattgggcgttcaagtgtgtgtgtgtgtgttaggtttagcgagaaacagcagcagttgcgtgtgctgaaaagcgaaggagtaatagagtgcataatgaaaatgaaaatgaaaatgaagcaaaagtagaaggcggaggagagcaacctgtgttccactagtagcgaatagtttagtctagtttcgtcaccaatcaaccttccaaccatcgttcaaccaatacctgagtcaacatcgtcatcgttatcgtgccacaactttattaaaaatgaaccttgtccgcgccaccgtagggtgatctaaggcgacctttcttacgggcgcgacccacatgccatcgtcaccttctccaatcaaaaccaacagcctgtaccgatggtgtgcaattgtgcgtgcgtgtgtgttattagcaaaaaaagagaaagagtcgacgagagagagatagatcgagatcgagagtacaaaagagcagtagaaatgttcgttgtttgtttttcgtaacacagttgtttagccaaaatgggaatttccaataatcccgggggcggggaaatgcgggaatactgcgtacacacatacatcaatcaaaaagaaaaatccttgcgctacatcactaccgtttgcgcggtgctgatctagagcagaccactttccactccactctacaatcaatcaatctgtgcagaaggtatggtaagacggcctttgagcgagtcacggtcgccaccataacgccgtccgacgagggctgaatgcgaactttgctaatcgattttccgctttctttttatcccacctccttttctctccctctctctcttttgcactgccccttgtaacccccaaaaaggtaaacgacacattaagacctacgaagcgttggtgaagtcatcgctcgatccgaacagcgaccggctgacggaggacgacgacgaggacgagaacatctcggtgacccgcaccaactccactattcggtcgaggtccagctcgctgtcgcggtcccggtcctgctcgcgccaggccgaaactccccgggccgacgatcgggccctgaaccttgacaccaaattcaaaccatctgccagcagcagcagcaccggctgcgatcgggacgacggtgactgcagcgcgttcgacgacagtgcctcggtggtgcgggggcacgggcggacggcccacagcaccggtagcaggggccgcagccactcgaaacggtaccacaccctcccggccgagcacatcgggagccacatggcggccgcccagagtcgatcgcccgccccggacgacgagccggtggtgtcggtgtccgtgtacgagagcctggtcgaagcggccagcaaaaagacgcgcaccttcagcccgccccggggggaggcggaagatttgcatgccgcacggaaagcatcgccccacgacgagcgggacgagccgaccccggcccagccctacgaagcgtacctggagtcggtgcggcggagtaaaaagtgcttcgcgctcaaggacagcgaggcgccgggcgaggagccgacgggctacgagaaggagaaggagccgcgcattccgtactcgctgccgaagagcaccttcgagcggctcgacctgctgaagaaaccgaacgggctgacgtttccgatgtacaagtacagcgggatcgagccgaacaactttgccctgccgctgctgctgcccgggctggaggcggtcaaccggacgctctactcgacgcccttcccggcccagctcctgccgtccagtctgtatccgtccgttagcagcgagtccacgacagtgcccatgttccacacgcactttctcgggtatcagccgccgctgcagctgccccacgtcgagcacttctatcggaaggagcagcagcagcagcagcagcagcagcagggattggccgaaccaaaggaaccgacgtcgtcgtcttcgccgggcagcaaccggcttacgccaccgaagggtgcatttttctacgcgagtgcggtggaaaattcgctcaccgcccagcaggcttccattgctaccatccattagatccacactgcgtccactcgctgtttgctgcagcgtaccgcggacagtgcagtgtaccgctgtacaaaaaggtaagtgtgggtagtaagcggtagggtgggatgggtagattagacagtaggcaagtggggatgcaaatttacagcccttttggtcactttaacagacacaacagacaagggacgctagcacgaatcatcgcaacaaaatggaatgaagcaaatggcctttggacattctttgatcttcacactgtttccgcgggctggggacgttattagaggaaaaacgccaatatgttgtcgtcaacattggttccgctcccagcctgggggctgctttacttctgccagtatcgatcatcgcctggtatcgctcggcattaaataaatcattcatggccaaatcaacgtttagttattgatatgggcaggaggaagcaaacaaacgaaaaaaaaacgggcacactccatcgaactggatactggaaactctgcaccctacgctcaccctcattgcaccctaccagagccgatatgctgcaaaattctaaataaaaataatccatgcgggtcgcgaagcaaataatttatttcctatttatatttatttttaatcacacacaaatatgggtgcatgcacgtgtgtgtgtgtgtgtgtgtgtgtgtgtgtgaccgagtatggacggacgatggacactgtggtgcaaatagcggtgagcggtcgtggccgaaggttggctaatgcaacgcgttgtgtcgcccgtttttccgagcgtgcctgatttccaatgcctatttttcactccactgccgctttggtcgccattgccttcggggggacctttttaaggcaaatgttgatttgcaccgacacacaccgaattgcacactgcacccagtcagtcaggcaggtggtgttgtttgaaaatggcgctctggagcaaccaacaaacgaacacaaaacaaaaaaaaaacaaatcaatagaaagaatcgagctgtttcgattattcaaaatttatacacaaaatatgcaacgtattccccggtggggtaccctcattgtccgacctactcccccccggtgcacctcaaacccaccggcagcaatcaatgtaataatggtaaagggtggcgtgccaaatactcccggaccattccgcgctcgacgtagggacatacagagagcgggagctgcagtgacacgagtgaaacaacctggagacccctgcattcgtcaggcggaaataaacaaatcaaaacaaacctcccgtctgatctcgcgaccctgccacccaccggcagccggcaaccagtcgtccaatttcggcactttggcggtgtgcaactttagcagtctatgcacatgcattgtaaatatgcatattgcacgagataaagagagacgggccgagagaaagggtctctgtgagcggggtagccagaagtatcgaacgacaaactatgcgcgtattacgagatgcgatcggtttgacactcggcattcgcactttggtggctatttttattcgcctgcttaactccgtcgctgtttgtgcgtggctgcgtgtatgtggccgggcgagcgtttgtttaatctggcacggtgcagtatgcagttcggatgccagcgctcgccgccccctgcaccactgaccacccgttccatgcccaacgacagcaacgtcccggcagagtgatcagcagaagaaaggcgtttcgtgccaattctgtcgtatacatcgtgcacggacgcggattgttgacgaaaggttttgtagcaaaccgggcggcgaacaagttatgaataaatttactccattcgttatccactgatgtatcattaatggcagccggtcagctatggggcgctatgggcagtacagtcggtcccgggtgtgccgatcggtaaataaagtgatttttgcattccgcttccgtggtagctaattttgtgtggcacactttggagcgaattgtttgattagggctcgtttgttcgcttgactgtaagctatcatccgatgaaagcgggcttaaatgctagatttactaggccgatcattttgacaggtagctctaggagcttttcattatgcctaattatattgtaaatatttagttgtgcatttaatgcaaacttccaacaaatgaaaaagtcattctgctcttttaagtattttaatcagtattttcaaagctttaagcacaaacgcttagaacgtttgatgtttttagtattttatctacttatttgtttattgagtgcccctgacattcgtcgctcacaaacaataaatatttttggacctggatctagtaaatgtacgacatagctcgaattgaaaatcaacgtcaatatctctctaattttatggtctaattgcatagagaagataaaaaactatctattatttaccgattagaaattaattctagtatcctcctgctagtgctcgaatcgaattcatttgcattccttctgcttgctagccgcaggtacagcaatatcggaaactctttctttaatataggtttaaagagcctctaatgtgcatctttgcgctgatcgtaacgtttcaccgaatcatcaacgagtgttgttttgccttctgcaatgaaaccatcctacactctcacgtgtttgaaagaggtccacggcacaccgggaatgcattatgcgctgacggcggtggtgttttgttcgaagttcgtgatgcaaccgccggggaagttgcacacagggatttaacgactcctcgtaaaacggtattatatatcgaggccgcagcgaaaggtaacgccgcagccgcagcaaacggctacacaaaagtaaacccctctctgccgcactcgttgcgcagtgccggaccgcatggcgcacatcttcgaccagttcgcgaggtcgctcaatacattaggaactaatatatattccaggcaataataattttctattttactgcccttcgtggggagatgctttgcgagtggtgctctgtgccaggagaggcagagaaggcatacccaccaaccacctccagggtttcaaacacgttccctgcgcttatcgtgaatcttttgcatcttttgatgatcgatactcctcgggcccgggacaagaccaacgccaaggtgcaccgtgtggaccaacatcgtagacgacaatccgtgcgttgcgttttggcaaggaggagctgtacgaggtgagatagagtgtgtgggagaaagatagggatagcacaaaagagtgtgtgagagagagagagagagcgcacctagaataacagctcgcctgactgacttgactgactggcaggccatagaatggtggcgagaaaaagcgtcttacaagacgcgctaaatgcaactttacaacggtcgtaaactaggtcgtaaatatctttgccagcataccttctgcaaaagagcagatcccgcaaacacacactgcgtacggcgcaacggctgccactcgtgatgcacttgtagtagaccggggcccgatccgaaccgtcccggacgcgttttgctgaccgaaacagacacgcacacagggtgcattttgctaatttttatgctaaatttttccaccaccgacatgggatagtttccagctgagagtgcaagtgcacttggggtgcaagttgtcgcatggagcgcgataacggacgcagtccactgctcatcttagccttatacctgctcctggaagatccgatatgtctccaatcagtatcgtcggcagtattttacgataatccgcagcgaacgggaaccggccgccttggtagcggtttgtcaaacggatctgcactccgcactaccgtcatgacgcgattagaggtagagcagcatgccgtactacgctaccacttgcaacggcaaacgtcgcggagcaacattgtggccgcagcgccgaagcaataaaagttggaggacatctgtgagcagataatttacaagctactttgtataatgaaaaacgcattaaaaaactacgcctggcaaaagttcctagttgttcttaggggggaggaagttggaggggggcaatcatttgcgaaccagactgcgaaactgttacaagacaaacccggagcatttccgggcgatcaactcatgattattgttagactcgcggtgacgagctgtgaagcgtcctgccttttcggacgttgtgcgaaatgtttcgcactgcagcacggcgggtgttcgatgccgtggtgtagttgcggtttttctacagctctcacatacacataaccggcatgaaacacggaatgcgagcgatgcgagctgggagttggcgcatcaaactccactaatgttgcacactgtgtggggtgggatcaacttcttcgccggcgtttgttaccgcggtggtgccgatgaaaagacgccatagatggattttagccaaagacacaccgttccatcgtggccgaacaacggttgcaacggtgcgctgggcagaaggtaatggaaccggttccggtactgatcggccattacgggctagtgaattttactagttttcagagataattttatgggtttccatttgtgggaattgctttttttattgcctcaactggctgtgaggtctctcttctgggccggtgtgttgtttcagcagtttcgttcctttgttcgagcggttttgtgcattgtgcttgatgatatgacaaacccagaaaacaaaacaaaaaaacgataactacatgcgtctggtttatctggctgtaaatttagtttgcagtccttcaacacacagacttacacaaacctcataccctaatcattgtgatggatatcgttcagtatcacgatgttattgaggtgtgttcacatattcctaatgaattacattttttgttttatccattttaaatgatgaataaatattctacaaacatgtataaactcatattaataaacctattgtccaaattaatattaagtggcgtgaaacgatacagcttatgcactacgcaaatattacgagaatatgatctaatttgcagtgaaaatttgttttccttggttccaatatttccacaaccttatatatcatgtgaattattttaaaataagttatcatcttagaaaaaaatcatcatcagatcaaacatcactagatctcaaagttacatcaagccgttcgctctgaattgtagttttatttcgagtgtttcaaataatttacttttttctcatcatacttatacactttttctcgatttctttccgcttcctcaaaatagatcgattggaaattcacgtcaatcatctgcaagcccgaaagatgctacctagtcgtccccagctgttgctactggagctttgcaagagatccagctttcgttccttatcgatgcacaaaaggcgcacccggaaacaaaacaaaaatccaacccactcgtcaacggcccacatggcgggttgcactggagaaactcccaccctcgtaagtgctatctaagcgttaaattaccttcgccctttgcggtagaacaaaatagaagcaaatgaaacaaaaaaatcattgccggaggcgcaagtgaacagcggaaagggaaagaaacccctgtcgaacagaaaacatgattattgatatttttcgatcgtgcaacgaaggtctacactgtgatacaaaatgttgtgtacaggataaatattagatttttttgtttggaaaacaaaaacacagctaaacggtaggaacaaaacaaggcaaaccgaacaaaacgaaacagtacgcacacggctcgttgtatgtaaatcaatctatgtgagcgtgtgtgtgtgtgtgatcgtatgtgattatgtgtgtggcgaacggtttcccattttctgtgagtaacgccccgttacgatcattgctgttggaaaaaaagctaaaaccaaaccttcatcgaaacgaatggcgcgcgttctttacttggcgcccaatttcccaccaaaattcaaacctgtttttaatagtgtaaaacgtaatgaaaatagtaaacgggcgtgtgttgtgtgtagcatggttcgatcacttggaaccaaaatctcaaaaaaaagcaaacagaaactcattggcagaaaggcagacacaccggaattgcgaagttgggaaagcagatcactttcttgttatgtctgcgtttatttctcgtgtgcgaatggaaggcaggaaattcagaggttcatctcccatggaagatgacggaaagagattaagaaattcgaaggcaaatctgttacaacggcgagcgattgtgttatggctagtaaagaattgaattgtgatacgtgcgcagtactgcatatttgttcaatttgtagcttgtaggtagatcgccgtcctcgtgttccgtgatccgggggcgggatgatagactccgccacttggagcgatatcccatgttgctgtactctcgtttcggtgcctttttttcttgctctttcgttttacaaaaaaagtaattatattgcttttgttttatgtgcgcacccgcacacacagctgcacacgatcgtacaagttaacgaatggtttagtttgcgctaagtttgattggttctagttcgctaagttagtctgtagagagattcgtttatcgttatgttcagcagcagtgtcaggaacgagattggaagataattacaggggcagggcagatgagcaaagggggtacggttaggggctggaagtcaaaatgctttagccatcctgcagtcgaatttaaacattaaaaaacaggtccgccttgacgaaacaaatacccccgaggagttcctgcgcccggcccctcgaatgtgcacgaaatggaataggtgttgtacaggcagaagacagttgtagaagcaagggtgtaatgttccaattgaaaagcgaagagaaaacctaatgtaactacaaggcagatatacagctgagagctatattttacgcagcgaaatacaatgtaatcccattttctccactcatcaaaccttcattagtccttcacatttcacacaagcaagttgtactataatgtagaaaaaagtagaacaagcaaaccatttgatgcatcatcgtcatccagcttgaaaacaaatagatcaaattacatagaactggcaatgtctattgatacgctgttcgagagacttttttttaacacaaccgtaacatcagtggtgccgcgtgaatgtatgtttatttctgagtataaagaaaaaacaacaatgtgcatatatactggtgtgcagtcagctctttctgagagaataaaaaccttaacatttcgctttgcacaaaccatgtcttgtaaaatattactccaacaagaaggacagtcaaagaaagaaacaagaaacaaaacgttaaacttaaatcaaaagctagaaatgcacatgtaccatacattattgcccagaaattatctcaacaaaggggagaacaaaacacagttacagccaacagaaaacagttacagcaaaggtgtacatagcatagagtcacaacacaatatgtacattttacccggttcaatatcaaaataaaatgaaaaaaaaaacgtcccgtccgctgatgacggagtaatgagacgaggcgtgaaaatgaaaatgcaacatcaacagttaagaatcaaaataacaaaaaacacccttatccggctccagtacacaatctattgatgacgaaacgtgtgctgcgaataatgttttaacaaaagatgaagtaagtagaacgtgtttgatggaagcgatgggcagcaaaggtaacgaaaacacacatgctaaacgtcatgtgtagcatgtgtataatagcaagaagaaatttcagagcaagacccaaggaaaagtatctttgattcgtcaaacgccgcaaaacgctgttttactgctgtaagtttgagggaaacaacctccggtaaaagagaaataaagtggaacaaagcaaacaaacaaacaaacaaacaaacataaataaattattaatattattactgaactccgtcgtgcgtgctgtatttcgagtcgctttgctcgccaatgtatgcgtccgaaacgatgtgtttatttagttatttttaccaccaacaaccagatggtggtgaagttcaagaaaaaagtagctgaacgcaacgctgcgtcaatttctctgtctccccaccgcctttctctctctctctctctctctctctctctctctctctctctctctcgctctctctcgctctctctctcactctctctctcactctctctctctctctctctctctttgatttcatcggatcagtctgaactttgccatccaaacaacatttaattacggtcgtcggtattgaggcatagttttatcaatcctggcagcgggactcgaatagagagatgcacttttcccttttccatcggagtaaggacgttgtgaggatggcaaaattaggttgactagtttagcaaagcggaggagaagagttttcaatggtttcaccgttcttagacgcgatttcttcttcccagctggatgagccacagtttgagccggtcgcattgtactgtgcaaggatatgaaccggaatggtggcggagatgagtcgtgctgatgcggttccatccagtctccagacccggtaatcggtccttggccctctacctttctgaaacggtcctctgcaaggtagaaaataggtggttttctaccccgttttgtcttctctcactcttgcgtcgttgtgtgcaaagtactaccagaagtacaggcaatcatgatgctgagatcgtgatgctgcatatccgtggcgcgagaacgaatcttcactttgcactgtacgggggaaattgccataaaatgcgacaagcggtacggtggaaaacaaaactgtgcattgtacgcttcaccgaaagatgccagcgaacgcgggcttgatgctttcgtacttcgggaagttttcttttttttatttctctctcaattggagtctgtccttcgtgccgtggaaaccccgtaatcatgcagcacggtaccgagagcgtggctcaggcacgaaccgtcgcaaacgtgagcatgtgtgtgggtgctgtgaaatgggaagcatcgatacgataagaaactccagcaatcgattgtgccagggcgcaaagccggagcaaacataaacatgcagctcatcaaggatgggttaaaggagtcggcaactaaccggctacagaacgaaacagtgaagcgcgaagaagcaattgctaaccgtgcggtcccttgcctgaccgaacaatagtgaagctcattttccaagcgacgttggttggctgtgtgggctatggggtaaattttaaaacttcttttggggaagtttttggaaggaaaatttcattacgtttcaccctattcctttgcaagagcgggtcgtgataagatctctcgatggggacgtgctgcgagacaggttgatagtggcgagaaaacgtttgacgagcgatatcattgaaaactatctgcaaaatgcttcaccagcggtgtgcacttagatgctagagtttagttttcgttgctaggtgtgcaagtgtgcaaaaaatattcttacaatcgcttgttacttaaattttattacagatagcgaacaaagaggatgttatgtttcagctacataaatttcattcaataagtacatttcaatggtaaaacatctcccttgtgttaaaatctgtacaattgttgagaaatttcaatgaagtttataggttactaattaccgtttattattcataaaataacaacttagcccctggacaattcacggatactaggatgtccaagggtatgtgtgtaactttatcatagaataatttgttatcctaattacttcgttttaacagtgtatcgctcagttctacgtcaactatccgtggttcagtagctgaattcccgcgttggaatcgcgttggttctaggttagtatctcatatgcagattggttaacatgatagtcaataatgtttaaatccatgactgaacattgaagaatatgatacattttatgctattgctattttttttaattcatcacataccacacggtacattattgatttcagaaaggcatattttgattattatataattaaaaattacagctatttttcaagtaaacaccaagctcatgcattaaaccacaataaaattgattttttaattacactcaacacgctaacattttttcaaaaaataacattacatccattacatgccgttgatgaatacataaattacgccttgtttttgatgcacgataatttttattttgcgcaccttttgcccccggtcctatacaacattaccatgattcgtacgtgttcccgctcggcaaatctcgctaatcaaccgttcaacaatccatacatacccgacgttgatcgcacacgatgtaacgcggaccggctggagcgattttggcttgcccgactcgacacaaccgatcgacatcaattgcagggattaccggcacgccatcatcaaccgacatcgcctcggcaaacgcagctccaatcagcaggggctaatcactcgaagcagggatgcccggggagcagagagaccagaaacgctacattatccacgcggctgctattaagtttcgcccacaaccagcgcgcacacaataatcgtcattgatcggcaccggcaaaattaaacattggcaaacacaacggcaactacaaaaactccgatcaaacggtcacggtctgaattgagctcaagggggatggagagcgagtgagaaagaggtgagatatcatattccaatcgattttattcaaattcttaaataacatttatcttcccgatagctgattcattgccgtcgctcacgcctgcttgtctgcttccgctccgttcgcgttctatttgctactgcattatttctgctgatgcacccaatcatcctatctcccaccctctctatctgtactgagcaccgggcagggcgaaaaagggggagcggcagcaaaatgcattccccggagaggaacaagaagaagaaggcggtgcaacaaaaaagcaaacccggatcatcccggctcggtggaaaatagattacattatttgtgtttcattttgtagtatatacgtgtgtgtgtgggtgtgagtgtttgtagtttgccttaaattgttttataattactcttgtgcgacaaaacgcccctgactagagtgggttgggagcgaacaccacaatcgtgaactggacgggagaacataatccgatgtcctcgggtgatttgatgtacgccagggaaagcggatcatcaaatggtgtatactggcaaatatgcaaaaacttcggaaaaggggaactggaacattgaaacaagctattatgcaccttgcactttgtcccaccaactgtccagcaattcgaaataaaatgacagaagcgaccgtacattacactcccatttttttgtcttattctacatttcaatacttttcgccgggtgtttgacgggaatggaaaaggtgtgaagcgcgttcaatcttcatcatcctttgcccacatctcgacctgcggacctggcgggccatgtccatcaacgggcaagctgcagcgcccatcaccgccgctttttgttacccgtcgactcatcttccggtgcgggccagtgcagtcttttccttttttacgctcgctctctctcttaaacgcttccaatatttgtgtttaattattcgaacggaatcctctctgcgacagcacatccgtacggggtgccagtagtgtgtgcgagtccgtgtttgtgtgtagccgtaattatgttgtgattgtcattgtcactcgatgcgcgataaacaatctacctacaatttatgcacccactgggcggcctcgcctcgtgatccagtccggtttgcaagtcgccgcaactccaattcaatgtcatccgttctcacagcgaacgaacagaacggaggggacacgaacgccaacaacagcaacagcggcaaaaaatgcacccaaagtcctggatgctggggatgacaagagccgccgatccggcctcccaccacacaccaaacgcacaatcgcagttggaattgcacggtttaaatatatacatgttgttgctgtttttttgttttgtttttggcgtgcaactgtgctgctcctgctcctatcgtgcgctatcgtggctggatcccgcggggctactcggtgcacggtctaacgcatccggacgagcgtttggtttggttccaatgttgcagttgcagttggagttcgggtcggggacaaaaaatcacttacttccactcgagcgccaccgcgccggaacgaacgcggaaacccgttccacggtccatcatactctctttcctccctccccaaccgtcgctcagttcaacatatggccgtggggatcgggattgggagctgtcaggtccaggtgccgcgggaagggatcctgcagggaagtatcaagcgccggaactggaagcacccgatgacagatggtgctcgaaagtgaactgtaaaactggacgcccatcaccaacaacatcacaccggcatgcagtgcgacaaaaaaaacacacccacactgagagagaaacaaaaatcacatccacgcccgtcgtcatcaggggcgaaaaaacaacaaaccacacaaccggctgagccaacagaaactaacacagcgcgcactgggctggccacaaaatgtagtactaactaaatccaatccaaataattatatttcaattgtttatgaacggcattatgcgaccggaccggaaagtcgctggctcgactcgtccgtccagtcccagcaacaatatcaacaataacacatgctcccggcctggaacggtgggtatgcgtcggcggcgtatgctgaccaacataatcaacgtatcctttgtggtgggattccgggattccggcaggatccgc

SEQ ID NO: 2 - DNA, 129 bp, Anopheles gambiae
cctttccattcatttatgtttaacacaggtcaagcggtggtcaacgaatactcacgattgcataatctgaacatgtttgatggcgtggagttgcgcaataccacccgtcagagtggatgataaactttc

SEQ ID NO: 3 - DNA, 54 bp, Anopheles gambiae
cctttccattcatttatgtttaacacaggtcaagcggtggtcaacgaatactca

SEQ ID NO: 4 - DNA, 23 bp, Anopheles gambiae
gtttaacacaggtcaagcggtgg

SEQ ID NO: 5 - DNA, 20 bp, Artificial Sequence
  Nucleotide sequence capable of hybridising to the intron-exon boundary of the female-specific exon of doublesex (dsx)gene
gtttaacacaggtcaagcgg

SEQ ID NO: 6 - DNA, 97 bp, Artificial Sequence
  Nucleotide sequence that is capable of hybridising to the intron-exon boundary of the doublesex (dsx) gene (i.e. a guide      RNA)
gtttaacacaggtcaagcgggttttagagctagaaatagcaagttaaaataaggctagtccgttatcaacttgaaaaagtggcaccgagtcggtgct

SEQ ID NO: 7 - DNA, 1074 bp, Artificial Sequence
  zpg promoter
cagcgctggcggtggggacagctccggctgtggctgttcttgcgagtcctcttcctgcggcacatccctctcgtcgaccagttcagtttgctgagcgtaagcctgctgctgttcgtcctgcatcatcgggaccatttgtatgggccatccgccaccaccaccatcaccaccgccgtccatttctaggggcatacccatcagcatctccgcgggcgccattggcggtggtgccaaggtgccattcgtttgttgctgaaagcaaaagaaagcaaattagtgttgtttctgctgcacacgataattttcgtttcttgccgctagacacaaacaacactgcatctggagggagaaatttgacgcctagctgtataacttacctcaaagttattgtccatcgtggtataatggacctaccgagcccggttacactacacaaagcaagattatgcgacaaaatcacagcgaaaactagtaattttcatctatcgaaagcggccgagcagagagttgtttggtattgcaacttgacattctgctgcgggataaaccgcgacgggctaccatggcgcacctgtcagatggctgtcaaatttggcccggtttgcgatatggagtgggtgaaattatatcccactcgctgatcgtgaaaatagacacctgaaaacaataattgttgtgttaattttacattttgaagaacagcacaagttttgctgacaatatttaattacgtttcgttatcaacggcacggaaagattatctcgctgattatccctctcgctctctctgtctatcatgtcctggtcgttctcgcgtcaccccggataatcgagagacgccatttttaatttgaactactacaccgacaagcatgccgtgagctctttcaagttcttctgtccgaccaaagaaacagagaataccgcccggacagtgcccggagtgatcgatccatagaaaatcgcccatcatgtgccactgaggcgaaccggcgtagcttgttccgaatttccaagtgcttccccgtaacatccgcatataacaaacagcccaacaacaaatacagcatcgag

SEQ ID NO: 8 - DNA, 2092 bp, Artificial Sequence
  nos promoter
gtgaacttccatggaattacgtgctttttcggaatggagttgggctggtgaaaaacacctatcagcaccgcacttttcccccggcatttcaggttatacgcagagacagagactaaatattcacccattcatcacgcactaacttcgcaatagattgatattccaaaactttcttcacctttgccgagttggattctggattctgagactgtaaaaagtcgtacgagctatcatagggtgtaaaacggaaaacaaacaaacgtttaatggactgctccaactgtaatcgcttcacgcaaacaaacacacacgcgctgggagcgttcctggcgtcacctttgcacgatgaaaactgtagcaaaactcgcacgaccgaaggctctccgtccctgctggtgtgtgtttttttcttttctgcagcaaaattagaaaacatcatcatttgacgaaaacgtcaactgcgcgagcagagtgaccagaaataccgatgtatctgtatagtagaacgtcggttatccgggggcggattaaccgtgcgcacaaccagttttttgtgcagctttgtagtgtctagtggtattttcgaaattcatttttgttcattaacagttgttaaacctatagttattgattaaaataatattctactaacgattaaccgatggattcaaagtgaataaattatgaaactagtgatttttttaaatttttatatgaatttgacatttcttggaccattatcatcttggtctcgagctgcccgaataatcgacgttctactgtattcctaccgattttttatatgcctaccgacacacaggtgggccccctaaaactaccgatttttaatttatcctaccgaaaatcacagattgtttcataatacagaccaaaaagtcatgtaaccatttcccaaatcacttaatgtattaaactccatatggaaatcgctagcaaccagaaccagaagttcaacagagacaaccaatttccgtgtatgtacttcatgagatgagattggacgcgctggtaaaattttatatgggatttgacagataatgtaaggcgtgcgatttttttcatacgatggaatcaattcaagagtcaattgtgcaggatttatagaaacaatctcttatttatgttttgttatcgttacagttacagccctgtcctaagcggccgcgtgaaggcccaaaaaaaagggagtccccaacgctcagtagcaaatgtgcttctctatcattcgttgggttagaaaagcctcatgtgacttctatgaacaaaatctaaactatctcctttaaatagagaatggatgtattttttcgtgccactgaactttcgttgggaagattagatacctctccctccccccccctccctttcaacacttcaaaacctaccgaaaactaccgatacaatttgatgtacctaccgaagaccgccaaaataatctggccacactggctagatctgatgttttgaaacatcgccaaattttactaaataatgcacttgcgcgttggtgaagctgcacttaaacagattagttgaattacgctttctgaaatgtttttattaaacacttgttttttttaatacttcaatttaaagctacttcttggaatgataattctacccaaaaccaaaaccactttacaaagagtgtgtggttggtgatcgcgccggctactgcgacctgtggtcatcgctcatctcacgcacacatacgcacacatctgtcatttgaaaagctgcacacaatcgtgtgttgtgcaaaaaaccgttcgcgcacaaacagttcgcacatgtttgcaagccgtgcagcaaagggcttttgatggtgatccgcagtgtttggtcagctttttaatgtgttttcgcttaatcgcttttgtttgtgtaatgttttgtcggaataatttttatgcgtcgttacaaatgaaatgtacaatcctgcgatgctagtgtaaaacattgctaattcccggtaagaacgttcattacgctcggatatcatcttacgaagcgtgtgtatgtgcgctagtacattgacctttaaagtgatccttttgttctagaaagcaag

SEQ ID NO: 9 - DNA, 849 bp, Artificial Sequence
  exu promoter
ggaaggtgattgcgattccatgttgatgccaatatatgatgattttgttgcatattaatagttgttgttatgttttattcaaatttcaaagataatttactttacattacagttagtgagcatattatctactacataaacacatagatcaaactggtttacataaattcaaaaagtttggattaaaatcgcagcaattggttatgaaaaaatatgtgcataacgtaaatatcaagtaaatttttgcattgcatatttatagactcctgttacaatttcggaaaaatgaaaaatgttaattaatcaaagaagaaaaaacaaagaaattaaatcattaggtagcacaaccacaagtacatatttttatggcatgaatattcctctacactaacatattttatagcaattctattgatcgccttagtatagcggaattaccagaacggcactatagttgtctctgtttggcacacgcaatcatttttcatcccagggttgccatagcagtttggcgacggtcacgtagcatgcgaaggatttcgttcgcacaggatcacttttattctaacgtttgaagaaggcacatctcagtgcaagcgctctggaagctgcttttaccgaacgaactaacttttcaagtaacctcaaaaacttgtctctaacgacaccacgtgctatccgcgagtttcatttcccgtgcaaagttccccgatttagctatcattcgtgaacatttcgtagtgcctctaccctcaggtaagaccattcgaggtttaccaagttttgtgcaaagaacgtgcacagtaattttcgttctggtgaaaccttctcttgtgtagcttgtacaaa

SEQ ID NO: 10 - DNA, 2291 bp, Artificial Sequence
  vasa2 promoter
atgtagaacgcgagcaaattcttttccttccatgacagcagcagctacagtgggaagccgaacgtcagacgtgtttgacatgccgaactgggcgggaaaattacagcgtgcgctttgttttcaagcaaatcacaactcgctgcaaacaaaaccgttgagaaattgattgttttataatttgtattgtattttatttgttataataaactaaaaagacatactttttgcatattttatacataaaaacatacatgcagcattataaaacacatataaaccctccctgtagagtcccgtatcgaaatcttccatcctagttgcacagtacgacggacgagtaggccgtgtccgtgcaaattccagcttttagcagtcttttgctcggagcactcgcggcgagtcggaggtttctgctgaggtgcttagcgctaaattagccaattgcttttgcaagtgaaataaccagccgaatagtacttcaaaactcaggtaagtgaactagttttatagaacaaatgtttgtttgttagaagttagtgaagtgtttgtgaaaaaaatctctcatttcggcaaaactaacgtaactgatttcaaattgaattattgttttgtgatgttatattatttcatccagttgattagtattttcttagttatgttcaaaatacagttaaattaaatttcatttcatttactcataaaataatctcttggcttatttaatttttctcgaattcgcttgtattgttcagtagcacgcgccattcgccctttgtttcattttgtacctgctcccactaacacactggcagtgcgaaacaaaagccttcgcacgcgttgctggtattagagtgtgtgcgtgtgtgtgttgagcgctctgtcaaaatcggctgttgccgccggtaccgaaattgcctgttcgcacgctgttcgtaaacattccgtggtgtgtatcgtgtgttgtgcatgttgcgcgcctccccccttttgatagcaggctgccgtggctgccgtggtgtgtggcgcagttgagtttttggattaattttctaaggaaatggcacgagaagagcggtggcagtgtgttggtttgctctgtcccttcctttctgtgtgaagtgttcttacagcacagcacgtatccaccaccgcacacagagcaggcaaggaagtggaagtgaacaagtgtgctgcgcatgcatgtgtgtggggggcattttagctgagatcgtcgttatttgagaagcggtataggggccagtcggtgtcgacgtacggaagcggtttagttttaatccaagcgtatcccgtcgtggagtggttgtgtggctctgtgtgctctcatatcagttccagagtgaggttagtagaatcacagtccttggcctttttcgttacaagatatccagaaggatggcgttatttccacagcttaccatggtgctcttgtttgctcgaatcaggggagaaaaacagtttcgtgtttcatgaaccgcagttggcactggagcggattcaaaagtcttcgatatgcaatagataagagagtcgttggggcatagttgggaagcctttccgagatgtggagtttccgagaggagaaatggtgctttcgtgcacgttccgggacagcgggccccgcgaagagcatctcgttgtcgttcatccggcaataattgatgcgaaaagcgcgcgcgccactggcttagcgcagtgtacacagtgatattcacctacacacacagaggcacacgccttcacacgcgcgcgtgcttcaaaggctacttcggtggcggtgtgtgaggtcgcttgcaatggacaatgaaaatttcgctggaaaataccatcgtctctttaggttgcaatgggtgcgggtagagcggtggtcgtcgatattggtggtgtagtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgcaacggcaattattttttgtaatatttcgaccatctttctttctctctctccacgtgctgctgctgttgctgctgctgctgcattgcatgttccactattcctctcggtttgtgcctgcggacgccattgctagtcgaaagagagtcgccgttagtcgcgcttcgagcaacggacacgttttttggttgaaaccaacagcttttttcatcttcgggagacacacagatctcgaatcgtacattcccataaggagaattgtcatcttccggtgaataaagaaaggaaac

SEQ ID NO: 11 - DNA, 297 bp, Artificial Sequence
  Nucleotide sequence coding for the nuclear localisation signal (NLS)-tagged Acr protein
atgccgaagaaaaagaggaaggtgagcggcggtagcaacattaatgatctcatacgggagatcaaaaacaaggattacaccgttaagctgagcggtacagactcgaacagtatcacgcagctgataatccgcgtgaacaacgacggaaacgaatacgtgatctcggagtccgagaacgaaagcattgtcgagaagtttatctcggccttcaaaaatggctggaatcaagagtacgaagatgaagaggaattctataacgacatgcagacgatcaccctgaagtccgagttgaattga

SEQ ID NO: 12 - DNA, 68 bp, Artificial Sequence
  RG427 primer
aacctcgagatgccgaagaaaaagaggaaggtgagcggcggtagcaacattaatgatctcatacggga

SEQ ID NO: 13 - DNA, 31 bp, Artificial Sequence
  RG428 primer
cgcttaattaatcaattcaactcggacttca

SEQ ID NO: 14 - DNA, 20 bp, Artificial Sequence
  RG1044 primer
atccgtcgatgcctaactcg

SEQ ID NO: 15 - DNA, 22 bp, Artificial Sequence
  RG187 primer
tcaggggtcttcaaactttatt

SEQ ID NO: 16 - DNA, 20 bp, Artificial Sequence
  5R1 primer
tgacacttaccgcattgaca

SEQ ID NO: 17 - DNA, 20 bp, Artificial Sequence
  RG1047 primer
aagataagggcttgcctcgg

SEQ ID NO: 18 - DNA, 53 bp, Artificial Sequence
  4050-Illumina-F primer
tcgtcggcagcgtcagatgtgtataagagacagacttatcggcatcagttgcg

SEQ ID NO: 19 - DNA, 54 bp, Artificial Sequence
  4050-Illumina-R primer
gtctcgtgggctcggagatgtgtataagagacaggtgaattccgtcagccagca

SEQ ID NO: 20 - DNA, 9044 bp, Artificial Sequence
  Anti-CRISPR construct (>C119_pBac[AttP(VasaNLS-AcrIIAa4_3xP3GFP)AttP]pBac)
cccccaactgagagaactcaaaggttaccccagttgggggatctcggatctgacaatgttcagtgcagagactcggctacgcctcgtggactttgaagttgaccaacaatgtttattcttacctctaatagtcctctgtggcaaggtcaagattctgttagaagccaatgaagaacctggttgttcaataacattttgttcgtctaatatttcactaccgcttgacgttggctgcacttcatgtacctcatctataaacgcttcttctgtatcgctctggacgtcatcttcacttacgtgatctgatatttcactgtcagaatcctcaccaacaagctcgtcatcgctttgcagaagagcagagaggatatgctcatcgtctaaagaactacccattttattatatattagtcacgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaaaaaagatttgcgaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtattttcgagacgttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgacacgaaaaacatattctcaataaatcctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacatgggtgaacactatcccatatcaccagctcaccgtctttcattgccatacggaattctggatgagcattcatcaggcgggcaagaatgtgaataaaggccggataaaacttgtgcttatttttctttacggtttttaaaaaggccgtaatatccagctgaacggtctggttataggtacattgagcaactgactgaaatgcctcaaaatgttctttacgatgccattgggatatatcaacggtggtatatccagtgatttttttctccatattcttcctttttcaatattattgaagcatttatcagggttattgtctcatgagcggatacatatttgaatgtatttagaaaaataaacaaataggggtcagtgttacaaccaattaaccaattctgatgcgcgtctctcccctttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtattcacgacagcaggctgaataataaaaaaattagaaactattatttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttaaacattctctcttttacaaaaataaacttattttgtactttaaaaacagtcatgttgtattataaaataagtaattagcttaacttatacataatagaaacaaattatacttattagtcagtcagaaacaactttggcacatatcaatattatgctctcgacaaataacttttttgcattttttgcacgatgcatttgcctttcgccttattttagaggggcagtaagtacagtaagtacgttttttcattactggctcttcagtactgtcatctgatgtaccaggcacttcatttggcaaaatattagagatattatcgcgcaaatatctcttcaaagtaggagcttctaaacgcttacgcataaacgatgacgtcaggctcatgtaaaggtttctcataaattttttgcgactttgaaccttttctcccttgctactgacattatggctgtatataataaaagaatttatgcaggcaatgtttatcattccgtacaataatgccataggccacctattcgtcttcctactgcaggccccaactggggtaacctttgagttctctcagttgggggttaattaaaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgatttgatctagagtcgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtggatcccgggcccgcggtaccgtcgactctagcggtaccccgattgtttagcttgttcagctgcgcttgtttatttgcttagctttcgcttagcgacgtgttcactttgcttgtttgaattgaattgtcgctccgtagacgaagcgcctctatttatactccggcggtcgagggttcgaaatcgataagcttggatcctaattgaattagctctaattgaattagtctctaattgaattagatccccgggcgagctcgaattaaccattgtggcctgcaggatgtagaacgcgagcaaattcttttccttccatgacagcagcagctacagtgggaagccgaacgtcagacgtgtttgacatgccgaactgggcgggaaaattacagcgtgcgctttgttttcaagcaaatcacaactcgctgcaaacaaaaccgttgagaaattgattgttttataatttgtattgtattttatttgttataataaactaaaaagacatactttttgcatattttatacataaaaacatacatgcagcattataaaacacatataaaccctccctgtagagtcccgtatcgaaatcttccatcctagttgcacagtacgacggacgagtaggccgtgtccgtgcaaattccagcttttagcagtcttttgctcggagcactcgcggcgagtcggaggtttctgctgaggtgcttagcgctaaattagccaattgcttttgcaagtgaaataaccagccgaatagtacttcaaaactcaggtaagtgaactagttttatagaacaaatgtttgtttgttagaagttagtgaagtgtttgtgaaaaaaatctctcatttcggcaaaactaacgtaactgatttcaaattgaattattgttttgtgatgttatattatttcatccagttgattagtattttcttagttatgttcaaaatacagttaaattaaatttcatttcatttactcataaaataatctcttggcttatttaatttttctcgaattcgcttgtattgttcagtagcacgcgccattcgccctttgtttcattttgtacctgctcccactaacacactggcagtgcgaaacaaaagccttcgcacgcgttgctggtattagagtgtgtgcgtgtgtgtgttgagcgctctgtcaaaatcggctgttgccgccggtaccgaaattgcctgttcgcacgctgttcgtaaacattccgtggtgtgtatcgtgtgttgtgcatgttgcgcgcctccccccttttgatagcaggctgccgtggctgccgtggtgtgtggcgcagttgagtttttggattaattttctaaggaaatggcacgagaagagcggtggcagtgtgttggtttgctctgtcccttcctttctgtgtgaagtgttcttacagcacagcacgtatccaccaccgcacacagagcaggcaaggaagtggaagtgaacaagtgtgctgcgcatgcatgtgtgtggggggcattttagctgagatcgtcgttatttgagaagcggtataggggccagtcggtgtcgacgtacggaagcggtttagttttaatccaagcgtatcccgtcgtggagtggttgtgtggctctgtgtgctctcatatcagttccagagtgaggttagtagaatcacagtccttggcctttttcgttacaagatatccagaaggatggcgttatttccacagcttaccatggtgctcttgtttgctcgaatcaggggagaaaaacagtttcgtgtttcatgaaccgcagttggcactggagcggattcaaaagtcttcgatatgcaatagataagagagtcgttggggcatagttgggaagcctttccgagatgtggagtttccgagaggagaaatggtgctttcgtgcacgttccgggacagcgggccccgcgaagagcatctcgttgtcgttcatccggcaataattgatgcgaaaagcgcgcgcgccactggcttagcgcagtgtacacagtgatattcacctacacacacagaggcacacgccttcacacgcgcgcgtgcttcaaaggctacttcggtggcggtgtgtgaggtcgcttgcaatggacaatgaaaatttcgctggaaaataccatcgtctctttaggttgcaatgggtgcgggtagagcggtggtcgtcgatattggtggtgtagtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgcaacggcaattattttttgtaatatttcgaccatctttctttctctctctccacgtgctgctgctgttgctgctgctgctgcattgcatgttccactattcctctcggtttgtgcctgcggacgccattgctagtcgaaagagagtcgccgttagtcgcgcttcgagcaacggacacgttttttggttgaaaccaacagcttttttcatcttcgggagacacacagatctcgaatcgtacattcccataaggagaattgtcatcttccggtgaataaagaaaggaaacctcgagatgccgaagaaaaagaggaaggtgagcggcggtagcaacattaatgatctcatacgggagatcaaaaacaaggattacaccgttaagctgagcggtacagactcgaacagtatcacgcagctgataatccgcgtgaacaacgacggaaacgaatacgtgatctcggagtccgagaacgaaagcattgtcgagaagtttatctcggccttcaaaaatggctggaatcaagagtacgaagatgaagaggaattctataacgacatgcagacgatcaccctgaagtccgagttgaattgattaattaagcggccgccttggggtggggttgttatgtgttgcgaacgagagtggatctctctcgacatttccttattttttttttctattgttaacacttacaacgaaacttcggaagagaagtttcctcattcgaaacgaggagtcaaactcctttccttgcttttgtgacatgcatgattattctttcatttactgacgtaacgatgtaaaacacacagaagaagcatgacacacagcaaagaattgttcgttaataaaacgttgatgaaaactttgaaaacataagaacttgcacttttattctataattcgtgaaagcttgcaccgattgtctttcattattcaatgtaatgtactgaaaggtgatttttcgcacttgtatactctagaactgaagtattctaacaatacgtcacctttaggtccattccaggataaaatacacaagtgaaggtagttgtacaaagtacttagactaacccaggtttcaaaaaagataacgacgaattgagcgtaactgcacaaagcccgttcattgtcaacatatcgttcaggggtcttcaaactttattatcagcccgcgggccgcaacaacgttgcttagagcaccatcttgatgtatcctttatctagtggcttatcgtttatgtctcgtttggctaagaaaatactaataacatattcaatttacggagcggtaccatggtacaaacgtcatactgctagacttgacaacatgcccatcgagggttcttacctcaaatggaccgatcccttgtaactggcggcgtaaactcagtaaattccaaaagccagtataggccaccatgaccgtctaactattccaaaaaggagaatatttgaaatttctatatctatagtattattactatatctatatgtttctttagtacaatattagagcttgattattttcctgaaaattgtaaaatatgcatgttagatggttgatacatcgttttaaaaaaaactgctgttaatggccacattttcg

SEQ ID NO: 21 - DNA, 41508 bp, Anopheles gambiae
  misc_feature (12994)..(15405): n is a, c, g, or t
  misc_feature (16603)..(16622): n is a, c, g, or t
  misc_feature (22229)..(22248): n is a, c, g, or t
  misc_feature (24177)..(24196): n is a, c, g, or t
  misc_feature (30640)..(30808): n is a, c, g, or t
  misc_feature (33478)..(33497): n is a, c, g, or t
  misc_feature (37449)..(39162): n is a, c, g, or t
aattagaagttgatggcaatagattaatatttacgagccgtcttgtggagaattaaatgataaaccagttataagcgaaatctggatttatttggcttgcattttgaaaaaaaaactaaatagtttaagtgtcggaaccgaatgtttttggttggtgtgtttttatgtgcttcttatcatcgtgcgtagtgatattgagataaaatatggtgaattttgtgcttatttgtgattggtgaacagtgctagtttaatacaagtgatgatgaattaattattgattttattgaaaaaaaaaaataggaaataagtaaaagaattcgataatataaccaccaatatgatagacataagtatgtacggaaatcattcttcgcacacaaatggttttcagaacagtgatcttgggtccggagggtacccttattttacagcaggtcaccatcatcaccaccattaccaccagcaccaaaggcaacctgtgcaacaacaacagcttactaattctgctactaacattgttagtggcccacctgtgaacaataccactagtaataccgtagtgacctctaatagcagctccaacaacaatgctaatccgcccacagccaatatccatcagacttcctatgttcacgcatctcacctatacagtccgtccgcaatcgaatacggaataactacctctagctctcccccaactgatttgtactttgaagccgatagccaaagttctatatactacggccctagtcatccaacaccggcggctccttttcaagaaaaccgtatcataagcgcggataacggattgagctatactaatttggactatgccatgtaccatcaggcacaggaatgcgcagatcctagttatctggcgcatcatcaagataacaaattacatctacatcgctttggagcctttcctggagctgtagcgtccgatgccactgttgatggggatcctcatcatcataatcttcttcatcagcattcgcatatttcacatcagcacagttccacatcccctaatgatgcacatggcggtggaactacctggggacaagtttactccgataacattcctcagtcacaacatcagcaactgcagccgtctcttaaccagcatacggtggctcagtgcagtcctaatgtttcttccgcattgctggactcgccccatgtaattctgagtgcgagtgtatccgacgatggtgacagtaatggtagtgttgctaatagtttttcgcgccttcaccaacatcatcaccatccccatcatcaccatcaccatgccacattgcagcaaaatatgcaagcgcagcagcatccggcgaatcagcaatcgcagcaacaaacacagcagcaacaacaacagcagcacgctcaacaaccccagaaccaacagaatgcacctacctacaagtggatgcaagtaaaacgaaacgtgccaaagcctccatgtaagtatttttctagctaaagttgtattgttgatgatacaacccgcactatctccagattatcacaatcccacagaggatagcgtcacactgagcttgctttacggtctcaccataaaacgatacaattcccacagggactttgagtcccaaagggtttgctttctcaatggaggctatattttttgtctgtgtgttttcctttctatccagcaatcacatacaacgacaaaaaaactcttttatatcggtaaaaaatatcaaacgcacctatttcggttagctacctttatcagcatcaaaaattcatacgcgttgcatagtttaaccccgtgatgaactataaaaatatatctaactggaccagtagagccataaattttgctcctaaaatcttcaatcctgcaccaatggcagtttgataaaggtgtaataattcaaattttccgtaaacatcttaccttcgccatgagcgttatcgtttgcatgttgataaatattacatatgactgctttaaagcaaagaagacaaaaacagtaaatatgaattctagcagacgttagatattaccgttaatcagatatcgttgtcgtattatgaagtactggcttattactacgtgataacattatgatctccatatgattttttttaatttagagacaattttatataaaagcaattacgctattctcgaaatagatttcaaaaatttcgatccttcccataagctttttcagcataccaaacattaaaattatgcctagaccacatactgcttaaagaacaacgcaaaacaaccccgattttgttaattaaaaactaccatagctggtgaggttaacaaactccccaattttaaccaccaacaataaaaacaacgctcacttattcttagtctatgttacgggtcaaagaggttgtacaccgtgcgtgacttgggtcgtttccatttgaatgaaattgccattatcgtccagaagtaagtggaccccaacaaaaacgtgtcgaggacagacgttttttactgcgatgccgtcaaggtcactaacgaaaaacgacggagcgatgactgaaaacgaaagtggattatgctagcatagaaacaattcttttcgttctctaccctgttcaacttctttggtggctgaaatagattcttctataataacgcgcaacagcaaaaaaaaaatacgcctacgatcgatattacaataccaacgaccccatgcagacgtgttgtggggtctcaatttactttcaattggctaactgtccactgtcaaaaatgtcacaaactgcactgtactgcaaagcttacccaattggtagtgagagaaaagacacgaggcagtaatttgaatgtcatagataataaaaacaaatattagattcaaacctccccctttgtagacactacattatggatacagttctgttatttcaatattaacatgcatgcctctgcatgcgctgttgcactgtacttgctggatatgtaacaaaatagattagtattgaaattaatatagcacaaccaccggcttacaaattaatttgtgggtagcactactccacactacactactcaccaatttgacttaatttcattcaatctttccgcgatcacttatcttttctataattccaatgctttcatagtgtcaaattaaaaatatctgtggcatagcaaaagatattttttttaacttgtaacttttcacatgttgagattccatttacctaaccgagaaatacatgcaaatacatcagtgtggcttgtagtcgtagcctgtaactgtagtcgaacattcactacaactcaatttgcatggtccatagtgtatacagaatagattttatcacaacatataattataatattttcacaacaacaacaacaacaggcaacacgcaaccaatcggtgaaacttctcccttaggggtttctttaattattacagcaaaccaaacattttgcagtatcaattgttttacattattaaaataattctaagcgatgccagtttaagcgattgtatttgacggataagaaatgccagttagttcataaaaaaaataaaatctgacatatatcatttaaattattttaacacatagtaccacacagtaactttaaagtaccattgtcaaatttgaattactcacacattttaaattgttatatgacatatattgacatatatcctaactagctctttatcttttcccaaaataaatatacgtaataaacttacatgtgttctaatccataccgctttgttaattacctacaggcataacttttaggaaaagaaacaacagtcaacaaacattcaacttctctgcgatgtcaaaaatttcattttcattagcggtaattaatgatatatcagtagctacaggcgacgctgaccataaaattgtcatatctgatcaactgaccaaacatgttttcatgatttgttgtatcaaacgtctcaatacgtttgacaatagctaatgaatcggtactatacggtcgacaatctactgtaagaagatgccaacgacccccttgacgtttataattcattaacgtacgaattatgtttcggtgcagaacttctcaaactgatttcttattttttgttttttttttttataaatattactgctcttactagactggtcagtcgtgtaacatgatctaaatttcttattacacatattcaatgtaaaaccatttccttccagtaatgcaatgatgataaggtgaattatatgtaattttatattgaatatttttttatttaatcctacaagaataaatttatatctgattgtgtactcccggatactgaccctgtagactttttatttacttcatacatttttgttaagctctttcctagccataaatgagctaaactgacaataaatttacagttaccaaggaattttgcctttttttaaatgaatgttgacaatgaatttattagttaacctacagcaaaatagccagtcctacgtataaggtgttcgatccatatgcagcttgatcctatgacggatatattgtgcagtatgacgaaagtaccaagagacctcccttttttaattttttagtgttttttatcaatcatgaaatattcaagccattagcgtattacttccttaaaagaccaaacaatttgcaaatatttacatacaacaggccatatcaaacaatattagttttttttataaaaatcatttttatatttcacatcagtctttacattatgaatatcgatgcagggcgtactggacaaaactaaattgatttttttttctgtaatgtagcaattaattccgaatttggatgaatcaattgcggagtttattagtaaccttaaatattttgtaatatttttttgcagaatgtaataaacctcttcatattcatccttttttcttataagtttataattaagttattagagccattggtaatgcgtaaacgtaaatgtgttaattgcctttaacaaatttacttagatgatgtagatgacttagatgacttagatagcatgaggtatagttacaatttgccagaacaatgtagcagtaaaacaaacgataatttattcttgtggctttttaactaaaatttaatatttaacaatcaaaccttaatgaacaaaggctagtttctttttcaattatttatatcagtgaacgtaaataaaaaaatgtaataggtaaatttatttaaaaaaaagtttgtataaaaaaaaatgttcttgaaaattatacggatggaatcaatcaaagacactgcagcataaatgaattactgaccgtcaataaggagtttatcattaaaatgataaatgatagtattaacatgttttacccactttctttcttactaacctactttactactcacttactaattagattacttataaaaagccagattatttgtcaaaaattgtaaaaaattggtaaaactttgcaaatcaaaagagttgcacttaattgggcctttcttatatttgatagttacataacttgtgaaataggaaatggagtaatgtgtataacattcttatgtgaatttgaaaaaaaaaaacgaataaaacattgattatcattttctatgatattctaagtacagcgaacgtgtcattggaacacgttgaaaccgattacaataattgttcaacaaattaaattatgtcatttccggtttatgatactatagatcaccatgcatgccatcaaagtatattactttaatacttaaattgtgagctgaaagtgctggaacctgtcgtatgatgaattaatgtatttgcgaaactgatcagggatattagagttagtaccgttaactacacctatgctgccgtagtctgaaaatcaatattaactttttaaatgaacctcaaataacatttttaattaaaaaaaaaaaaacaaaacaaagtataattacagttgtgcgagacagtgttaaacgttaaaatatttaaaagcgtataaatttgttcatttcacaagtataacacttttgcacgtaatttatacaattggctataattttaatattgagtataatttatctcaaaatacttgtatattaaagtaaatatattttggaaatatgttgctttaggtatcgaagaagtcaaaaatctataatcgtgaaaaaaatgtgcaatgagaacgtatcattcgttcaagagtttaagcggtacgaatatagactgtcttacagcagccacaagcgtggtaattctatatcatacgtggtgttatattttatcttatagcttgtcggtcatgttacatttttatttacaccgcactagtaccccaacgttgaaattctgtcaacaacttctctattatgttgatgaaaatcctacgatcgcatgacgtaaccgaaactcaagaaaacttgcacagttcgttcccctaaccgctttgggtaagttcaagcaacacgcgacacgaacgggtttattgtttatcatagatcgtcattcaatatcacagtctctcatcagcaagtcaagtgcctaagcgaatcactgtgctgtgacttctcttacaaatgtctaagttgaatacagctacctcagaatgtgtatcgtagcatcgatggttgcaatcgtttttcaaggattacgcttaccgatatgcaatccatataccaccgttgcccatcaaaacaatatttttactccctatcccttagcaatagttcctttatcgatcaaagattacgtttaccgtgttgatacagccgtactatattctataggtatctattactgtacaaataagaaacgcttttagacttttattctcaatgcaacacattttgtttgcgtatgatgtgtacaattaaaccaaaactttgcgttaatatcgctcaacaaaaaaaacatgtgcattttcccccattcctattgttgttacatttgagcaatcgaacatgtcaaaggtcatcttgcaatcttaacacaaacatatgataaacctactatgagcatgattaaggtcagaaaaatataacacttctacgcgttttcgcttttccatcttccatacaacataaaagaaggcttgaaagtatgtattaaaatgggcaaaagttaaacctccatgttgtactttccttgcttaaggaattctccttgtctaacctacctcatcctccgttatttgtgggagtgatccaccatcaacgtccatcttcgacataatcctaactttcgccagacatccctattatacagtataatctattaattaagcctgtcagttgccgcacttgggtcgccgcgggatggttgagtcgtgcgacctatctgactgccggctcgtaaattagcgcttgtttgaaacttcaacaagttcacaactcccttctagctcccccttttagcttacttttgtttgtttatggtttggttaaaaggtatcacgcatgtcagtgggttgttcgggaaattgttatatttgcaagcgagacaatttccatcgtagcataaacaagaaacccaggttttaaactgatgtttttctgctatcagattgagtgcatttatgttcatgtagattttacggtttattttgaagcggtagtaaactatttcaacaaaactttaggacattttaaatgacacaattcgcaaatcgaaaacgaaattgtacaaaaaggctgcgaatgtttatagtgatgatcagtgatcaacattcctggactgatttcaatgcaagatagagataagggcataagcataaaacttcgattatcatttttgacatgaaaggaaaaagattcgccacatgcaagagaaacaatcaaaagagacctaaagacgtctaatacttatgttatatgaagctattatatacattttctgttaatattagtttactccaattaaaattaaaatctgtatgaatcaactcacaaataactaaaccttgatttgataaggaaaatattccttatatcgaattcctttaaaatataaatcgaagtgggaacgatcgaagcggtgtcaaaaattatataaaattgtggtttcttgatgttttagatgttttagagcattgtgccatattgtaggcattgctgaacagggcactgatgaaataaattaaagtaaccattgtacactggccactgcaactatcgttcatcgtcgatggagctttggaaagtgactgattgtcttcttatgcgtaatgatctaaaagttcaagcttataccacatttttaattctgttttggagaaaacattccagtttttatttaaatcttatttggtgtgcacaggagaccctcaaaatacgacccatgcgggttacatggcgttagttgttagttgttagttggcgttacaatgatttttatagctaaaagttgtcatttggaagaaattgttttttttatttaatttttaattattacagtcacatacacttttatgaagaattccaaatatccaagcttgaatatttatttagcataaatgacttattcaatataatgttaaacaaattaatgattttctttattaacccagatagctaataaatataataagtagttcgggtttccgtttagtcgcaagttttttttttttttgattatagctatagcattacgtgtacttatatatatatacatgattttccagaagtttttaagctctaaatatgtttcttaactggaataaattgaagtgtttagccatgtaaagtcaagaggacatggcggtctattagtgtcagttgaactatttattgtgtattaaatatatgtatctaaatgccatacatcataaagatatggcatataaataaatatttttatcatacaaacgtaaaataagctgattttttttagtaaagatgcattcattgttgcatgaaatatatctcctatccaaataggagcttatataagcaaaggtgtatttttggttatagatacgattaaaatacacaatatcgtaggtctcgattctgaaacttcgattataaactgttgtcagatatccaaagcgtcactttttggacttacaccttagatttgaacattttttggtgaagagaaaagccgcgtttcgacggaacgatttctgcataaaaactcctgtcaaaatcaccgtttcccatggcaacgaaaaatcgagaagagttcagtgaatcttattatacacactcagacactccatatctctcttggcaaatctttccccttcctctagaaacaagtttcaaataaagatttgccaatattttgcatttacacaccacgaatctttgcaggattcaattatcgtaaattaacaaaatatttcgttaaacacgggttcttgttgtaagaaatgcacaatatacaacctttagaacatttagaacttggaagcaatcgaaaacaaatgtttaaagacaaaaaaacacgatatttctctcggcctattaaaaatagcaagcaaatatttttgttatccattccgcgatggatagcttggtttgctcgaaaaagattcgccatagactgctagacttcttctacctcagtgaacaagtgtgtttgaaagttatttgctagcagaatatgccgtgtctgaacgcggctattcgcagtgtggatggcacacatctttgcttgtaatcttttggcagatgcaaagaaaactgtaaattttgatatttcaacttatttttgggatcgctgcacatgggagaacattcttctaaaggttattggtgatatccatgcattaaaaaaacagttcgcgactttttttttactatccagcgagatacctgttttgctatcagctcaaattaagcagaataaatagtttccaaaataaagtcaaaatgctcaaatagaaccaacaaataattcgtttctacgaattattgaattcgcagaaacgaattatttgttggttctatttcggaaaagaccccaatagcaggatatcttgaaccccgttctaactctatgaacgatagcaatgtgttgtataataatacacattattatttttgacacgaaaaatttcttgaatcaagaaataatttagacaataatcaaaattcaactaaagtgtatacgtttcacaaattgaagtagcatttttagtttcattttgtttattaacatttgcatgctaactacactaattacaggttatctttatatcataataattgtaaattttcaaccttcttatttattttggcaaaaaccgtagtcggtcaaggcgtgcctgtaccactggtgggcctggctttctttgacttaaaggttaccatagccggatagccagtccaacgtatgggggcacggtctgtaccacgggttgttacaattgtgtatccgaccctaaaactataccagatttctcatttcccgacttattgattcttcttgtattatttttgtattctttttattctttctgcctctacaggctcccgaaattttccgatggcactgtgagcgggcctctacacgtcacggcttagagtaccgtaccgttcctttttgcatgcagagtttgactgtacggtacaccacacgaccctggtacgtgtggaggcccagtcatgtgctaattcgtgctatatatcgcttatatatcacaaactgaatcacatatttacaaagggaaagaatgttatattttcgcgttaaaataatacaatattcgaacgtacggtatgagaggacgctagggtctgccagtcggacggaaagccaaaattgattcagttgaaaaatattacatcgggctagctcgcgacgtgtgagcccgctaaaaattgatgaagttatcgaatgtgggagctcattgatacacaaaactcattgatttaaaattagagaactgttattttattaatttcattagctatttctaaaatcaatagtgttatcattttttaactctgatcgctatggaaaataagcctaaattaacttctaacaatgcaatctacacacttcctttgatttatatttgtctatagtatctctgccatcgcattcacaacaattacacagaccacaacctaatgcttgctcaacctattaaattttgttttagtttatcgttcctaatgtgtttgtatgatcacttttggatgcccgatatggcactatcagcgtatgaaaaaaaaaatacacgagcgctgagaatcaacccatcgtgttcgaaacagtagcaccaaggcttatcaataaataaatgtttatcgcaacaactgcagcaccacacatggctgcagacagttttcaggtttgaggaagggttaagactttgtcacattagatactcgtaatcgcaaacttggtactagggactatgaacagtgagggcttacaaccgcgtcacattgcagtaagacataataaagtgaaccaaatatttgatgtgcttcctatagataaacacgatgcgcaagctagagtaatgacaaaaaatcttacaaatgttcaggtatacagtccgagtcagggtatttgttcagatagtgaaaacgactgtaggaagtatcgatgtttcgtgtatccgattgacatcatatcaagataacgttcgtgaagcttcctgtccgcattatttgttataatcgatggtagggtgctaaaacttagagttgttctgtgtctagataactgtcactgtataaattctatcgcaagcccatttcagtccacagcttcattaaagtactttagataaagcgaaaaataatcactttgtatgtatatatttgtacattactaattaaataaaatgttgaacatgaaaaatagcatccttaagtaaaacatatagtctgattagtgcacttctcttagtcttgctatgaggttggtgcagaatcttctaaatgtcaactgtagtgcagctgcaattcttttgcatttgtatataattgctgacctattggacaaatgtaaactcatcaacaaatggggctctttgattcaatggtaatagcaattttgcaacattgaaagtttattgttcagagttttagtacaaggaaggtcgcagaacaagatgtaagtcgaaataatcgtgtgtcaaatatccatcaatacattgttcacgcctcaagttgaaaagtcgaaatcaagattatcgggtattgcacttgaaggaacaaaaaataaatctttctaaaaaaatgtcaagctagccaccacctgcagcctttgaggtcatccggtgcgacccgcgaccagtcgattaaaaactttatttgtaattctgtttccttgattattatcaaagccatcaaccaatgaaattttagaacataagagcaatttcattgaaaataatataataataataatataatgatagctgactcgacaaacttagctattccaaaaaaaatattttattattgatatgttacttgggatagttgtatcgtgcccgtatcccctcagaattcgatgatcgagtgtaaaccgccaggctcctcacaccaagtgtcaaagtgccgtatacaacacaacaacaatcctgctctttgtatgggagttagaaaatcatttttaaattaaatttagtgtaacatctgaacgatttttaagtcttaaaacctattctcataccaccataaggtgtgtgcaaagtttcgttgaaaatgatccagccgtttcggagtttgctcgcaacaaacaccgtgacacgagatttttatatatatagaattcactaaaattttcttacgacaaaatttacaaacgaatgcaattttcatatacacatataaataagcatcagaagtttcttcttctttttcttctttttggcacaacaaccgatgtaggccaagacctgtctgtaccacttgcgaggttgactttcattgacttattgatttacccccagtcagtctagtcctacgtatggtggcacggtccatttggggattgaacccatgacgggcatgttgttaagtcgtacgagtcagcttcagacatttacgagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtggattaaaaatatcaggtggtggattagggcctttggggggtcgccatagttgagggactttacgtcattttagaaccgttaaccattggtttccgcacacaaagcttagcaaacagaacagatttctttgttattatgtgtatttttgtgtgtctattgattttatcgaaaaaatgagatatattaacaaaagatttgatgatttttttatgcacgaaatttaaaatcatgtgagtataagttctaaactcacgtaaattgtccatgcggctcgtagataatgaggaatcaatgagaaaattgaaaaaaataatgatttcttagtttttatcatcaaaaatctcgaaaacacaatgaattataaaataaattcatggaataaaacaaaataacacactttaatccatgttttaatgtttaataagaactcgcaaagtccaaaatatatttttgaagaatatttaatcgaaaaaatggggtagataaattatttgaacaaatttaattaatgtaaacaaagtttgaatcctggggaccttacgtcaagtgacgaattgtcaaaatgcactagagtccaccacctgatatttttaaatccaccttgatctagactgaaaatttgcaggctagttttattggtagtttttctatggagtgtttatatgatttccacagcctccagctgtcaaactcaatacaagaaactgactaaaatcgtgaaatcggcccctatgttcgaatattccacaattattttcattattttaatttcatttagttttataattcttttaatatcacgcagtgtaagtttgcaatgtgaattaatcattccagaacagtctggtaaatcgtataacacaaaatacaaataacacaatacacaaatagcacacatattctattttttatatcactctaccgatatatttcaatcccaaagccaattattttatcatggtacgattaattttactgatggtgatatttactgtttaataaatgcttgatgcttggatttattatttcatgaaaaggcataaataaactgtctattatgaattgaatataataaagcaataataaaatgttgtttgcgttgatatttcacctattgtaaaggtagcgtcaaaatggcccttgactacgttaattgtgacgtaatgcggcccgcgaacctaaaagttcggagacnnnnnnnnnnnnnnnnnnnnaaaatcgtttgaattatgtgttttcaaaaatgtttgaacaatgtcaatgtatttatgggaatgggtaaaacatatgcgattaactgttaataaatcgtttaatatagggttactgtacctgtattggacaggttagtgttttttttttatttgcacattttaatttattttttagagatattcgtgaaaattcaaacactgtttttgctcctatttttggccgttttttcctattttcggaaggctatgctcctattttcggcaacgcaaatacgagtcggaaaatgtttcaatcaatacaaatagatagagaaaatgtttaattaggtacgttctcaaaatccttctttcttcgcgaatatttccccttccaccagaaacaagtgtcaaaaaaagattcgccaatatttttgcgttcacacaccaacaatctttgctatatacaataattataatttaacccaataattcgtaattccctacatactcttgaatatgcccatataacggatcactgcatttaactgctctactctgagcaaaaaaaatagcactaccatgttttttttagtaattgtagatttaccctaaagataacgatgttttagttgagaatgttggaataacttgtattacacctttcaagcacattgctttaatgtatttattgaataatttataattatggggccctttcctttttaagttcgctcgctgaaatttcagcctgtcacctgtttgcattgtatagcagttttcgagcagctatctaagggggtataatatacaagtggggttataaggtgtatgaatttagaagcttctgtttctgaatgaagattttaagaatgttttgagtgttcatcaagcatccagaaagcttgtttgagcaaaagttttcacccgttctgtcaaaaagagatgctcaaatttggttatgaaaaacatgatatgagaccaactggattacatacactttgattctatattccatcaccagatctctttaatgcaccttggtaaaaatgtaaacaccacattgttttgccatttttcgagcaggtactcgaatctaactctagctttgaggctagaataatctagactgaaaatttgcaggctagttttattggtagtttttctatggagtgtttatatgatttccacagcctccagctgtcaaactcaatacaagaaactgactaaaatcgtgaaatcggcccctatgttcgaatattccacagttattttcattattttaatttcatttagttttataattcttttaatatcacgcagtgtaagtttgcaatgtgaattaatcattccagaacagtctggtaaatcgtataacacaaaatacaaataacacaatacacaaatagcacacatattctattttttatatcactctaccgatatatttcaatcccaaagccaattattttatcatggtacgattaattttactgatggtgatatttactgtttaataaatgcttgatgcttggatttattatttcatgaaaaggcataaataaactgtctattatgaattgaatataataaagcaataataaaatgttgtttgcgttaatatttcacctattgtaaaggtagcgtcaaaatggcccttgactacgttaattgtgacgtaatgcggcccgcgaacctaaaagttcggagacctctggtgtagagtacttacagggttttccaaggcgcttttaaatgtaaacattgttattcatcgcgtgcaacatgttaatccacatcaatctgatatttcgtttccatcatcattatttttaatttcttcagcataattttcaatacgattgggtttgacagtgttttgttatgtgttgaaaatcatgtgatgaaactgcaatttcattttgaaccaaatacaccatttgacagctgttgaaaaacatcttggatgcagtgaataattatgtgcacattggaaaatgacttggaaaataaattaaattcaatgatttaattcaacaaaaatccggaatcgcctcctaataactccgccaaagtggtagatagatgtatttactataaaaacaaaagtggttatgtgactgttatccaatgatgaaatcaaatgaattacttacaggattttcaaagtcactttcgaatgtacagtgcaagtatgtacgaatggaagtgtttcgaaacacttcatgggtcttctccgtctatcatattgtcacagagggtttgaagccggatttcatcacccgttcaaatgttgaaaatcatgtggaagaaattaaaaattattttaataaaaatgaaatatcagagagatttggaataatttcttgcatgcggtgaaaaactatgttttcatttgaaagtgacttgaaaccctgtaataatttttaatttctcaagcacgattttcaacacttcaacgatctattgttggcccaaagtctctataaaaataaagaaaaaaaaggtctattgcaatcgttttttcgaggcataaagccgcatctcattaggattttgacaacattttccaaattgatataacttaaatttgtgacttggaaaaccccgttagtctctgaaatctgctagcggtacaggcaggccttgaccggcaacaattgttgttccaaatatataacctataaaatgtcaacatccgttgtatccggaaatcgtcttaactacaatgcgtcctatgtttaggatctcctgtatggatgtatgagtgaatttttcatcagacaacgtttctacacaattgagcgagaacaatccaatcattaagtttgagtctgccgcgcgtgatacttcctaaaaaacggaatcacttgcaaatttggaacgcttgtgtatttttttaaaaattcacaagatcattggcccaaaataaatcaacttagcgctgctttttggcttaaacaattcatataacagcttaaaaggcatggcaacataacgcttagaacaacgcaatttaagattgtttatttgttaaattaacataataacttttcatcgctacagacaatgagcaggacattttaccatggtgtgcgtgcacccgtaaattcactaaaaaggaatactttatccgagtgttttaaaataagaggtgagggcctgaaccagtactctccagcttccataaaaaatatacggtgcgctctcagcgagaatccacatgacgcccacgggcctgcccacgtccgaaggcagagccgatggcatacgattctatcttcaccgtaaacataagagggacagagcttacaggatttcacactttatctcaagaccgcgggaccccttcccgaatctgtcttagccaaagccaagactagtgtatgatgcttgaaaacgttgatgatacctgaaaacgttgatgatacctgaattcatcgaatcgttttcatcgaaatcgtgaaaaacacgaaaagattaattaggtgtttccagtacactgatgtacacaaaggttatatgtgtaaaaaaatattcgaccgcatgttctcggcgcggagcatcgtgttcgagaaatgtgccgcagattcaacattgcatccgatgaattcaggaatcatcaacgttttcaagcatcactccgcagtcttggcattggctaagacagattcgggaaggggtctcgctgtcttgagataaattgtgaaaccctgtctatgtatctttgataaagggccctttggacggtagcaacgcaataagcaatgcaatcagtaacccctgaatatatgtaaattttgaataacgtcttcccgtattaatacacgatgcgcaatctcggattgcgcatatattcattttatcaaaccatgtgtcatttcgcgtagtcaaccctgagctataaacgtcatttccatacaatttcagattgcgccaagattgcgcataaatttacaagattatatgtccgagatatataattttttttgacagatcaatatccgtttgacagataaatatatgcgcaatctgagattgcgcatcgtctattgctacggttcgatttatgtccaattcaaggtgtttggatattaggaggtgaagtgcttcagagcaaattgacatttcacgacttgacataacaatactgggggcccggtattaaaatagggaagggctggaggggggtatagaaattttctatgcgatttgacataaccggcctcagcacaatttttcgatcgaaaaaaatcgatcgatccggctgtcattttggtgtcatttgacactcatttcaacgtcaggtctttttgaaaactggtataccatgacactcacgagcaaaatgaactacagtctgttcccgagttacgcggtttctgcgttcccaacgaatccgcgactctcgaatatccgcgtaagtcaaatttcacgttttttgaataaagttctattgaatactccgagtttttgatttaatgtagtacttttatacactttatcatttatttgatatgattcgtgcagaaaattctaatatttctggcttttaagcggtttcaatttgttagcgaaaatgcaatttataccgatcttgaagcaaattgtattgatttgacatttaatctgtcaaatttagaaaaccgcgtatctccgaatccgcgtaagtcgagaaccgtgtaactcgggaacagactgtatgctacaaaaattcgatcgttccgctttgtatggggaaaaatgggggcgacgctttgagctgcggaaattatcgaatataaaaaaatgtcataaatcaaggttgatgttttgaaaaacgttatgtaaaagcaagttggtaaatttgttgattgttttttaatatgttgggtgataaaaaaatatttattaattattttgaaaattgtttacatggggcgggggcgactttatgtaggcgtataatagaaatagttatactgtcagttttacgtgagcgcctatcgaattttttcgatcgaaaatttgtgctgatgccgaacaatactcaattatggcgcccggcaaacgcgctaatacttatacgcggattcggagatacgcggttttctaaatttgacaattctttgagcaaattgtactgatttgacacatcaattgcaaattgccaaataatttccgttttgatcgaatgttaaaaactatttcaaaaggtttaaaacagttatattcagtcagaatcacatcaaataattcataaagtgactaaaaccaccccctacctgcaaaattacacgaaaatttgtgttattttagctggaaatcacgagattcgacttacgcggaaattcgagatacgcggtattttgcggccgttttcggtccccattaaccgtgtatctcggggaccgcctgtactcaattatggcgcccggcaaacgcgctaataattcaccttctaaataaacacaccttggtccaattgacatcaatgtcacgatgcgttctggattgcatacgcacaggctaaacactgttccaaacggttgcattgctaaatgcgttgctaccgcatgaagtacagtatgttcccgaggtacgcggtttaacgttcccgaggaatccgcgtaactcgaattacacattttnnnnnnnnnnnnnnnnnnnntgtttccagtacactgatgtacacaaaggttatatgtgtaaaaaaatattcgaccgcatgttctcggcgcggagcatcgtgttcgagaaatgtgccgcagattcaacattgcatccgatgaattcaggaatcatcaacgttttcaagcatcactccgcagtcttggcattggctaagacagattcgggaaggggtctcgctgtcttgagataaattgtgaaaccctgtctatgtatctttgataaagggccctttggacggtagcaacgcaataagcaatgcaatcagtaacccctgaatatatgtaaattttgaataacgtcttcccgtattaaaacacgatgcgcaatctcggattgcgcatatattcattttatcaaaccatgtgtcatttcgcgtagtcaaccctgagctataaacgtcatttccatacaatttcagattgcgccaagattgcgcataaatttacaagattatatgtccgagatatataattttttttgacagatcaatatccgtttgacagataaatatatgcgcaatctgagattgcgcatcgtctattgctacggttcgatttatgtccaattcaaggtgtttggatattaggaggtgaagcaagttggattaaaaatatcaggtggtggattagggcctttggggggtcgccatagttgagggactttacgtcattttagaaccgttgaccattggtttccgcacaccaagcttagcaaatataacagatttctttgttattatgtgcattttagtgtgtctattgattttatcgaaaaaacgtaatatattaacaaaagatttgatgatttgtttatgcacgaaatttaaaatcatgctagcatgagttctaatctcaagtaaattgcggccatgcggctcgtagataatgaggaattaatgtaaaaattgaaaaaaaataatgatttcttaatttttatcatcaaaaatgtcgaaaacacaaagaattctagaataaattcacagaataacacaaaataacacactttaatgtatgtttcaatgttaaataagaactcacaaagtccaaaatatatttttaaagaatatttattcgaaaaaatggggtagataaattatatgaacaaatttaattaatgtaaacaaagtttgaatcctggggaccttacgtcaagtgacgaattgtcaaaatgcactagagtccaccacctgatatttttaaatccaccttggaggtgaagtgcttcagagcaaattgacatttcacgacttgacataacaatactgggggcccggtattaaaatagggaagggctggaggggggtatagaaattttctatgcgatttgacataaccggcctcagcacaatttttcgatcgaaaaaaatcgatcgatccggctgtcattttggtgtcatttgacactcatttcaacgtcaggtctttttgaaaactggtataccatgacactcacgagcaaaatgaactacagtctgttcccgagttacgcggtttctgcgttcccaacgaatccgcgactctcgaatatccgcgtaagtcaaatttcacgttttttgaataaagttctattgaatactccgagtttttgatttaatgtagtacttttatacactttatcatttatttgatatgattcgtgcagaaaattctaatatttctggcttttaagcggtttcaatttgttagcgaaaatgcaatttataccgatcttgaagcaaattgtattgatttgacatttaatctgtcaaatttagaaaaccgcgtatctccgaatccgcgtaagtcgagaaccgtgtaactcgggaacagactgtatgctacaaaaattcgatcgttccgctttgtatggggaaaaatgggggcgacgctttgagctgcggaaattatcgaatataaaaaaatgtcannnnnnnnnnnnnnnnnnnncgtgttctacattgattgcattcccgctgcgcaaagcgacggaagaaatcaaggcgtttggagaattttctcatgccttctttttctctccaacggcaaatttgtcaagcagctcgtcgagcttcccgttcctggctccgcctcacacccctcgcctgagcgcgctgtcgaaggtttggatgtgttggtgcgtcaggcggccaatcgctgtcagccgtcgtccgtgctttttccctccatagcgccatctctttctcgcgtgtggtcaatgctcgcgagctgttcgatcccttggtaggcttctgctacataggagagccgcagaccaatgatgtttttatcatcagcgtatgccaggatctgggtagacttatagaagatggttcccatagtctccaccctcgagtcgcggatggccctctctagcgccaggttgaataggaaacaggcaagcccgtccccctggcgctgacccttggtggtagcaaaagatcctgagagttttccatccaccctcacctggcatgtgacgttggtcatagtcattctaactagccttatcagtttgggccgggacagtgttgttaactgttgacatttttcatgacagtcaccgtaaacagccggtcaaaataatttttggctgatgacattcaatgcttccacgacacgactgtcattaaacgaaattcattcgtgtcgctaccgtcagggctgatgcaatgacatgaaatgtaaacaatgtagtctgaatgtcatttgacattttttgaacaacacaaatcgatacgatcggaggattatgaagtgtatcgtcgctgaattctagtgaaatggcaaaaactagtgaaatgaaagcctttgcaccgcacgctctgttgtagtttgacatgaatacggccgtttgcaatacgacacagttcacgattcatacaggttggcgaatgtcaaatgaatgtatcttgaaaatgtcttcatgtcaaatgacattttttgacgggaatgtcaaatgacagagagagatgggaatgggaagcttcaaatcgaatgaatgtttacaattttatgtcgatgaacgtcaccgttcgcaacactgggccgggattccaaatgagctcatagcgtcgtacagttttaccctggctatgatatcttatgcggctttgaagtcaatgaagagttggcatgtgtcgtgtctgtattcagccatcttctccaagatctgccccatggtgaagatctgatcagtggtagattttccgtttcggaatcctctttgatagtttcctactatctcttcgacgtgcgggacaaggcgatactgaaggatcagggagaatattttatacgcggtaatcaacatcgtaatacccctgtagttgttgcagtccaacctgtctcccttcttgtatatggggtacatgatgccgagattccagtcacaagtcatcgattcgctatcccacacctcagtaacaatttgatgaatctcgttttctagtcgtgcacctcctttcttgaccagtttagcggcaattccgtcggttccgggtgccttgttatttttcagccgacggatagcctttcgtgtttcttctatgctaggtggcagtagcatgacactatcggctagtggcgcttctagctgctcgttgaactggtcgttgagtaattcatcaaagttcattgagtaattcatctgagcccatcgcaagaggacctctggatggttactaaccagatcttcatccttgttgcgacagcaggttaccttaggtacaacgttgtttcggtgacctgctatcgcttggtaaaactttcgtgtcggtccgtacgcctctctggtttgctcgagttcccgcatgttttgctcttgcaaagcatgcttcttggagcggtgaactcgtttctcttcgcgtctgagccgtgaatattcctctgcgcatgcccgcgttctatgccattgctgcattgctcggtatgcagtattcttacgttcggtcacttgtctccattcatcgtcgaaccagccagatttggtgttgccacgacgtggtgggagtatatttcttgcacagttttttttttgtttttagagcgttccacctcttgctcgtagttttatatctgttttctggtagtagagactcttctaaagcggctttgaattcctgttggacagtaatgtcccttagagagtccgtgcgattctacaacgtatcactaagccaaccaagtagtgatcagaatcgatattggctcctcgatatgttctgacatttaacagactcgactgtcgtcggcggcttattaacacgtagtcgatctggtttgaaggattcgccatccgggtgcgcccacgtcattttgtggatgtccctccgcgcaaatttggtacttcctacaaccagattgttcgctgcggctaactggaccaatctactatcattatcgttactgtgctcatgcaatactgtgtattggcagtacattggctccctaccgacttttgcgttgaagtcccccaggatgattaagaggtcatgcctggggcacgcatctatagttctagcgaggccgccgtaaaaaaggtccttctcctcttcttctttttcttcggtaggggcgtgaacgttaatgaggcttatattaaagaatgtgtctcacatgcgcagggtacataacctatcgtttatagccttgaaatctatgattacggatttcaaccagggaccttcggcgaaacccgtttcgagcacgcggtggcggtcgtggcagctgtagtaaatgtcgtagcattgcttaccacgcctgttgtgcacaccgttctctagccaccgaatctcttgtagagctacgaggttcatgctcagtgtgactagggagtcatcaagttgtttcaaggctccggctttgctaagagtgcgtacgttccatgagcccattttgatcgttgtccgggggcattgcgtagggtcggtatcgttaggtccgttttgtaaatcccttcttccatgctttctgtagtcgtttattccgtgagactgggtgactggccctgcgctcacgtggccgttttatttagcgtcgggttgccccccgacgttcaggcacccagtttctcgggatacccaccccgctcctggttcgcccaaggggttggatctagcccgtgtacccaagctaggatatatagaggcacatgttaccactctgcacgacgaggacgtgtcggaataggagttagatgggagagcctgtattatctctcggagggcttcggatcccatcccattgcagagtagtttaatgttttttttaacatatcgttgtaaaattagaattttaaaactcctatcaatatttttttcaatcaaaaatgtactcgaactccaacacatcaaccggccttgccacatgttttcagaggatgcttatgtctttttattttctttcaaatgttgtattacagtgtaacaatattttgaaattagcactgaaatcatctcttttatatgataccaaccactacacgcaaacgaaatagaattggctgaaagatagcacacacattcatacttttgtcatagtgttactgccgaaaagggatgggtgttttggagcgcaccagtggctccgaagtcggctctgcacccacggctcccggctctggagcccacggccgcaccaacagctccggagccggctccgtaccaacagccccggagccggcgacgaatcaatggctccggaccaacgaccctggactaacgtttcaatagagacggcattgtatgatagccgaaaaaacgattctatcttctcgcaagtgtagaagctaacacccgatgtgttgtgtgatgtgatttgaacaatgttcaacacttattgatttttttatagattcttttcaacattatttactcagctaatgatgtatcttcccgattgaaaatttattgaaaatgaattttttggtgcaatctgtaaaaaaccggctccggagccgtaagtgcggagtcgttgctggagaggagtcagcataggagccgtgggtgcggtgccggctactttgggttggagtggaggaggtttcgaagttgtctggagtcggtcggagccggtttttaatattaaacattatgagcatattttatagatcgtcaacatatatttttatacaacaagcaaaacgattgaaaaatgtatataacttttgaaagaaacgtaagtaaaacttagaaaaagactaatcatccacttaaaacatgcggcctggccagttatggtggtaaagttagaacatatttttgattgaaaaaaatcttgataggatttttaacattcttactttatttatatgttataaaactttattatgaacctttaagacagtttacattcaaatcttacaagaaattcaaaagatatactcttttaaccacgaaaaacatcattgttccatacaaaatgtatggcctacccgggtaggcggttgtacgattgcgtgaagttttttggtcgtacacaagacggttaagcagttctggcgtctcatgaaaaatgtcgaatgacattcattctacattttgttcatgtcatcgcaaaagcattgattgttattgcacaaatgattgtcgcttttggaaagtcgtgtcatggtagccaagaatatcatgataaaaaaaatgattttggcagccgcttacttcgaatatcataaaaaatgtcgacaattaacaacactgcccttaactgagaaagctgctccagcagctgttcatctgactcttcaaaacggcacggtttggcctggaagagcagggtttgctgtttcctcactcgtctataagcggacgatcttgttcggctgcagtgctgatggcttgttccaccatacgccaatggtctgcgaggggcatcgctgcgatgttattgtcggccggtggcgtttccccgagcggtgtcgcgtatccctccgccaaattagcacacttcaaccgatccaggttgagcattggagtgggctgactccgttggttgttgaccacggagagtttctggtgcagctttatcatgaccaggtctgagtcgacgtttgcgcctctataccatttaatgtcaataatatccgagaagtgcctcctggcaatcagaacgtggtcgatttgatacattgaactaccgctagggtgtctccaggtgtacctttggcggcgtgtatgctgaaagaaggtggggatgctcatgtgcttggaggaggcaaagtttatcagcctgagaccgttgtcgtttgccagctggtggacgctgaagcatccaaacgctggtttatatgcctcctcctgtccgacctgagcattaaggtctcctatgacgaccttcacgtcatgcttcgggcagcggtcgtactccctctccaactgcaagaaggcctccttctcgtcatcggtgctcccaaggtgcgaactgtgcacatttataaagctcaggttgaagaatatgccatgaatcctcatcctgcacattcgttcgttgatcggccaccacccgatcactctctgttgcatcgcacctagcaccataaatcgagctcgcgcgtctcaccaccgctctggtagatcgtgcagttgctacggtaccggcgcaccgtcacgcctttctacacagcaaattttctcatcctgcttcagtaaagttttttactgaaacggttcagtattagaatattttactgattttcagcataaatgtcatttttttactgattttcagtaaagcaatttactgaatgcatttcagtaatacatatattactgaaaatcagtaaaataggtgtcaaattagtcgtttcactggatatcagtaaaacaaattactgaaaatgcagcaatccattctgtcaaatcgacctgtcagtcagatcatcaaaacaaaacaatgcggtagccacttgctcgtgatgtgcaaaaagttgattttgtaggcgtttacaggcaccctggtgaaatttcaggtggtatttcggcttacgggagtaatttaaaacaattggcagccgtgttggtgtgtaaaatgtttgctacaatccactgtgcgtgctgaaattgcgtggtgtctgtgagcgcttactgaaccatctacacttgcggcggtgaaatacagtacaaaaactttataaaacaacacgaaacatgtatttatatgaactgagcgactggcaatatctgcgcatcaataaaaactaaatttaaaagaagtatttcgtcattttttaatcgctaccaactactgaataatcagtaatgtgagaatggctttactgggatttcagtaaacgagctgtcattttggtgagcatcggacattactgaatgattcagtaaacattctttttactgaaaggattactgaaacttcagttaaaaaaatttcagtaaaatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaattagcatgttaagcagtttactgaatattccttggaatatgcccaccaaacacaatttttctttcatttatgtatgcttccaccctttatctatggattaaagtcatcacatagaatgaaaataacttaaaagatgaaaaagtattaaataaatgttgacgttttacgagaagctattttaacacgcagggcaaaatgggcatatgtaaacatgctgtgaaacgtcaaaatactggggcaatatggtaatatagtggaacgtcgattatccggacagttcgggaccgggcggttgccggttaatcgatttgcacagataatggtccaagaaatgtcaaattcatataaaaatttaaaaaatccactagttttatgattaattcaccttgaatcaatcgataaatcgttagtagaatattattttaatcaataacgataggtttaaccactgttcatgaacaaaaataaatttcgaaaacaccactagacactacagacctgcacaagaaactggttgcccacttgactatcgcagcaaatgctcgctgtacgtttgaacagctgtcacatttatacgcacggttaagccacccgccggttaatccgcccccggatgatcgacgttcattgtataactgcgcttagataatggtctatgcatttgcgcacgtttcgtgaaatgtattttcgctttatcttttgttttgctgtccagccctttaaattcttccaaaatacgttttcaaaattaaaacagtttttacacgtttaaaactacaaaattgaatgttttgaagctgtgcccgttatcattattgaggcgacaaatactacaccatagcctcaccaaacgtcgtatgtcaaaagcatctgcccattttgccccacgtgacgcatttttgtattttttgttacacagtagaacgtcgattatccggggacggaataaccggcggacggcttaaccgtgcgcataaatctgacagctgttcatacgtacggcgaaagtttgcagcgataatcaattgggtaacgagtttcttgtgcagctctgtagtgtctaggggtattttcgaaattcatttttgttaatgaacagttcttaaacctacagttcttgattaaaataatattctactaacgattaatcgattgattctaggtgaattcatgataaaactagtgaatttcatatgttttataggcgtttgacatttcttggaccattatccgtgcaattcgattaaccgccaaccgtccggtaaaatatatcatgtaaaacttcaacgcgtccctgtgacactggtttaagattattttcgaagtggaaaaaattaaatgaatatgccaccaaaccctaatttgaaattttcttttttatttgttatgtaagggttatgaaactcacatccctacccgcaaatttccgttaaatgccttctaatcgccttgtaatcgccggcgaatttaaggcgatcagcagtgcttttagcagtaattaaatgcctttgaaatatgtcaatgaaaaatttcgcttttaatttgaaatttgaaattgcaactgtcaaaagaaaaccttacaagcgtcttcagcactgcactgttgcacgattgctgataataaaatcatatagttgctgatagcacgatttacgtgttatgttttcttgcgttaagctcagagctatttcactttattaatgatggtctgcattaatcggatgttaaataccaacccgttgtaaggttttaatatatcaaactcatttcgataactctaataaaaggagaaatgagatacatatttctcccatcctgataataaaaggtgaacatagtgttattattatgtttttaaaaagatattaattaatttagcttcacataaattttgtttgtattgatttttttttctatttgggctataatggtggtatggttcctatagtcgtcgtgttgtgttcctatagtggtggtacacaaagatgcctcaaaaactgtgtaatatacatggaactaagttttgcagctgttttcgtatgaattttttttataaaacatatatatttgcgagtaaagtctaatgcttatgaactaatttatgtacaaaaacagaatgacacagggaaaaaaataacatattttataacgttagacaacaattgagtacgcctagatcaatcataccatcgttgttaccaacgatgtggatgatatagtttgcaaacaaacgcaaaacacctattctctgttcagtactagctcaggctagtactgggaattgcaaacagttaaaagaacgagcacaacctggagctagcgcagtaattttctgttgcaatagctcccaagccgcagaaactcttctgcaacatgaaaatttatgctccagggtttccccgaacgaacaaaacaaacacgagttggtcacaattcgccccattcggaaaaggagatcgcatgttggaatttttccgttgaccaacaggaaaagtgtcgaacgttgaagggatgataaggctggacaattgagatttctccagatccttctccagctgtgttgtcgtgtctcacgcactatcttagcgtctggtaatctgctcgtaagcatacgccacaacagccgagacgatggcctttgaccgtactctggtggcacgtacgcaannnnnnnnnnnnnnnnnnnngaaattcctattgacgcgatcaaaagcatgcggaaggtcaaaggagatgagcttaccacactttcgtttcctctgcagatcaatcactctctccttaacggaaagagcagcttgaaaaatgttgcttggtttattacaacatttctgcgctgccgaaataatctcccacttcccgatgatgccatcgagtctgtgtttgaccatccgcgacagaagcttgtaatctgcgttaaggagtgatatcggtcgaaatgcagacatggtacagcctcctcccttcttcctaaccaacactatgaccccgtcaacgaaactttcaggaatctttcctaagagggcctcattaagaactagaagaagctctctatttatgatgctgaatgcacggtgataaaactctttgggaattccatccgggccaggggactttctcgaaggtgactttttaattgcatcgaaaagctcaccataagtaaactcgtccatggaattttgttggcttcgcagtcctcgggaataacgcatgtgcttgtgaatgttgtttctgtggtcattgtggatgtgtcggatgttgtgtatagattcctgaaaaagccctcaacatgaacatttatatcctttagctcagttaaggacgatccgtcatcaaccgtcagcttgtcgatcaccgttcgtctcctcctttgctcccctaactgaaagaccgacatgctctctccgcatacgcgtgtttcattaatgcgggtgaagtcctcggaaatacatctctgaaagagaagcatttgtccttttatacgatttatattcactaactcgttagggtccgtgacatatcgatcataggcggacttcaaccccctgtagagcagttcatgatgcaagcgaaaactttggtaccgttcattcgttttccaactgaaaacgactttattttaggcttcgcgagctcaacccaccactgcattcacgagccgtaatttctacgctgtctggtccaatatttccacttgcatccgaactcctccaggtttgcgtcagttaatacatgtggtctcagtttccaataaccattgctgtgcgaacagctcggcgggattggaaggcaaacacgaacggtaagcgccttgtgatcggaaaaggacaagacatgcatactgcttgctcttaactgtgttttaagtgaagaggagacataacaacgatcgatacgtgaacccgaaccacttgtgacataagaaaactccactgagttacctctgagaagctcccagctgtcgcacatgcccatgttgctttaagcgttttgcaaagaaagactaaaatttctagcgcccgtcacatctttcggctgcaacacgcagttaaagtcacccgcgagaataacatggttacatgcattccgcaaatagaaaggtaacgttcgcccgaaaaaatcctccctctccgctctccgctgactgcccgaaggagcgtcaacattgcacagggtggcattgttctcgagtcgaacgcaaatcagacgagaatctaagctgcgctcgacatgggaaaattttaaatgttgacgtaaagcgatcgccgtacccctccccgtcacatcaacattgcaaattacgttatacccaggaagcgatagatctgaaatacatacttcttgcagaaatatgatgtccaggtccatcgtcctaatatatgttttgagggcttccagctttgtcgcgttagaaatcgcattaatattgatagtagctatattatagctactaccaagattactacttaccgggttaggatccatcgcaacaaaacttaattaatctgttgtgaagttgtgtgtttttttggcggtcgaccaggcttacgcttggacgcgctcataacactaaatgttgacgaacatgaggcgtcactctcattatccgtttccggattttgcttgcgtttaaccgcaagcggttcgtcgtgggaagggatggaaaaggatgtggaagggttgcgagcggctctttgaacattaaggcacctgaaagcgcccggcattccaccctcatgggctcagatgcagccgcatcgttagcatcagtggcagcatccgtagcagcggcagcatcggcaacagcagcggaaacagcggcattgatagtagcggcagcggcagcatcattggcagcaggagcagtaaaagtaggagcatcgtcattttctacatgcgttgcgacggcaccaacggcagcacgcatcttctcctcgggttgctcaccactggctttcactggcttcggacccgaaggcggagcgagagttcgcggctcgatcgggatcacaagccgattagccactttcgatgcagtgctcgctggtggcttagtagtgtttgtaagggaagtgattttgaccttccctttacgccgtcccgtggtgttagtacctgcggtactagttgatgctgtgttcgcatcagatgtttgtgctggtgcttgtttaacggtacttgcgtacgattttgccgcaccgttgttgaggagctgacccacacggttttggatgcagctaataccgtgatgcaaagcttcattgcagtgacggcaagtttgccgggcaagttcacccacacagtaatgaaggaagggatgggtttcgccagcttcatgcgcactatgcgcacacccgaaggaatcccggtaagccgcgtttcggctccccaaacaccgggcacaatagtgagcacttcaccgaagcggttcagctcagcggaaacgttttcgttggggattcctggccgaagatctcggattttcacctccatcgcaccatccaccatctcgatcggaatcggatactgcttcccctcgtgcgtgatgcagtgcttccccgaacgttccttcactttgctttccgcacgttcaagcgttggcatagtgacgtagacggcactttgcgcagtgctgtgttgcaccatcttcacatccacacccgtttctagctcttcaaaaaggaagttcacggtttcttctagggtgggtcgcttaggagcctccgaatagtcgatacggaaggtgttccttccatatctttcggtcgtacacattatggcttgcaaatctaaacaacaacacgtcctttctcgttgacgtttgcaagcgaactataattgtagcaagaaaatattaatttaaaaagaaaaaaacacaaaaaagataataaaaatcaggatttgaacacacgtattctcggttcggaactaaaagcgttgtcactgctctatttagcagttaaacctgtaagggtgtaaaaccagtatataaataggctaagatggcggcaagctatctgttctcgttgaatcaaaaagttgaaagatttcgaagagaacccgttacagccgtgaaagtttcggttgaaatctttattcgccttctatggcgactaaggccttaaatgccttctgaatgccttcaaagccgtttaatgctggtagggatggtgtttatagaacactctaatgaatactttttgagcattgccccgctttcccctattaatacggttagagaaatgagatacatcctcctgcgagccatatccgatttgcaagggcacgtaagggctcgcgcgccatataagttcacagccccagagcccttgcattaaagagcttgcgagcctaggcatttttatccccgataaacatttccgtcaaacattcccgccgtgcaattgacttcaaaatgtaaaattgaataatttcacattttattcgttcaattataaaaataaaattaaataacatcccaagcgccacagattaagctggaacgactggaaaatcgaatatccatagaaaaaatgaaaccttcatagcgttactggtttaggcagcggtcagaacctcgccgcatgcgattttgccacaagcttttgtatgggatttgacgttaaagacaacacttgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaacgtgagaaaaggtattaaattgatgtggtccactgaaagattgactgtattcattttcacgtattcaaaatgaccacgtagcatgccaatcttgaccaatatggtccttaaatcattcaataccagtaataccaataagtaaaaggtccatcgactagtttgggtggtcatctgtgttatgaattgctcttttactttatttaaacacttaaaactttaacattataataaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatcccaatccaatagaaatgggattaagctttaaagtaaaccattttttctactcaatattcaatataagtcatcgtcaagacatctcgattgcaagaataaataagcctcctaataaaataagtagtgaatatagaaatattattaaaattaatgctttcttttctttttattctttaatagtgactaaatctttacagttatcaactacaccagaatatcatattcctacgcatgtactcgacccgttaagagttcctaatcacacgacagttggtatgtatatacataaatgtttttttaatgtttttcgactacgaagtcctttttcacattgaactttgtagcctgcattactctttgcttcctagggatttataataccaacaaataatgatttcaatgatacaatgataatgatactctttcttcatttcagtagcccctagtgctgtatcacctcatcaatcaagttttatgataaacaataacagcacaggccgtacaaatttcaccaataagcagttaacagaactggagaaagaattccatttcaacaagtatttgactcgggcaaggcgaatagaaatagcaaatgctttacatttaaacgaaacacaagtaatgaaatatttcacgcagctgcatatttgtaatcagtgtttatgaaataatttttgtttttatttcacttaggtaaaaatttggttccaaaatagacgcatgaaacaaaagaagcgagtgaaggaaggtttaatacctcctgaaatacagaaccaatctcccaaacactcttccctgataaacagcaatgattcctcctcggcaagctcgtcaacagcaacgttaacagctataccttctgcctctggagcttctgcaacgaacacgtctacgttaaacgaaagcaatgaaaacagccgtgaatcaattactatgtagaacaaatgctttttcaaaacatgaaaagactctctttttattagttagaaccaattgaaagacattaatttacaacagaatttgcaaattatcagaacacttttagggctgatcgtcttattcaaccatacggttaatagatgagtaattttaaaattatcagtcaaattatctttcacattttctttaaatcactaaactatatgacttcatggatggaagttatatatttattttaagaccaccaggttcatgactgcttgacttgaggtattacgaacaattttgaactacaatatgattgcattgttttgttccatatgccgcatgctagtcttttaaataaggatttatacgtctatacaatatttactcgattaatctattagtttccattcgggataaacgatatatacaaagcaagaaacacatacaaaaatgaatagctcaaaagattccaatgttcctctttgctagatattaaacaaaccaaatgtttcctatttgataatagacttattaaagctaacccatgtacatgaggcagtgagcatcgagaattagtaaagtttatttgagaaacacgttttttcttactaaagctatttccaaagctgtgcagttgcatttcaacaaaaccactagcacatttacgtttcgttgcacaataatgttgaatgcatttaatactgtacaaaataagaattaggatttataaaattgctcatattctgccggcactggttatatggataatgttaaacatacaaaaccccaattcagcaaaaagacaaaacaaatttaatttaagataattcctatcgaatacaaagatacattt

SEQ ID NO: 22 - DNA, 26 bp, Anopheles gambiae
gggatttgacgttaaagacaacactt

SEQ ID NO: 23 - protein, 16 aa, Artificial Sequence
  NLS1 sequence
APKKKRKVGIHGVPAA

SEQ ID NO: 24 - protein, 16 aa, Artificial Sequence
  NLS2 sequence
KRPAATKKAGQAKKKK

SEQ ID NO: 25 - protein, 8 aa, Artificial Sequence
  NLS3 sequence
MPKKKRKV

SEQ ID NO: 26 - protein, 4 aa, Artificial Sequence
  Linker sequence
SGGS